(19) United States
(12) Patent Application Publication
(10) Pub. No.: US 2018/0188195 A1
(43) Pub. Date: Jul. 5, 2018

(54) SYSTEMS AND METHODS FOR MULTIPLEXED HEMOSTASIS TEST PANELS

(71) Applicant: T2 Biosystems, Inc., Lexington, MA (US)

(72) Inventors: Eugenio DAVISO, Andover, MA (US); Lucius (Tad) FOX, Groton, MA (US); Thomas Jay LOWERY, Jr., Belmont, MA (US); Joseph MARTURANO, North Billerica, MA (US); Vyacheslav PAPKOV, Waltham, MA (US); Al SCHROFF, Dracut, MA (US); Roger SMITH, Dedham, MA (US); Zhixiang LUO, North Billerica, MA (US)

(21) Appl. No.: 15/738,754

(22) PCT Filed: Jun. 27, 2016
(86) PCT No.: PCT/US16/39611

§ 371 Date: Dec. 21, 2017

Related U.S. Application Data
(60) Provisional application No. 62/185,249, filed on Jun. 26, 2015.

(51) Int. Cl. G01N 24/08; G01R 33/44; C12Q 1/56

(52) U.S. Cl. CPC G01N 24/088; G01N 2333/974; C12Q 1/56; G01R 33/448

(57) ABSTRACT

The invention features a diagnostic platform utilizing T2 magnetic resonance to directly measure integrated reactions in whole blood samples such as clotting, fibrinogen, clot contraction, and fibrinolysis to provide a comprehensive assessment of hemostatic parameters on a single instrument in minutes. The methods of the invention can be performed with less than 1 ml of blood and minimal sample handling.

### BACKGROUND OF THE INVENTION

Clinical hemostasis involves the controlled rapid transformation of blood flowing under pressure to a highly localized, largely impermeable seal at sites of vascular damage followed by containment and then dissolution of clot formation. These ordered sequential changes in clot structure are required to prevent untoward bleeding in vivo while limiting the risk of thrombotic vascular occlusion.

Thrombosis and bleeding are among the foremost causes of morbidity and mortality. The introduction of novel anticoagulants has increased the need for rapid and accurate assessment of their activities. However, laboratory assessment of hemostasis remains difficult for some common clinical situations. Contemporary clinical laboratory methods are based on measuring components of hemostasis (e.g., prothrombin time, activated partial thromboplastin time, platelet aggregometry) or global function as reflected in mechanical clot strength (e.g., thromboelastography, thromboelastometry). These methods successfully identify many, but not all, bleeding disorders. Additionally, existing methods often provide little insight into the risk of thrombosis; lack sensitivity towards measuring fibrinolytic activity; require complex mechanical instrumentation; and typically require specialized technical expertise not available in most hospital laboratories. Existing methods can require blood draws of 1-25 ml and 30-150 minutes for sample processing and measurement.

There is a clinical need for a diagnostic platform that can measure both individual hemostatic parameters and integrated hemostasis, while eliminating sample modification prior to analysis, producing data output in as little as a few minutes with the option to monitor samples for hours, and reducing volume requirements over existing methodologies.

### SUMMARY OF THE INVENTION

The present invention features methods for detecting a change in a blood sample using time-resolved relaxation time acquisition methodology. The provided methods for measuring hemostasis are simple to practice, rapid, and reliable.

This invention features a method of monitoring water in a coagulating blood sample comprising the steps of: (i) providing a blood sample from a test subject and mixing a clotting activation reagent with the blood sample to initiate a coagulation process, (ii) making a series of T2 relaxation rate measurements of the water in the blood sample, wherein the measurements provide a plurality of decay curves, each decay curve measured using a Carr-Purcell-Meiboom-Gill (CPMG) sequence having a predetermined dwell time and each decay curve characteristic of a time point in the coagulation process, (iii) applying a mathematical transform to the plurality of decay curves to identify one or more water populations in the blood sample at one or more time points in the coagulation process to produce one or more T2 values and intensities, wherein decay curves for time points at the beginning of the process are measured using a CPMG sequence having a predetermined dwell time that is faster than the predetermined dwell time of the CPMG sequence used to measure decay curves at time points at the end of the process.

In one embodiment, the predetermined dwell time of the CPMG sequence used to measure decay curves at the beginning of the process is a fast dwell time of from 1 to 6 seconds. In another embodiment the predetermined dwell time of the CPMG sequence used to measure decay curves at the beginning of the process is a fast dwell time of from 2 to 4 seconds.

In certain embodiments, a fast dwell time is used to measure decay curves during the coagulation process for at least 1 minute following the completion of step (i). In another embodiment, a fast dwell time is used to measure decay curves during the coagulation process for at least 2 minutes following the completion of step (i), if the clotting activation reagent is an extrinsic pathway activator. In another embodiment a fast dwell time is used to measure decay curves during the coagulation process for at least 5 minutes following the completion of step (i), if the clotting activation reagent is an intrinsic pathway activator.

In any embodiment, the predetermined dwell time of the CPMG sequence used to measure decay curves at the end of the process can be a long dwell time of from 8 to 20 seconds (e.g., 8-12 seconds or 10-20 seconds). In one aspect, the long dwell time can be used to measure decay curves during the coagulation process after more than 2 minutes following the completion of step (i), if the clotting activation reagent can be an extrinsic pathway activator. In another aspect, the long dwell time is used to measure decay curves during the coagulation process after more than 5 minutes following the completion of step (i), if the clotting activation reagent is an intrinsic pathway activator. In yet another aspect, the long dwell time is used to measure decay curves during the coagulation process after more than 30 seconds (e.g., more than 1 minute) following the completion step (i), where the clotting activation reagent is a fibrin forming reagent such as those isolated from snake venom including batroxobin (reptilase) or ecarin, with or without FXIIIa, and optionally in combination with a platelet activator, such as TRAP, epinephrine, AA, collagen, or ADP.

In one aspect, at each time point, each T2 intensity for a given water population is proportional to the amount of the water population in its micro environment within blood sample at the time point.

In some embodiments, the method further includes identifying from the series of T2 relaxation rate measurements a first phase, wherein the first phase includes one or more T2 values measured during the clotting process prior to any observable clot formation. Next a second phase can be identified from the series of T2 relaxation rate measurements, and the second phase can include one or more T2 values measured during the clotting process at time points during which the sample comprises a loosely bound clot. During the first phase, a CPMG sequence having a predetermined dwell time that is faster than the predetermined dwell time of the CPMG sequence can be used to measure decay curves during the second phase.

In some embodiments, the method further includes collecting the series of T2 relaxation rate measurements during a first phase and prior to a second phase, wherein the first phase includes one or more T2 values measured during the clotting process prior to any observable clot formation. The region prior to a second phase can include one or more T2 values measured during the clotting process prior to time points during which the sample comprises a loosely bound clot. Next, a curve having a maximum asymptote can be fitted to the one or more T2 values from the one or more T2 values of the first phase. The beginning of the lower asymptote in the T2 values defines the completion of the transition from the first phase to the second phase. The first derivative of the curve can be calculated to identify the half-time of the loosely bound clot reaction (e.g., the transition from the first phase to the second phase). Following the inflection point, the time at which the slope of the first derivative reaches zero can be identified (i.e., the beginning of the second phase). Prior to a second phase, or at the beginning of the second phase, a CPMG sequence having a predetermined dwell time that is faster than the predetermined dwell time of the CPMG sequence can be used to measure decay curves after the time identified in the previous step.

In some embodiments, the method further includes collecting the series of T2 relaxation rate measurements during a first phase and prior to a second phase, wherein the first phase includes one or more T2 values measured during the clotting process prior to any observable clot formation. The region prior to a second phase can include one or more T2 values measured during the clotting process prior to time points during which the sample comprises a loosely bound clot. Next, a curve having a maximum asymptote can be fitted to the one or more T2 values from the one or more T2 values of the first phase. The slope of inflection in the T2 values can define the transition from the first phase to the second phase. The second derivative of the curve can be calculated to identify the inflection point, which can identify the half-time of the loosely bound clot reaction (e.g., the transition from the first phase to the second phase). Following the inflection point, the time at which the slope of the second derivative reaches zero can be identified. Prior to a second phase, or at the beginning of the second phase, a CPMG sequence having a predetermined dwell time that is faster than the predetermined dwell time of the CPMG sequence can be used to measure decay curves after the time identified in the previous step.

For one or more decay curves characteristic of a time point in the coagulation process, the data can further involve: (iv) fitting the decay curve to a mono-exponential, bi-exponential, and tri-exponential fit; and (v) using a non-linear least squares algorithm to identify the best fit. In one embodiment, step (v) comprises selecting the bi-exponential fit over the mono-exponential fit if the error sum of squares (SSE) of the two calculated T2 values for the bi-exponential fit is less than 75% of the SSE calculated for the mono-exponential fit. In another embodiment, step (v) comprises selecting the tri-exponential fit over the bi-exponential fit if the SSE for the three calculated T2 values of the tri-exponential fit is less than 90% of the SSE calculated for the two calculated T2 values of the bi-exponential fit. In another aspect, step (v) can involve constraining the non-linear least squares analysis to consider only T2 values not less than 50 ms and not greater than 2000 ms.

In one embodiment, for one or more decay curves characteristic of a time point in the coagulation process, the data can further involve following step (i), if step (v) identifies a bi-exponential or tri-exponential fit as the best fit for one or more decay curves measured using a CPMG sequence having a fast dwell time of from 1 to 6 seconds for one or more consecutive time points in the coagulation process, then the CPMG sequence used to measure one or more additional decay curves during the clotting process is characterized by a long dwell time of from 8 to 20 seconds (e.g., 8-12 seconds or 10-20 seconds).

In another embodiment, for one or more decay curves characteristic of a time point in the coagulation process, the data can further involve following step (i), if (a) step (v) identifies a bi-exponential or tri-exponential fit as the best fit for one or more decay curves measured using a CPMG sequence having a fast dwell time of from 1 to 6 seconds for one or more consecutive time points in the coagulation process, and (b) a T2 intensity for a water population characteristic of a clot micro environment reaches a predetermined threshold, then the CPMG sequence used to measure one or more additional decay curves during the clotting process is characterized by a long dwell time of from 8 to 20 seconds (e.g., 8-12 seconds or 10-20 seconds).

In another embodiment, for one or more decay curves characteristic of a time point in the coagulation process, the data can further involve following step, one the basis of the T2 values determining whether a clot has formed and, if so, then the CPMG sequence used to measure one or more additional decay curves during the clotting process is characterized by a long dwell time of from 8 to 20 seconds (e.g., 8-12 seconds or 10-20 seconds).

In another embodiment, the method further comprises: (ia) identifying from the series of T2 relaxation rate measurements a first phase, wherein the first phase comprises one or more T2 values measured during the clotting process prior to any observable clot formation; (ib) identifying from the series of T2 relaxation rate measurements a second phase, wherein the second phase comprises one or more T2 values measured during the clotting process at time points during which the sample comprises a loosely bound clot; (ic) calculating the difference between the one or more T2 values of the first phase and the one or more T2 values of the second phase; and (id) on the basis of step (ic), determining the fibrinogen level of the blood sample.

In another embodiment, the method comprises: (xi) fitting the one or more T2 values of the first phase and the one or more T2 values of the second phase to a curve; (xii) identifying a maximum asymptote from the one or more T2 values of the first phase; (xiii) identifying a minimum asymptote from the one or more T2 values of the second phase; and (xiv) calculating the difference between the maximum asymptote and the minimum asymptote; and (xv) on the basis of step (xiv), determining the fibrinogen level of the blood sample.

In another embodiment, for one or more decay curves characteristic of a time point in the coagulation process, the data can further involve (yi) identifying from the series of T2 relaxation rate measurements a first phase, wherein the first phase comprises one or more T2 values measured during the clotting process prior to any observable clot formation; (yii) identifying from the series of T2 relaxation rate measurements a second phase, wherein the second phase comprises one or more T2 values measured during the clotting process at time points during which the sample comprises a loosely bound clot; (yiii) calculating the minimum value of the first derivative, the maximum value of the second derivative, or the inflection point from T2 values spanning the transition from first phase to the second phase of the clotting process; and (yiv) on the basis of step (yiii), determining the clotting time of the blood sample.

In another embodiment, the method includes (zi) fitting the one or more T2 values of the first phase and the one or more T2 values of the second phase to a curve; (zii) identifying a maximum asymptote from the one or more T2 values of the first phase; (ziii) identifying a minimum asymptote from the one or more T2 values of the second phase; and (ziv) calculating the minimum value of the first derivative, the maximum value of the second derivative, or the inflection point from the curve spanning the transition from first phase to the second phase of the clotting process; and (zv) on the basis of step (ziv), determining the clotting time of the blood sample.

In another embodiment, for one or more decay curves characteristic of a time point in the coagulation process, the data can further involve (ai) identifying from the series of T2 relaxation rate measurements a third phase, wherein the third phase comprises one or more T2 values having one or more T2 intensities greater than a predetermined threshold and characteristic of a serum-like environment, and where each of the one or more T2 values is measured during the clotting process at time points during which the sample comprises a mixture of serum and clot microenvironments; and (aii) on the basis of at least one of the one or more T2 values and the one or more T2 intensities, determining the platelet activity of the blood sample.

In another embodiment, for one or more decay curves characteristic of a time point in the coagulation process, the data can further involve (bi) identifying from the series of T2 relaxation rate measurements a third phase, wherein the third phase comprises one or more T2 values characteristic of a serum-like environment, each of the one or more T2 values having a T2 intensity greater than a predetermined threshold, and where each of the one or more T2 values is measured during the clotting process at time points during which the sample comprises a mixture of serum and clot microenvironments; (bii) calculating the difference between the one or more T2 values of the third phase and a predetermined lower bound at a predetermined time, or calculating a T2 curve characteristic of the third phase and calculating an area between that curve and a predetermined lower bound for a predetermined time period during the clotting process; and (biii) on the basis of step (bii), determining the platelet activity of the blood sample.

In one embodiment, the platelet activity is determined by calculating the difference between the one or more T2 values of the third phase and a predetermined lower bound at a predetermined time and the lower bound and the lower bound comprises one of the following: (ci) one or more T2 values of a first phase, wherein the first phase comprises one or more T2 values measured during the clotting process prior to any observable clot formation; (cii) one or more T2 values of a second phase, wherein the second phase comprises one or more T2 values measured during the clotting process at time points during which the sample comprises a loosely bound clot; (ciii) one or more T2 values of a fourth phase, wherein the fourth phase comprises one or more T2 values characteristic of a clot microenvironment, wherein each of the one or more T2 values is measured during the clotting process at time points during which the sample comprises a mixture of serum and clot microenvironments; (civ) a maximum asymptote of the first phase, wherein the maximum asymptote is determined by fitting the one or more T2 values of the first phase and the one or more T2 values of the second phase to a curve, and identifying a maximum asymptote from the one or more T2 values of the first phase, or identifying a minimum asymptote from the one or more T2 values of the second phase; and (cv) a minimum asymptote of the second phase, wherein the maximum asymptote is determined by fitting the one or more T2 values of the first phase and the one or more T2 values of the second phase to a curve, and identifying a maximum asymptote from the one or more T2 values of the first phase, or identifying a minimum asymptote from the one or more T2 values of the second phase.

In one embodiment, the platelet activity is determined by calculating an area under a T2MR curve for the third phase and a predetermined lower bound for a predetermined time period during the clotting process and the lower bound comprises one of the following: (di) one or more T2 values of a first phase, wherein the first phase comprises one or more T2 values measured during the clotting process prior to any observable clot formation; (dii) one or more T2 values of a second phase, wherein the second phase comprises one or more T2 values measured during the clotting process at time points during which the sample comprises a loosely bound clot; (diii) one or more T2 values of a fourth phase, wherein the fourth phase comprises one or more T2 values characteristic of a clot microenvironment, and where each of the one or more T2 values is measured during the clotting process at time points during which the sample comprises a mixture of serum and clot microenvironments; (div) a maximum asymptote of the first phase, wherein the maximum asymptote is determined by fitting the one or more T2 values of the first phase and the one or more T2 values of the second phase to a curve, and identifying a maximum asymptote from the one or more T2 values of the first phase, or identifying a minimum asymptote from the one or more T2 values of the second phase; and (dv) a minimum asymptote of the second phase, wherein the maximum asymptote is determined by fitting the one or more T2 values of the first phase and the one or more T2 values of the second phase to a curve, and identifying a maximum asymptote from the one or more T2 values of the first phase, or identifying a minimum asymptote from the one or more T2 values of the second phase.

In another embodiment, the method to determine platelet activity includes (ei) identifying the difference between the T2 values of the third phase and a predetermined lower bound; (eii) identifying one or more T2 intensities of the third phase or the lower bound; and (eiii) on the basis of step (ei) and step (eii), determining the platelet activity.

In another embodiment, the method to determine platelet activity includes (fi) identifying an area defined by a T2MR curve of the third phase and a predetermined lower bound; (fii) identifying one or more T2 intensities of the third phase or the lower bound; and (fiii) on the basis of step (fi) and step (fii), determining the platelet activity.

In another embodiment, the method to determine platelet activity includes (gi) identifying the maximum T2 intensity of the third phase; (gii) identifying an area defined by a T2MR curve of the third phase and a predetermined lower bound; and (giii) on the basis of step (gi) and step (gii), determining the platelet activity.

In another embodiment, the method to determine platelet activity includes (hi) identifying the maximum T2 intensity of the third phase; (hii) identifying the time associated with the maximum T2 intensity of the third phase; (hiii) identifying an area defined by a T2MR curve of the third phase and a predetermined lower bound that terminates at the time associated with the maximum T2 intensity of the third phase; and (hiv) on the basis of step (hiii), determining the platelet activity.

In a variation on previous embodiments involving the calculation of platelet activity mentioned above, the one or more T2 values of the third phase comprise the maximum T2 values observed during the clotting process.

In a variation on previous embodiments involving the calculation of platelet activity mentioned above, the method can further include multiplying the difference by the T2 intensity of the one or more T2 values of the third phase to produce a value, and on the basis of the value determining the platelet activity of the blood sample.

In another embodiment, the method further involves (i-i) identifying from the series of T2 relaxation rate measurements a third phase, wherein the third phase comprises one or more T2 values characteristic of a serum-like environment, each of the one or more T2 values having a T2 intensity greater than a predetermined threshold, and where each of the one or more T2 values is measured during the clotting process at time points during which the sample comprises a mixture of serum and clot microenvironments; (i-ii) identifying from the series of T2 relaxation rate measurements a fifth phase, wherein the fifth phase comprises one or more T2 values characteristic of a serum-like environment, and where each of the one or more T2 values is measured during the clotting process at time points during which the sample comprises a clot micro environment undergoing lysis; (i-iii) calculating the difference between the one or more T2 values of the third phase and the one or more T2 values of the fifth phase; and (i-iv) on the basis of step (i-iii), determining the level of the fibrinolysis of the blood sample.

In some embodiments, the method further includes identifying from the series of T2 relaxation rate measurements a third phase, wherein the third phase comprises one or more T2 values characteristic of a serum-like environment. Each of the one or more T2 values can have a T2 intensity greater than a predetermined threshold, and each of the one or more T2 values can be measured during the clotting process at time points during which the sample comprises a mixture of serum and clot microenvironments. A fifth phase can be identified from the series of T2 relaxation rate measurements. The fifth phase can include one or more T2 values characteristic of a serum-like environment, and each of the one or more T2 values can be measured during the clotting process at time points during which the sample comprises a clot micro environment undergoing lysis. An area above a T2MR curve for the fifth phase and a predetermined upper bound can be calculated for a predetermined time period during the clotting process, wherein, e.g., the upper bound is defined by the one or more T2 values of the third phase projected over the curve for the fifth phase. On the basis of the area above the T2MR curve, the level of the fibrinolysis of the blood sample can be determined. In some embodiments, the upper bound is defined by the maximum T2 value of the third phase projected over the curve for the fifth phase. Any of the previous methods can additionally include (ki) measuring the T2 of the blood sample prior to step (kii) to produce a first T2 value; and (ib) on the basis of the measured first T2 value, determining the hematocrit level of the blood sample.

One embodiment includes (ki) identifying from the series of T2 relaxation rate measurements a first phase, wherein the first phase comprises one or more T2 values measured during the clotting process prior to any observable clot formation; (kii) on the basis of the T2 values of the first phase determining a value for a hematocrit parameter, wherein the value of the hematocrit parameter is characteristic of the hematocrit concentration.

One embodiment further comprises (kiii) identifying from the series of T2 relaxation rate measurements a first phase, wherein the first phase comprises one or more T2 values measured during the clotting process prior to any observable clot formation; (kiv) identifying from the series of T2 relaxation rate measurements a second phase, wherein the second phase comprises one or more T2 values measured during the clotting process at time points during which the sample comprises a loosely bound clot; (kv) calculating the difference between the one or more T2 values of the first phase and the one or more T2 values of the second phase; and (kvi) calculating a corrected fibrinogen value based on the difference between the first phase and the second phase and the hematocrit parameter.

Another method can additionally include (kvii) fitting the one or more T2 values of the first phase and the one or more T2 values of the second phase to a curve; identifying a maximum asymptote from the one or more T2 values of the first phase; and identifying a minimum asymptote from the one or more T2 values of the second phase; (kviii) calculating the difference between the maximum asymptote and the minimum asymptote; (kix) on the basis of the hematocrit and step (kviii), determining the fibrinogen concentration.

In any method, the blood sample can be diluted from 0.1-60% prior to initiating coagulation (e.g., prior to step (i)). The blood sample can be diluted about 50%.

In any method wherein the blood sample can be a whole blood sample. The blood sample can comprise an anticoagulant. The anticoagulant can be at least one of citrate, heparin, and corn trypsin inhibitor. The blood sample can also be a plasma sample. The plasma sample can additionally include citrate. In one embodiment the clotting activation reagent is a dried clotting activation reagent.

In one embodiment, a method of monitoring water in a coagulating blood sample includes the steps of: (i) providing a blood sample from a test subject and mixing a clotting activation reagent with the blood sample to initiate a coagulation process, (ii) making a series of T2 relaxation rate measurements of the water in the blood sample, wherein the measurements provide a plurality of decay curves, each decay curve measured using a Carr-Purcell-Meiboom-Gill (CPMG) sequence having a predetermined dwell time and each decay curve characteristic of a time point in the coagulation process, (iii) applying a mathematical transform to the plurality of decay curves to identify one or more water populations in the blood sample at one or more time points in the coagulation process to produce one or more T2 values and/or T2 intensities, (iv) identifying from the series of T2 relaxation rate measurements a first phase, wherein the first phase comprises one or more T2 values measured during the clotting process prior to any observable clot formation; (v) identifying from the series of T2 relaxation rate measurements a second phase, wherein the second phase comprises one or more T2 values measured during the clotting process at time points during which the sample comprises a loosely bound clot; (vi) fitting the one or more T2 values of the first phase and the one or more T2 values of the second phase to a curve, and identifying a maximum asymptote from the one or more T2 values of the first phase, or identifying a minimum asymptote from the one or more T2 values of the second phase; (vii) calculating the difference between the maximum asymptote and the minimum asymptote; and (viii) on the basis of step (vii), determining the fibrinogen value of the blood sample.

Another embodiment can further include (ix) calculating the minimum value of the first derivative, the maximum value of the second derivative, or the inflection point from T2 values spanning the transition from first phase to the second phase of the clotting process; and (x) mathematically combining the minimum value or the maximum value determined in step (ix) with the value determined in step (viii); and (xi) on the basis of step (x) determining a fibrinogen value of the blood sample.

Another embodiment can further include (ix) mathematically combining the value of the maximum asymptote with the value from step (vii); (x) on the basis of the value of step (ix), determining a fibrinogen value.

Another embodiment can further include (a) identifying from the series of T2 relaxation rate measurements a first phase, wherein the first phase comprises one or more T2 values measured during the clotting process prior to any observable clot formation; (b) identifying from the series of T2 relaxation rate measurements a second phase, wherein the second phase comprises one or more T2 values measured during the clotting process at time points during which the sample comprises a loosely bound clot; (c) fitting the one or more T2 values of the first phase and the one or more T2 values of the second phase to a curve, and identifying a maximum asymptote from the one or more T2 values of the first phase, or identifying a minimum asymptote from the one or more T2 values of the second phase; (d) calculating the difference between the maximum asymptote and the minimum asymptote; and (e) mathematically combining the value of the maximum asymptote, the maximum T2 value and the value from step (vii) (e.g., a composite value, such as deltaR2, % deltaT2/(MaxT2), or 1/T2max−1/T2min); and (f) on the basis of the value of step (e), determining a fibrinogen value.

Another embodiment can further include (1) on the basis of step (iv) determining a value for a hematocrit parameter, wherein the value of the hematocrit parameter is characteristic of the hematocrit concentration; (2) on the basis of the fibrinogen value and the hematocrit parameter, determining a corrected fibrinogen concentration.

One embodiment includes a method of monitoring water in a coagulating blood sample comprising the steps of: (i) providing a blood sample from a test subject and mixing a clotting activation reagent with the blood sample to initiate a coagulation process, (ii) making a series of T2 relaxation rate measurements of the water in the blood sample, wherein the measurements provide a plurality of decay curves, each decay curve measured using a Carr-Purcell-Meiboom-Gill (CPMG) sequence having a predetermined dwell time and each decay curve characteristic of a time point in the coagulation process, (iii) applying a mathematical transform to the plurality of decay curves to identify one or more water populations in the blood sample at one or more time points in the coagulation process to produce one or more T2 values and/or T2 intensities, (iv) identifying from the series of T2 relaxation rate measurements a first phase, wherein the first phase comprises one or more T2 values measured during the clotting process prior to any observable clot formation; (v) identifying from the series of T2 relaxation rate measurements a second phase, wherein the second phase comprises one or more T2 values measured during the clotting process at time points during which the sample comprises a loosely bound clot; (vi) fitting the one or more T2 values of the first phase and the one or more T2 values of the second phase to a curve, and identifying a maximum asymptote from the one or more T2 values of the first phase, or identifying a minimum asymptote from the one or more T2 values of the second phase; (vii) calculating the value of the first derivative or the inflection point of the curve between the maximum asymptote and the minimum asymptote; and (viii) on the basis of step (vii), determining the clotting time of the blood sample.

One method can further comprise: (ix) calculating the minimum value of the first derivative, the maximum value of the second derivative, or the inflection point from T2 values spanning the transition from first phase to the second phase of the clotting process; and (x) on the basis of step (ix), determining the clotting time of the blood sample.

Another method can further comprise (xi) identifying from the series of T2 relaxation rate measurements a third phase, wherein the third phase comprises one or more T2 values characteristic of a serum-like environment, each of the one or more T2 values having a T2 intensity greater than a predetermined threshold, and where each of the one or more T2 values is measured during the clotting process at time points during which the sample comprises a mixture of serum and clot microenvironments; (xii) calculating the difference between the maximum asymptote and the one or more T2 values of the third phase, or calculating the difference between the minimum asymptote and the one or more T2 values of the third phase; (xiii) on the basis of step (xii), determining the platelet activity of the blood sample.

Another method further includes: (xiii) identifying from the series of T2 relaxation rate measurements a third phase, wherein the third phase comprises one or more T2 values having one or more T2 intensities greater than a predetermined threshold and characteristic of a serum-like environment, and where each of the one or more T2 values is measured during the clotting process at time points during which the sample comprises a mixture of serum and clot microenvironments; and (xiv) on the basis of the one or more T2 values or the one or more T2 intensities, determining the platelet activity of the blood sample.

One method further includes: (xv) identifying from the series of T2 relaxation rate measurements a fifth phase, wherein the fifth phase comprises one or more T2 values characteristic of a serum-like environment, and where each of the one or more T2 values is measured during the clotting process at time points during which the sample comprises a clot micro environment undergoing lysis; (xvi) calculating the difference between the one or more T2 values of the third phase and the one or more T2 values of the fifth phase; and (xvii) on the basis of step (xvi), determining the level of the fibrinolysis of the blood sample.

In some embodiments, the method further includes determining the level of the fibrinolysis of the blood sample by identifying a third phase, wherein the third phase includes one or more T2 values characteristic of a serum-like environment, each of the one or more T2 values having a T2 intensity greater than a predetermined threshold, and where each of the one or more T2 values is measured during the clotting process at time points during which the sample comprises a mixture of serum and clot microenvironments. The method may further include identification of a fifth phase, wherein the fifth phase includes one or more T2 values characteristic of a serum-like environment, and where each of the one or more T2 values is measured during the clotting process at time points during which the sample comprises a clot micro environment undergoing lysis. The method may include calculating an area above a T2MR curve for the fifth phase and a predetermined upper bound for a predetermined time period during the clotting process, wherein the upper bound is defined by the one or more T2 values of the third phase projected over the curve for the fifth phase. In some embodiments, on the basis of the preceding step, the level of the fibrinolysis of the blood sample is determined.

In another aspect, the invention provides a method of monitoring water in a coagulating blood sample by providing a blood sample from a test subject and mixing a clotting activation reagent with the blood sample to initiate a coagulation process and making a series of T2 relaxation rate measurements of the water in the blood sample. The measurements may provide a plurality of decay curves, each decay curve measured using, e.g., a Carr-Purcell-Meiboom-Gill (CPMG) sequence having a predetermined dwell time. Each decay curve may be characteristic of a time point in the coagulation process. The method may further include applying a mathematical transform to the plurality of decay curves to identify one or more water populations in the blood sample at one or more time points in the coagulation process to produce one or more T2 values and/or T2 intensities and identifying from the series of T2 relaxation rate measurements a third phase. The third phase may include one or more T2 values characteristic of a serum-like environment, and each of the one or more T2 values may have a T2 intensity greater than a predetermined threshold. Each of the one or more T2 values can be measured during the clotting process at time points during which the sample comprises a mixture of serum and clot microenvironments. Next, a fifth phase can be identified, wherein the fifth phase includes one or more T2 values characteristic of a serum-like environment. Each of the one or more T2 values can be measured during the clotting process at time points during which the sample comprises a clot micro environment undergoing lysis. The method may further involve calculating an area above a T2MR curve for the fifth phase and a predetermined upper bound for a predetermined time period during the clotting process, wherein the upper bound is defined by the one or more T2 values of the third phase projected over the curve for the fifth phase. On the basis of the area over the curve, the level of the fibrinolysis of the blood sample can be determined. In some embodiments, the upper bound is defined by the maximum T2 value of the third phase projected over the curve for the fifth phase.

In any embodiment CaCl₂ can be added to the blood or plasma sample prior to measurement of T2.

Several different types of clotting activation reagents can be used. One embodiment includes a clotting activation reagent that comprises an extrinsic activator. The extrinsic activator can be reagents such as tissue factor, thromboplastin, Innovin®, Readiplastin®, and EXTEM®. In another embodiment, the clotting activation reagent comprises an intrinsic activator. The intrinsic activator can include reagents such as ellagic acid, celite, kaolin, and INTEM. In another embodiment the clotting activation reagent can be a global activator. This global activator can be thrombin.

The blood sample of any of the described methods can be derived from a pediatric or neonatal subject. This pediatric subject can exhibit symptoms associated with a hemostatic disorder. The hemostatic disorder can be one of the group of disorders that includes: hemophilia, von Willebrand disease, hypercoagulable state, thromotic thrombocytopenic purpura, thrombocytopenia, primary thrombocythemia, induced thrombocytopenia, disseminated intravascular coagulation, procoagulant afibrinogenemia/dysfibrinogenemia, protein C deficiency, protein S deficiency, antithrombin III deficiency, factor V Leiden deficiency, activated protein C resistance (aPCR), Anticoagulant afibrinogenemia/dysfibrinogenemia, Factor V deficiency, Factor VII deficiency, Factor X deficiency, Factor XI deficiency, Factor XII deficiency, Factor XIII deficiency, hypoprothrombinemias, cryoglobulinemias, multiple myeloma, Waldenstrom macroglobulinemia, Henoch-Schonlein purpura, hyperglobulinemic purpura, cavernous hemangioma, hereditary hemorrhagic telangiectasia, pseudoxanthoma elasticum, Vitamin K deficiency, Shwartzman phenomenon, Wiskott-Aldrich syndrome, sepsis, and hemolytic disease of the newborn.

In one embodiment of the assay, the method is completed in 65 minutes or less, (e.g., 54 minutes or less), or in 45 minutes or less. In another embodiment the method includes calculating a clotting time within 5 minutes of initiating clotting.

For any of the described methods, the T2MR values can be reported in real time. The following parameters can be reported in real time: percent hematocrit, clotting time, fibrinogen concentration, platelet activity, and fibrinolysis.

The method comprises calculating the platelet activity within 20 minutes, within 25 minutes, within 30 minutes, within 35 minutes, or within 40 minutes.

In one embodiment a method of monitoring water in a coagulating blood sample comprises the steps of: (i) providing a blood sample from a test subject and mixing a clotting activation reagent with the blood sample to initiate a coagulation process; (ii) making a series of T2 relaxation rate measurements of the water in the blood sample, wherein the measurements provide a plurality of decay curves, each decay curve measured using a Carr-Purcell-Meiboom-Gill (CPMG) sequence having a predetermined dwell time and each decay curve characteristic of a time point in the coagulation process; (iii) applying a mathematical transform to the plurality of decay curves to identify one or more water populations in the blood sample at one or more time points in the coagulation process to produce one or more T2 values and/or T2 intensities; (iv) identifying from the series of T2 relaxation rate measurements a third phase, wherein the third phase comprises one or more T2 values having one or more T2 intensities greater than a predetermined threshold and characteristic of a serum-like environment, and where each of the one or more T2 values is measured during the clotting process at time points during which the sample comprises a mixture of serum and clot microenvironments; and (v) on the basis of the one or more T2 values or the one or more T2 intensities, determining the platelet activity of the blood sample.

In one embodiment the platelet activity is determined by calculating the difference between the one or more T2 values of the third phase and a predetermined lower bound at a predetermined time and the lower bound and the lower bound comprises one of the following: (vi) one or more T2 values of a first phase, wherein the first phase comprises one or more T2 values measured during the clotting process prior to any observable clot formation; (vii) one or more T2 values of a second phase, wherein the second phase comprises one or more T2 values measured during the clotting process at time points during which the sample comprises a loosely bound clot; (viii) one or more T2 values of a fourth phase, wherein the fourth phase comprises one or more T2 values characteristic of a clot microenvironment, wherein each of the one or more T2 values is measured during the clotting process at time points during which the sample comprises a mixture of serum and clot microenvironments; (ix) a maximum asymptote of the first phase, wherein the maximum asymptote is determined by fitting the one or more T2 values of the first phase and the one or more T2 values of the second phase to a curve, and identifying a maximum asymptote from the one or more T2 values of the first phase, or identifying a minimum asymptote from the one or more T2 values of the second phase; and (x) a minimum asymptote of the second phase, wherein the maximum asymptote is determined by fitting the one or more T2 values of the first phase and the one or more T2 values of the second phase to a curve, and identifying a maximum asymptote from the one or more T2 values of the first phase, or identifying a minimum asymptote from the one or more T2 values of the second phase.

In another embodiment, the platelet activity is determined by calculating an area under a T2MR curve for the third phase and a predetermined lower bound for a predetermined time period during the clotting process and the lower bound comprises one of the following: (vi) one or more T2 values of a first phase, wherein the first phase comprises one or more T2 values measured during the clotting process prior to any observable clot formation; (vii) one or more T2 values of a second phase, wherein the second phase comprises one or more T2 values measured during the clotting process at time points during which the sample comprises a loosely bound clot; (viii) one or more T2 values of a fourth phase, wherein the fourth phase comprises one or more T2 values characteristic of a clot microenvironment, and where each of the one or more T2 values is measured during the clotting process at time points during which the sample comprises a mixture of serum and clot microenvironments; (ix) a maximum asymptote of the first phase, wherein the maximum asymptote is determined by fitting the one or more T2 values of the first phase and the one or more T2 values of the second phase to a curve, and identifying a maximum asymptote from the one or more T2 values of the first phase, or identifying a minimum asymptote from the one or more T2 values of the second phase; and (x) a minimum asymptote of the second phase, wherein the maximum asymptote is determined by fitting the one or more T2 values of the first phase and the one or more T2 values of the second phase to a curve, and identifying a maximum asymptote from the one or more T2 values of the first phase, or identifying a minimum asymptote from the one or more T2 values of the second phase.

An embodiment of the platelet activity determination includes (a) identifying the difference between the T2 values of the third phase and a predetermined lower bound; (b) identifying one or more T2 intensities of the third phase or the lower bound; and (c) on the basis of step (a) and step (b) determining the platelet activity.

In another embodiment of the platelet activity determination, the method further includes (a) identifying an area defined by a T2MR curve of the third phase and a predetermined lower bound; (b) identifying one or more T2 intensities of the third phase or the lower bound; and (c) on the basis of step (a) and step (b), determining the platelet activity.

In another embodiment of the platelet activity determination (d) identifying the maximum T2 intensity of the third phase; (e) identifying an area defined by a T2MR curve of the third phase and a predetermined lower bound; and (f) on the basis of step (d) and step (e), determining the platelet activity.

In another embodiment the platelet activity includes (g) identifying the maximum T2 intensity of the third phase; (h) identifying the time associated with the maximum T2 intensity of the third phase; (i) identifying an area defined by a T2MR curve of the third phase and a predetermined lower bound that terminates at the time associated with the maximum T2 intensity of the third phase; and (j) on the basis of step (i), determining the platelet activity.

In an embodiment of any of the above methods, the method can include monitoring a clotting process in a whole blood sample including: (a) providing uncoagulated whole blood, fibrinogen, and a clotting activation reagent; (b) combining the fibrinogen, the clotting activation reagent, and the uncoagulated whole blood to form a reaction mixture that includes from 50% (v/v) to 100% (v/v) (i.e., 60±10%, 70±10%, 80±10% (v/v), or 90±10%) whole blood and a fibrinogen concentration greater than or equal to about 0.5 mg/mL (i.e., the added amount of fibrinogen in the reaction mixture is 0.65±0.15, 0.75±0.15, 0.85±0.15, 0.95±0.15, 1.05±0.15, or 1.25±0.25 mg/mL); (c) making a series of magnetic resonance relaxation rate measurements of water in the reaction mixture; and (d) on the basis of the results of step (c), determining the clotting time.

In another embodiment of any of the above methods, the method can include method for monitoring a clotting process in a platelet rich plasma sample including: (a) providing uncoagulated platelet rich plasma, fibrinogen, and a clotting activation reagent; (b) combining the fibrinogen, the clotting activation reagent, and the uncoagulated platelet rich plasma to form a reaction mixture that includes from 50% (v/v) to 90% (v/v) (i.e., 60±10%, 70±10%, or 80±10% (v/v)) platelet rich plasma and a fibrinogen concentration greater than or equal to about 0.5 mg/mL (i.e., the added amount of fibrinogen in the reaction mixture is 0.65±0.15, 0.75±0.15, 0.85±0.15, 0.95±0.15, 1.05±0.15, or 1.25±0.25 mg/mL); (c) making a series of magnetic resonance relaxation rate measurements of water in the reaction mixture; and (d) on the basis of the results of step (c), determining the clotting time.

In a further embodiment of any of the above methods, the method can include method for monitoring a clotting process in a platelet poor plasma sample including: (a) providing uncoagulated platelet poor plasma, fibrinogen, and a clotting activation reagent; (b) combining the fibrinogen, the clotting activation reagent, and the uncoagulated platelet poor plasma to form a reaction mixture that includes from 50% (v/v) to 90% (v/v) (i.e., 60±10%, 70±10%, or 80±10% (v/v)) platelet poor plasma and a fibrinogen concentration greater than or equal to about 0.5 mg/mL (i.e., the added amount of fibrinogen in the reaction mixture is 0.65±0.15, 0.75±0.15, 0.85±0.15, 0.95±0.15, 1.05±0.15, or 1.25±0.25 mg/mL); (c) making a series of magnetic resonance relaxation rate measurements of water in the reaction mixture; and (d) on the basis of the results of step (c), determining the clotting time.

In any of the above methods, the fibrinogen and/or the clotting activation reagent can be provided as a solution.

In any of the above methods, the method can be performed without the addition of fibrinogen, and only the addition of a clotting activation reagent with or without recalcification or with recalcificaiton only.

In any of the above methods, the clotting activation reagent can be selected from RPF (reptilase and factor XIII), RPF plus arachidonic acid (AA), RPF plus adenosine diphosphate (ADP), kaolin (CK), RPF plus thrombin receptor activating peptide (TRAP), RPF plus epinephrine, RPF plus collagen, thromboplastin/tissue factor (TF), celite, ellagic acid (EA), prothrombin activator, factor XI and calcium, factor VII, factor IX and factor VIII and calcium, polyphosphates, and thrombin, or any other clotting activation agent described herein or commonly known in the art.

In particular embodiments of any of the above methods, the method further includes repeating steps (a)-(d) to produce a replicate value of the clotting time (i.e., making measurements in, for example, duplicate or triplicate). A clotting time value for a particular sample and coagulation conditions can be the average of the replicate measurements.

In another embodiment of any of the above methods, a clotting time having coefficient of variation of less than 7%, 6%, 5%, 4%, or 3.5% when the clotting time is measured at least 10 times can be measured. The methods of the invention can reduce the variability observed in clotting time measurements relative to measurements made on samples to which no fibrinogen has been added.

In particular embodiments of any of the above methods, step (c) includes making a series of magnetic resonance relaxation rate measurements of water in the reaction mixture within a sample tube, wherein the inner surface of the sample tube controls fibrin adhesion. The sample tube can be any type of tube or coating described herein for the control of fibrin adhesion.

In any of the above methods, step (c) can include (i) making a plurality of T2 relaxation rate measurements of water in the reaction mixture to produce a plurality of decay curves, and (ii) calculating from the plurality of decay curves a plurality of T2 relaxation spectra.

In a related aspect, the invention features a method of evaluating a blood sample from a subject including (i) performing any one or more of the methods described above on the blood sample, or an extract thereof, to determine the clotting time; and (ii) on the basis of step (i), determining whether the subject is hypercoagulable, hypocoagulable, or normal.

The invention also features a method of evaluating a blood sample from a subject including (i) performing any one or more of the methods described above on the blood sample, or an extract thereof, to determine the clotting time; and (ii) on the basis of step (i), determining whether the subject is at risk of thrombotic complications or the subject is resistant to antiplatelet therapy.

The invention further features a method of evaluating a blood sample from a subject including (i) performing any one or more of the methods described above on the blood sample, or an extract thereof, to determine the clotting time; and (ii) on the basis of step (i), determining whether the subject has a coagulopathy. The coagulopathy can be any coagulopathy described herein.

In any of the above-referenced methods, the blood sample can have a volume of from 5 μL to 50 μL (e.g., 10±5 μL, 15±5 μL, 20±10 μL, 30±10 μL, or 40±10 μL) to which clotting activation reagents, either introduced as a dried reagent, or a liquid have a volume of from 5 μL to 25 μL (e.g., 10±5 μL, 15±5 μL, or 20±5 μL) are added.

In a related aspect the invention features an NMR device (e.g., a T2MR unit) including a computer equipped with an algorithm for implementing an NMR pulse sequence according to any of the above methods or performing a calculation (e.g., a clotting time, fibrinogen level, platelet activity, or fibrinolysis measurement) according to any of the above methods.

As used herein, “citrated blood” is blood that has been treated with trisodium citrate (9:1) following standard procedures that minimize platelet activation to prevent coagulation.

As used herein, the term “clotting activation reagent” refers to a clotting initiator or activator. Non-limiting examples include calcium chloride, citrated kaolin, RPF, AA, ADP, CK, TRAP, epinephrine, collagen, tissue factor, celite, ellagic acid, and thrombin.

As used herein, the term “coagulopathy” refers to a condition in which the blood's ability to clot (coagulate) or lyse is impaired.

As used herein, the terms “fibrinogen level” and “fibrinogen value” refers to the level of fibrinogen activity (i.e., the Estimated Fibrinogen Function (EFF), functional fibrinogen, fibrinogen value, or fibrinogen level) in a sample.

As used herein, the term “hypercoagulable” refers to an abnormality of blood coagulation that increases the rate of coagulation and/or extent of coagulability, and may increase the risk of thrombosis.

As used herein, the term “hypocoagulable” refers to an abnormality of blood coagulation that reduces the rate of coagulation and/or extent of coagulability.

As used herein, the term “NMR relaxation rate” refers to any of the following in a sample: T1, T2, T₁ᵣₕₒ, T₂ᵣₕₒ, and T₂*. NMR relaxation rates may be measured and/or represented using T1/T2 hybrid detection methods. Additionally, apparent diffusion coefficient (ADC) can be determined and evaluated (Vidmar et al. NMR in BioMedicine, 2009; and Vidmar et al., Eur J Biophys J. 2008). Additionally, pulsed field gradients with measurement of echo attenuation as a function if the square of gradient strength, Hahn echo sequence, spin echo sequence, FID signal ratios.

As used herein, the term “platelet rich plasma” refers to blood plasma that has been enriched with platelets relative to platelet poor plasma (i.e., greater than 10×10³, 10×10⁴, 50×10⁴, or 75×10⁴ platelets per microliter of plasma).

As used herein, the term “platelet poor plasma” refers to blood plasma with a very low number of platelets relative to the whole blood from which it is derived. For example, the platelet poor plasma can have less than 10×10³ platelets per microliter of plasma.

As used herein, the term “reader” or “T2reader” refers to a device for detecting coagulation-related activation including clotting and fibrinolysis of samples. T2readers may be used generally to characterize the properties of a sample (e.g., a biological sample such as blood or non-biological samples such as an acrylamide gel). Such a device is described, for example, in International Publication No. WO 2010/051362, which is herein incorporated by reference.

As used herein, the term “resistant to antiplatelet therapy” refers to a weak response, or no response, to an antiplatelet drug in a sample or a subject. For example, resistance to antiplatelet therapy can be monitored by observing platelet function in the presence of an antiplatelet drug, such as an inhibiter of cyclooxygenase 1/thromboxaneA2 receptors (e.g., aspirin), adenosine diphosphate receptors (e.g., clopidogrel), or GPIIb/IlIa receptors (e.g., abciximab, tirofiban).

As used herein, the term “thrombotic complications” refers to complications arising from the formation of thrombosis in a subject.

As used herein, the term “whole blood” refers to the blood of a subject that includes red blood cells. Whole blood includes blood which has been altered through a processing step or modified by the addition of an additive (e.g., corn trypsin inhibitor, heparin, citrate, a nanoparticle formulation, fibrinogen, tissue plasminogen activator (TPA), collagen, antithrombotic agents such as abciximab, or other additives, such as anticoagulants or therapeutic drugs).

As used herein, the term “uncoagulated” refers to a whole blood sample, or a fraction thereof, which has not undergone a coagulation reaction. Typically, an uncoagulated sample is capable of undergoing a coagulation reaction upon addition of a clotting activation reagent, or, in the case of a hemostasis deficiency, is capable of undergoing a coagulation reaction upon addition of both a clotting activation reagent and one or more clotting factors necessary to correct a hemostasis deficiency.

As used herein, the term “dwell time” refers to the amount of time between the start of each successive T2MR measurement.

As used herein, the term “extrinsic pathway” refers to the extrinsic coagulation pathway. Correspondingly, an “extrinsic activator” is a clotting activation reagent that initiates the extrinsic pathway. Extrinsic activators include reagents, such as natural tissue factor, recombinant tissue factor, thromboplastin, Innovin®, Readiplastin®, EXTEM®, and other tissue factors derived from human and/or animal sources.

As used herein, the term “intrinsic pathway” refers to the intrinsic coagulation pathway. Correspondingly, an “intrinsic activator” is a clotting activation reagent that initiates the intrinsic pathway. Some examples of intrinsic activators are ellagic acid, celite, kaolin, polyphosphates, and INTEM.

As used herein, the “first phase” refers to a micro environment characterized by (i) a uniform distribution of water throughout a single homogenous liquid environment and (ii) corresponding T2MR values for the micro environment. The first phase is present in a sample prior to clot formation.

As used herein, the “second phase”” refers to a micro environment characterized by a single uniform fibrin clot distributed throughout the sample and corresponding T2MR values for the micro environment. The T2MR values for the second phase are lower than the T2MR values of the first phase.

As used herein, the “third phase” refers to a serum-like micro environment that typically appears in the sample after platelet activation and platelet induced clot contraction. The third phase is characterized by high T2MR values, including the maximum T2MR value observed during the coagulation reaction (also referred to herein as the “high T2” peak or the “serum” peak).

As used herein, the “fourth phase” refers to a clot micro environment that contains loosely bound red blood cells. The fourth phase is characterized by low T2MR values (also referred to herein as the “low T2” signal or the “clot” signal). The third phase and the fourth phase can be present in the sample simultaneously at one or more time points in the coagulation reaction.

As used herein, the “fifth phase” refers to a serum-like micro environment that can form following fibrinolysis or clot lysis, and corresponding clot degradation and release of red cells. The fifth phase is characterized by a decrease in T2MR values for the serum signal.

As used herein, the “sixth phase” refers to a tight clot micro environment that contains tightly bound red blood cells. The sixth phase is characterized by very low T2MR values (also referred to herein as the “tight clot” signal). The third phase, fourth phase, and sixth phase can be present in the sample simultaneously at one or more time points in the coagulation reaction.

As used herein, the term “platelet activity metric” or “PAM” is a metric characteristic of the platelet activity of the sample as measured by T2MR. For example, the PAM can be a measure derived from the T2MR intensities or T2MR values arising from the third phase and the fourth phase, or it can incorporate combinations of T2 time and intensity.

As used herein, the term “DiffT2” refers to the difference in T2 values between the third phase (i.e., the T2 value for the serum micro environment) and the fourth phase (i.e., the T2 value for the clot micro environment) in a sample at one or more predetermined points in the coagulation reaction. The DiffT2 is calculated according to the formula: DiffT2=T2serum−T2 clot. DiffT2 can be measured at one or more predetermined points in the coagulation reaction calculated either as a single value (e.g., T2_clotA, T2_clotB, etc.), or as an average difference over a predetermined time range.

As used herein, the term “deltaT2” refers to the difference in T2 values between the first phase (i.e., the T2 value for the uncoagulated sample) and the second phase (i.e., the T2 value for the uniform fibrin clot distributed throughout the sample) in a sample at one or more predetermined points in the coagulation reaction.

As used herein, the term “% deltaT2” refers to the difference in T2 values between the first phase (i.e., the T2 value for the uncoagulated sample) and the second phase (i.e., the T2 value for the uniform fibrin clot distributed throughout the sample) in a sample at one or more predetermined points in the coagulation reaction normalized by the T2 value of the first phase. For example, the difference can be calculated by fitting the T2MR curve for the first and second phases to identify a maximum asymptote and minimum asymptote in the curve fit, where the maximum asymptote represents the highest T2 value in the first phase, and the minimum asymptote is the lowest T2 value at time points following the beginning of second phase. The % deltaT2 can be calculated using the equation below:

% deltaT2=(maximum asymptote−minimum asymptote)/(maximum asymptote)

The % deltaT2 value, in combination with other measures (i.e., hematocrit value), can be used to determine the fibrinogen concentration of the sample.

As used herein, the term “deltaR2” refers to the difference in 1/T2 values between the first phase (i.e., the 1/T2 value for the uncoagulated sample) and the second phase (i.e., the 1/T2 value for the uniform fibrin clot distributed throughout the sample) in a sample at one or more predetermined points in the coagulation reaction. For example, the difference can be calculated by fitting the T2MR curve for the first and second phases to identify a maximum asymptote and minimum asymptote in the curve fit, where the maximum asymptote represents the highest T2 value in the first phase, and the minimum asymptote is the lowest T2 value at time points following the beginning of second phase. The deltaR2 can be calculated using the equation below:

deltaR2=(1/maximum asymptote−1/minimum asymptote)

The deltaR2 value, in combination with other measures (i.e., hematocrit value), can be used to determine the fibrinogen concentration of the sample.

As used herein, the term “T2MR signature” refers to the change in T2 values for one or more water populations in a sample undergoing a clotting process. The clotting process can be characterized by the emergence of different phases in the sample over time, each phase characterized by one or more water populations having a T2 value and a T2 intensity observable by T2MR.

Other features and advantages of the invention will be apparent from the following Detailed Description, the drawings, and the claims.

### BRIEF DESCRIPTION OF THE DRAWINGS

FIGS. 1a-1d depict how T2MR data is acquired and how T2MR kinetic spectra (e.g. T2MR signatures) are formed by algorithms such as numerical inverse Laplace transform or multi-exponential fitting. FIG. 1a demonstrates two relaxation curves generated by the CPMG signal acquisition at a single point in time of unclotted blood and a single point in time for clotted blood. FIG. 1b shows T2 vs. intensity spectra of unclotted and clotted whole blood. These spectra can be generated by fitting the relaxation curves in FIG. 1a with an inverse Laplace transform algorithm. FIG. 1c demonstrates the assembly of spectra into a 3D plot to generate a time series of the T2 vs. intensity spectra. These form a T2MR signature that contains intensity and peak width information. FIG. 1d depicts an alternative method for viewing T2MR signatures composed of only T2 values and time. This can be arrived at by either a data simplification of the data in FIG. 1c wherein the T2 value corresponding to the center of each peak in each T2 spectrum is calculated by averaging over the encompassed T2 values, which are then plotted as a function of time to create T2 relaxation signatures or by fitting the time series of relaxation curves with multi-exponential curve fits and displaying only the T2 values output from the fit as a function of time.

FIG. 2 shows an example of dynamic whole blood hemostasis monitoring with T2MR. Clotting was initiated with 3 U/ml thrombin prior to time zero. Part (a) shows a single exchange-averaged water population. Part (b) demonstrates initiation of clot contraction that resolves the erythrocyte water population from the serum. Part (c) demonstrates a steep increase in the upper peak as serum is extruded from the clot. Part (d) shows the completion of contraction and plateau of the upper peak showing that the T2 value of the serum like micro environment has stabilized. Part (e) shows the plateau of the middle peak for loosely bound erythrocytes showing that the T2 value for the loosely bound clot has stabilized. Part (f) demonstrates a low T2MR signal corresponding to water trapped inside a tightly contracted clot. Fibrinolysis caused by the addition of tPA at 30 min (g) releases erythrocytes back into solution lowering the T2 value of the serum peak, which causes the loosely bound clot to release erythrocytes, leaving only the signal associated with the more tightly bound erythrocytes (h and i).

FIGS. 3a and 3b represent evaluations of the peak assigned to the loose clot structure. The sample compartment generating the signal at 300 ms that dropped to 200 ms was assessed by testing two conditions: (3a) re-calcified citrated whole blood activated with thrombin to form a contracted clot, and (3b) re-calcified citrated whole blood activated with thrombin followed by addition of tPA. Both samples were mixed with a pipette tip after 190 minutes of incubation to demonstrate which signals are susceptible to mixing or sheer forces. In the absence of tPA induced fibrinolysis the serum and loose clot peak remain distinct indicating some of the loose clot integrity is preserved. However, in the presence of tPA induced fibrinolysis only one peak remains after mixing indicating complete mixing of the serum and loose clot microenvironments likely due to the degradation of the fibrin mesh.

FIG. 4 shows a method comparison between the data collected by T2MR and the Stago ST4 system using Innovin® as an activator for the PT analysis of citrated blood. The best-fit line parameters were slope=1.05, intercept=7.51 and correlation value of 0.97 (R²=0.94).

FIG. 5 shows comparisons of analysis methods for measuring clot strength between T2MR and TEG for activation of citrated blood with calcium and kaolin. The DiffT2 value was calculated by taking the difference between the upper and middle T2 values in the T2MR signature at a time point 13 minutes after activation. Data are shown here using samples from 3 healthy donors where clot strength was adjusted by ex vivo addition of abciximab (ReoPro) at a level of 0, 4, or 8 μg/ml prior to measurement. All samples containing abciximab were at DiffT2 values of <100 ms or TEG MA values less than 40.

FIGS. 6a and 6b demonstrate the dependence of T2 (ms) and 1/T2 (s⁻¹) on percent hematocrit. (6a) Reconstituted blood samples prepared to span a wide range of hematocrit were measured in triplicate by T2MR to generate a T2 value and in duplicate by the Sysmex pocH-1001 hematology analyzer to determine the hematocrit. Measured values (black circles) followed the same trend as expected values calculated from equation 10 (gray line). (6b) Samples prepared to span a wide range of hematocrit values were measured in triplicate by T2MR to generate a 1/T2 value and in duplicate using a complete blood count analyzer. Measured values (black circles) followed the same trend as expected values for equation 10 (gray line).

FIG. 7a-7b depicts a T2MR signature collected with a slow dwell time (10 seconds) and analyzed with a multi-exponential fitting methodology. FIG. 7a shows the T2 vs. time plot that shows platelet-induced clot contraction initiated at about 5 min and produced two T2MR peaks, one labeled “High T2” that is associated with the serum micro environment and is characteristic of the third phase and one labeled “Low T2” that is associated with the clot micro environment and characteristic of the fourth phase. The difference between these two values is DiffT2. FIG. 7b shows the corresponding intensity values for the T2 peaks in panel FIG. 7a. Between 0 and 5 minutes there is only one intensity peak at 100%, which corresponds to a single T2MR signal in FIG. 7a. In FIG. 7b starting at 5 minutes there are two peaks in the intensity plot that correspond to the two T2 peaks in FIG. 7a. In FIG. 7b, the peak starting at 80% intensity corresponds to the “Low T2” peak (fourth phase) and the peak that starts at ˜20% intensity corresponds to the “High T2” peak (third phase). The platelet activity metric (PAM) utilizes both the T2 and Intensity values shown in FIG. 7a and FIG. 7b. FIG. 7c shows a plot depicting the CPMG SSE vs. time, which is a plot of the fit error over time and is used as a tool for evaluating the quality of the fit and selecting the optimal multi-exponential fit.

FIG. 8a is a schematic that depicts the T2MR signature of a citrated blood sample mixed with either an extrinsic or intrinsic clotting pathway activator. The initial T2MR value shown in part (i) corresponds to a uniform distribution of water throughout a single homogenous liquid environment prior to clot formation (phase 1). This single T2MR value shown at part (ii) has shifted downward upon clot formation due to the formation of a single uniform fibrin clot distributed throughout the sample (phase 2). After platelets are activated, the single T2MR value shown at part (iii) splits into two values that corresponds to a serum-like micro environment shown at part (iv) (phase 3) and clot micro environment containing loosely bound red blood cells shown at part (v) (phase 4). When clot lysis occurs, the T2MR value shown at part (vi) for the serum-like micro environment decreases due to clot degradation and release of red cells (fifth phase).

FIG. 8b is a schematic that depicts how various parameters are derived from a T2MR measurement on a citrated whole blood sample. Clotting is initiated by the addition of activator at time zero and the instrument measures T2MR values for the sample at successive time intervals during the clotting process. FIG. 8b shows a plot of T2 signal over time involved in hemostasis reactions that demonstrates how quantitative and semi-quantitative results are derived from a T2MR signature. Measurements of various parameters involved in hemostasis can be inferred from different areas of the curve. For samples activated with an extrinsic activator, prothrombin time (PT) is derived from the transition between the unclotted and clotted T2MR values, shown as CT in the figure. For samples activated with an intrinsic activator, activated partial thromboplastin time (aPPT) is derived from the transition between the unclotted and clotted T2MR values, shown as CT in the figure. Fibrinogen (Fg) is derived from the decrease in T2MR signal upon clotting. Platelet Function (PLT) is derived from the T2MR signals from the serum and clot microenvironments and fibrinolysis (LYS) from the decrease in the serum phase T2MR signal.

FIG. 9a represents a T2MR signature that highlights the clotting time (CT) and fibrinogen portion of the signature. FIG. 9b depicts actual T2MR data (black dots) for a clotting time of a normal sample with a gray arrow depicting from which part of the data the clotting time is derived. FIG. 9c depicts actual T2MR data (black dots) for a clotting time of an abnormal sample with a gray arrow depicting from which part of the data the clotting time is derived. For clotting time measurements the T2 values are normalized to the initial starting T2MR value.

FIG. 10a depicts a T2MR signature schematic highlighting the clot time and fibrinogen portion of the signature. FIG. 10b represents actual T2MR data (black dots) for a fibrinogen measurement of a normal sample with a gray arrow depicting from which part of the data the fibrinogen is derived. FIG. 10c represents actual T2MR data (black dots) for a fibrinogen measurement of an abnormal high fibrinogen sample with a gray arrow depicting from which part of the data the fibrinogen is derived. For fibrinogen measurements the T2 values are normalized to the initial starting T2MR value.

FIG. 11a shows a T2MR signature schematic highlighting the platelet (PLT) portion of the signature. FIG. 11b depicts actual T2MR data (black dots) for a platelet measurement on a normal sample with a gray arrow depicting from which part of the data the platelet function can be derived.

FIG. 12a shows a T2MR signature schematic highlighting the fibrinolysis (LYS) portion of the signature. FIG. 12b depicts actual T2MR data (black dots) for a fibrinolysis measurement on an abnormal fibrinolytic sample with a gray arrow depicting from which part of the data the fibrinolysis is derived.

FIG. 13 depicts typical data from T2 clotting measurement, showing dependence on T2 relaxation time with time after clotting initiation. Raw data are black points, and red line represents minimum error solution from 5-parameter logistic regression fit (5PL). The MaxT2 and MinT2 are the maximum and minimum asymptotes from the 5PL fit, and the % deltaT2 is calculated from the difference between the MaxT2 and MinT2.

FIGS. 14a-14c show clotting time data for three donor samples treated with intrinsic activator and various amounts of tPA or aprotinin.

FIGS. 15a-15d shows T2MR data that can be used to derive the platelet activity and fibrinolysis levels of the samples. FIG. 15a-15c show intrinsic activator measurements where ellagic acid was added to blood from three different donors. FIG. 15d shows measurements where tissue factor was used as an activator to activate the extrinsic pathway.

FIGS. 16a and 16b depicts T2MR data for clotting time measurements from two samples that were treated with different amounts of intrinsic activator. FIG. 16a depicts measurements obtained from the addition of 4.8 μL of ellagic acid activator and 35.2 μL of citrated whole blood, while 16b depicts measurements obtained from the addition of 20 μL of the same ellagic acid activator and 35.2 μL of citrated whole blood. The clotting time shifts according to the amount of activator added. FIG. 16a shows a final sample dilution of 88% concentration and FIG. 16b shows a 64% final sample concentration. The lower the final sample concentration (e.g. higher dilution) corresponds to a higher starting T2 value.

FIG. 17a depicts a fibrinogen assay calibration curve using intrinsic pathway activation using ellagic acid (T2In assay) and a linear fit. FIG. 17b depicts a fibrinogen assay calibration curve using extrinsic pathway activation and a polynomial fit (T2Ex assay calibration curve). FIG. 17c depicts a fibrinogen assay calibration curve using an extrinsic pathway activator and a linear fit (T2Ex assay calibration curve).

FIG. 18 is a plot depicting a method comparison between clotting time measurements taken via the T2Ex CT test vs. the STAGO Plasma PT test when using extrinsic pathway activator.

FIG. 19 is a plot showing a method comparison between clotting time measurements taken via the T2In CT test vs. the STAGO Plasma PT test when using intrinsic pathway activator.

FIGS. 20a and 20b depict platelet activity for the T2Ex test. FIG. 20a depicts an overlay of several T2Ex runs showing a dose response of the T2MR signature to a titration of Cytochalasin-D (0, 1, 2, 3.5, and 5 μM Cytochalasin-D), while FIG. 20b shows a plot of platelet activity metric derived from these curves vs. cytochalasin D concentration. The platelet activity is shown in arbitrary units and is for PAMj. The platelet activity was monitored for 30 minutes.

FIG. 21a-21c depict platelet activity measurements that compare results from ROTEM® to T2MR for measurements taken using an intrinsic pathway activator, known as the T2In panel assay. Each figure depicts a different donor. The platelet activity measurement (PAMj) derived from T2MR data is on the y-axis and the ROTEM® data is on the x-axis. The plots reveal significant correlation between the data collection methods for the samples tested with R² values 0.9. Each figure is a titration of the platelet inhibitor Reopro (Abciximab) across a range of concentrations. As is evident in the figure, the PAMj metric can reach a zero value for non-zero values of ROTEM MCF. This is because the ROTEM MCF is a measure of both platelet and fibrinogen activity whereas the PAMj metric is a measure of only platelet activity.

FIGS. 22a-22d show the determination of % deltaT2 as a measure of fibrinogen concentration and its dependence on hematocrit. FIG. 22a depicts example data showing the mean % deltaT2 as compared to fibrinogen concentration for fibrinogen measured in plasma (solid circles) and fibrinogen measured in blood with T2MR and using a system of linear equations correction function (open squares, Equations 10 & 11). FIG. 22b is a demonstration of data corrected for hematocrit by using a ratio of the % deltaT2 and T2Max. FIG. 22c is data showing the dependence of % deltaT2 on both hematocrit (Hct) and fibrinogen for values that have not been treated with a correction function. FIG. 22d shows the dependence of T2Max on hematocrit and fibrinogen for values that have not been treated with a correction function.

FIG. 23a represents a method comparison of T2MR whole blood fibrinogen measurement vs Stago plasma fibrinogen measurement. The samples measured in this data set ranged from 19.4% to 50.6% Hematocrit concentration. The fibrinogen values were obtained using the calibration curve of % DT2/maxT2 vs plasma fibrinogen as shown in FIG. 23b.

FIGS. 24a-24d depict multi-dimensionality of a T2MR hemostasis data set acquired with the mixed algorithm setting and the relationships between the different types of T2MR data. Experimental data was obtained in whole blood (40 μL) with the T2MR technology. FIG. 24a reveals the clot time (sec) at the inflection point of the curve, the fibrinogen concentration and the hematocrit values can also be derived from this curve. The data in FIG. 23a (characteristic of the first phase and the first part of the second phase) were obtained using a short dwell time and mono-exponential fit. When overlayed with data collected with a long dwell time and multi-exponential fit the data in FIG. 23b were obtained. The transition between the short and long dwell time data is at 2 minutes. These data show that both the number of observed peaks and the effective mean T2 value can differ between the short and long dwell time data acquisition settings. The data in FIG. 24b after two minutes show multiple T2 values as a function of time enabling measurement of the platelet induced clot contraction. The data points in FIG. 24b and FIG. 24c can be used to calculate the PAMj metric shown in FIG. 24c. This metric accumulates over time, consistent with it being an integral function. The data that is incorporated into 24c is characteristic of the third phase. FIG. 24d shows the T2MR intensity values as a function of time. The first 2 minutes show 100% intensity in one water population. After 2 minutes two water populations are evident and their intensities change as a function of time. The intensity and T2 values are used to calculate a cumulative metric of platelets allowing the discernment of platelet pathologies or dysfunctions (FIG. 24c), as well as fibrinolysis (FIG. 24b).

FIGS. 25a-25c depict T2MR data obtained from an extrinsic activator assay using a thrombin inhibitor (rivaroxaban). In FIG. 25a, the clotting time was compared using T2MR vs the STAGP plasma PT. In FIGS. 25b and 25c clotting time data is depicted if 0 ng/mL or 800 ng/mL of rivaroxaban, a blood thinner and anti-coagulant, is added.

FIGS. 26a-26c depict clotting time curves showing the effect of varying fibrinogen levels on the size of the T2 response upon transition from the first phase to the second phase. The clotting was initiated with ellagic acid. A larger % deltaT2 is observed with increasing fibrinogen levels.

FIG. 27a-27d represent platelet activity measurements taken with intrinsic activator and the addition of ReoPro, a platelet activity inhibitor. FIG. 27a includes no ReoPro addition. FIG. 27b includes the addition of 5 μg/mL ReoPro, and 10 μg/mL of ReoPro was added in FIG. 27c. One can see the inhibition of platelet activity with ReoPro addition. FIG. 27d depicts the platelet activity measurements of three separate donors with titrations of ReoPro.

FIG. 28 depicts the R2 area representing the lysis matrix (Lys5) over a graph of T2MR data obtained over time. The area between the inverse of the upper boundary defined by the maximum T2 value of the third phase and inverse of the T2 value of the fourth phase (T2up) is taken as the R2 area.

FIG. 29 is a graph showing the relation between the percent coefficient of variation (% CV) and Lys5 (R2 area) at 45 minutes into the coagulation process. Each data point corresponds to a separate experiment.

FIG. 30 is a series of graphs, each of which corresponds to a separate experiment and shows the Lys5 value as a function of increasing concentrations of tissue plasminogen activator (tPA) at 45 minutes into the coagulation process.

FIG. 31 is a series of graphs, each of which corresponds to a separate experiment and shows the comparison between Lys4 and Lys5 values as a function of increasing concentrations of tPA, where Lys4 is determined by the area between an upper boundary defined by the maximum T2 value of the third phase and the T2 value of the forth phase (T2up).

FIGS. 32a-32c are model T2MR data highlighting relationship of T2 and R2 values at an early region in the coagulation process. FIG. 32a is a 4 parameter logistic fit (4PL) of T2MR data from the beginning of coagulation to the lysis stage. FIG. 32b is a close-up image of a series of R2 values representing the degradation of fibrinogen into fibrin. FIG. 32c is a close-up image of a series of T2 values, indicating the determination of clot time.

FIGS. 33a and 33b are standard curves showing the correlation between T2MR measured fibrinogen concentration and standard Clauss Fibrinogen measurements. The T2MR data shown in FIG. 33a were acquired as part of an intrinsic T2MR (T2In) experiment, whereas the T2MR data shown in FIG. 33b were acquired as part of a thrombin-induced T2MR (THR) experiment.

FIGS. 34a and 34b are T2MR data obtained before (FIG. 34a) and after (FIG. 34b) the end of complete fibrin formation. FIG. 34 is fit with a 4PL function.

FIG. 35 is a representative 4PL function that estimates T2MR data during fibrin formation. A max asymptote limit, inflection point, and min asymptote limit are indicated.

FIG. 36 is a representative a derivative of a 4PL function that estimates T2MR data during fibrin formation. A max asymptote limit, inflection point, and min asymptote limit are indicated.

FIGS. 37a-37f is a series of graphs showing that the clot time, as determined by the algorithm, remained constant as the upper asymptote varied. The upper asymptotes successively increase from FIGS. 37a to 37f.

FIG. 38 shows T2MR data collected after switching CPMG sequences in response to detection of the end of fibrin formation. These data show that platelet retraction begins after switching.

FIG. 39 is a process map of the smart switch response process.

### DETAILED DESCRIPTION

The methods and devices of the invention can be used to assess the risk and occurrence of thrombotic events, including myocardial ischemic events in a patient having or suspected of having vascular disease, particularly in patients who have undergone percutaneous intervention and may be at acute risk of, for example, stent thrombosis, vessel restenosis, myocardial infarction, or stroke. For example, the methods and devices of the invention can be used to assess platelet reactivity (i.e., relative concentration of platelet-associated water molecules in a clot), clotting kinetics, clot strength, clot stability, and time-to-fibrin generation (i.e., R), as indices for risk of a thrombotic event, such as myocardial ischemia, independent of responsiveness to drug therapy (e.g., as assessed by a change in platelet reactivity following administration of an anti-platelet drug such as clopidogrel). These indices can also be used to prevent complications arising from surgical and percutaneous vascular procedures (e.g., stent placement or balloon angioplasty) such as stent thrombosis or re-stenosis. Furthermore, the methods and devices of the invention can be used to identify a safe and effective therapy (e.g., dose, regimen, anti-platelet therapy, among others) for a patient at risk of a thrombotic event or undergoing a surgical procedure.

Historically, a variety of technologies have been developed that address each of the above needs across different instrument systems with varying levels of automation and method related complexity. Tests like prothrombin time (PT), activated partial thromboplastin time (aPTT), fibrinogen, and a range of individual clotting factor assays are available to diagnose defects in both the intrinsic and extrinsic coagulation pathways. These tests are performed using citrated plasma samples and are typically implemented on a larger random access instrument found in the hospital laboratory. They typically require 40 to 60 minutes for sample processing. Platelet light transmission aggregometry remains the gold standard for the assessment of platelet activation and function in clinical practice. This gold standard is a highly manual process and requires expert users to follow more extensive protocols than most other coagulation methods. Platelet aggregometry is typically performed at a specialty coagulation lab and takes 3 hours or longer to complete sample processing, measurement, and data interpretation. Finally, while all of these methods are available today, they require multiple instruments, different sample types, varying levels of user effort and expertise to perform, and time to results can vary significantly.

Thromboelastography methods (TEG® & ROTEM®) have been developed to provide a more global and dynamic assessment of the clotting process. These methods follow clot formation and dissolution (fibrinolysis) in real time by measuring the clot stiffness of a whole blood sample using different clotting activators and inhibitors. Although these clot stiffness indices can be used to infer clinical parameters, their relationship to clinically established parameters like platelet function and fibrinogen, for example, is limited.

We have developed and characterized a new diagnostic platform that enables both standard hemostasis measurements as well as measurements that provide novel insights into the dynamics and physical states of blood during clotting and lysis. Assignment of the different T2 Magnetic Resonance (T2MR) signals to distinct blood and clot constituents permits continuous monitoring of the dynamic states of blood components over a wide range of platelet counts, fibrinogen concentrations, hematocrit levels, activator concentrations and other contributors to clotting in whole blood. The T2MR platform allows real-time assessment of the transition of fibrinogen to fibrin polymer, clot contraction by platelets, formation of tightly contracted clots, and fibrinolysis.

Initial correlation and precision studies also demonstrate the potential for clinically relevant measurement of hematocrit, clotting time, platelet reactivity and clot strength with this platform. T2MR may provide novel insights into overall platelet health because clot contraction requires not only the signaling and membrane receptor functions assessed by platelet aggregometry, but also the interaction between the platelet cytoskeleton and fibrin. Preliminary studies suggest that T2MR may show residual platelet capacity to cause clot retraction in whole blood in the presence of inhibitors that block platelet aggregation and may thus find a place in the monitoring of aspirin and other anti-platelet agents to which biological resistance is encountered in the absence of a laboratory correlate.

The T2MR platform combines the flexibility of conforming to standard measurements of hemostasis with analysis of integrated coagulation in whole blood, including the contribution of leukocytes, microparticles and other factors difficult to assess at present. The relative simplicity of the instrumentation and methodology involving a single transfer of whole blood from a test specimen should permit rapid testing requiring no sample preparation and minimal sample volumes.

Lastly, this platform permits rapid and sensitive analysis of whole blood clotting across a spectrum of conditions ranging from impaired hemostasis to hypercoagulable states that cannot be readily assayed using currently available methodology. The unique small sample volume requirement is particularly advantageous for pediatric populations, studies of thrombotic and bleeding disorders in small animal models, pediatrics, neonates, and point-of-care testing. Neonates and children can also have greater variability in plasma concentrations of coagulation proteins, partially because they have rapidly changing systems, requiring multiple reference samples. These requirements place an even higher premium on the ability to efficiently use small sample volumes. Congenital and acquired hemostasis disorders often present during childhood, and accurate diagnosis is essential in order to implement optimal therapies. It is very important to be able to understand the clinical outcomes such as bleeding and thrombosis in pediatric patients undergoing complex congenital cardiac surgical procedures, and use of a panel requiring low blood volumes and fast turn-around times are important due to the small blood volume of these patients. Additionally, the measurement of the different panel parameters could aid in the diagnosis in infants, children, or adults of many hemostatic disorders. Some of these are hemophilia, von Willebrand disease, Bernard-Soulier syndrome (giant platelet syndrome), hypercoagulable state, thromotic thrombocytopenic purpura, thrombocytopenia, primary thrombocythemia, induced thrombocytopenia, disseminated intravascular coagulation, procoagulant afibrinogenemia/dysfibrinogenemia, protein C deficiency, protein S deficiency, antithrombin III deficiency, factor V Leiden deficiency, activated protein C resistance (aPCR), Anticoagulant afibrinogenemia/dysfibrinogenemia, Factor V deficiency, Factor VII deficiency, Factor X deficiency, Factor XI deficiency, Factor XII deficiency, Factor SIII deficiency, hypoprothrombinemias, cryoglobulinemias, multiple myeloma, Waldenstrom macroglobulinemia, Henoch-Schonlein purpura, hyperglobulinemic purpura, cavernous hemangioma, hereditary hemorrhagic telangiectasia, pseudoxanthoma elasticum, Vitamin K deficiency, Shwartzman phenomenon, Wiskott-Aldrich syndrome, sepsis, and hemolytic disease of the newborn.

Clotting Initiation

For performing the methods of the invention, clotting may be initiated using a variety of techniques. Kaolin (CK), ellagic acid and celite are common initiators for contact factor pathway activation (intrinsic pathway) commonly used for aPTT (activated partial thromboplastin time) and whole blood clotting times. These intrinsic pathway activators can be used with or without added lipid formulations. Kaolin, celite, or ellagic acid-activated samples are characterized by clot formations where platelets and fibrin contribute to the clot. Alternatively, an activator, RPF may be used to initiate clotting with or without the addition to a platelet activator such as TRAP, epinephrine, AA, collagen, batroxobin (reptilase), ecarin, factor XIlIa plus platelet agonist, or ADP. RPF activated samples are characterized by clot formations where fibrin rather than platelets contribute primarily to the clot. Alternatively ADP (or ADP+RPF) may be used to activate the clot. ADP-activated samples are characterized by clot formations where fibrin contributes primarily to the clot and platelets contribute to lesser degree. The signal response observed under different activation conditions can be diagnostic of the hemostatic condition of a subject. Extrinsic pathway activators are also used to initiate coagulation. Some extrinsic pathway activators are tissue factor, thromboplastin, and reagents known as Innovin, readiplastin, and EXTEM®. Tissue factor is a common initiator for PT, diluted PT measurements, and extrinsic pathway activation such as that done by EXTEM®, a thromboelastometry test. Tissue factor activated samples can lead to clot strength, clot time, fibrinogen, platelet activity and fibrinolysis measurements like samples activated with contact factor activators.

Other blood clotting activators that can be used in the methods of the invention include but are not limited to collagen, epinephrine, ristocetin, thrombin, calcium, tissue factor, prothrombin, thromboplastin, kaolin, serotonin, platelet activating factor (PAF), thromboxane A2 (TXA2), fibrinogen, von Willebrand factor (VFW), elastin, fibrinonectin, laminin, vitronectin, thrombospondin, and lanthanide ions (e.g., lanthanum, europium, ytterbium, etc.). Combinations of activators and samples can be used, for example, to aid in identifying an underlying hemostatic condition that results in a subject's blood sample being hypocoagulable.

Calcium chloride (CaCl₂) is routinely used to recalcify samples that contain anti-coagulants in combination with buffers along with the activator or agonist to reverse the inhibitory effects of an anticoagulant such as citrate, heparin, corn trypsin inhibitor or other anti-coagulants that are commonly found in clinical samples.

Signal Acquisition and Processing

MR relaxometry is a powerful technique that is used to detect dynamics of blood. The experiments developed enable the detection of platelet activity, fibrinolysis, fibrinogen level, clot time and hematocrit. All of the experiments can be performed using a Car-Purcell-Meiboom-Gill sequence (CPMG sequence). The pulse sequence scheme is (90)x-τ1-[(180)y-τ2]n- . . . where the n and the repetition time changes with the type of experiment (see below). The inter-pulse delays are usually set respectively to 249.6 μs for τ1 and 499.2 μs for τ2 delays. The nutation frequency (γB₁) of ¹H is at about 25 kHz.

The number of 180 pulses can be set from 2000 to 14,000 with a relaxation delay from 1 to 3 seconds yielding a total experimental time from 5 to 30 seconds for a single data point (dwell time). The detection of fibrinogen, clot time, and hematocrit can be performed by detecting either the intrinsic (T2In) or extrinsic (T2Ex) pathway in whole blood. As these two phenomena might have different dynamics, the CPMG parameters can be set as follows: (i) T2In: 3-10 second dwell time, 5 minutes total experimental time; and (ii) T2Ex: 1-10 second dwell time, 2-5 minutes total experimental time. Fibrinogen level and clot time can also be detected in plasma. For this experiment the dwell time is between 1 to 10 seconds and the total experimental time is 2 minutes. The settings described herein were found to be optimal for these assays, but other optimal settings may be found for other types of assays. A range of settings can be used. We have used an inter-pulse delay as short as 92 μs and up to 3,000 μs. The number of echoes and relaxation delay can be configured to set a dwell time can be as short 0.5 seconds or as long as several minutes.

In particular embodiments, the parameters of the CPMG sequence vary during the clotting process (i.e., utilizing faster or longer dwell times, depending, e.g., upon the presence or absence of clot and/or serum microenvironments in the sample at the time the T2 data is collected).

The T2MR curve for a sample can be characterized by a transition from the first phase to the second phase of the clotting process. The changes in T2 values and intensities for this transition permits determination of a clotting time for the sample, and so is referred to herein as the “clotting time portion” of the T2MR curve. For the clotting time portion of the T2MR curve the CPMG signal acquisition settings commonly used are 500-4,200 180° pulses (i.e., echoes), inter-echo delay of 0.5-5 ms. These signal acquisition settings can be utilized for a period of about 2-5 minutes (or longer) and permit numerous T2 measurements at points in the clotting process where rapid changes in T2 values and intensities may be observed and may be critical to the evaluation of a particular sample.

For the clotting time portion of the T2MR curve, the mono-exponential fitting procedure can be used to measure the T2 values characteristic of the clotting time of, e.g., whole blood or plasma. Once that the CPMG curves are deconvoluted using a mono-exponential fit, the raw T2 data that form the T2MR curve can be fitted using an inverted population function with a 4 parameter logistic regression (4PL) fitting procedure. The 4PL fit can be minimized by employing the non-linear least square (NLLS) algorithm. The fitting routine is executed in two steps. The first fit estimates the portion of time in the T2MR curve when the clot occurs and the second fit calculates the clotting time by selecting the region calculated by the first fit. Specifically, the data points used by the second 4PL fit are the T2 points corresponding to approximately twice the clotting time. The output provides information about the clotting time, the fibrinogen value, and the hematocrit value of the sample. The clotting time parameter helps to detect the dynamics of the polymerization of fibrinogen into fibrin formation and the subsequent change in viscosity of the blood detected as a drop of the T2 value (i.e., characteristic of the second phase). The difference between the T2 value before and after the clot formation is proportional to the fibrinogen level. For this measurement a calibration curve is needed. The initial T2 value is proportional to the amount of hemoglobin present in the red cells and correlates to the hematocrit value.

In one embodiment of the data acquisition process, a filter (smart response) is used to automatically switch the CPMG parameters from a faster to a longer dwell time after the clot is formed. For example, the filter can be triggered by comparing the sum of square error (SSE) between a mono and a multi exponential fit, to determine whether, for a given time point of the T2MR curve, the decay curve fits to either a mono or a multi-exponential decay (i.e., one or multiple water populations). After the clot is formed, separate clot and serum microenvironments can be observable by T2MR as separate signals having different T2 values. When the number of observable water populations is greater than one, the filter switches to the longer dwell time. Alternatively, the filter can be triggered by implementing a 4PL routine that is performed automatically every predetermined number of data points in the T2MR curve (i.e., every 5-10 points). The analysis and comparison of the SSE of the fits reveal when the fit is fully minimized and when the clotting time portion of the T2MR curve is completed. After the clotting time is observed, the filter switches to the longer dwell time.

The T2MR curve for a sample can be characterized by phases observable after the clotting time portion of the T2MR curve (i.e., the third and fourth phases, and the fifth phase). The changes in T2 values and intensities for these phases permit determination of a platelet activity and clot lysis for the coagulated sample, and so is referred to herein as the “hemodynamics portion” of the T2MR curve. These signal acquisition settings can be utilized for a period of about 30-60 minutes (or longer) and permit more accurate decay curves to be collected at points in the clotting process multi-exponential fits are required to observe changes in T2 values and intensities for two or more water populations, where each water population can be critical to the evaluation of a particular sample.

For the hemodynamics portion of the T2MR curve, a variety of different fitting protocols may be utilized. For example, a multi-exponential fitting can be used to detect the dynamics of platelet activity utilizing an NLLS fit to the CPMG decay curves. The fit considers a linear combination of exponential functions up to three sets, the final fit yielding one, two, or three T2 values and intensities. Each fit is assessed and the best fit selected. This evaluation of each fit can be made, e.g., by comparing the SSE between three fits categorized as mono, bi- or tri-exponential, or by utilizing predetermined thresholds for identifying the best fit. In one protocol, (i) any bi-exponential fit that doesn't have a T2 value greater than 20 ms is discarded and a mono-exponential is chosen instead; (ii) the bi-exponential fit is selected over the mono-exponential fit if the SSE of the two calculated T2 values is less than 75% of the SSE associated from the mono-exponential fit; (iii) the tri-exponential fit is selected over the bi-exponential fit if the SSE of the tri-exponential fit is less than 90% of the bi-exponential fitting SSE; (iv) if the SSE of the tri-exponential fit is >90% of the bi-exponential fit and the bi-exponential fit SSE is >75% of the mono-exponential fit, then the mono-exponential fit is selected; and (v) if the SSE of the tri-exponential fit is >90% of the bi-exponential fit and the bi-exponential fit SSE is <75% of the mono-exponential fit, but the difference between the calculated T2s of the bi-exponential fitting is less than 20 ms, the algorithm selects the mono-exponential fit.

After collection of the CPMG is completed, the raw data consists of an exponential decay curve that contains one or more exponential functions having either one or multiple T2 components. As a first step, a mono-exponential fit is performed over all the data points (i.e., ca. 14,000 points). This fitting identifies the dominant T2 component that has the largest contribution to the exponential decay. The dominant T2 value is used to select a region of the raw data for subsequent fitting by discarding any data points at time values longer than twice the dominant T2 value. The down-sampled raw data curve is then fitted with bi-exponential and tri-exponential fitting routines. This action is performed in order to mitigate artifacts that may be introduced by the noise that can be present at the end of the raw data relaxation curve and to improve the robustness (accuracy and precision) of the filtering between single and multi-exponential data sorting as described above.

Standard radiofrequency pulse sequences for the determination of nuclear magnetic resonance parameters are known in the art, for example, the Carr-Purcell-Meiboom-Gill (CPMG) is traditionally used if relaxation constant T₂ is to be determined. Optimization of the radiofrequency pulse sequences, including selection of the frequency of the radiofrequency pulses in the sequence, pulse powers and pulse lengths, depends on the system under investigation and is performed using procedures known in the art.

Nuclear magnetic resonance parameters that can be obtained using the methods of the present invention include but are not limited to T1, T2, T1/T2 hybrid, T₁ᵣₕₒ, T₂ᵣₕₒ and T₂*. Typically, at least one of the one or more nuclear magnetic resonance parameters that are obtained using the methods of the present invention is spin-spin relaxation constant T2.

As with other diagnostics and analytical instrumentation, the goal of NMR-based diagnostics is to extract information from a sample and deliver a high-confidence result to the user. As the information flows from the sample to the user it typically undergoes several transformations to tailor the information to the specific user. The methods and devices of the invention can be used to obtain diagnostic information about the hemostatic condition of a subject. This is achieved by processing the NMR relaxation signal into one or more series of component signals representative of the different populations of water molecules present, e.g., in a blood sample that is clotting or clotted. For example, NMR relaxation data, such as T2, can be fit to a decaying exponential curve defined by the following equation:

$$f ( t ) = {\sum }_{i = 1}^{n} {A}_{i} exp ( - t / T ( i ) ) , \qquad ( 1 )$$

where f(t) is the signal intensity as a function of time, t, A, is the amplitude coefficient for the ith component, and T₍ᵢ₎ the decay constant (such as T2) for the ith component. For relaxation phenomenon discussed here the detected signal is the sum of a discrete number of components (i=1, 2, 3, 4 . . . n). Such functions are called mono-, bi-, tri-, tetra- or multi-exponential, respectively. Due to the widespread need for analyzing multi-exponential processes in science and engineering, there are several established mathematical methods for rapidly obtaining estimates of A, and (T), for each coefficient. Methods that have been successfully applied and may be applied in the processing of the raw data obtained using the methods of the invention include Laplace transforms, algebraic methods, graphical analysis, nonlinear least squares (of which there are many flavors), differentiation methods, the method of modulating functions, integration method, method of moments, rational function approximation, Padé-Laplace transform, and the maximum entropy method (see Istratov, A. A. & Vyvenko, 0. F. Rev. Sci. Inst. 70:1233 (1999)). Other methods, which have been specifically demonstrated for low field NMR include singular value decomposition (Lupu, M. & Todor, D. Chemometrics and Intelligent Laboratory Systems 29:11 (1995)) and factor analysis.

There are several software programs and algorithms available that use one or more of these exponential fitting methods. One of the most widely cited sources for exponential fitting programs are those written and provided by Stephen Provencher, called “DISCRETE” and “CONTIN” (Provencher, S. W. & Vogel, R. H. Math. Biosci. 50:251 (1980); Provencher, S. W. Comp. Phys. Comm. 27:213 (1982)). Discrete is an algorithm for solving for up to nine discrete components in a multi-component exponential curve. CONTIN is an algorithm that uses an Inverse Laplace Transform to solve for samples that have a distribution of relaxation times. Commercial applications using multiexponential analyses use these or similar algorithms. In fact, Bruker minispec uses the publicly-available CONTIN algorithm for some of their analysis. For the invention described here, the relaxation times are expected to be discrete values unique to each sample and not a continuous distribution, therefore programs like CONTIN are not needed although they could be used. The code for many other exponential fitting methods are generally available (Istratov, A. A. & Vyvenko, 0. F. Rev. Sci. Inst. 70:1233 (1999)) and can be used to obtain medical diagnostic information according to the methods of the present invention. Information is available regarding how the signal to noise ratio and total sampling time relates to the maximum number of terms that can be determined, the maximum resolution that can be achieved, and the range of decay constants that can be fitted. For a signal to noise ratio of ˜10⁴ the theoretical limit as to the resolution of two decay constants measured, independent of the analytical method, is a resolution 6=(Tᵢ/Tᵢ₊₁) of >1.2 (Istratov, A. A. & Vyvenko, O. F. Rev. Sci. Inst. 70:1233 (1999)). Thus it is believed that the difference between resolvable decay constants scales with their magnitudes, which is not entirely intuitive and is unlike resolution by means of optical detection. The understanding of the maximum resolution and the dependence on resolution on the signal-to-noise ratio will assist in assessing the performance of the fitting algorithm.

The methods of the invention can be compared to systems and devices known in the art, such TEG®, ROTEM®, or SONOCLOT®, or other device to measure a rheological change. Further the methods of the invention can be used on a benchtop NMR relaxometer, benchtop time domain system, or NMR analyzer (e.g., ACT, Bruker, CEM Corporation, Exstrom Laboratories, Quantum Magnetics, GE Security division, Halliburton, HTS-111 Magnetic Solutions, MR Resources, NanoMR, NMR Petrophysics, Oxford Instruments, Process NMR Associates, Qualion NMR Analyzers, SPINLOCK Magnetic Resonance Solutions, Xigo Nanotools, Magritek, Niumagor Stelar, Resonance Systems).

The CPMG pulse sequence used to collect data with a T2reader is designed to detect the inherent T2 relaxation time of the sample. Typically, this is dictated by one value, but for samples containing a complex mixture of states (e.g., a sample undergoing a clotting process or dissolution process), a distribution of T2 values can be observed. In this situation, the signal obtained with a CPMG sequence is a sum of exponentials. One solution for extracting relaxation information from a T2reader output is to fit a sum of exponentials in a least-squares fashion. Practically, this requires a priori information on how many functions to fit. A second solution is to use the Inverse Laplace Transform (ILT) to solve for a distribution of T2 values that make up the exponential signal observed. Again, the results of the CPMG sequence S(t), is assumed to be the sum of exponentials:

$$S ( t ) = {\sum }_{i} {A}_{i} {e}^{- t / T {2}_{i}} , \qquad ( 2 )$$

where A, is the amplitude corresponding to the relaxation time constant T2ᵢ. If, instead of a discrete sum of exponentials, the signal is assumed to be a distribution of T2 values, the sum over states can be represented as:

S(t)=∫₀^{∞}A(1/T2)e^{-t/T2}d(1/T2) (3)

This has the same functional form as the ILT:

F(t)=∫₀^{∞}A(s)e^{-st}ds (4),

and can be treated as such. The ILT of an exponential function requires constraints to solve. A few methods that can be used to impose constraints are CONTIN, finite mixture modeling (FMM), and neural networks (NN). An Inverse Laplace Transform may also be used in the generation of a 3D data set. A 3D data set can be generated by collecting a time series of T2 decay curves and applying an Inverse Laplace Transform to each decay curve to form a 3D data set. Alternatively, a 2D Inverse Laplace Transform can be applied to a pre-assembled 3D data set to generate a transformed 3D data set describing the distribution of T2 times.

In a heterogeneous environment containing two phases, several different exchange regimes may be operative. In such an environment having two water populations (a and b), rₐ and r_{b} correspond to the relaxation rates of water in the two populations; fₐ and f_{b} correspond to the fraction of nuclei in each phase; τₐ and τ_{b} correspond to residence time in each phase; and a=(1/τₐ)+(1/τ_{b}) corresponds to the chemical exchange rate. The exchange regimes can be designated as: (1) slow exchange: if the two populations are static or exchanging slowly relative to the relaxation rates rₐ and r_{b}, the signal contains two separate components, decaying with time constants T₂ₐ and T_{2b}; (2) fast exchange: if the rate for water molecules exchanging between the two environments is rapid compared to rₐ and r_{b}, the total population follows a single exponential decay with an average relaxation rate (rₐy) given by the weighted sum of the relaxation rates of the separate populations; and (3) intermediate exchange: in the general case where there are two relaxation rates r₁ and r₂ with r₁ equal to rₐ in the slow exchange limit rₐ<r_{b}, Amp₁+Amp₂=1, and where r_{1,2} goes to the average relaxation rate in the fast exchange limit, the following equations may be applied:

$$\begin{matrix}{r}_{1} = ( 1 / 2 ) ( {r}_{a} + {r}_{b} + a ) - ( 1 / 2 ) \sqrt{{( {r}_{b} - {r}_{a} + a )}^{2} - 4 {af}_{b} ( {r}_{b} - {r}_{a} )} & ( 5 ) \\ {r}_{2} = ( 1 / 2 ) ( {r}_{a} + {r}_{b} + a ) + ( 1 / 2 ) \sqrt{{( {r}_{b} - {r}_{a} + a )}^{2} - 4 {af}_{b} ( {r}_{b} - {r}_{a} )} & ( 6 ) \\ {Amp}_{1} = \frac{{r}_{2} - {r}_{av}}{{r}_{2} - {r}_{1}} & ( 7 ) \\ {Amp}_{2} = \frac{{r}_{av} - {r}_{1}}{{r}_{2} - {r}_{1}} & ( 8 )\end{matrix}$$

The invention also features the use of a pulsed field gradient or a fixed field gradient in the collection of relaxation rate data. The invention further features the use of the techniques of diffusion-weighted imaging (DWI) as described in Vidmar et al. (Vidmar et al., NMR Biomed. 23: 34-40 (2010)), which is herein incorporated by reference, or any methods used in porous media NMR (see, e.g., Bergman et al., Phys. Rev. E 51: 3393-3400 (1995), which is herein incorporated by reference).

Other systems can be used to practice the invention, including High resolution benchtop NMR magnets and spectrometers (e.g. Magritek's ultra-compact spectrometer, picospin45, NanalysisNMReady 60p cover the range of 40 MHz-60 MHz), high resolution cryogenic systems, and magnetic resonance imaging systems. With sufficient magnetic field homogeneity, NMR spectroscopy can be used to monitor the chemical shift of more than one water population in a blood sample during clotting. Using this method, unique chemical shift signals can be associated with a tightly bound clot. The different chemical shifts of clot and non-clot signals arise from inherent chemical shifts of nuclei, slowing of water diffusion within a tightly bound clot, as well as microscopic inhomogeneities due to paramagnetic centers in heme within red blood cells. The paramagnetic effect has been shown to induce changes in chemical shift be several reports, as known in the art; such as the Evans NMR method and others (see Chu et al., Magn Reson Med, 13:239 (1990).

Alternatively, when the methods of the invention are carried out using the measurement of the T2*, or free induction decay, rather than T2, the relaxation properties of a specific class of, for example, water protons in the sample can be made using an on or off resonance radiation (i.e., radiation that is not precisely at the Larmor precession frequency). The output can be in the form of the height of a single echo obtained with a T2 measuring pulse sequence rather than a complete echo train. In contrast, normal T2 measurements utilize the declining height of a number of echoes to determine T2. The T2* approach can include the steps of shifting the frequency or strength of the applied magnetic field, and measuring the broadness of the water proton absorption peak, where broader peaks or energy absorption are correlated with higher values of T2. The methods can be carried out using techniques for measuring water diffusion, or utilizing the slope of an echo train. In particular embodiments the measurement is made using a CPMG sequence, or a portion thereof, for example, to remove signals associated with a sample holder.

In general, ILT is likely to detect an erroneous number of peaks if the difference between two T2 values is less than 200 ms for a bi-exponential decay. Remarkably, this corresponds to the limit suggested by Istratov (Istratov, A. A. and O. F. Vyvenko (1999). “Exponential analysis in physical phenomena.” Review of Scientific Instruments 70(2): 1233-1257) regarding the theoretical limit of the accuracy achievable by the inverse Laplace transform for a bi-exponential fit. Istratov claims that for an infinite dataset for a infinite domain size g(λ), the limit of the resolution for a dataset having a S/N ratio of 1000 fitted by the ILT transform is λₘₐₓ, λₘᵢₙ=1.88 which corresponds to 1/1.88=0.53 compared to our metric T2c(L)/T2c(U). A Non-Linear Least Squares (NLLS) algorithm can be compiled in a MATLAB executable. It can also be compiled and coded in other programming languages, as necessary for deployment in diagnostic and research products.

The same methodology and simulations used for ILT were adopted to evaluate the performance of the NNLS algorithm for T2 search. Unlike ILT, NNLS finds the correct number of peaks as the percent error of the number of T2 points is always zero. This is consistent with the parameter space of NLLS being more limited compared to ILT and because of that, it can be made more robust, or, in other words, less likely to encounter errors. The limit of the algorithm is that it detects up to 4 peaks since the current search is limited only to 1, 2, 3, and 4 discrete subsets. Increasing such parameter space will affect substantially the computational speed and reduce robustness. While ILT can be used to evaluate new assays where the number of T2 signals is unknown as it searches up to 500 discrete values, NLLS gives overall more reliable T2 values if the space-search is known well enough to apply constraints around the number of peaks and possible T2 values. While ILT must determine how many peaks can be produced to minimize the error (usually between 1 to 6), the NLLS algorithm fits for a limited set of specific number of peaks and then evaluates which fit is most appropriate. This evaluation is determined by considering the sum of square error (SSE) between three fits categorized as mono, bi- or tri-exponential. In addition, the ILT algorithm is able to quantify the heterogeneity of the population of nuclei or spins in terms of the different microenvironments, signal broadening, or T2 values that they experience. This can be evaluated through the peak width of ILT. Unlike ILT, NLLS is readily suitable to evaluate the relative signal intensities associated with each reported T2 value, which is a measure of the relative water population associated with each microenvironment.

Database of Signature Curves

In one embodiment, the invention features data processing tools to transform the raw relaxation NMR data into a format that provides signature curves characteristic of hemostatic conditions. Preferred transforms include the Laplace or Inverse Laplace Transform (ILT) and the NLLS algorithm. The data for each T2 measurement may be transformed from the time dimension where signal intensity is plotted verses time to a “T2 relaxation” dimension. The ILT provides not only information about the different relaxation rates present in the sample and their relative magnitudes but also provides the breadth of distribution of those signals.

For NLLS fitting, each acquired relaxation curve has a corresponding two dimensional signature that maps both the T2 values and the populations of water experiencing different relaxation environments. These curves can be compiled to form a 3D data set by stacking the plots over the duration of the clotting time dimension. This can be used to generate a 3D surface that shows how the T2 and different populations of water change as a function of time.

The T2 signatures and the population contributing to the T2 values may become even more clinically relevant in cases whereby underlying pathology is not discriminated by current techniques. For example, patients that have abnormal PT or aPTT values are often worked up with additional studies that includes PT, aPTT, or PT and aPTT analysis using a 1:1 mixture of a patient blood with normal plasma (to rule out a factor deficiency), and the results may point to a specific factor or von Willebrand factor deficiency. However, frequently patients having a clotting factor deficiency have more than one deficiency or have an unbalance or unchecked clotting cascade. In these patients, a single test for one factor deficiency will not reveal the full dysfunction and the clinician must rely on clinical symptomology (excessive bleeding or clotting) and, unfortunately, time may lead to a deleterious outcome. The ability to detect T2 signatures (for patients having normal or abnormal hemostatic conditions) will allow for rapid understanding of complex pathophysiological coagulation cascade conditions and improve clinical outcomes.

Management of Patients

The methods and the devices of the invention can be used to provide a point-of-care evaluation of the hemostatic condition of a patient (e.g., for coagulation management of patients undergoing surgery, to identify patients at risk of thrombotic complications, to identify a patient resistant to antiplatelet therapy, to monitor anticoagulation therapy in a patient, to monitor antiplatelet therapy in a patient, and/or to monitor procoagulant therapy in a patient, for identification of abnormal coagulopathies associated with trauma such as trauma induced coagulopathy, acute coagulopathy; such measurements can be used to inform transfusion decisions).

There are medical circumstances for which a coagulation test is requested including: 1) finding a cause for abnormal bleeding or bruising, 2) in patients with an autoimmune disease, 3) in patients with an underlying cardiovascular disorder, 4) before procedures or surgeries where too much bleeding may be a concern, 5) monitoring anti-coagulant therapy, 6) monitoring peri-operative and trauma patients, and 7) identifying patients with sepsis or septic shock.

Coagulation management of patients undergoing cardiac surgery is complex because of a balance between anticoagulation for cardiopulmonary bypass (CPB) and hemostasis after CPB. Furthermore, an increasing number of patients have impaired platelet function at baseline due to administration of antiplatelet drugs. During CPB, optimal anticoagulation dictates that coagulation is antagonized and platelets are prevented from activation so that clots do not form. After surgery, coagulation abnormalities, platelet dysfunction, and fibrinolysis can occur, creating a situation whereby hemostatic integrity must be restored. The complex process of anticoagulation with heparin, antagonism with protamine, and postoperative hemostasis therapy can be guided by the method and devices of the invention (a point of care test) that assess hemostatic function in a timely and accurate manner.

Problems associated with poor liver function (e.g., decreased synthesis and clearance of clotting factors and platelet defects) can lead to impaired hemostasis and hyperfibrinolysis. Systemic complications, such as sepsis and disseminated intravascular coagulation, further complicate a preexisting coagulopathy. Marked changes in hemostasis in orthotopic liver transplantation occur during the anhepatic phase and immediately after organ reperfusion, mainly a hyperfibrinolysis resulting from accumulation of tissue plasminogen activator due to inadequate hepatic clearance and a release of exogenous heparin and endogenous heparin-like substances. Thus, patients undergoing hepatic surgery, and particularly orthotopic liver transplantation, may have large derangement in their coagulation, making the method and devices of the invention useful for monitoring this patient population.

The method and devices of the invention can be used to guide heparin therapy, among other anticoagulation therapies. For example, the methods of the invention can be carried out with heparinase to assess the coagulation status in the absence of the anticoagulatory effects of heparin. Further, the methods of the invention can be utilized to assess protamine therapy, i.e. to monitor coagulation after protamine therapy and to treat a heparin or protamine induced hemostatic condition. Similarly, analysis could be done pre- and post-surgery to determine the anticoagulant or hemostatic status of a surgical patient.

The method and devices of the invention can also be used to guide antiplatelet therapies and identify resistance to antiplatelet therapies. Antiplatelet therapy is increasingly being prescribed for primary and secondary prevention of cardiovascular disease to decrease the incidence of acute cerebro- and cardiovascular events. Antiplatelet drugs typically target to inhibit cyclooxygenase 1/thromboxaneA2 receptors (e.g., aspirin), adenosine diphosphate receptors (e.g., clopidogrel), or GPIIb/IlIa receptors (e.g., abciximab, tirofiban). Although antiplatelet drugs are thought to work primarily by decreasing platelet aggregation, they also have been shown to function as anticoagulants. Because platelets play a key role in overall coagulation, the assessment of the platelet function (more than their number) is critical in the perioperative setting.

The method and devices of the invention can also be used to monitor and/or guide anticoagulant therapies. Anticoagulant therapies (e.g., rivaroxaban, dabigatran, among others) can be monitored for efficacy and compliance, and to ensure avoidance of adverse side effects and/or adverse events (e.g., bleeding events). Dosing adjustments for such therapies have been reported to control bleeding in large, randomized studies. Specifically, dosing of anticoagulants, including direct Factor Xa inhibitors can be used to assist maintenance of a therapeutic window and lead to a reduction of risk of stroke in atrial fibrillation and deep vein thrombosis in patients.

The method and devices of the invention can be used to identify patients resistant to anticoagulant therapy. Anticoagulant therapies include aspirin, plavix, and prasugrel, among other anticoagulants. The method includes (i) administering the anticoagulation therapy to the subject; (ii) evaluating the hemostatic condition of the subject using a method of the invention; and (iii) if the subject is found to be prothrombotic, identifying the subject as a non-responder to the anticoagulation therapy. The identification of non-responders can permit a physician to identify a safe and efficacious anticoagulant to which the patient is responsive, thereby reducing the risk of adverse events (i.e., thrombi formation and stroke).

The method and devices of the invention can be used to monitor procoagulant therapy. The modern practice of coagulation management is based on the concept of specific component therapy and requires rapid diagnosis and monitoring of the pro-coagulant therapy. It has been shown, for example, that platelet transfusion in the perioperative period of coronary artery bypass graft surgery is associated with increased risk for serious adverse events. Clinical judgment alone may not predict who will benefit from a platelet transfusion in the acute perioperative setting. Accordingly, the transfusion of coagulation products should be preferably guided by a point of care test, such as the test provided by the method and devices of the invention.

The method and the devices of the invention can be used to provide a companion diagnostic analysis or test to monitor the effects of a therapeutic compound in a clinical trial or in medical use. The diagnostic analysis may include determining whether or not the subject of the trial or the patient responds to therapy or does not respond to therapy.

The method and the devices of the invention can be used to determine the perfusion through clots, hypercoagulation, hyperclotting, or clotting that is deleterious in a human, as in stroke or cardiac arrest.

The method and the devices of the invention can be used as part of a panel of analyses. The panel can include (i) an immunoassay to proteins that are involved in the coagulation cascade; (ii) an immunoassay to detect fibrin degradation products; (iii) an immuno assay to detect antiphospholipid antibodies; (iv) an assay to detect heparin or warfarin or other anticoagulant to assess therapeutic concentration; (v) a PT or aPTT or PTT assay that monitors the plasma prothrombin time; (vi) a genetic test to assess the polymorphic differences in genes encoding proteins that are relevant to (a) the formation or dissolution of thrombin, (b) the coagulation cascade, (c) heparin binding, or (d) therapeutic activity.

The methods and the devices of the invention can be used to manage medical devices with implications towards coagulopathies. An example is a ventricle assist device often used as a bridge for patients awaiting a heart transplant. Patients with such an implant may have clot formation within and outside of the device as a result of the function of the device, and these clots may cause a stroke or another thrombus related event. It may also lead towards infections and bleeding events. A way to avoid these issues is to monitor multiple diagnostic markers that impact the success of the device. For instance, routine testing of PT-INR would allow tighter monitoring of the patients coagulation state, thus, providing tight control of bleeding and clotting events.

The INR is the ratio of a patient's prothrombin time to a normal (control) sample, raised to the power of the International Sensitivity Index value for the analytical system used. A high INR level (e.g., INR=5) indicates that there is a high chance of bleeding, whereas if the INR=0.5 then there is a high chance of having a clot. Normal INR range for a healthy person is 0.9-1.3. For people on warfarin therapy the INR range is typically 2.0-3.0. The target INR may be higher in particular situations, such as for those with a mechanical heart valve, or bridging warfarin with a low-molecular weight heparin (such as enoxaparin (Lovenox)) perioperatively.

Monitoring platelet function, fibrinolysis, clot strength and other factors are equally important in improving outcomes. Understanding the physiologic concentration or activities of these factors are important not just for their interplay with the device, but because they are modulated by the many different therapies often prescribed to patients on these devices (aspirin, rivaroxaban, plavix, warfarin, among others). Another measure that is used with these types of devices is hematocrit, which is often used to adjust the functioning of the device (speed, intensity, etc.) to maintain the function of the heart. The methods and the devices of the invention can provide all of these results (hematocrit, platelet, PT, PT-INR, etc.), potentially simultaneously, and it may provide additional information with respect to clot formation and dissolution. The standard measures above may be combined into an index or signature that identifies the status of the patient and efficacy of the device.

The methods and the devices of the invention can be utilized and configured in multiple ways. They can be used as a laboratory device (e.g., in a central laboratory or STAT laboratory), point-of-care system, or even an implantable monitoring system. For example, as an implantable monitoring system, the sample can consist of continually monitored blood; a Vacutainer® with whole blood, serum, or plasma; or a finger stick, among other sample fluids.

For example, the methods and the devices of the invention can be utilized for monitoring peri-operative and trauma patients (e.g., providing measures or surrogate measures for PT/INR, aPTT, ACT, Hct, platelet activity, and fibrinolysis). There is a need with these patient populations to quickly and efficiently determine if and what type of a transfusion is needed as the patients can exhibit an approximately 6-fold increase in mortality, ischemic events, infection, early onset of complications, and increased ICU/hospital stays. Specifically, determination of the root cause of bleeding events (coagulation cascade vs. platelet activation) can lead to prompt and focused therapy (i.e., transfusion management, anticoagulation monitoring, antiplatelet reactivity, and/or predicting thrombosis risk, among others). Packed red blood cells, platelets, specific clotting factors, fibrinogen therapy, and fresh frozen plasma can all be transfused. For instance, low hematocrit may lead toward red blood cell replacement, low fibrinogen may lead to fibrinogen treatment or fresh frozen plasma (FFP) administration, abnormal clot time may lead to administration of clotting factors or FFP, and abnormal platelet activity will suggest a platelet transfusion or appropriate medication.

Regardless of the context in which the methods and the devices of the invention are utilized, that the methods of the invention can be used to rapidly measure small volumes is particularly important for platelet function, which previously were difficult to measure using other systems due to the initiation of clotting at the site of the blood draw.

The Clotting Mechanism

For clotting to occur there must be activation of coagulation cascade culminating in fibrin deposition through the action of thrombin on fibrinogen. The coagulation system is composed of a proteolytic cascade that amplifies an initial stimulus with an elegant feedback regulation mechanism to keep the overall process in check and balance. There are two interconnected routes of clotting activation: (i) contact activation (intrinsic pathway); and (ii) tissue factor activation (extrinsic pathway). Both pathways rely on a variety of coagulation factors. Prothrombin is coagulation factor II, thrombin is coagulation factor Ila, fibrinogen is coagulation factor I, and fibrin is coagulation factor Ia. In addition to the coagulation factors, platelets are critical both for the induction and formation of an adequate blood clot. Platelets act as a phospholipid surface upon which prothrombinase complexes are formed and act as a physical scaffold for the developing clot. After thrombin production and fibrin formation platelets activate and play an active role in platelet mediated clot contraction.

The intrinsic coagulation cascade pathway is normally activated by contact with collagen from damaged blood vessels, but many negatively charged surfaces can stimulate this pathway. The intrinsic pathway normally requires platelet activation in order to assemble a tenase complex involving factors VIIIa, IXa, and X. The activation process is linked to the inositol triphosphate (IP3) pathway and involves degranulation and myosin 1c kinase activation in order to change the platelet shape to ultimately allow adherence.

Clotting may alternatively be activated via the extrinsic coagulation cascade pathway which requires a tissue factor from the surface of extravascular cells. The extrinsic pathway involves complex formation of coagulation factors V, VII, and X. The chief inducer of coagulation in vivo is Tissue Factor (TF), a 47 kDa glycoprotein. The only cells capable of expressing TF in the bloodstream are endothelial cells and monocytes. By contrast, many cells outside the bloodstream, including adventitial fibroblasts, constitutively express TF and thus form an “extravascular envelope” capable of initiating coagulation in the event of a disruption in vascular integrity.

The final stages of the cascade are common to both pathways which involves a tenase complex, the activating complex. Tenase is a contraction of “ten” and the suffix “-ase”, signifying that the complex activates its substrate (inactive factor X) by cleaving it. Intrinsic tenase complex contains the active factor IX (IXa), its cofactor factor VIII (VIIIa), the substrate (factor X), and they are activated by negatively charged surfaces (such as glass, active platelet membrane, sometimes cell membrane of monocytes, or red blood cell membranes). Extrinsic tenase complex is made up of tissue factor, factor VII, the substrate (factor X) and Ca²⁺ as an activating ion.

Activation of factor X, to factor Xa, through either the extrinsic or the intrinsic pathway, leads to the proteolytic conversion of prothrombin to thrombin which, in turn, activates the initiation of the formation of a clot and activates platelets. Factor VIII then catalyzes a transglutaminase reaction to crosslink the fibrin monomers to form a crosslinked network.

The crosslinked fibrin multimers in a clot are broken down to soluble polypeptides by plasmin, a serine protease. Plasmin can be generated from its inactive precursor plasminogen and recruited to the site of a fibrin clot in two ways, by interaction with tissue plasminogen activator at the surface of a fibrin clot, and by interaction with urokinase plasminogen activator at a cell surface. The first mechanism appears to be the major one responsible for the dissolution of clots within blood vessels. The second, although capable of mediating clot dissolution, may normally play a major role in tissue remodeling, cell migration, and inflammation.

Clot dissolution is regulated in two ways. First, efficient plasmin activation and fibrinolysis occur only in complexes formed at the clot surface or on a cell membrane; proteins free in the blood are inefficient catalysts and are rapidly inactivated. Second, both plasminogen activators and plasmin itself are inactivated by specific serpins, proteins that bind to serine proteases to form stable, enzymatically inactive complexes. Pharmacologically, the clot buster tissue plasminogen activator (TPA) and streptokinase or urokinase are used to activate this internal fibrinolytic mechanism.

Medical Conditions

The methods and the device of the invention as herein described may be used for the detection of rheological changes of various liquids, in particular blood samples, for the diagnosis of coagulation, thrombotic disorders, and thrombotic disorders as a result of disease, e.g., sepsis and disseminated intravascular coagulation (DIC), Hemophilia A, Hemophilia B, Hemophilia C, Congenital deficiency of other clotting factors Factor XIII deficiency, Von Willebrand's disease, hemorrhagic disorder due to intrinsic anticoagulants, defibrination syndrome, acquired coagulation factor deficiency, coagulation defects, other, purpura and other hemorrhagic conditions, allergic purpura, Henoch-Schönlein purpura, thrombocytopenia, immune thrombocytopenic purpura, idiopathic thrombocytopenic purpura, secondary thrombocytopenia, sickle cell anemia, and non-specific hemorrhagic conditions.

The cardiovascular system requires tightly regulated hemostasis. Excessive clotting may cause venous or arterial obstructions, while failure to clot may cause excessive bleeding; both conditions lead to deleterious clinical situations. In most human subjects, the clotting balance is more or less static. However, there are many different clinical parameters (such as hereditary disorders, disease states, therapeutic drugs, or pharmacological stressors) that can alter hemostasis and lead to cardiovascular malfunction.

There are many different known coagulation disorders that are a result of non-functional clotting factors, such as hemophilia (factors VIII (hemophilia A), IX (hemophilia B), XI (hemophilia C)), Alexander disease (factor VII deficiency), prothrombin deficiency (factor II deficiency), Owren's disease (factor V deficiency), Stuart-Prower deficiency (factor X deficiency), Hageman factor deficiency (factor XII deficiency), fibrinogen deficiency (factor I deficiency), and von Willebrand's disease.

The activation of the coagulation cascades appears to be an essential component in the development of multi-organ failure that occurs in end-stage sepsis. Current therapies for sepsis specifically target these cascades for modulation of the progression of the end stages and to prevent organ failure.

The methods and devices of the invention may be used to determine the hematocrit of a blood sample. The hematocrit is a measure of the percent volume occupied by red blood cells in a subject's blood. For newborns, hematocrit levels are 55% to 68%; for babies one (1) week of age hematocrit levels are typically 47% to 65%; for babies one (1) month of age hematocrit levels are typically 37% to 49%; for babies three (3) months of age hematocrit levels are typically 30% to 36%; for babies one (1) year of age hematocrit levels are typically 29% to 41%; for children ten (10) years of age hematocrit levels are typically 36% to 40%; and for adult men, hematocrit levels are typically, 42% to 54% or 33%-43% or 38.8% to 50%; and for adult women, hematocrit levels are typically 38% to 46% or 31%-39% or 34.9% to 44.5%. Abnormal hematocrit values are usually found above or below these ranges. The hematocrit depends on both the number of red blood cells in a sample and the size of the red blood cells. The measurement of hematocrit may be useful in establishing a variety of physiological conditions in a subject. Thus, the methods of the invention may be used in the diagnosis of any condition associated with a lower than normal hematocrit or a higher than normal hematocrit. A lower than normal hematocrit may be indicative of anemia, sickle cell anemia, internal bleeding, loss of red blood cells, malnutrition, nutritional deficiencies (e.g., iron, vitamin B12, or folate deficiencies), or over hydration. A higher than normal hematocrit may be indicative of congenital heart disease, dehydration, erythrocytosis, pulmonary fibrosis, polycythemia rubra vera, or abuse of the drug erythropoietin.

The methods of the invention can be used to monitor factors and related coagulopathies associated with disease, disorder or dysfunction such as cancer, autoimmune disorders, lupus erythematosus, Crohn's disease, multiple sclerosis, amyotrophic lateral sclerosis, deep vein or arterial thrombosis, obesity, rheumatoid arthritis, Alzheimer's disease, diabetes, cardiovascular disease, congestive heart failure, myocardial infarction, coronary artery disease, endocarditis, stroke, emboli, pneumonia, ulcerative colitis, inflammatory bowel disease, chronic obstructive pulmonary disease, asthma, infections, transplant recipients, liver disease, hepatitis, pancreas disease and disorders, renal disease and disorders, endocrine disease and disorders, obesity, diseases or disorders associated with thrombocytopenia, and medical (stents, implants, major surgery, joint replacements, pregnancy) or therapeutic (cancer chemotherapy) induced coagulopathy/ies, and risk factors such as heavy smoking, heavy alcohol consumption, sedentary lifestyle. The methods of the invention may also be used to evaluate genomic and proteomic changes that affect coagulation and blood properties.

The methods of the invention can also be used to monitor patients being undergoing anti-coagulant and/or anti-platelet therapy. Examples of anti-thrombotics (e.g., thrombolytics, anticoagulants, and antiplatelet drugs) that can be monitored using the methods of the invention include, without limitation, vitamin K antagonists such as acenocoumarol, clorindione, dicumarol, diphenadione, ethyl biscoumacetate, phenprocoumon, phenindione, tioclomarol, and warfarin; heparin group (platelet aggregation inhibitors) such as antithrombin III, bemiparin, dalteparin, danaparoid, enoxaparin, heparin, nadroparin, parnaparin, reviparin, sulodexide, and tinzaparin; other platelet aggregation inhibitors such as abciximab, acetylsalicylic acid (aspirin), aloxiprin, beraprost, ditazole, carbasalate calcium, cloricromen, clopidogrel, dipyridamole, epoprostenol, eptifibatide, indobufen, iloprost, picotamide, prasugrel, ticlopidine, tirofiban, treprostinil, and triflusal; enzymes such as alteplase, ancrod, anistreplase, brinase, drotrecogin alfa, fibrinolysin, procein C, reteplase, saruplase, streptokinase, tenecteplase, and urokinase; direct thrombin inhibitors such as argatroban, bivalirudin, desirudin, lepirudin, melagatran, and ximelagatran; other antithrombotics such as dabigatran, defibrotide, dermatan sulfate, fondaparinux, and rivaroxaban; and others such as citrate, EDTA, and oxalate.

Sepsis and Disseminated Intravascular Coagulation

The methods and devices of the invention can be used to assess the hemostatic condition of subjects suffering from sepsis or disseminated intravascular coagulation.

In sepsis, an overwhelming inflammatory response causes extensive collateral damage to the host's microcirculation. Damage to the endothelium exposes tissue factor and in sepsis, which may occur on a large scale. Tissue factor, in turn, binds to activated factor VII. The resulting complex activates factors IX and X. Factor X converts prothrombin into thrombin, which cleaves fibrinogen into fibrin, inducing the formation of a blood clot. At the same time, the fibrinolytic system is inhibited. Cytokines and thrombin stimulate the release of plasminogen-activator inhibitor-1 (PAI-1) from platelets and the endothelium. When a clot forms in the human body, it is ultimately broken down by plasmin, which is activated by tissue plasminogen activator (TPA). PAI-1 inhibits TPA. Consequently, subjects suffering from severe sepsis are treated with an anticoagulant such as protein C (blood coagulant factor XIV).

Disseminated intravascular coagulation (DIC) is a complex systemic thrombohemorrhagic disorder involving the generation of intravascular fibrin and the consumption of procoagulants and platelets. The resultant clinical condition is characterized by intravascular coagulation and hemorrhage. DIC is not an illness on its own but rather a complication or an effect of progression of other illnesses and is estimated to be present in up to 1% of hospitalized patients. DIC is always secondary to an underlying disorder and is associated with a number of clinical conditions, generally involving activation of systemic inflammation. DIC has several consistent components including activation of intravascular coagulation, depletion of clotting factors, and end-organ damage. DIC is most commonly observed in severe sepsis and septic shock. Indeed, the development and severity of DIC correlates with mortality in severe sepsis. Although bacteremia, including both gram-positive and gram-negative organisms, is most commonly associated with DIC, other infections including viral, fungal, and parasitic infections may cause DIC. Trauma, especially neurotrauma, is also frequently associated with DIC. DIC is more frequently observed in those patients with trauma who develop the systemic inflammatory response syndrome. Evidence indicates that inflammatory cytokines play a central role in DIC in both trauma patients and septic patients. In fact, systemic cytokine profiles in both septic patients and trauma patients are nearly identical.

DIC exists in both acute and chronic forms. DIC develops acutely when sudden exposure of blood to procoagulants occurs, including tissue factor (tissue thromboplastin), generating intravascular coagulation. Compensatory hemostatic mechanisms are quickly overwhelmed, and, as a consequence, a severe consumptive coagulopathy leading to hemorrhage develops. Abnormalities of blood coagulation parameters are readily identified, and organ failure frequently occurs in acute DIC. In contrast, chronic DIC reflects a compensated state that develops when blood is continuously or intermittently exposed to small amounts of tissue factor. In chronic DIC, compensatory mechanisms in the liver and bone marrow are not overwhelmed, and there may be little obvious clinical or laboratory indication of the presence of DIC. Chronic DIC is more frequently observed in solid tumors and in large aortic aneurysms.

Exposure to tissue factor in the circulation occurs via endothelial disruption, tissue damage, or inflammatory or tumor cell expression of procoagulant molecules, including tissue factor. Tissue factor activates coagulation by the extrinsic pathway involving factor VIla. Factor Vlla has been implicated as the central mediator of intravascular coagulation in sepsis. Blocking the factor Vlla pathway in sepsis has been shown to prevent the development of DIC, whereas interrupting alternative pathways did not demonstrate any effect on clotting. The tissue factor-Vila complex then serves to activate thrombin, which, in turn, cleaves fibrinogen to fibrin while simultaneously causing platelet aggregation. Evidence suggests that the intrinsic (or contact) pathway is also activated in DIC, while contributing more to hemodynamic instability and hypotension than to activation of clotting. Decreased levels of antithrombin correlate with elevated mortality in patients with sepsis. Thrombin generation is usually tightly regulated by multiple hemostatic mechanisms. Antithrombin function is one such mechanism responsible for regulating thrombin levels. However, due to multiple factors, antithrombin activity is reduced in patients with sepsis. First, antithrombin is continuously consumed by ongoing activation of coagulation. Moreover, elastase produced by activated neutrophils degrades antithrombin as well as other proteins. Further antithrombin is lost to capillary leakage. Lastly, production of antithrombin is impaired secondary to liver damage resulting from under-perfusion and microvascular coagulation.

Tissue factor pathway inhibitor (TFPI) depletion is evidence in subjects with DIC. TFPI inhibits the tissue factor-Vila complex. Although levels of TFPI are normal in patients with sepsis, a relative insufficiency in DIC is evident. TFPI depletion in animal models predisposes them to DIC, and TFPI blocks the procoagulant effect of endotoxin in humans. The intravascular fibrin produced by thrombin is normally eliminated via a process termed fibrinolysis. The initial response to inflammation appears to be augmentation of fibrinolytic action; however, this response soon reverses as inhibitors of fibrinolysis are released. High levels of PAI-1 precede DIC and predict poor clinical outcomes. Fibrinolysis cannot keep pace with increased fibrin formation, eventually resulting in under-opposed fibrin deposition in the vasculature.

Protein C, along with protein S, serves in important anticoagulant compensatory mechanisms. Under normal conditions, protein C is activated by thrombin and is complexed on the endothelial cell surface with thrombomodulin. Activated protein C combats coagulation via proteolytic cleavage of factors Va and VIIIa. However, cytokines (e.g., tumor necrosis factor α (TNF-α) and interleukin 1 (IL-1)) produced in sepsis and other generalized inflammatory states largely incapacitate the protein C pathway. Inflammatory cytokines down-regulate the expression of thrombomodulin on the endothelial cell surface. Protein C levels are further reduced via consumption, extravascular leakage, reduced hepatic production, and by a reduction in freely circulating protein S.

Inflammatory and coagulation pathways interact in substantial ways. Many of the activated coagulation factors produced in DIC contribute to the propagation of inflammation by stimulating endothelial cell release of proinflammatory cytokines. Factor Xa, thrombin, and the tissue factor-Vila complex have each been demonstrated to elicit proinflammatory action. Furthermore, given the anti-inflammatory action of activated protein C, its impairment in DIC contributes to further dysregulation of inflammation.

Components of DIC include: exposure of blood to procoagulant substances; fibrin deposition in the microvasculature; impaired fibrinolysis; depletion of coagulation factors and platelets (consumptive coagulopathy); organ damage and failure. DIC may occur in 30-50% of patients with sepsis.

The methods and devices of the invention may find use in monitoring subjects with a variety of DIC-associated conditions such as: sepsis/severe infection; trauma (neurotrauma); organ destruction; malignancy (solid and myeloproliferative malignancies); severe transfusion reactions; rheumatologic illness; obstetric complications (amniotic fluid embolism, abruptio placentae, hemolysis, retained dead fetus syndrome); vacular abnormalities (Kasabach-Merritt syndrome, aneurysms); hepatic failure; toxic reactions, transfusion reactions, and transplant rejections. Similarly, the invention may be used with respect to subjects having hemostatic conditions characterized by acute DIC associated with bacterial infections (e.g., gram-negative sepsis, gram-positive infections, or rickettsial), viral infections (e.g., associated with HIV, cytomegalovirus, varicella, or hepatitis), fungal infections, parasitic infection (e.g., malaria), malignancy (e.g., acute myelocytic leukemias), obstetric conditions (e.g., eclampsia placental abruption or amniotic fluid embolism), trauma, burns, transfusion, hemolytic reactions, or transplant rejection.

The NMR-based methods of the invention may be used to monitor any and all of the blood-related conditions described above. Time-domain relaxometry, particularly T2 relaxation measurements, can be used to measure a change in the clotting state of a sample. This measurement relies on measuring NMR parameters of the hydrogen nuclei that are sensitive to changes in the macroscopic clotting state of the sample. Most of the hydrogen nuclei are in the bulk water solvent, but an appreciable fraction of them are in the biological macromolecules and cells and platelets in the sample. As such, the measurement of the average NMR signal from all hydrogen nuclei can be conducted such that the signal changes in an appreciable manner when the clotting state of the sample changes for any of the clinical reasons described above. The NMR measurement can be a T2 relaxation measurement, or an “effective” T2 relaxation measurement (e.g., a T2 relaxation measurement where the parameters of the signal acquisition are such that they are set for optimal readout of the clotting event and not for the most accurate measurement of a T2 relaxation value). Other “time domain” relaxation measurement methods can be applied to measure changes in clotting behaviors. These may include time-domain free-induction decay analyses amongst other measurements. Any of the NMR time domain measurements described herein can be acquired in a repeated fashion to get a dynamic read-out of the NMR signal over the course of time as the clotting or dissolution properties of the sample change.

Subjects Having Normal and Abnormal Hemostatic Profiles

The methods of the invention can be used to discriminate between subjects having normal and abnormal hemostatic profiles. For example, the NMR relaxation parameter value and/or T2 signature characteristic of normal and abnormal hemostatic profiles can be determined and used in the differential diagnosis of a subject, such as a subject having sickle cell anemia. Abnormal hemostatic profiles can include profiles for subjects sharing a common deficiency in one or more clotting factors, clotting cofactors, and/or regulatory proteins (e.g., factor XII, factor XI, factor IX, factor VII, factor X, factor II, factor VIII, factor V, factor III (tissue factor), fibrinogen, factor I, factor XIII, von Willebrand factor, protein C, protein S, thrombomodulin, and antithrombin III, among others). The distinction of normal versus abnormal subjects can be indicative of disease states that are not from factor deficiencies.

A deficiency in antithrombin is seen in approximately 2% of patients with venous thromboembolic disease. Inheritance occurs as an autosomal dominant trait. The prevalence of symptomatic antithrombin deficiency ranges from 1 per 2000 to 1 per 5000 in the general population. Deficiencies results from mutations that affect synthesis or stability of antithrombin or from mutations that affect the protease and/or heparin binding sites of antithrombin. The methods of the invention can be used to discriminate between normal subjects and subjects having a deficiency in antithrombin.

A deficiency in factor XI confers an injury-related bleeding tendency. This deficiency was identified in 1953 and originally termed hemophilia C. Factor XI deficiency is very common in Ashkenazic Jews and is inherited as an autosomal disorder with either homozygosity or compound heterozygosity.

The methods of the invention can be used to discriminate between normal subjects and subjects having a deficiency in factor XI.

Von Willebrand disease (vWD) is due to inherited deficiency in von Willebrand factor (vWF). vWD is the most common inherited bleeding disorder of humans. Deficiency of vWF results in defective platelet adhesion and causes a secondary deficiency in factor VIII. The result is that vWF deficiency can cause bleeding that appears similar to that caused by platelet dysfunction or hemophilia. vWD is an extremely heterogeneous disorder that has been classified into several major subtypes. Type I vWD is the most common and is inherited as an autosomal dominant trait. This variant is due to simple quantitative deficiency of all vWF multimers. Type 2 vWD is also subdivided further dependent upon whether the dysfunctional protein has decreased or paradoxically increased function in certain laboratory tests of binding to platelets. Type 3 vWD is clinically severe and is characterized by recessive inheritance and virtual absence of vWF. The methods of the invention can be used to discriminate between normal subjects and subjects having a deficiency in von Willebrand factor.

Several cardiovascular risk factors are associated with abnormalities in fibrinogen. Elevated plasma fibrinogen levels have been observed in patients with coronary artery disease, diabetes, hypertension, peripheral artery disease, hyperlipoproteinemia and hypertriglyceridemia. In addition, pregnancy, menopause, hypercholesterolemia, use of oral contraceptives and smoking lead to increased plasma fibrinogen levels. There are inherited disorders in fibrinogen, including afibrinogenemia (a complete lack of fibrinogen), hypofibrinogenemia (reduced levels of fibrinogen) and dysfibrinogenemia (presence of dysfunctional fibrinogen). Afibrinogenemia is characterized by neonatal umbilical cord hemorrhage, ecchymoses, mucosal hemorrhage, internal hemorrhage, and recurrent abortion. The disorder is inherited in an autosomal recessive manner. Hypofibrinogenemia is characterized by fibrinogen levels below 100 mg/dL (normal is 250-350 mg/dL) and can be either acquired or inherited. The methods of the invention can be used to discriminate between normal subjects and subjects having abnormalities in fibrinogen.

Platelet Monitoring

Platelets play an integral role in hemostasis. Aggregation or contraction of platelets occurs shortly after activation. Fibrinogen mediates the processes of platelet aggregation and clot retraction in vitro and plays a role in maintaining hemostasis in vivo. In vitro platelet activity is often evaluated with light transmission aggregometry that is based on measuring the platelets ability to aggregate via binding to fibrinogen. However, their role in vitro is more often involved in binding to fibrin and mediating clot stiffening or clot contraction. Prior to this step, thrombin acts on fibrinogen which causes fibrinogen to polymerize into fibrin. Platelets bind to fibrin to form blood clots a vascular wound site. The contraction of platelets has been shown to drive the contraction (or retraction) and stiffening of clots. The clot contracts through the action of cytoplasmic motility proteins inside platelets. The contraction of the platelet leads to clot contraction because the platelet is bound to fibrin via IIb/IlIa receptors. This process leads to a reduction in the overall volume of the fibrin clot and extrusion of serum. Clots made from platelet rich plasma or whole blood generate a bulk contractile force that begins shortly after clotting and increases over time. The function of clot contraction is not fully understood, but it appears to reinforce hemostasis by forming a seal, promoting wound healing, and restoring blood flow by decreasing the area obstructed by intravascular clots. The understanding of platelet aggregation and contraction can aid in the diagnosis of many clotting disorders. It is thought that measurement of platelet activity via clot contraction may represent a measure of platelet activity that more closely mirrors what occurs physiologically. For further information on platelet monitoring, see Cines, Douglas B., et al. “Clot contraction: compression of erythrocytes into tightly packed polyhedra and redistribution of platelets and fibrin.” Blood 123:10 (2014): 1596-1603, and Skewis, Lynell R., et al. “T2 magnetic resonance: a diagnostic platform for studying integrated hemostasis in whole blood—proof of concept.” Clinical Chemistry 60:9 (2014): 1174-1182.

The methods and device of the invention can be used to determine platelet function and be compared to platelet aggregometry (see, e.g., Harris et al., Thrombosis Research 120:323 (2007)). There are generally two detection methods used in instruments with FDA clearance for performing platelet aggregometry: optical and impedance measurements. For example, the methods of the invention can be used to identify any platelet activity or diagnose any platelet dysfunction in a subject that may be measured by platelet aggregometry. Platelet aggregometry is a functional test performed on a whole blood or platelet-rich plasma sample. Generally, platelet aggregometry methods involve adding a platelet activator to the sample and measuring the induced platelet aggregation. Platelet aggregometry can be performed by immersing an electrode in the blood sample being tested. Platelets adhering to the probe form a stable monolayer. When an activator is added, platelet aggregates form on the electrode and increase the resistance to a current being applied across the electrode. The instrument monitors the change in electrical impedance, which reflects the platelet aggregation response. Aggregometry methods also include techniques based on monitoring the release of ATP from aggregating platelets by luminescence. Optical detection of platelet aggregation is based on the observation that, as platelets aggregate into large clumps, there is an increase in light transmittance. Different aggregation-inducing agents stimulate different pathways of activation and different patterns of aggregation are observed. The main drawback of the optical method is that it is typically performed on platelet rich plasma (PRP), necessitating the separation of platelets from red blood cells and adjustment of the platelet count to a standardized value.

As in platelet aggregometry, the methods of the invention may be used assess the platelet count from a blood sample of a subject or to diagnose a condition of thrombocytopenia (platelet count <150,000/μL) or thrombocytosis (platelet count >400,000/μL) in a subject. Such a diagnosis may be used as the basis of a decision to provide the subject with a platelet transfusion or an anticoagulant. Similarly, the methods of the invention may be used to evaluate the response of a subject to a platelet transfusion or an anticoagulant.

The platelet activity can be measured using a platelet activity metric. The platelet activity metric (PAM) is a feature extraction technique designed to quantify the extent of platelet activity measured by T2MR. During platelet induced clot contraction, the distance between RBCs entrapped in the fibrin clot decreases, and a serum phase separates from the remainder of the contracting fibrin clot. Due to the paramagnetic properties of hemoglobin in RBCs, the result is a faster relaxation of hydrogen nuclei in water molecules inside the clot (lower T2 time), and a slower relaxation of hydrogen nuclei in water molecules in the serum (higher T2 time). In particularly strong clots with significant RBC compaction, one or more additional peaks are detected in the clot, yielding two or more clot peaks. In effect, this T2MR method detects and identifies separate populations of water molecules, each with different T2 relaxation times. In addition to T2 times, T2MR also quantifies the relative molarity or volume fraction of each population, using the intensity component for each T2 time resolved from the fits of the raw CPMG.

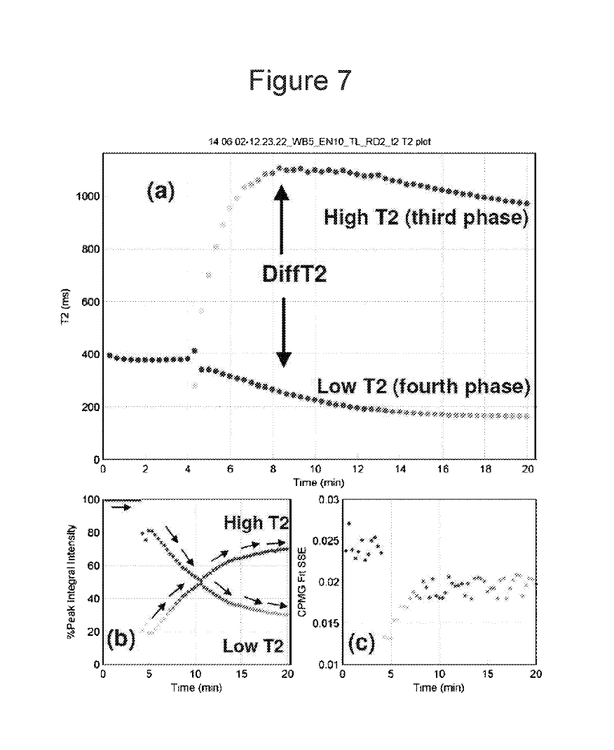

In one embodiment, the PAM incorporates only the T2 values of the serum and clot phase. To quantify the magnitude of separation between the serum and clot phases, the difference between the serum and clot is defined as the “DiffT2”, where DiffT2=T2serum−T2clot, where DiffT2 may use any number of T2 peaks for each water population. A plot depicting DiffT2 is shown in FIG. 7a. For instance, T2clot may be from one, two or any number of detected T2 times, defined as T2_clotA, T2_clotB, etc. Using this definition, in one embodiment, the PAM may be defined as the DiffT2 at a specific time (PAMa), or the time integral (PAMb). See Table 1.

TABLE 1
Platelet Activity Metrics

| PAM | Defined as (=to) |
|---|---|
| PAMa | DiffT2 |
| PAMb | ∫DiffT2 |
| PAMc | Intensity_serum |
| PAMd | Intensity_clotA |
| PAMe | Intensity_clotB |
| PAMf | ∫Intensity_serum |
| PAMg | ∫Intensity_clotA |
| PAMh | ∫Intensity_clotB |
| PAMi | DiffT2 × intensity_serum |
| PAMj | ∫DiffT2 × intensity_serum |
| PAMk | DiffT2 × intensity_serum × time |
| PAMl | ∫DiffT2 × intensity_serum × time |
| PAMm | DiffT2 × intensity_serum × (1/time) |
| PAMn | ∫DiffT2 × intensity_serum × (1/time) |
| PAMo | DiffT2 × intensity_serum × (1 + intensity_clotB) |
| PAMp | ∫ DiffT2 × intensity_serum × (1 + intensity_clotB) |
| PAMq | DiffT2 × intensity_serum × time × (1 + intensity_clotB) |
| PAMr | ∫DiffT2 × intensity_serum × time × (1 + intensity_clotB) |
| PAMx | DiffT2 × intensity_serum × (1/time) × (1 + intensity_clotB) |
| PAMt | ∫DiffT2 × intensity_serum × (1/time) × (1 + intensity_clotB) |
| PAMu | ∫(1/T2Clot) |
| PAMv | ∫(1/T2Clot) × time |
| PAMw | ∫((1/T2Clot) − (1/T2Serum))/(1/T2Serum) |
| PAMy | ∫(((1/T2Clot) − (1/T2Serum))/(1/T2Serum)) × time |

In another embodiment, the PAM does not use T2 times, but rather employs the relative intensities of each peak, either at a specific time, or the time integral, as seen in PAMc-PAMh in Table 1. Other combinations of the above are also possible, including ratios of one intensity versus another. In other embodiments, the PAM incorporates combinations of T2 time and intensity. The principal being that additional sensitivity to platelet function may be attained utilizing combinations of T2 time and intensity, compared to either one alone. Specifically, clots which show very high T2 values and intensity in the serum phase may be different than either high T2 or intensity alone. A PAM value that incorporates both T2 and intensity may have additional diagnostic sensitivity. In one embodiment, the PAM is a combination in the following form, as either a single time point or time integral as seen in PAMi or PAMj in Table 1. Other embodiments are also possible, which incorporate either T2serum, T2clot, or intensities of serum or clot formation. In another embodiment, the PAM is weighted by time, so that activity at later time accounts for more of the total signal than earlier time, and in an alternative embodiment, activity earlier accounts for more of the total signal, in the following form, as either a single time point or time integral as seen in PAMk through PAMn in Table 1. In still other embodiments, the PAM is weighted by the intensity of the lower clot peak (intensity clotB), to account for the additional RBC compaction that the lower clot peak represents. Expressions that represent these scenarios are present in PAMo-PAMt in Table 1. In still other embodiments, the PAM incorporates the reciprocal of the T2 value of the clot (T2clot), serum (T2serum), or both. These signals are then integrated with experiment time, to account for the kinetics of platelet contraction. These PAM metrics may also be weighted by experiment time, to provide more emphasis to signals that form later in the process. The PAM may also be calculated as the difference in reciprocal T2 values between the clot and serum, normalized to the serum reciprocal T2 value. Expressions that represent these scenarios are present in PAMu—PAMv and PAMy in Table 1. Other approaches are also possible, which incorporate either T2serum, T2clot, or intensities of serum or clot formation. The PAM incorporates the T2 relaxation time and/or intensity (relative molarity) of the serum and/or clot phases to quantify the extent of platelet contraction in whole blood. By incorporating either or both of these signals, and potentially with time integrals, dose responses of platelet inhibitors can measured, and differences between normal or abnormal platelet responses, or activated and inhibited samples, may be resolved.

T2MR Multiplexed Hemostasis Panel

Unlike other hemostasis measurement tools, T2MR enables multiplexed hemostasis measurements for different hemostasis parameters that are normally analyzed as single assays on separate and distinct platforms. These multiplexed measurements enable novel combination of diagnostic assays that may not been possible on a single instrument or assay panel, especially at the point of care. The T2MR based assay panel can be conveniently used at point of care and in a hospital laboratory.

A specific T2MR assay panel has been designed, among other applications, to aid in the diagnosis and treatment of patients suffering from trauma, undergoing treatment in the operating room, or who have an underlying complicated disease or disorder that requires a multifaceted diagnostic analysis. The information provided by these assays aids in the appropriate decisions for transfusion, administration of therapeutics to restore hemostasis, and other medical interventions.

The T2MR multiplexed hemostasis panel allows for measurement of multiple categories of hemostasis parameters in whole blood, eliminating the need for time-consuming sample preparation. These include (1) clotting time parameters, (2) hematocrit or hemoglobin levels, (3) global platelet activity/inhibition measurements, (4) fibrinogen measurements, and (5) fibrinolysis measurements. These parameters are multiplexed in the diagnostic panel. Multiplexing can take place as either (1) a single-reaction multiplexed result, (2) measurements obtained in parallel from multiple aliquots of the same sample, or (3) measurements obtained in succession on the same instrument with multiple aliquots of the sample.

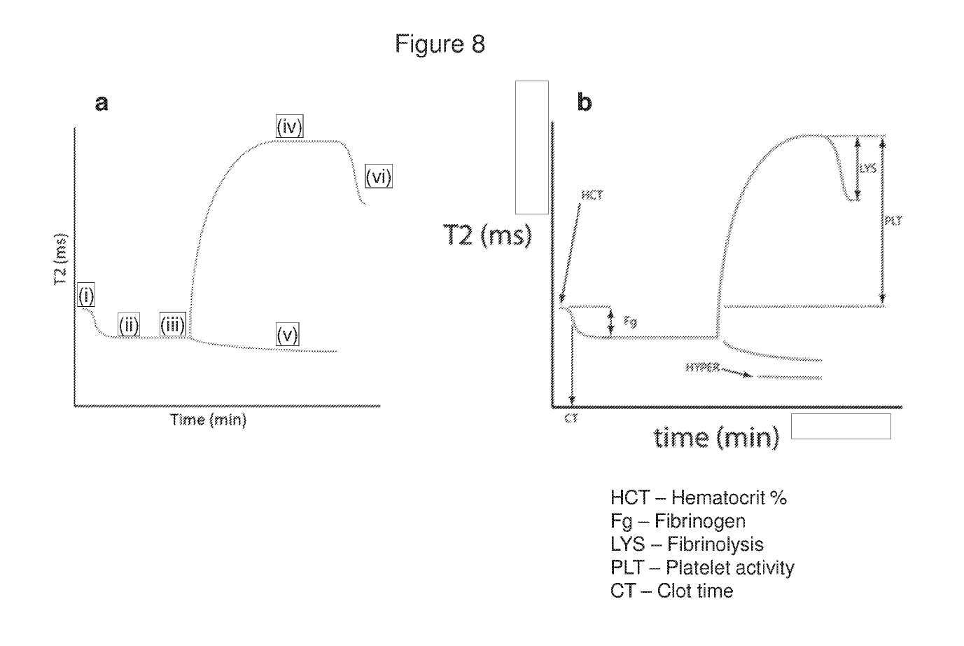

The multiplexed panel can allow the user to obtain multiple T2 measurements that correspond to various coagulable conditions. These can be visualized or analyzed in a single diagram that combines multiple T2 measurements. One such diagram is found in FIGS. 8a and 8b. FIG. 8a depicts the T2MR signature of a citrated blood sample mixed with an extrinsic clotting pathway activator. Phase 1 of the plot corresponds to a uniform distribution of water throughout a single homogenous liquid environment prior to clot formation. The T2MR value at Phase 2 shifts downward upon clot formation due to the formation of a single uniform fibrin clot distributed throughout the sample. After platelets are activated, the single T2MR value splits into two values that corresponds to a serum-like micro environment shown at part (phase 3) and clot micro environment containing loosely bound red blood cells (phase 4). When clot lysis occurs, the T2MR value for the serum-like micro environment decreases due to clot degradation and release of red cells (fifth phase).

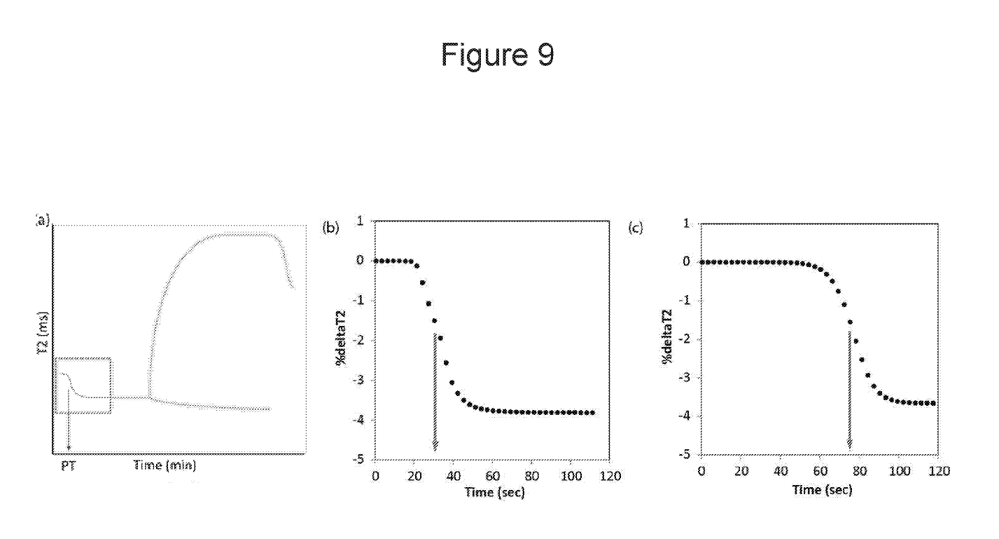

The different transitions visible in the single diagram can be utilized to determine the levels of hematocrit, fibrinogen, clot time, platelet activity and fibrinolysis. (See FIG. 8b.) T2MR measurement of clotting time is similar to clotting time measurements on other platforms in that a physical parameter of the sample that corresponds to fibrin formation is being monitored as a function of time after the addition of an activator of coagulation. FIGS. 9a-9c show T2MR data collected immediately after the addition of an activator and depicts how clotting time is derived.

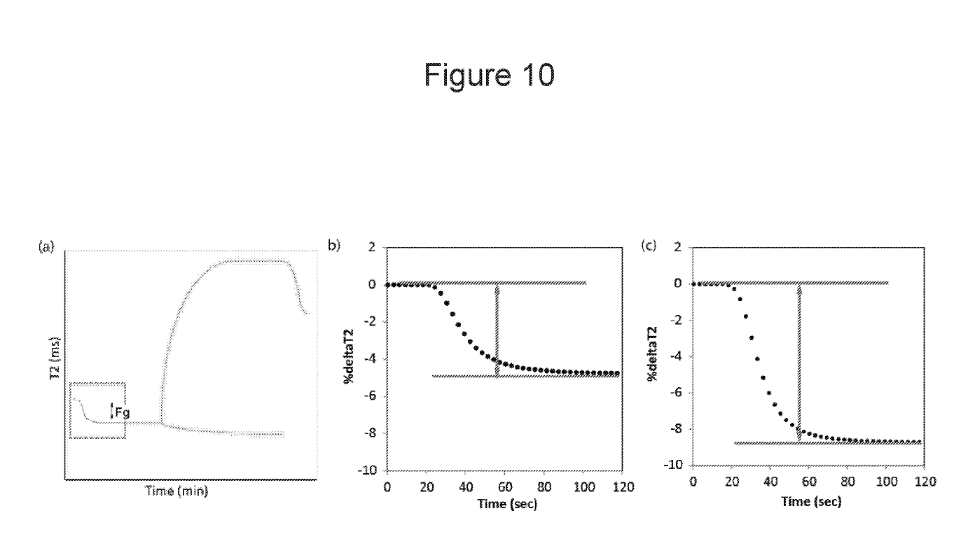

Activation of whole blood with extrinsic or intrinsic activators leads to thrombin production and the conversion of fibrinogen to fibrin. The amount of fibrinogen in the sample can be derived from the magnitude of the change in the T2MR signal upon clot formation; the higher the concentration of fibrinogen the larger the change in T2MR signals. For an illustration of how a fibrinogen measurement can be made with T2MR after the addition of an activator, see FIGS. 10a-10c.

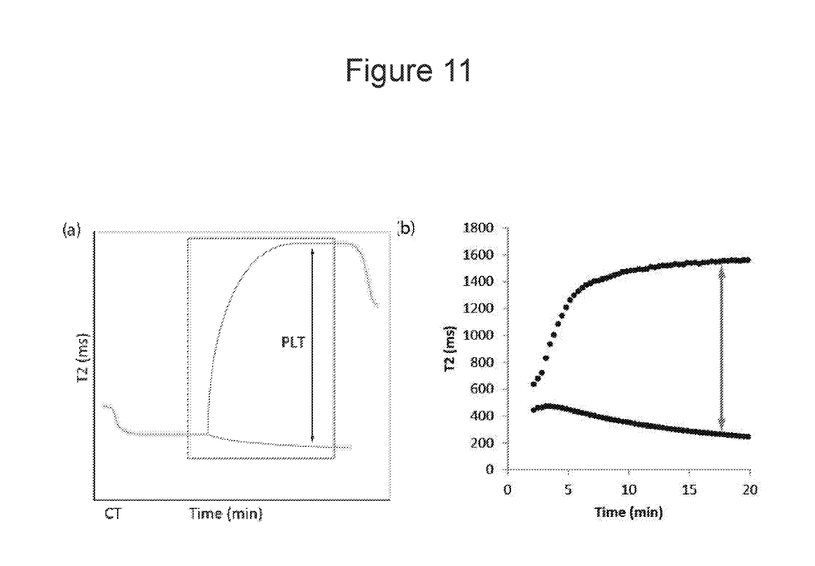

After addition of an activator to a whole blood sample, thrombin is generated and the platelets become activated leading to platelet-induced clot contraction. As the process of clot contraction progresses, water is extruded from the contracting clot into a separate serum compartment within the sample producing two distinct microenvironments, exemplified by the emergence of a second T2MR peak with a distinct T2 relaxation time (See FIGS. 11a and 11b). The lower peak is water associated with the clot, while the upper peak is water from the serum phase extruded from the contracting clot. As the platelets contract the clot, the T2MR signal of the upper peak increases until it levels off at the T2 value of serum. The “Platelet Function” can be derived from the relative T2MR signals of the upper and lower peaks.

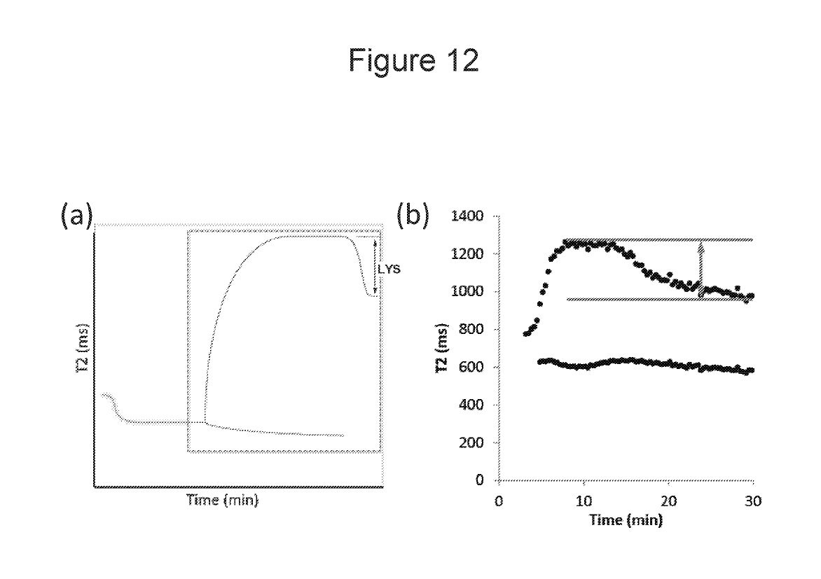

Fibrinolysis is the breakdown of blood clots due to the cleavage of fibrin by plasmin. Fibrinolysis can be measured with T2MR by measuring the change in the serum-associated (upper) T2MR signal after platelet induced clot contraction, as shown in FIGS. 12a and 12b, where a normal blood sample was spiked with tissue plasminogen activator (tPA) prior to activation. After clot formation and platelet mediated clot contraction, plasmin begins to cleave the fibrin strands, which results in a decrease in the upper peak as erythrocytes are released from the clot.

The T2MR coagulopathy panel measures multiple clinical parameters that are important for the management of patients that have experienced or are suspected of trauma, including surgical trauma.

Assessing these parameters for patients who have experienced or are suspected of trauma is valuable for two reasons: 1) diagnosing acute coagulopathy, which is often caused by trauma, and 2) directing appropriate therapy for patients that are in need of transfusion products. Trauma may occur in many settings: 1) accidents—auto and otherwise, 2) combat, 3) results of violent acts, including gunshots, 4) birth, 5) sporting events, 6) surgery, and any event that may lead to blunt or penetrating wounds. Given the importance of identifying trauma induced coagulopathy early to assist with improved outcomes, identifying these factors is valuable at the point of trauma (battlefield, site of injury—sporting event, auto accident, point of gunshot wound, operating room), along the path towards treatment (medivac, helicopter, ambulance), at the point of hospital admission (trauma triage center, emergency room), or in a centralized setting (hospital laboratory). There are many causes and mechanisms leading to coagulopathy as a result of trauma (see Hess et al., J. Trauma 65:748 (2008), incorporated herein by reference). For example, there are known clinical syndromes occurring after trauma: dilutional coagulopathy, the fatal triad of shock, acidosis and hypothermia, and acute coagulopathy of trauma-shock or ACoTS. While trauma leads to a majority of coagulopathies, it is also known that coagulopathy can be associated with other disease/disorders, medications, and genetic predispositions.

This T2MR comprehensive panel can identify an acute coagulopathy, which will provide the information necessary to prompt an intervention, while the specific data will also direct the appropriate transfusion product. For instance, low hematocrit may lead toward red blood cell replacement, low fibrinogen may lead to fibrinogen treatment or fresh frozen plasma (FFP) administration, abnormal clot time may lead to administration of clotting factors or FFP, and abnormal platelet activity will suggest a platelet transfusion or appropriate medication. Because the effect of trauma on the coagulation state on a specific patient is unknown, each measured parameter may be used individually or in combination with all others. Based on the results of factor deficiency, abnormal activity, or other abnormalities in results, the specific therapy may be chosen. A broad assessment of clotting time, hematocrit levels, fibrinogen levels and platelet activity, along with other factors will provide the most appropriate transfusion decisions or therapeutic actions, and algorithms will vary based on clinical evidence; however, a potential approach is described in Maegle et al., World J. Emerg. Med. 1:12 (2010).

The T2MR primary assay configuration that enables a simple multiplexed coagulopathy panel is an assay configuration where a single activator is used to trigger clotting in whole blood. This activator not only triggers enzymatic coagulation and subsequent fibrin formation but also triggers platelet activity and subsequent clot contraction. The former allows for measurement of clotting time and defects or inhibition in the enzymatic cascade and the latter allows for measurement of platelet activity or inhibition as well as abnormally low fibrinogen levels. This multiplex capability distinguishes T2MR technology from thromboelastography, which is unable to measure PT-like clotting times and has been shown to be insensitive to measurement of fibrinolysis and is unable to easily measure and distinguish fibrin contribution to clot strength from platelet contribution to clot strength. Thromboelastography, as well as other platforms that measure fibrinogen in whole blood, suffer from interference from variations in the hematocrit level. This interference arises from variations in hematocrit impacting the overall volume of plasma and hence the amount of available fibrinogen in the measured sample. Additionally, measurement of the T2MR signals during the initial portion of the reaction enable determination of hematocrit. This allows for the auto-correction for hematocrit interference. Lastly, if desired, the measurements can be used to monitor deficiencies in fibrinolysis by monitoring signals after clot contraction or monitoring deficiencies in clot formation or contraction and in this case an additional reagent may be required in the assay, such as aprotinin to inhibit fibrinolysis and compare signature to those obtained in the absence of aprotinin. In essence, this clotting activator cocktail enables measurement of global hemostasis performance including hematocrit, enzymatic cascade, platelets, and fibrinogen.

The activation cocktail is an important feature of the T2MR coagulopathy panel. The activation cocktail may be composed of one or more activators, initiators, or compounds required for the reaction to occur. The activators employed can be global activators, or activators of the extrinsic or intrinsic pathways. In one embodiment it consists of a diluted PT reaction, which can be initiated with Innovin at a specific concentration. Innovin can be replaced more generally with any preparation of ‘tissue factor’ and lipid′; tissue factor to include both recombinant and non-recombinant, and lipid to include defined and undefined mixtures of phospholipids as well as Cephalin or any other natural substitute for platelet phospholipid. In another embodiment of the invention the activation cocktail initiates an EXTEM® reaction. In this embodiment, the standard ratio of EXTEM® to citrated whole blood can be used at volumes up to and including our sampling limit (typically 40-60 μL); for example, 2.4 μL EXTEM® reagent plus 35.3 μL citrated whole blood plus 2.4 μL STAR-TEM produces an activation signal within 10 minutes of the start of the reaction. In still another embodiment the activation cocktail initiates an activation of the intrinsic pathway. In this example, 34 μL of citrated whole blood can be mixed with 1 μL of the kaolin solution (Haemonetics) and 2 μL of 0.2M CaCl₂ solution. Normal sample activation is observed in 4 to 8 minutes). In this assay format, the kaolin+/−calcium solution may be in a dried form in the reactant tube.

In other embodiments, the activator can be tissue factor (recombinant human, rabbit, other animal, or other naturally derived or recombinant tissue factor), contact factor/aPTT reagent (such as celite, ellagic acid, or kaolin), tissue factor or contact factor activator plus cytochalasin D or abciximab (Reopro) (which blocks platelet activation), tissue factor+aprotinin (which blocks fibrinolysis), phospholipid, celite, or thrombin, among others. All of these activators can be combined with calcium for use with citrated blood. With this set of tests, the main pathways of clot formation and fibrinolysis can be measured. These activators can be combined with levels of protamine or heparinase as an aid in identifying heparin-mediated affects, or with ADP, arachidonic acid, serotonin, epinephrine, ristocetin, collagen, as well as the application of heat, cold, or vigorous mixing to cause platelet activation. Protamine will reverse the effects of heparin by binding quantitatively to it; the addition of 1 mg/ml protamine sulfate for each 100 IU/ml heparin will reverse the anticoagulant activity of heparin. A cocktail that contains protamine sulfate is expected to reverse the effects of heparin anticoagulation but have little effect on other mechanisms that inhibit clotting, such as factor deficiencies.

The activator selection is critical so that the desired sensitivities are achieved. For example, the activator will determine whether the clotting phenomenon being measured is a fast clotting test (like PT) or moderate (like PTT, or ACT) or slow clotting tests (like R—as in TEG parameter R). Additionally, the selection of the activator will determine to which anti-coagulants the clotting time measurement will be sensitive. These may include different combinations of warfarin, rivaroxaban, dabigatran, heparin, hirudin, or direct thrombin inhibitors, among others. The multiplex coagulopathy panel can be tailored to different patient states; for example, a cardiac bypass patient on unfractionated heparin (UFH) might require a cocktail with low-level protamine, whereas a patient infused with blood diluents may require a higher level of tissue factor to adequately monitor the state of the patient during surgery. The method can accommodate a variety of cocktail mixtures.

Optionally, the coagulopathy panel assay is carried out using a disposable reaction tube or cartridge that is loaded with reaction activators and sample using a pipette. In another embodiment the disposable has been pre-loaded with a dried or frozen activator cocktail and the sample is loaded with a pipette. In still another embodiment the disposable allows addition of blood in a non-quantified manner and the disposable combines the blood with the reactions in a controlled fashion. The mixing of the sample may be done either by the instrument, or by a technician before placement into the instrument. The disposable may, for example, have the ability to split the sample between different reaction tubes to permit either simultaneous or concurrent tests to be performed on the same sample of blood.

In one embodiment a panel is used with an extrinsic activator such as tissue factor. The clotting time T2MR measurement for normal blood can be 30-40 seconds. It can also be 8-12 seconds, 10-20 seconds, 10-40 seconds, 30-40 seconds, 30-50 seconds, 40-60 seconds, 50-60 seconds or 20-60 seconds. The international normalized ratio for normal blood can then be 0.8-1.2, or 0.87-1.13. The abnormal range of T2MR measurement of clotting time is above or below the normal range. The total range easily measured is 8-300 seconds though a measurement up to or beyond 1000 seconds could be performed with modified acquisition. If aprotinin is added in addition to tissue factor, in normal blood the clotting time is often between 27.9 and 41.6 seconds.

In another embodiment a coagulopathy panel is used with an intrinsic activator such as ellagic acid. The common range of clotting time is often between 100 and 120 seconds for normal blood. However, it can be 22-30 seconds, 30-44 seconds, 44-60 seconds, 60-80 seconds, 80-100 seconds, up to 2 times to 4 times the range of normal T2MR clotting times for the extrinsic activator. The abnormal clotting time ranges are above or below the normal range. The total range easily measured is 8-300 seconds though a measurement up to or beyond 1000 seconds could be performed with modified acquisition. If aprotinin is added in addition to intrinsic activator, clotting times can often be from 110.9-151.0 seconds in normal blood.

In another embodiment of the panel either extrinsic or intrinsic activator is added and fibrinogen is measured. Concentrations determined for fibrinogen in normal blood are 150-300 mg/dL, 200-400 mg/dL or 150-400 mg/dL. Abnormal blood is above or below the ranges for normal blood. The total range detectable is 75-600 mg/dL.

In one embodiment the clotting time is measured within 2 minutes and then the sample is allowed to clot and then go through fibrinolysis for an additional 45 or more minutes. The sample can be optionally removed after 2 minutes if only the clotting time and fibrinogen concentration are desired. Alternatively, the sample could be truncated after the reporting of the platelet activity measurement if the fibrinolysis measurement is not desired for a run time of 20-30 minutes.

In certain embodiments the clotting time is measured within 5 minutes and then the sample is allowed to clot and then go through fibrinolysis for an additional 45 or more minutes. The sample can be optionally removed after 5 minutes if only the clotting time and fibrinogen concentration are desired. Alternatively, the sample could be truncated after the reporting of the platelet activity measurement if the fibrinolysis measurement is not desired for a run time of 20-30 minutes.

In one embodiment the data from the panel can be acquired and fit using a mixed algorithm that has two different segments with different signal acquisition parameters and fitting algorithms. The signal acquisition settings of the first portion of the process utilizes 4,200 echoes of 180° pulses with an inter-echo delay of 499.2 μs that totals to a total CPMG collection time of 2.1 seconds. A recycle delay of 911 ms is used to yield a total dwell time of 3 seconds. This is repeated for a total experimental time of 2-5 minutes.

A mono exponential fit is used to fit the data acquired above to detect the values of T2 during the clotting of both whole blood and plasma. Once that the CPMG curves are deconvoluted using a mono-exponential fit, the raw T2 data is fitted using a population function with a 5 parameter logistic regression (PL) fitting procedure. The output can provide information about clot time, fibrinogen, and hematocrit. The clotting time parameter helps to detect the dynamics of the polymerization of fibrinogen into fibrin formation and the subsequent change in viscosity of the blood detected as a drop of the T2 value. The difference between the T2 value before and after the clot formation is proportional primarily to the fibrinogen level and secondarily to the amount of hematocrit. For this measurement a calibration curve and correction function is needed, as described herein. The initial T2 value is primarily proportional to the amount of hematocrit or hemoglobin present in the sample and secondarily proportional to the total protein in the sample.

The signal acquisition settings of the second portion of the process utilizes 14,000 echoes of 180° pulses and 0.5 ms of inter-echo delay that totals to 7 seconds of CPMG collection time, along with 3 seconds of recycle delay. This totals to a dwell time (time between T2 points) of 10 seconds. These are values are typically collected for a total of 30-45 minutes.

The data from the second portion of the process is fit using a multi-exponential fit. This second portion of the process is designed to detect the dynamics of platelet activity using the NLLS (non-linear least square) algorithm to fit CPMG decay curves. The program considers a linear combination of exponential functions up to three sets. The final fit yielding 1, 2, or 3 T2 and intensity values is assessed by evaluating which fit is minimized the most. This evaluation is determined by comparing the sum of square error (SSE) between three fits categorized as mono, bi- or tri-exponential. This approach utilizes the following parameters. Any bi-exponential fit that has only one T2 value greater than 30 ms is discarded and a mono-exponential is chosen instead. The bi-exponential fit is selected over the mono-exponential fit if the SSE of the two calculated T2 values is less than 75% of the SSE associated from the mono-exponential fit. Additionally, the difference between the T2 values of the bi-exponential fitting must be larger than 50 ms. The tri-exponential fit is selected over the bi-exponential fit if the SSE of the tri-exponential fit is less than 90% of the bi-exponential fitting SSE. If the SSE of the tri-exponential fit is >90% of the bi-exponential fit and the bi-exponential fit SSE is >75% of the mono-exponential fit then the mono-exponential fit is selected. If the SSE of the tri-exponential fit is >90% of the bi-exponential fit and the bi-exponential fit SSE is <75% of the mono-exponential fit but the difference between the calculated T2s of the bi-exponential fitting is less than 35 ms, the algorithm selects the mono-exponential fit.

Hematocrit Monitoring for Fibrinogen Measurement Correction

As mentioned above, hematocrit can interfere with the measurement of fibrinogen. The inherent sensitivity of T2MR for hematocrit can be used to correct for this interference. By enabling measurements in whole blood, the invention dramatically reduces sample volume and preparation time, by eliminating centrifugation steps. This enables faster diagnoses of fibrinogen abnormalities and therefore faster treatment times, which can be critical in certain trauma situations.

Fibrinogen is a key protein in the blood clotting cascade, and is the precursor protein to fibrin, which forms a stiff gel-like network that prevents further blood loss. Currently, the commercially available devices that measure fibrinogen concentration require plasma, likely because red blood cells (RBCs) interfere with the measurement. The specific reasons for this interference depend on the instrument, but the primary mechanism is the volume displacement from RBCs, since fibrinogen does not cross the cell barrier (Amukele, T. K., et. al. Am J Clin Pathol, 2010, 133: 550-556). As a result, the total moles of fibrinogen and other clotting factors in a given volume are a function of hematocrit, which can affect clot time and other parameters.

To provide an accurate measure of fibrinogen in whole blood, it is necessary to have an independent characterization of hematocrit, to compensate for the changes in available blood volume with cell density. Using T2MR relaxation measurements, the absolute relaxation time (T2) of water in whole blood tends to be primarily a function of hematocrit, whereas the change in T2 during the fibrin clot tends to be primarily a function of fibrinogen. While fibrinogen concentration is a significant contributor to the hematocrit measurement, and vice versa, by solving a system of equations, it is possible to correct for hematocrit and arrive at an accurate measure of fibrinogen in whole blood.

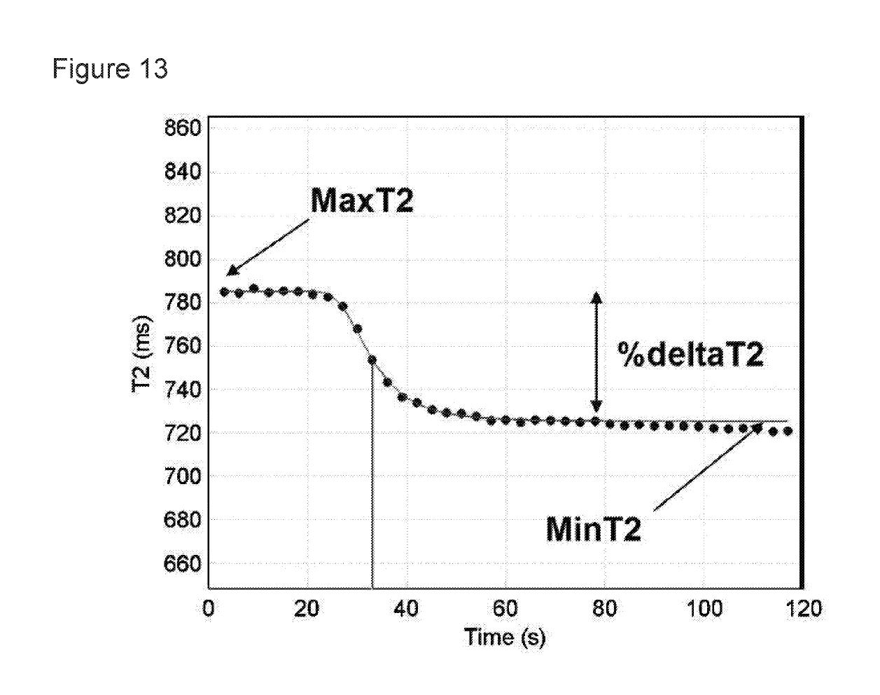

The T2MR measurements acquired are a “T2Max” and “% deltaT2” measurement, which primarily depend on hematocrit and fibrinogen, respectively. To acquire these data, blood clotting is initiated by adding calcium and one or a combination of several activators to citrated whole blood. The activators may include any agent that initiates the extrinsic, intrinsic, or other pathways, including tissue factor, ellagic acid, or kaolin, or may be agents that bypass these pathways and directly convert fibrinogen to fibrin, including batroxobin or ecarin. During fibrin formation in whole blood, the T2 values of water decreases in a rapid and non-linear fashion, due to differences in water diffusion interactions with the newly formed gel-like matrix (FIG. 13). To quantify the change, a 5 parameter logistic regression function is fit to the raw T2 data, and the following parameters are defined from the fit:

T2Max=maximum asymptote % deltaT2=(maximum asymptote−minimum asymptote)/(maximum asymptote) (9)

The T2Max and % deltaT2 parameters are acquired across a range of hematocrit and fibrinogen levels. From these values, a model can be developed using the general linear model, where in one embodiment, a linear (first order) equation is used to describe the T2Max and % deltaT2 values as a function of hematocrit and fibrinogen, and in other embodiments, second or third order equations are used. The order of model and incorporation of all terms are based on an analysis of variance (ANOVA) and calculation of predicted Pearson correlation coefficients (predicted R²) when individual data points are sequentially removed, the model is re-fit, and the adjusted sum of squared error is calculated from each re-fit (adj. SSE). Another approach the % deltaT2 and T2Max values are not incorporated to a model, but rather simply divided by each other. In effect, this simultaneously takes into account changes in fibrinogen and hematocrit and their effects on T2 and relaxivity.

For instance, in one embodiment, the system of equations is linear with an interaction term, in the following form:

% deltaT2=a*Hct+b*Fbn+c*Hct*Fbn+d (10)

T2Max=v*Hct+w*Fbn+x (11)

Where a, b, c, d, v, w and x are statistically significant coefficients that yield the highest predicted R² from the models, Fbn is fibrinogen concentration, and Hct is hematocrit level. These two equations (Eq. 10 and 11) are then solved for Fbn, excluding Hct.

In another embodiment, the system of equations is second order non-linear with interaction terms, in the following form:

% deltaT2=a*Hct²⁺b*Fbn²⁺c*Hct*Fbn+d*Hct+e*Fbn+f (12)

T2Max=v*Hct²⁺w*Hct*Fbn+x*Fbn+y*Hct+z (13)

Where a, b, c, d, e, f, v, w, x, y and z are statistically significant coefficients that yield the highest predicted R² from the models, Fbn is fibrinogen concentration, and Hct is hematocrit level. These two equations are then solved for Fbn, excluding Hct.

The order of models varies depending on the specific agonist used and final blood dilution relative to the agonist concentration. In one embodiment, the models incorporate other non-linear terms besides polynomials, including exponential, log, or exponents raised to various powers.

Using these systems of equations, the inputs are only % deltaT2 and T2Max from T2MR, and provide a more accurate fibrinogen level, due to the additional estimation and compensation from hematocrit using T2Max.

In another embodiment, the % deltaT2 and T2Max values are not incorporated to a model, but rather simply divided by each other. In effect, this simultaneously takes into account changes in fibrinogen and hematocrit and their effects on T2 and relaxivity, in the following form:

% deltaT2/T2Max=(maximum asymptote−minimum asymptote)/(maximum asymptote)² (14)

A linear fit between Eq. 14 and fibrinogen is then used to predict new values of fibrinogen from whole blood samples. In other embodiments, different combinations of maximum and minimum asymptote are used to normalize the T2 signals, including:

$$\% deltaT 2 / T 2 Min = \frac{( maximum asymptote - minimum asymptote )}{( maximum asymptote ) * ( minimum asymptote )} \qquad ( 15 )$$

In other embodiments, other combinations of Eq. 14 and 15 are also possible, including changing the polynomial order and subtraction order of the maximum and minimum asymptote terms.

Alternatively, another means to estimate fibrinogen (aka, the Estimated Fibrinogen Function (EFF), functional fibrinogen, or fibrinogen level) is to use the difference in the reciprocals of the maximum asymptote and minimum asymptote, by taking into account the blood volume and hematocrit estimate, in the following form:

Fibrinogen_by_difference=((c*V*(1000/T2Min)−(1000/T2Max)))/((1−h)) (16)

In Eq. 16, c is a constant derived empirically through least square regression or another statistical learning method, vis the reciprocal of the whole blood fraction in the final reaction, T2Min and T2Max represent the minimum and maximum asymptotes as previously described (Eq. 9), and h represents an estimate of hematocrit derived using the maximum asymptote. This method differs from other methods in that it accounts for the volume of whole blood in the sample, and therefore is more readily applicable to different blood fractions in the final reaction. The estimate of h may be simply based on the maximum asymptote as previously described, or may be derived through other models. In one embodiment, the estimate of h in Eq. 16 is defined with a simple linear model as:

h=a*(1000/T2Max)−b (17)

In Eq. 17, h is the estimated hematocrit, a and b are constants derived empirically through least square regression or another statistical learning method, and T2Max is the maximum asymptote defined previously. Other estimates of hematocrit are also possible that involve the minimum asymptote or non-linear models. The estimate of hematocrit may be used to account for hematocrit bias and provide for a more accurate measure of fibrinogen using any of the above methods (e.g., Eq. 9-16) or other embodiments and methods.

In summary, the multiplexed nature of T2MR measurements allows for correction of hematocrit bias through a variety of methods. T2MR measurements can be used that were acquired on the same or different aliquot of the blood sample. For example, a second measurement can be taken with a blood sample that is at a different dilution than the sample used to activate fibrin formation. T2MR signals from this measurement can be used to normalize the % deltaT2 measurement to correct for the hematocrit interference. In short, hematocrit interference with a fibrinogen measurement can be corrected using T2MR measurements on a patient sample using one or more aliquots from the blood sample at a same or different sample dilution.

Because T2MR provides a measure of hematocrit, similar approaches can be used to correct for hematocrit bias to clot times.

Sample Tubes

The methods of the invention include measures for evaluating hemostatic conditions and parameters through the observation of platelet-induced clot contraction. These include platelet activity, hyper and hypocoagulability states, and clot lysis, among others. The kinetics and signals associated with these reactions depend on at least three categories of variables: (1) the inherent biology within the sample, such as platelet activity, factor deficiencies, and therapeutic agents; (2) the type and concentration of specific activator used to initiate clotting in the sample; and (3) variation in how the clot forms and contracts within the sample tube. One goal of the methods of the invention is to ensure that the variability in the observed experimental values reflects only variability in the inherent biology of the sample (category 1). To this end, standard reagent formulations and sample dilution volumes and ratios can be used to control and reduce variability arising from the predetermined condition of clot initiation (category 2) for any given sample measurement. We have observed that variability in fibrin adhesion to the inner surface of the sample tube (category 3) can sometimes introduce variability in the sample measures that can reduce the sensitivity and reproducibility of the methods of the invention. To reduce this source of variability the methods of the invention can be performed in a sample tube having an inner surface that controls fibrin adhesion. The use of sample tubes that control fibrin adhesion can result in more robust, sensitive, and reproducible clot-contraction based assays, thereby producing more accurate data that correlates better with reference methods and clinical outcomes.

The sample tubes used in the methods of the invention can include an inner surface of the sample tube that controls fibrin adhesion. This can be achieved through the selection of an appropriate material from which the entire sample tube is made, or by coating the inner surface of a sample tube (covalently or non-covalently) with a material that controls fibrin adhesion. For example, the inner surface can include a fluorinated material or a pegylated material or a material that increases the hydrophilicity of the inner surface to impart resistance to fibrin adhesion. The inner surface can include a substrate coated with a material that reduces fibrin adhesion in comparison to the substrate uncoated. The substrate can be, for example, glass or a base polymer (e.g., polypropylene, polycarbonate, polystyrene, polyallomer, or another base polymer suitable for making into a sample tube). The substrate can be a glass coated by silanization with a material that reduces fibrin adhesion in comparison to unsilanized glass. Alternatively, the material includes a surfactant, a polynucleotide, a protein, a polyethyle glycol, a fluorinated material (e.g., fluorocarbon coating), hydrophilic polymers (e.g. polyacrylates, polyvinyl alcohol, etc.), a carbohydrate (e.g., agarose, cellulose, carboxymethyl cellulose), or a mixture thereof.

The sample tubes used in the methods of the invention can include an inner surface conditioned/processed (e.g., silanization, siliconization, thin film deposition, plasma etching, plasma cleaning, etc.) to resist fibrin adhesion. Such processing can include plasma cleaning (i.e., corona treatment) to remove contaminants from the inner surface of the tube, or to prepare the surface for coating with a material that resists fibrin adhesion, or to produce a smoother substrate surface that controls fibrin adhesion. For example, the sample tubes used in the methods of the invention can include an inner surface patterned with hydrophilic and hydrophobic groups on the underlying substrate of the sample to tube, a feature reported to reduce fibrin adhesion in contact lenses (see Sato et al., Proc. SPIE 5688, Ophthalmic Technologies XV, 260 (2005)). Alternatively, the sample tubes can include a thin film deposited onto the surface, such as a thin film including polyethylene glycol, fluorinated material, or a noble metal (e.g., silver, gold, platinum, palladium). In still another approach, the inner surface of the sample tube can be subjected to chemical vapor deposited poly(p-xylylene) polymers (i.e., a parylene coating).

The sample tubes used in the methods of the invention can include an inner surface bearing one or more materials having an extremely low coefficient of friction to provide a non-stick surface, such as polytetrafluoroethylene (Teflon®), fluorinated ethylene-propylene (FEP), perfluoroalkoxy polymer resin (PFA), parafilm (i.e., a surface coated with paraffin wax), or silicone. The sample tubes used in the methods of the invention can include an inner surface formed from a base polymer free of additives (e.g., lubricants, plasticizers, colorants, and other commonly used additives) which can migrate to the surface of the base polymer and alter its surface properties. For example, the inner surface can be formed from high purity polystyrene (e.g., Dow 666U), or a high purity polyacrylic acid (e.g., PMMA). The base polymer optionally can be selected to provide a hydrophilic inner surface, or is covalently modified (e.g., by oxygen plasma coating, air plasma coating, UV activated coating, or direct oxidation, e.g., with permanganate, to produce surface carboxylate groups) to provide a hydrophilic inner surface. The hydrophilic inner surface can be produced by controlling the presence of electronegative functional groups, such as functional groups containing nitrogen and/or oxygen.

The sample tubes used in the methods of the invention can include an inner surface including a substrate (e.g., glass or a base polymer, such as polypropylene, polycarbonate, polystyrene, polyallomer, or another base polymer suitable for making into a sample tube) coated with a surfactant. The surfactant may be selected from a wide variety of soluble non-ionic surface active agents including surfactants that are generally commercially available under the IGEPAL trade name from GAF Company. The IGEPAL liquid non-ionic surfactants are polyethylene glycol p-isooctylphenyl ether compounds and are available in various molecular weight designations, for example, IGEPAL CA720, IGEPAL CA630, and IGEPAL CA890. Other suitable non-ionic surfactants include those available under the trade name TETRONIC 909 from BASF Wyandotte Corporation. This material is a tetra-functional block copolymer surfactant terminating in primary hydroxyl groups. Suitable non-ionic surfactants are also available under the VISTA ALPHONIC trade name from Vista Chemical Company and such materials are ethoxylates that are non-ionic biodegradables derived from linear primary alcohol blends of various molecular weights. The surfactant may also be selected from poloxamers, such as polyoxyethylene-polyoxypropylene block copolymers, such as those available under the trade names Synperonic PE series (ICI), Pluronic® series (BASF), Supronic, Monolan, Pluracare, and Plurodac; polysorbate surfactants, such as Tween® 20 (PEG-20 sorbitan monolaurate); nonionic detergents (e.g., nonyl phenoxypolyethoxylethanol (NP-40), 4-octylphenol polyethoxylate (Triton-X100), Brij nonionic surfactants); and glycols such as ethylene glycol and propylene glycol.

The surfactant can be, for example, a polyethylene glycol alkyl ether or polysorbate surfactant. Polyethylene glycol alkyl ether surfactants can be used to coat the sample tubes utilized in the methods of the invention, and include, without limitation, Laureth 9, Laureth 12 and Laureth 20. Other polyethylene glycol alkyl ethers include, without limitation, PEG-2 oleyl ether, oleth-2 (Brij 92/93, Atlas/ICI); PEG-3 oleyl ether, oleth-3 (Volpo 3, Croda); PEG-5 oleyl ether, oleth-5 (Volpo 5, Croda); PEG-10 oleyl ether, oleth-10 (Volpo 10, Croda, Brij 96/97 12, Atlas/ICI); PEG-20 oleyl ether,oleth-20 (Volpo 20, Croda, Brij 98/99 15, Atlas/ICI); PEG-4 lauryl ether, laureth-4 (Brij 30, Atlas/ICI); PEG-9 lauryl ether; PEG-23 lauryl ether, laureth-23 (Brij 35, Atlas/ICI); PEG-2 cetyl ether (Brij 52, ICI); PEG-10 cetyl ether (Brij 56, ICI); PEG-20 cetyl ether (Brij 58, ICI); PEG-2 stearyl ether (Brij 72, ICI); PEG-10 stearyl ether (Brij 76, ICI); PEG-20 stearyl ether (Brij 78, ICI); and PEG-100 stearyl ether (Brij 700, ICI). Polysorbate surfactants can be used to coat the sample tubes utilized in the methods of the invention. Polysorbate surfactants are oily liquids derived from pegylated sorbitan esterified with fatty acids. Common brand names for Polysorbates include Alkest, Canarcel and Tween. Polysorbate surfactants include, without limitation, polyoxyethylene 20 sorbitan monolaurate (TWEEN 20), polyoxyethylene (4) sorbitan monolaurate (TWEEN 21), polyoxyethylene 20 sorbitan monopalmitate (TWEEN 40), polyoxyethylene 20 sorbitan monostearate (TWEEN 60); and polyoxyethylene 20 sorbitan monooleate (TWEEN 80).

In some cases, an RF coil maybe integrated into a disposable sample tube and be a disposable component of the system used to perform the methods of the invention. The coil may be placed in a manner that allows electrical contact with circuitry on the fixed NMR setup, or the coupling may be made inductively to a circuit.

T2MR Units

The systems for carrying out the methods of the invention can include one or more NMR units. A bias magnet establishes a bias magnetic field B₀ through a sample. An RF coil and RF oscillator provides an RF excitation at the Larmor frequency which is a linear function of the bias magnetic field B₀. In one embodiment, the RF coil is wrapped around the sample well. The excitation RF creates a nonequilibrium distribution in the spin of the water protons (or free protons in a non-aqueous solvent). When the RF excitation is turned off, the protons “relax” to their original state and emit an RF signal that can be used to extract information about the water populations in the blood sample. The coil acts as an RF antenna and detects a signal, which based on the applied RF pulse sequence, probes different properties of the material, for example a T2 relaxation. The signal of interest for some cases of the technology is the spin-spin relaxation (generally 10-2000 milliseconds) and is called the T2 relaxation. The RF signal from the coil is amplified and processed to determine the T2 (decay time) response to the excitation in the bias field B₀. The well may be a small capillary or other tube with nanoliters to microliters of the sample, including the blood sample and an appropriately sized coil wound around it. The coil is typically wrapped around the sample and sized according to the sample volume. For example (and without limitation), for a sample having a volume of about 10 ml, a solenoid coil about 50 mm in length and 10 to 20 mm in diameter could be used; for a sample having a volume of about 40 μL, a solenoid coil about 6 to 9 mm in length and 3.5 to 7 mm in diameter could be used; and for a sample having a volume of about 0.1 nL a solenoid coil about 20 μm in length and about 10 μm in diameter could be used. Alternatively, the coil may be configured within, about, or in proximity to the well or sample tube. An NMR system may also contain multiple RF coils for the detection of multiplexing purposes. In certain embodiments, the RF coil has a conical shape with the dimensions 6 mm×6 mm×2 mm.

The NMR unit includes a magnet (i.e., a superconducting magnet, an electromagnet, or a permanent magnet). The magnet design can be open or partially closed, ranging from U- or C-shaped magnets, to magnets with three and four posts, to fully enclosed magnets with small openings for sample placement. The tradeoff is accessibility to the “sweet spot” of the magnet and mechanical stability (mechanical stability can be an issue where high field homogeneity is desired). For example, the NMR unit can include one or more permanent magnets, cylindrically shaped and made from SmCo, NdFeB, or other low field permanent magnets that provide a magnetic field in the range of about 0.5 to about 1.5 T (i.e., suitable SmCo and NdFeB permanent magnets are available from Neomax, Osaka, Japan). For purposes of illustration and not limitation, such permanent magnets can be a dipole/box permanent magnet (PM) assembly, or a Halbach design (See Demas et al., Concepts Magn Reson Part A 34A:48 (2009)). The NMR units can include, without limitation, a permanent magnet of about 0.5T strength with a field homogeneity of about 20-30 ppm and a sweet spot of 40 μL, centered. This field homogeneity allows a less expensive magnet to be used (less tine fine-tuning the assembly/shimming), in a system less prone to fluctuations (e.g. temperature drift, mechanical stability over time-practically any impact is much too small to be seen), tolerating movement of ferromagnetic or conducting objects in the stray field (these have less of an impact, hence less shielding is needed), without compromising the assay measurements (relaxation measurements and correlation measurements do not require a highly homogeneous field).

The basic components of an NMR unit include electrical components, such as a tuned RF circuit within a magnetic field, including an MR sensor, receiver and transmitter electronics that could be including preamplifiers, amplifiers and protection circuits, data acquisitions components, pulse programmer and pulse generator.

The NMR system may include a chip with RF coil(s) and electronics micro-machined thereon. For example, the chip may be surface micromachined, such that structures are built on top of a substrate. Where the structures are built on top of the substrate and not inside it, the properties of the substrate are not as important as in bulk micromachining, and expensive silicon wafers used in bulk micromachining can be replaced by less expensive materials such as glass or plastic. Alternative embodiments, however, may include chips that are bulk micro-machined. Surface micromachining generally starts with a wafer or other substrate and grows layers on top. These layers are selectively etched by photolithography and either a wet etch involving an acid or a dry etch involving an ionized gas, or plasma. Dry etching can combine chemical etching with physical etching, or ion bombardment of the material. Surface micromachining may involve as many layers as is needed.

In some cases, an inexpensive RF coil maybe integrated into a disposable sample tube of the invention, or into a disposable cartridge. The coil could be placed in a manner that allows electrical contact with circuitry on the fixed NMR setup, or the coupling could be made inductively to a circuit.

Where the relaxation measurement is T2, accuracy and repeatability (precision) will be a function of temperature stability of the sample as relevant to the calibration, the stability of the assay, the signal-to-noise ratio (S/N), the pulse sequence for refocusing (e.g., CPMG, BIRD, Tango, and the like), as well as signal processing factors, such as signal conditioning (e.g., amplification, rectification, and/or digitization of the echo signals), time/frequency domain transformation, and signal processing algorithms used. Signal-to-noise ratio is a function of the magnetic bias field (B₀), sample volume, filling factor, coil geometry, coil Q-factor, electronics bandwidth, amplifier noise, and temperature.

In order to understand the required precision of the T2 measurement, one should look at a response curve of the assay at hand and correlate the desired precision of determining the water populations present in the blood sample and the precision of the measureable, e.g., T2 for some cases. Then a proper error budget can be formed. The NMR units for use in the systems and methods of the invention can be those described in U.S. Pat. No. 7,564,245, incorporated herein by reference. The NMR units of the invention can include a small probehead for use in a portable magnetic resonance relaxometer as described in PCT Publication No. WO09/061481, incorporated herein by reference.

The systems of the invention can include a disposable sample tube or sample holder for use with the MR reader that is configured to permit a predetermined number of measurements (i.e., is designed for a limited number of uses). The disposable sample tube or sample holder can include none, part, or all, of the elements of the RF detection coil (i.e., such that the MR reader lacks a detection coil). For example, the disposable sample tube or sample holder can include a “read” coil for RF detection that is inductively coupled to a “pickup” coil present in the MR reader. When the sample container is inside the MR reader it is in close proximity to the pickup coil and can be used to measure NMR signal. Alternatively, the disposable sample tube or sample holder includes an RF coil for RF detection that is electrically connected to the MR reader upon insertion of the sample container. Thus, when the sample container is inserted into the MR reader the appropriate electrical connection is established to allow for detection. The number of uses available to each disposable sample tube or sample holder can be controlled by disabling a fusible link included either in the electrical circuit within the disposable sample holder, or between the disposable sample tube or sample holder and the MR reader. After the disposable sample tube or sample holder is used to detect an NMR relaxation in a sample, the instrument can be configure to apply excess current to the fusible link, causing the link to break and rendering the coil inoperable. Optionally, multiple fusible links could be used, working in parallel, each connecting to a pickup on the system, and each broken individually at each use until all are broken and the disposable sample tube or sample holder rendered inoperable. Preferably, the disposable sample tube is a coated tube of the invention.

### Examples

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

We demonstrate that the transverse relaxation time of the nuclear magnetic resonance signal of water, referred to here as T2MR, can be utilized to probe microenvironments of water molecules in blood ex vivo formed during hemostatic processes in a reagent-free manner. Our results show that T2MR allows the physical states of blood to be monitored by continuously measuring the spin-spin (T2) relaxation times of water (and other hydrogen nuclei) in a whole blood sample. Hydrogen nuclei (predominantly from water) are a sensitive and general magnetic resonance probe of the diverse and distinct microenvironments that develop during clot formation and structural rearrangement. For example, addition of an activator such as thrombin to whole blood initiates platelet aggregation and fibrin polymerization, generating a clot that subsequently undergoes platelet-mediated contraction. Contraction of the fibrin clot impacts microenvironments of water around the various components within the blood sample, including soluble proteins, erythrocytes, and the fibrin network itself, leading to the formation of multiple water compartments. These compartments and their formation over time can be discerned by applying an algorithm to resolve multiple time constants from a single T2MR relaxation curve. The sensitivity of the T2MR diagnostic platform to the hemostatic potential of blood arises from measuring these heterogeneities in the microenvironments of multiple water compartments that develop during clotting, contraction and clot lysis.

Here we describe how T2MR reports on the integrated contributions of plasma, platelets and other blood cells to hemostasis. This mix-and-read platform requires minimal sample volumes (less than 50 μl) compared with conventional methods and enables the measurement of both established and newly described hemostatic parameters on a single, simple to use instrument using water to probe the coagulative behavior of blood. This methodology can be used to measure both individual hemostatic parameters and integrated hemostasis. Major advantages over existing methods for measuring standard parameters include ease of performance by eliminating sample modification prior to analysis, data output in as little as a few minutes with the option to monitor samples for hours, and volume requirements that are 10-100 times less than existing methodologies.

Magnetic Resonance Relaxation Data

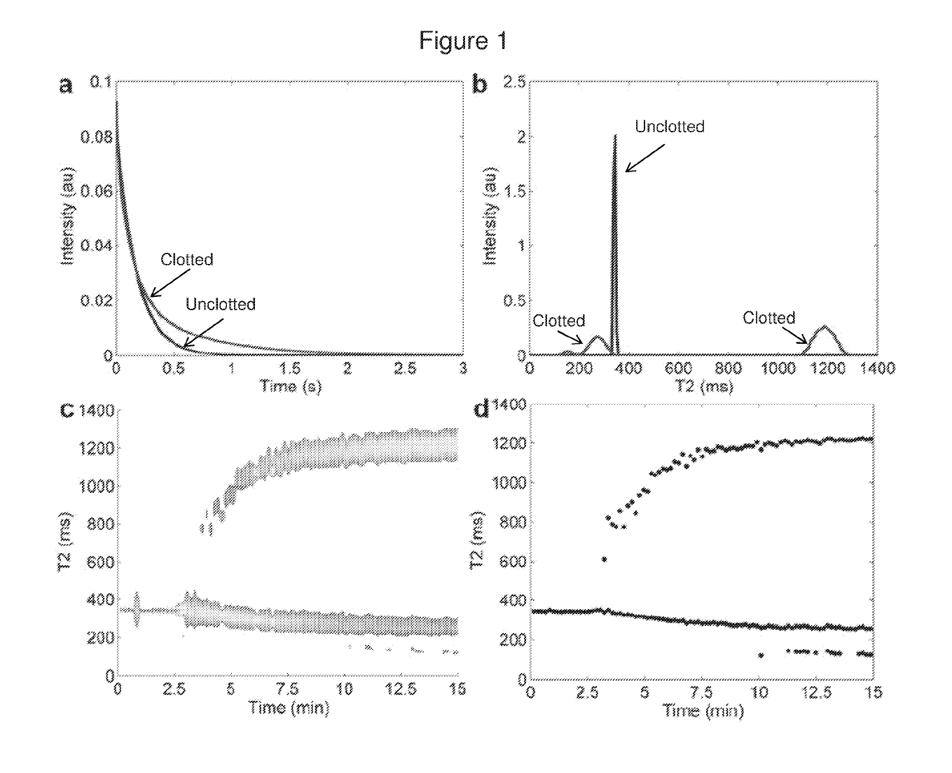

The relaxation mechanisms for magnetic resonance measurements of aqueous samples depend on chemical and diffusive exchange of water. A single relaxation value is measured when exchange is rapid, but multiple relaxation values can be measured when there is a barrier to exchange between microscopic environments. Key to applying T2MR to monitor micro environment changes is the ability to resolve specific T2 relaxation values of multiple water compartments within a sample. One way to do this is to implement an algorithm based on the inverse Laplace transform, which has been applied previously to estimate component decay constants in exponential decay curves. Inverse Laplace transform processing of CPMG spectra produces a multi-exponential fit of the relaxation data shown in equation 18:

$$S ( t ) = {\sum }_{i} {A}_{i} {e}^{- t / T {2}_{i}} + O \qquad ( 18 )$$

where S(t) is the relaxation signal acquired with the CPMG sequence, A, is the amplitude corresponding to the relaxation time constant, T2ᵢ, and O is the offset term. FIGS. 1a-1d show how kinetic spectra can be formed from numerical inverse Laplace transform.

The precision and reproducibility of multi-component relaxation measurements across three T2MR instruments was characterized using mineral oil, which generates a two-component signal. Average T2 relaxation times (30 min measurements at sampling rate of 10 s) were 278 ms and 116 ms; average within-run precision (coefficient of variation (% CV)) values were 2.94% and 5.07% for the higher and lower component, respectively; day-to-day reproducibility (34 runs spanning 6 months) values were 3.4% and 7.6% for the higher and lower component, respectively.

General Procedures for Blood Sample Collection and Fractionation.

Blood was obtained from healthy volunteers not taking aspirin, non-steroidal anti-inflammatory drugs or other medications known to inhibit platelet function for least 7-10 days, with informed consent and approval by Perelman School of Medicine-University of Pennsylvania Institutional Review Board. Blood was drawn via venipuncture into 3.2% trisodium citrate (9:1) following standard procedures that minimize platelet activation. Samples were kept at room temperature and were studied within 4 hr after the blood draw. A complete blood count was performed on an automated hematology analyzer (HemaVet 950FS, Drew Scientific, Dallas, Tex.).

For embodiments requiring fractionation and reconstitution of samples, 12 ml of blood was placed in 15 ml polypropylene tubes (Corning, Tewksbury, Mass.) and centrifuged for 15 min at 210 g at ambient temperature (22° C.). Platelet-rich plasma (PRP) was recovered from upper layer in the tube following centrifugation and transferred to a new tube. The residual blood preparation was centrifuged again at 900 g for 10 min at ambient temperature. The platelet-poor plasma (PPP) fraction was collected from the top layer and transferred to a new tube. Any remaining volume of PPP along with the buffy coat layer was removed, and the upper 1 ml of packed erythrocytes was aspirated and transferred to a new tube. To obtain concentrated platelets, PRP was centrifuged at 900 g for 10 min at ambient temperature. To prevent platelet aggregation, prostaglandin E1 (PGE1) was added (final concentration 5 μM). The supernatant was aspirated and discarded, and the platelet pellet was resuspended in PPP not containing PGE1 to generate a concentrated platelet suspension. Reconstituted samples were prepared by mixing concentrated erythrocytes, concentrated platelets, and PPP at desired levels.

Instrument Fabrication and Pulse Sequence Parameters

A small, portable T2MR instrument (35×15×18 cm, 9 kg) was designed to measure the proton T2 relaxation times within blood samples. The instrument consists of a 0.54 T (approximately 23 MHz) permanent magnet assembly, radiofrequency probe, single-board spectrometer, and peripheral electronics within a 37° C. temperature controlled enclosure. The radiofrequency probe accommodates 10 40 μL samples contained within a standard 0.2 ml polypropylene tube. For FIGS. 1a-1c, 2, 3a-3b, and 5, a Carr-Purcell-Meiboom-Gill (CPMG) pulse sequence was applied to generate relaxation curves from which T2 values are extracted. The parameters of pulse sequence experiments were: inter-echo spacing (t_{E})=500 μs and a dwell time of 1-10 s depending on the application. This acquisition method removes the effects of static field inhomogeneities, enabling the use of a small, inexpensive magnet which is shimmed only once during manufacturing.

### Example 1. Blood Clotting, Retraction, and Lysis with Thrombin and Tissue Plasminogen Activator

In one embodiment, blood clotting was initiated by addition of 2 μL of a 0.2 M CaCl₂ solution and 2 μL of thrombin (Sigma-Aldrich, St. Louis, Mo., final concentration 0.1-3.0 U/ml) to 34 μL of blood in a 200 μL PCR tube (Eppendorf, Hauppauge, N.Y.). All components were pre-warmed for 1 min at 37° C. before mixing. Samples were mixed by three aspiration and dispersion cycles using a pipette, then put into the T2MR reader for measurement. Typical run length was 30 min with a 10 sec sampling rate. For some experiments, data collection time was extended to 1 hr.

To establish T2MR signatures for fibrinolysis, tissue plasminogen activator (tPA, Alteplase, Genentech, South San Francisco, Calif.) was added to samples clotted by thrombin. Blood clotting was initiated as described above, and the sample was incubated for 60 min to allow for complete clot contraction. Then, 0.5-1 μM tPA was added to clotted and contracted samples. Care was taken not to disturb clots, which were usually attached to the tube wall. The pipette tip was carefully placed into the visible serum layer on the tube side opposite to the contracted clot, and 3 μL of tPA solution was added with a single dispensing of the pipettor. The tPA solutions were made from a stock solution prepared according to manufacturer instructions using 0.15 M sodium chloride, pH 7.4.

### Example 2. Real-Time Monitoring of Clot Formation, Contraction and Fibrinolysis

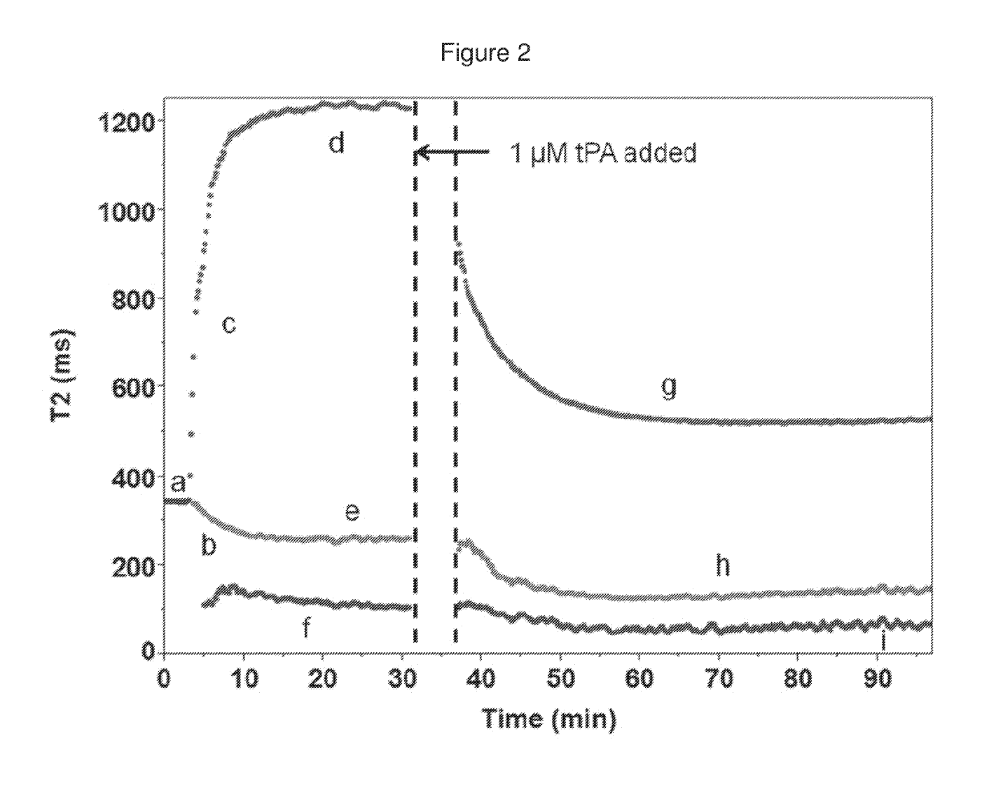

In one embodiment, the dependencies of the T2MR signals during clotting of re-calcified citrated blood samples from healthy donors initiated by adding 3 U/ml thrombin was measured. Thrombin activates platelets and cleaves fibrinogen to form a three-dimensional fibrin network stabilized by factor XIIIa. Addition of thrombin led to rapid formation of a gelatinous meshwork that filled the sample volume accompanied by a small, rapid decrease in the T2MR signal over tens of seconds due to the sample transitioning from a liquid to gel state. In the initial gel state, only one relaxation rate was observed (FIG. 2, part a), reflecting uniform distribution of erythrocytes and other blood components. Approximately four minutes after thrombin addition, the T2MR signal split into two peaks representing distinct water populations in slow exchange with each other. One peak decreased in T2 value (FIG. 2, part b), indicating increasing erythrocyte concentration in one compartment, while the T2 value of the other peak increased rapidly, consistent with depletion of erythrocytes (FIG. 2, part c). Approximately 20 minutes after addition of thrombin, the upper peak reached a plateau (FIG. 2, part d). The lower peak at ˜300 ms decreased in T2 value, associated with visible clot contraction, until around 10 minutes when it reached a plateau at ˜275 ms (FIG. 2, part e). A third peak first appeared at 6 minutes at a lower T2 value (˜100 ms) (FIG. 2, part f).

We then assessed the sensitivity of the T2MR platform to fibrinolysis by adding tissue-type plasminogen activator (tPA) to the clotted samples 30 minutes after thrombin. After tPA addition, the T2 value of the upper peak decreased rapidly (FIG. 2, part g), the middle peak decreased from 250 to 175 ms (FIG. 2, part h), while the third peak at ˜100 ms persisted (FIG. 2, part i).

### Example 3. Analyzing Isolated Sample Components

In one embodiment the T2 values of individual components of blood were determined using samples fractionated as described above or using clotted whole blood components. All samples were pre-warmed at 37° C. for 1 min before transferring to a T2MR reader for measurement. For plasma, 40 μL of PPP was measured. For serum, 200 μL of whole blood was clotted by addition of 2 U/ml thrombin to re-calcified blood. After a 30 min incubation at 37° C., the tube was centrifuged for 1 min at 10,000 g and 40 μL of the upper (serum) fraction was measured. To measure isolated retracted clots, re-calcified blood was allowed to clot for 1 hr following addition of 2 U/ml thrombin at 37° C. Then, erythrocytes excluded from clot were removed by washing the clot with 100 μL of PPP by gentle pipetting. The liquid was aspirated and disposed. This washing protocol was repeated two more times. To measure the isolated clot, all liquid was aspirated after the washing steps.

To interpret the T2MR signals during clot formation, contraction, and lysis, the major biological components of the system were measured in isolation and upon recombination (Table 1). Consistent with relaxation theory, T2MR signals were highest for serum, intermediate for plasma and lowest for whole blood and contracted clots. The range of T2 values for whole blood from healthy donors, 400-285 ms, corresponds to hematocrit values of 35%-55% and the range for reconstituted samples, 575-189 ms, corresponds to hematocrit values of 21%-83%. The higher T2MR signals in serum relative to whole blood arise from the lack of erythrocytes (and associated hemoglobin), which accelerates relaxation of water protons. The T2MR signal of plasma is lower than that of serum due to the relatively higher concentration of proteins that increase relaxation rates by exchange between free and protein bound water (Table 1).

TABLE 2
T2 values of isolated components of clotted blood for
N = 6 donor samples.

| Isolated | |
|---|---|
| Component | T2 (ms) |
| Serum | 1000-1200 |
| Plasma | 800-1000 |
| Homogenous whole | 400-285 for 35-55% |
| blood | hematocrit |
| Loosely bound or | 200-300 |
| unbound erythrocytes | |
| Contracted clot | 75-175 |

We next measured the T2MR signals of isolated contracted clots. One hour after re-calcified citrated whole blood was clotted with 2 U/ml thrombin, contracted clots were removed, washed with platelet poor plasma and T2MR signals were measured. Clots remained intact during manipulation indicating tight contraction. The T2MR signals generated by isolated clots ranged from 100-150 ms (n=6), consistent with this signal arising from a tightly contracted clot with a hematocrit approaching 100% based on equation 19.

$$T {2}_{o} = {( \frac{{X}_{e}}{T {2}_{e}} + \frac{{X}_{p}}{T {2}_{p}} )}^{- 1} \qquad ( 19 )$$

where T2ₒ is the observed T2 value, T2ₑ and T2ₚ are the intrinsic relaxation time constants for the erythrocyte and plasma compartments, and Xₑ and Xₚ are the mole fraction of total water in each compartment.

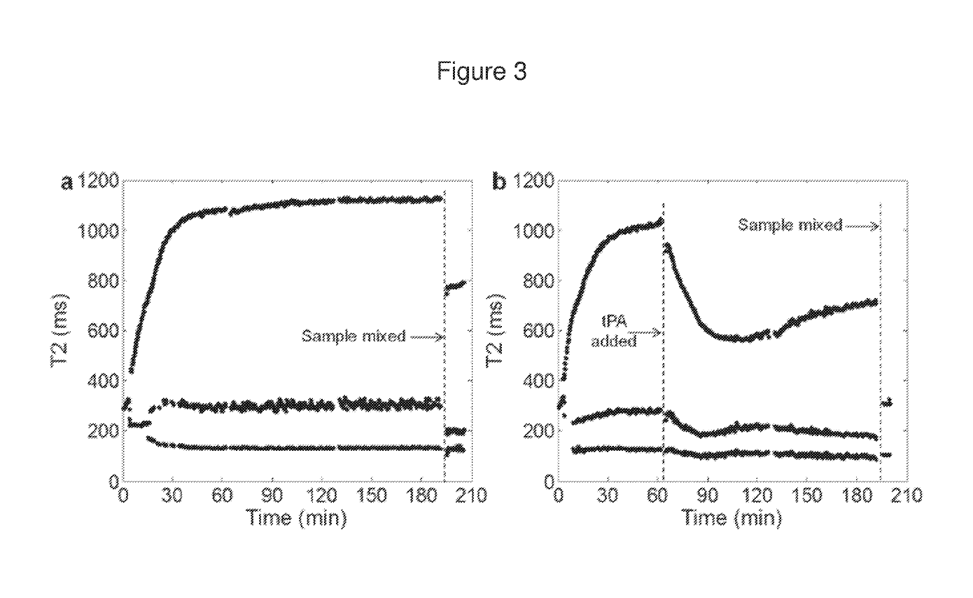

The compartment generating the signal in FIG. 2, part b, at 300 ms that dropped to 200 ms was assessed by testing two conditions: (1) re-calcified citrated whole blood activated with thrombin to form a contracted clot and (2) re-calcified citrated whole blood activated with thrombin followed by addition of tPA. After incubation, samples were analyzed before and after mixing with a pipette to re-suspend unbound erythrocytes. In the sample clotted with thrombin, the 200-300 ms signal remained after mixing, but the T2 value of both the upper peak and this peak decreased as some unbound erythrocytes were dislodged by mixing (FIG. 3a). In the sample clotted with thrombin then lysed with tPA, the 200-300 ms signal disappeared altogether after mixing. The upper T2 peak decreased in T2 value as the erythrocytes that were released from the fibrin network during clot lysis were resuspended by mixing (FIG. 3b). These data support the conclusion that the T2MR signal at 200-300 ms originates from erythrocytes loosely bound to platelets and fibrin that is susceptible to tPA-induced fibrinolysis. The observation that the lowest T2MR signal in clotted samples persists after tPA addition is consistent with the signal emanating from a tightly compacted clot resistant to fibrinolysis.

### Example 4. Clotting Reconstituted Samples with Calcium and Kaolin

The combined effect of hematocrit and platelet count on the T2MR signal was explored by generated 96 reconstituted samples of varying hematocrit and platelet count. These samples were prepared as described previously. The clotting experiments were performed by mixing 34 μL of reconstituted blood, 2 μL 0.2 M CaCl₂, and 2 μL kaolin solution (Haemonetics, Braintree, Mass.). All reagents were pre-warmed at 37° C. prior to T2MR measurement.

### Example 5. T2 Relaxation and Hematocrit

In one embodiment, hematocrit levels are measured. A single T2 value was observed for the measurement of unclotted blood, consistent with previous studies with similar magnetic fields and short inter-echo CPMG delays. The dependence of T2 relaxation on the blood oxygenation state at higher fields and much longer echo times (tens of ms) has been successfully used for in vivo MRI. The diminished dependence of blood oxygenation state on T2 relaxation at low magnetic fields and short echo times (hundreds of microseconds) has been previously studied and suggests the difference between oxygenated and deoxygenated blood to be <25 ms under our measurement conditions. Further experiments and optimization will be necessary to fully characterize this dependence. T2MR signal dependence on hematocrit can be modeled by equation 19 (above), where T2ₒ is the observed T2 value, T2ₑ and T2ₚ are the intrinsic relaxation time constants for the erythrocyte and plasma compartments, and Xₑ and Xₚ are the mole fraction of total water in each compartment. Using equation 19, measured data were fitted best when T2ₚ=1000 ms and T2ₑ=165 ms.

### Example 6. Prothrombin Time Method Comparison

In one embodiment, a T2MR citrated blood prothrombin time (PT) assay was developed using Innovin® as a reagent and measuring the time at which the T2MR signal changed due to clot formation. Dade® Innovin® (Siemens Healthcare Diagnostics, Newark, Del.) was prepared according to manufacturer instructions. A stock solution of fibrinogen (60 mg/ml) was prepared in saline. To measure the clotting time using T2MR, 150 μL of citrated blood was mixed with 2.6 μL of the fibrinogen solution. All components were incubated for 2 minutes at 37° C. prior to T2MR measurements. Blood and fibrinogen (40 μL) was positive pipetted into the 20 μl of Innovin® and the T2MR readings were initiated immediately. T2 values were collected at a sampling rate of 2 sec for 2 min. The resulting T2 vs. time data was fit with a 5 parameter logistic, and the clotting time was calculated using the “half maximal effective dose” (EC50) equation commonly used to determine the potency of drugs when concentration is plotted versus time instead of T2 value. The reference method clotting time was obtained by running the same samples on the Stago ST4 system using PRP following the manufacturer's protocol.

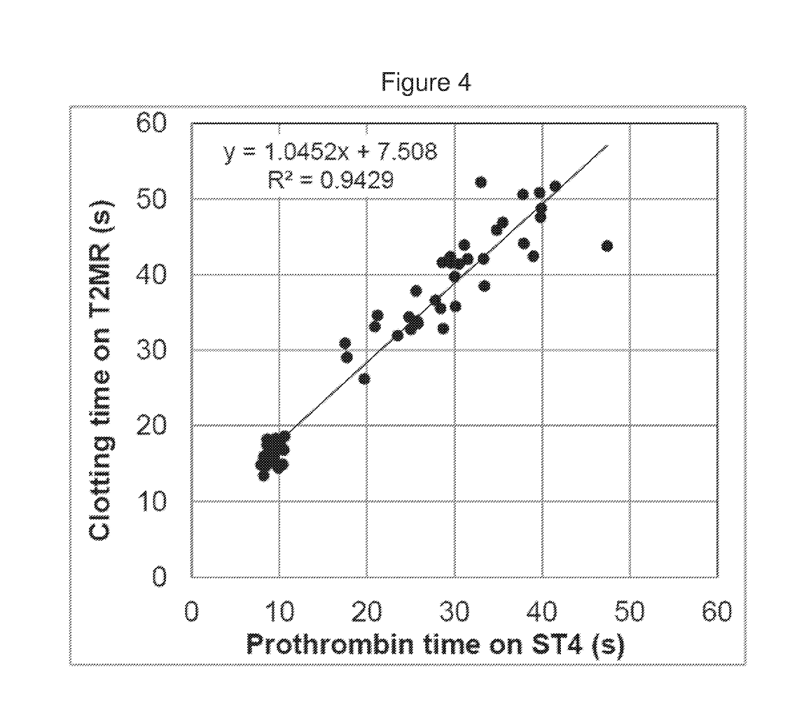

A T2MR citrated blood prothrombin time (PT) assay was developed using Innovin® as a reagent and measuring the time at which the T2MR signal changed due to clot formation. The 2:1 sample to reagent dilution used in this assay formulation necessitated the addition of a fibrinogen reagent to ensure adequate changes in the T2MR signal upon clotting. This increased the robustness and precision of the assay, while still producing PT times that correlated well with the reference method. Fibrinogen was not added for other assays where sample dilution was less. The T2MR PT assay gave % CV=3.5% for 10 replicates across 23 donor samples (Table 3) and a correlation of R²=0.94 over 68 donor samples from normal and anti-coagulated donors when compared with measurement in plasma using the Stago ST4 system (FIG. 4).

TABLE 3
Precision of PT measurements using T2MR.

| | Average T2MR PT | T2MR % CV |
|---|---|---|
| Sample | n = 10 (sec) | n = 10 |
| 1 | 16.0 | 2.6% |
| 2 | 14.5 | 5.2% |
| 3 | 13.8 | 3.8% |
| 4 | 17.0 | 4.3% |
| 5 | 15.2 | 2.7% |
| 6 | 16.3 | 2.2% |
| 7 | 14.5 | 4.0% |
| 8 | 17.9 | 4.5% |
| 9 | 17.0 | 4.1% |
| 10 | 15.9 | 2.8% |
| 11 | 44.9 | 4.3% |
| 12 | 32.2 | 1.9% |
| 13 | 53.1 | 3.6% |
| 14 | 36.9 | 2.8% |
| 15 | 50.9 | 3.8% |
| 16 | 41.1 | 3.6% |
| 17 | 47.3 | 2.5% |
| 18 | 41.9 | 2.5% |
| 19 | 36.2 | 5.0% |
| 20 | 36.6 | 2.9% |
| 21 | 42.6 | 2.0% |
| 22 | 31.2 | 4.9% |
| 23 | 44.6 | 4.9% |

### Example 7. Measurement of Clot Strength

To demonstrate correlation of T2MR to the thromboelastography maximum amplitude (TEG MA) parameter, citrated blood samples were titrated with abciximab (ReoPro, Eli Lilly and Company, Indianapolis, Ind.), an inhibitor of the platelet glycoprotein αIIbβ3 receptor that binds fibrin and is essential for clot contraction. A 0.5 mg/ml solution of abciximab was prepared by diluting the stock 10 mg/5 ml solution by 1:4 in saline. The abciximab-treated blood samples were incubated for at least 5 min prior to clotting. Clotting was initiated by adding 2 μL 0.2 M CaCl₂ and 2 μL TEG kaolin to 34 μL of abciximab-treated blood sample. To compare the T2MR signal to TEG MA, a DiffT2 parameter was calculated by taking the difference in T2 between the upper and middle peaks at a time point 13 min after adding calcium and kaolin. The TEG MA values were measured on the same samples following manufacturer instructions.

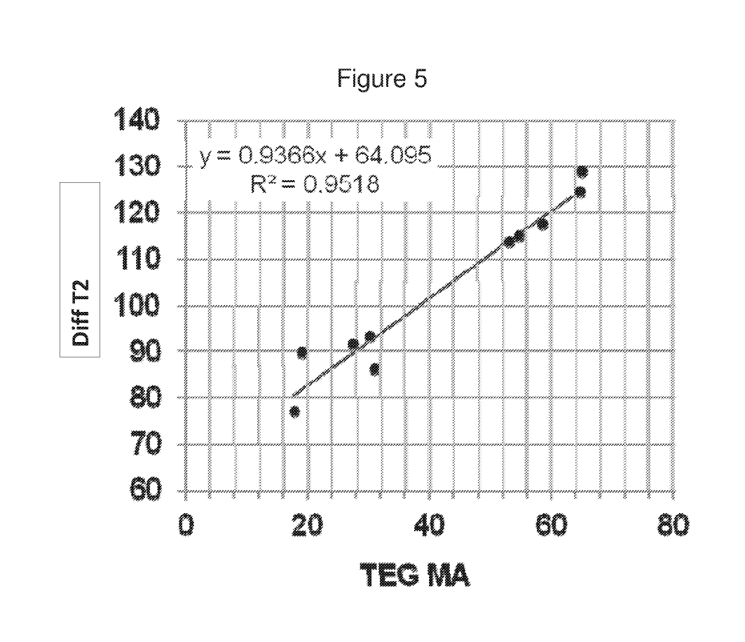

For comparison between T2MR and TEG, calcium and kaolin activation of citrated blood was used and normal donor samples were treated with various amounts of abciximab, an inhibitor of the platelet glycoprotein αIIbβ3 receptor that binds fibrin and is essential for clot contraction. The difference in T2 value between the peaks associated with serum and loosely compacted clot showed a strong correlation (R²=0.95) with the TEG MA values across 10 samples from 3 donors at varying amounts of added abciximab (FIG. 5).

### Example 8. Measurement of Platelet Activity

To isolate the T2MR signal response to platelet activity stimulated by adenosine diphosphate (ADP), we used a reagent mix containing final concentrations of 10 mM CaCl₂, 20 U/ml heparin to inhibit thrombin, and 1/38 dilution of the standard preparation of Activator F, a proprietary mix of reptilase and factor XIlIa to quickly generate a fibrin network, and 5 μM ADP. Alternatively, it is possible to generate fibrin clots using batroxobin and FXIIIa purchased separately and recombined into a working assay reagent. To perform the test, 34 μL of citrated blood with or without 100 μM 2-methylthioadenosine 5′-monophosphate (2-MeSAMP) was added to 4 μL of activation reagent in a PCR tube and T2MR signals were monitored for 10 min. The platelet function of PRP with platelet count matched to that of whole blood from the same samples was tested concurrently with LTA on a Chrono-log optical aggregometer. To assess correlation, we used the criteria of maximum percent aggregation by LTA over 6 min and the maximum percent change in T2MR signal of the upper peak over 10 min. The cutoffs for a positive result were 55% signal change for LTA and 100% signal change for T2MR.

Whereas measurement of platelet function by light transmission aggregometry (LTA) measures platelet-platelet interactions, T2MR measures platelet function via platelet-mediated clot contraction, an integrated activity that includes platelet activation, aggregation, adhesion to the clot, and cell-mediated contraction. To demonstrate the configurability of the T2MR platform for platelet function assays, we compared T2MR with citrated blood and LTA performed with platelet rich plasma using adenosine diphosphate (ADP) as a platelet activator. To isolate the signal response to platelet activation, we used a reagent mix containing ADP, heparin to inhibit thrombin, and reptilase and factor XIlIa to quickly generate a fibrin network. We compared T2MR with LTA across samples tested with ADP in the presence and absence of the inhibitor 2-methylthioadenosine 5′-monophosphate (2-MeSAMP). Positive agreement between T2MR and LTA for 20 samples was 100%, and negative agreement over 8 samples was 75% (6/8), giving an overall agreement of 93% (26/28) (Table 4).

Other platelet activators can be substituted for ADP to yield similar overall performance. These include but are not limited to arachidonic acid, epinephrine, thrombin receptor agonist peptide (TRAP), or collagen.

TABLE 4
Contingency table comparing ADP platelet activity measurements
on T2MR and LTA.

LTA

| | | Yes | No | Totals |
|---|---|---|---|---|
| T2MR | Yes | 20 | 2 | 22 |
| | No | 0 | 6 | 6 |
| | Totals | 20 | 8 | 28 |

### Example 9. Correlation of T2MR with Other Diagnostic Tests

While T2MR can be used to obtain new insights into the physical states of microenvironments within blood samples, it can also be configured to measure standard hemostasis parameters. To demonstrate this, we performed method comparison studies against the Sysmex pocH-1001 hematology analyzer for hematocrit where an R²=0.95 for 40 donor samples and an average precision of % CV=4.8% for N=10 replicates across 13 donor samples; for prothrombin time (PT) against the Stago ST4 system, where a correlation of R²=0.94 over 68 donor samples from normal and anti-coagulated donors and a % CV=3.5% for N=10 replicates across 23 donor samples was observed; for thromboelastography (TEG) clot strength, a correlation of R²=0.95 between T2MR and TEG MA values across 10 samples was observed; and for platelet function measurements an overall agreement of 93% was observed between T2MR and light transmission aggregometry (LTA) for activation by ADP.

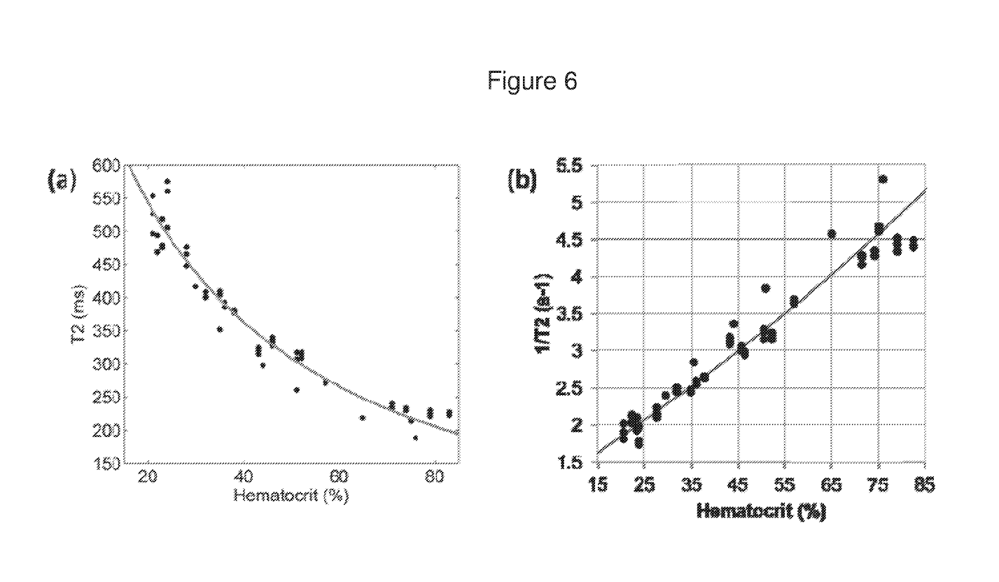

Hematocrit measurements can also be performed via T2MR. A method comparison study between T2MR and the Sysmex pocH-1001 hematology analyzer for determining hematocrit revealed high levels of correlation. Samples were generated from reconstituted blood from 40 independent donors in Table 4. A T2MR value was measured for each sample and converted to hematocrit using the calibration curves shown in FIGS. 6a and 6b, and the hematocrit was measured on the Sysmex platform. The two methods correlated with R²=0.95. T2MR hematocrit measurements also show a great deal of precision. Data in Table 5 depict T2MR values collected for 10 repetitions from each of 13 independent donor samples and converted to hematocrit values using the calibration curves shown in FIGS. 6a and 6b. The average % CV was 4.8%.

A T2MR citrated blood prothrombin time (PT) assay was developed using Innovin as a reagent and measuring the time at which the T2MR signal changed due to clot formation. The 2:1 sample to reagent dilution used in this assay formulation necessitated the addition of a fibrinogen reagent to ensure adequate changes in the T2MR signal upon clotting. This increased the robustness and precision of the assay, while still producing PT times that correlated well with the reference method. Fibrinogen was not added for other assays where sample dilution was less. The T2MR PT assay gave % CV=3.5% for 10 replicates across 23 donor samples (Table 2) and a correlation of R²=0.94 over 68 donor samples from normal and anti-coagulated donors when compared with measurement in plasma using the Stago ST4 system (FIG. 4).

For comparison between T2MR and TEG, calcium and kaolin activation of citrated blood was used and normal donor samples were spiked with various amounts of abciximab, an inhibitor of the platelet glycoprotein αIIbβ3 receptor that binds fibrin and is essential for clot contraction. The difference in T2 value between the peaks associated with serum and loosely compacted clot showed a strong correlation (R2=0.95) with the TEG MA values across 10 samples from 3 donors at varying amounts of added abciximab (FIG. 5).

Whereas measurement of platelet function by light transmission aggregometry (LTA) measures platelet-platelet interactions, T2MR measures platelet function via platelet-mediated clot contraction, an integrated activity that includes platelet activation, aggregation, adhesion to the clot, and cell-mediated contraction. To demonstrate the configurability of the T2MR platform for platelet function assays, we compared T2MR with citrated blood and LTA performed with platelet rich plasma using adenosine diphosphate (ADP) as a platelet activator. To isolate the signal response to platelet activation, we used a reagent mix containing ADP, heparin to inhibit thrombin, and reptilase and factor XIlIa to quickly generate a fibrin network. We compared T2MR with LTA across samples tested with ADP in the presence and absence of the inhibitor 2-methylthioadenosine 5′-monophosphate (2-MeSAMP). Positive agreement between T2MR and LTA for 20 samples was 100%, and negative agreement over 8 samples was 75% (6/8), giving an overall agreement of 93% (26/28) (Table 4).

TABLE 5
Method comparison for hematocrit measurement on T2MR and
Sysmex pocH-100i hematology analyzer.

| | T2MR | Sysmex |
|---|---|---|
| | hematocrit | hematocrit |
| Sample # | (%) | (%) |
| 1 | 43.7 | 22.3 |
| 2 | 38.1 | 20.0 |
| 3 | 34.3 | 18.7 |
| 4 | 54.5 | 29.2 |
| 5 | 23.4 | 14.2 |
| 6 | 39.0 | 22.5 |
| 7 | 58.3 | 32.6 |
| 8 | 28.9 | 18.4 |
| 9 | 33.2 | 21.1 |
| 10 | 67.0 | 38.5 |
| 11 | 28.2 | 19.6 |

TABLE 6
Precision of hematocrit measurements on T2MR.

| | Average T2MR | |
|---|---|---|
| Sample # | hematocrit (%) | % CV (10 reps) |
| 1 | 38.5 | 5.0 |
| 2 | 43.0 | 3.7 |
| 3 | 41.8 | 6.6 |
| 4 | 35.4 | 3.2 |
| 5 | 40.7 | 4.2 |
| 6 | 30.0 | 5.0 |
| 7 | 32.7 | 5.2 |
| 8 | 46.7 | 4.5 |
| 9 | 35.8 | 4.5 |
| 10 | 37.3 | 3.4 |
| 11 | 39.3 | 7.6 |
| 12 | 39.6 | 4.5 |
| 13 | 46.0 | 4.6 |

With respect to their overall analytical processes, T2MR technology and traditional thromboelastography share a number of similarities. Both technologies are able to present a global assessment of the clotting behavior seen in citrated whole blood samples by following the clotting process from initiation to fibrinolysis. Thromboelastography achieves this end by examining the macroscopic stiffness of the clot as it forms. Clot stiffness values and other parameters recovered from various points throughout a thromboelastogram are then used to infer various patient specific properties including clotting time, fibrinogen, platelet function, and clot lysis.

T2MR technology utilizes the signals from hydrogen nuclei in water molecules in the patient sample, to resolve the steps in the clot formation and contraction process in a manner that provides results that correlate more strongly with established reference methods while still providing a global assessment of the sample's hemostatic status. Unlike thromboelastography, T2MR can measure fibrinogen independent of platelet function by detecting the formation of the fibrin network directly. It can also detect in real time the activation of platelets by following platelet-mediated clot contraction. Finally, it can directly sense the dissolution of clotted blood during the fibrinolysis process. This ability to differentiate individual aspects of the clotting process and assign specific metrics to each process step, allows T2MR to generate metrics that more directly correlate to values recovered from gold standard methods like plasma PT, Clauss fibrinogen, and platelet aggregometry.

### Example 10. Hemostasis Multiplexed Panel

There is a clinical need for a global hemostasis diagnostic that can identify coagulable conditions and correlate with laboratory gold standards for clot time, fibrinogen, platelet activity, and fibrinolysis measurements. T2 magnetic resonance (T2MR) technology enables monitoring of the physical states of blood during coagulation for determination of these factors. Several important hemostatic parameters can be identified from a single whole blood sample and single reagent activator (TF or contact factor) in a newly developed multiplexed panel. Reported parameters for the panel include fibrinogen, fibrinolysis, platelet activity, clot time, and percent hematocrit. An additional factor can be measured in a third T2MR peak indicative of a tighter clot that can indicate a hypercoagulable state. Multiplexing can take place as either (1) a single-reaction multiplexed result, (2) measurements obtained in parallel from multiple aliquots of the same sample, or (3) measurements obtained in succession on the same instrument with multiple aliquots of the sample. A depiction of the T2MR measurements that can be taken as part of the multiplexed panel and what those measurements represent is found in FIGS. 8a and 8b. Another depiction is the individual data panels shown in FIGS. 9-13. The T2MR values are used to calculate each quantitative test result, which is displayed on the user interface as a clotting time value (e.g. PT or aPTT) in seconds, fibrinogen measurement in mg/dL, platelet function in percent activity, fibrinolysis in percent lysis, and hematocrit in percent hematocrit or concentration of hemoglobin. Prothrombin time (PT) is derived from the transition between the unclotted and clotted T2MR values when tissue factor is used as an activator. Activated partial thromboplastin time (aPTT) is derived from the transition between the unclotted and clotted T2MR values when a contact factor activator like ellagic acid is used as an activator. Fibrinogen (Fg) is derived from the decrease in T2MR signal upon clotting. Platelet Function (PLT) is derived from the T2MR signals from the serum and clot microenvironments and fibrinolysis (LYS) from the decrease in the serum phase T2MR signal.

T2MR measurement of clotting time is similar to clotting time measurements on other platforms in that a physical parameter of the sample that corresponds to fibrin formation is being monitored as a function of time after the addition of an activator of coagulation. FIGS. 9a-9c show T2MR data collected immediately after the addition of an activator and depicts how PT is derived. The examples shown in FIGS. 9a-9c, 10a-10c, 11a-11b, 12a-12b and described below are for the samples that were treated with extrinsic activator, but samples that have been treated with intrinsic activator behave similarly.

Activation of whole blood with an extrinsic or intrinsic activator leads to thrombin production and the conversion of fibrinogen to fibrin. The amount of fibrinogen in the sample can be derived from the magnitude of the change in the T2MR signal upon clot formation; the higher the concentration of fibrinogen the larger the change in T2MR signals. For an illustration of the fibrinogen measurement process, see FIGS. 10a-10c.

After addition of an extrinsic or intrinsic activator to a whole blood sample, thrombin is generated and the platelets become activated leading to clot contraction. As the process of clot contraction progresses, water is extruded from the contracting clot into a separate serum compartment within the sample producing two distinct microenvironments, exemplified by the emergence of a second T2MR peak with a distinct T2 relaxation time (See FIGS. 11a and 11b). The lower peak is water associated with the clot, while the upper peak is water from the serum phase extruded from the contracting clot. As the platelets contract the clot, the T2MR signal of the upper peak increases until the signal levels off as the T2 of the upper peak approaches that of serum. The “Platelet Function” can be derived from the relative T2MR signals of the upper and lower peaks. A third peak that can be visible under certain conditions can indicate a hypercoagulable state.

Fibrinolysis is the breakdown of blood clots due to the cleavage of fibrin by plasmin. Fibrinolysis can be measured with T2MR by measuring the change in the serum-associated (upper) T2MR signal after platelet induced clot contraction, as shown in FIGS. 12a and 12b, where a normal blood sample was spiked with tissue plasminogen activator (tPA) prior to activation. After clot formation and platelet mediated clot contraction, plasmin begins to cleave the fibrin strands, which results in a decrease in the upper peak as erythrocytes are released from the clot.

General Assay Methods

The general extrinsic activator protocol was as follows (T2Ex panel): an extrinsic activator solution was prepared by mixing tissue factor reagent with CaCl₂. To 36.7 μL of citrated whole blood (warmed to approximately 37° C. for 3 minutes and mixed) was added 5 μL of the extrinsic activator solution (also been warmed to approximately 37° C. for one minute). The resulting solution was mixed and subjected to T2 measurements for 50 minutes. For a given sample, a clotting time measurement was typically obtained within 5 minutes.

The general intrinsic activator protocol is as follows (T2In panel): an intrinsic activator solution was prepared by mixing tissue factor reagent with CaCl₂. To 36.7 μL of citrated whole blood (warmed to approximately 37° C. for 3 minutes and mixed) was added 5 μL of the intrinsic activator solution (also been warmed to approximately 37° C. for one minute). The resulting solution was mixed and subjected to T2 measurements for 50 minutes. For a given sample, a clotting time measurement was typically obtained within 5 minutes. Some examples of activators are tissue factor with phospholipids and ellagic acid with phospholipid. The reagents were placed in a tube that was subsequently placed in the magnetic field. Citrated whole blood was added and mixed. The blood was diluted 50%. The T2MR of the sample was measured for 30 minutes.

The T2Ex or the T2In protocols were also used with the addition of aprotinin (T2Ex-A and T2In-A) respectively. For the intrinsic pathway measurement (T2In), intrinsic activator solution was prepared by mixing ellagic acid stock and CaCl₂. For the intrinsic pathway measurement that includes aprotinin (T2In-A), add 41.7 μL of a 1 mg/mL stock of aprotinin to 1 mL intrinsic activator solution. 36.7 μL citrated whole blood that had been warmed to approximately 37° C. for three minutes was added and mixed. 5 μL of intrinsic activator solution with aprotinin that had been warmed to approximately 37° C. for one minute was added. The sample was mixed and read in the T2reader. The clotting time measurement was obtained after 5 minutes and then the platelet activity and fibrinolysis measurements were obtained over the following 60 minutes. In a separate assay the effects of the amounts of activator were tested.

A range of clotting times (CT) were produced by spiking in rivaroxaban. A range of fibrinogen levels were produced using blood cells that were reconstituted with fibrinogen-depleted plasma spiked with fibrinogen. Platelet activity studies utilized titrations of the platelet inhibitor abciximab (ReoPro). Fibrinolysis was induced by addition of varying amounts of tissue plasminogen activator (tPA). Reference values were obtained using Stago ST4, Chrono-Log, and Thromboelastography.

Optionally, the blood can be added to dried reagents and to create virtually undiluted samples, or may be diluted varying amounts to yield a sample that is about 40% to about 100% blood or be added to liquid reagent in volumetric ratios that is about 40% to 100%.

Data Acquisition and Fitting

The signal acquisition settings can be varied during the coagulation reaction as described herein. For example, the first portion of the process can utilize 4,200 180° pulses with an inter-echo delay of 0.5 ms that totals to a total CPMG collection time of 2.1 seconds. A recycle delay of 911 ms is used to yield a total dwell time of 3 seconds. This is repeated for a total experimental time of 2-5 minutes (i.e., the typical period during which the first phase and second phase are present in the sample during the coagulation reaction). A mono exponential fit is used to fit the data acquired above to detect the values of T2 during the clotting of both whole blood and plasma. Once that the CPMG curves are deconvoluted using a mono-exponential fit, the raw T2 data is fitted using a population function with a 5 parameter logistic regression (PL) fitting procedure. The output provides information about: (i) clotting time of the sample, (ii) the fibrinogen level of the sample, and (iii) the hematocrit of the sample.

The signal acquisition settings of the second portion of the process utilizes 14,000 echoes of 180° pulses and 0.5 ms of inter-echo delay that totals to 7 seconds of CPMG collection time, along with 3 seconds of recycle delay. This totals to a dwell time (time between T2 points) of 10 seconds. This collection setting is used for a total of 30-45 minutes after the first portion of clot formation has occurred (i.e., the typical period during which the second-sixth phases are present in the sample during the coagulation reaction). The data from the second portion of the process is fit using a multi-exponential fit. This second portion of the process is designed to detect the dynamics of platelet activity using the NLLS (non-linear least square) algorithm to fit CPMG decay curves. The program considers a linear combination of exponential functions up to three sets. The final fit yielding 1, 2 or 3 T2 and intensity values is assessed by evaluating which fit is minimized the most (using any of a variety of methods described herein).

Optionally, the data collection parameters utilize a short dwell time at the beginning of the coagulation reaction, and switch to a long dwell time at some predetermined time during the coagulation reaction. For example, a CPMG sequence utilizing a fast dwell time of 1-6 seconds can be used at point in the coagulation reaction during which the first phase and/or the second phase are present in the sample, and during which the a mono-exponential fit for the T2 decay curves is appropriate. Subsequently, the CPMG sequence used to measure the additional decay curves has a long dwell time of 8-12 seconds to maximize the signal to noise ratio during portions of the coagulation reaction where multiple water populations are distinguishable in the sample. The measurements taken using a long dwell time can be fitted using a multi-exponential algorithm.

Reported Assay Results

Clotting time was reported in seconds, along with reference ranges for normal samples, standardized to correlated methods (plasma PT and aPTT). Fibrinogen values correlated to standard method with calibration curve to report mg/dL. Relative amounts along with reference ranges for normal samples may also be reported. Platelet activity was reported as a relative numeric scale of function (0-100%), and may also be correlated to standard methods. Fibrinolysis was reported as a percent lysis or relative numeric scale of lysis.

Activated blood samples produced T2MR signals that correlated to each diagnostic result from the respective comparative method. T2MR was able to detect a range of CTs with both tissue factor and ellagic acid reagents with a precision of <5% and a correlation to plasma CT of R²>0.94. T2MR detected 100-600 mg/dL fibrinogen with a correlation to Clauss measured plasma fibrinogen of R²>0.95. T2MR reported platelet activity that titrated with the platelet inhibitor abciximab. T2MR detected clot contraction and subsequent clot lysis with the addition of tPA to the sample.

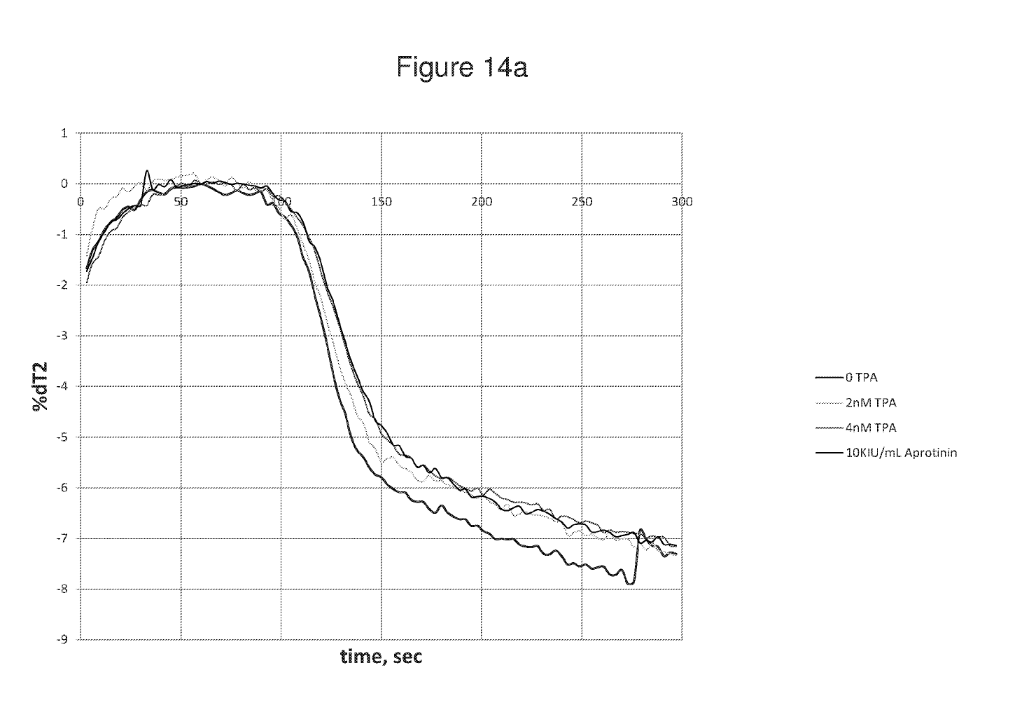

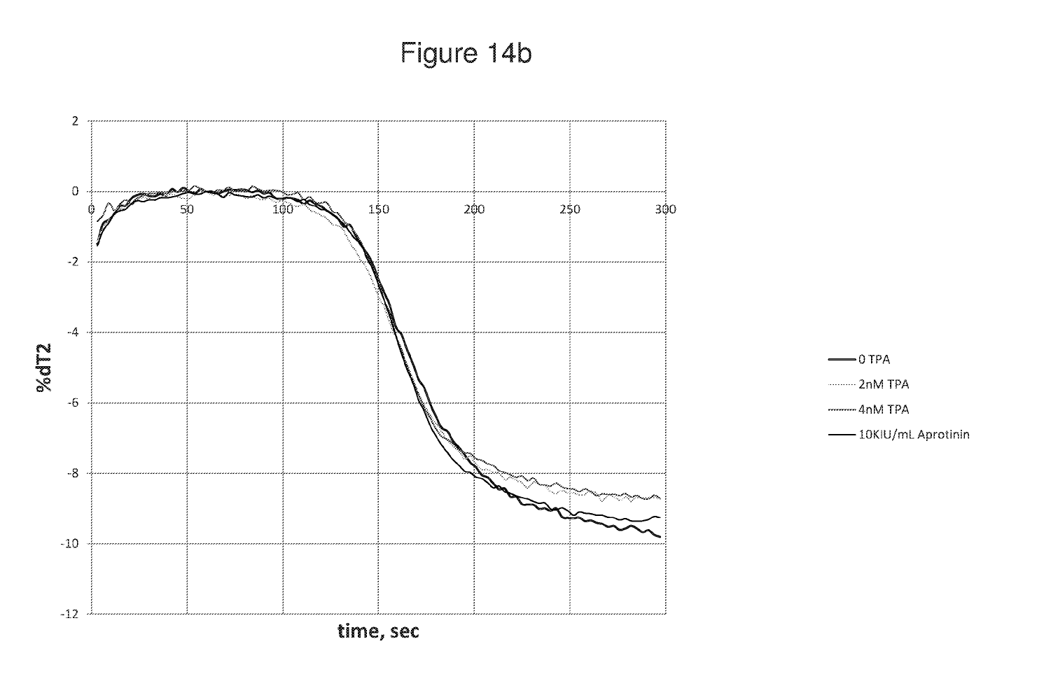

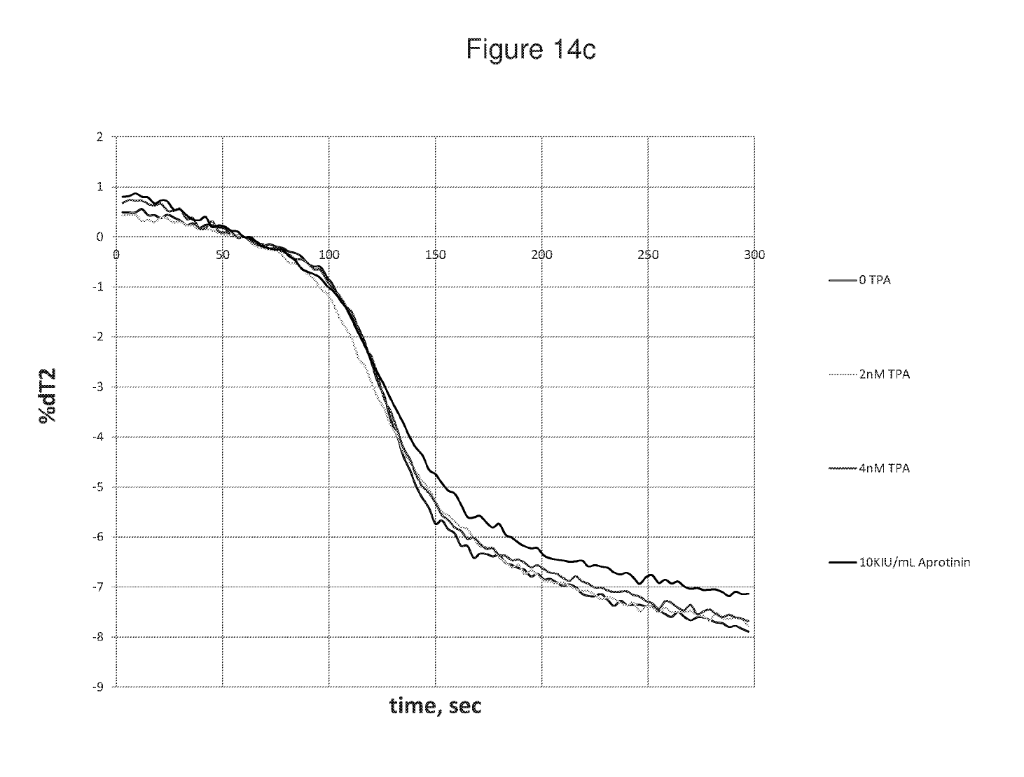

Clotting time measurements from the T2In samples are shown from the 3 different donors in FIG. 14a-14c. The plot shown represents the % deltaT2. It should be noted that the different curves with different tPA concentrations, whether or not aprotinin was added, appear to overlay. The % deltaT2 values for clotting time results are normalized for comparison.

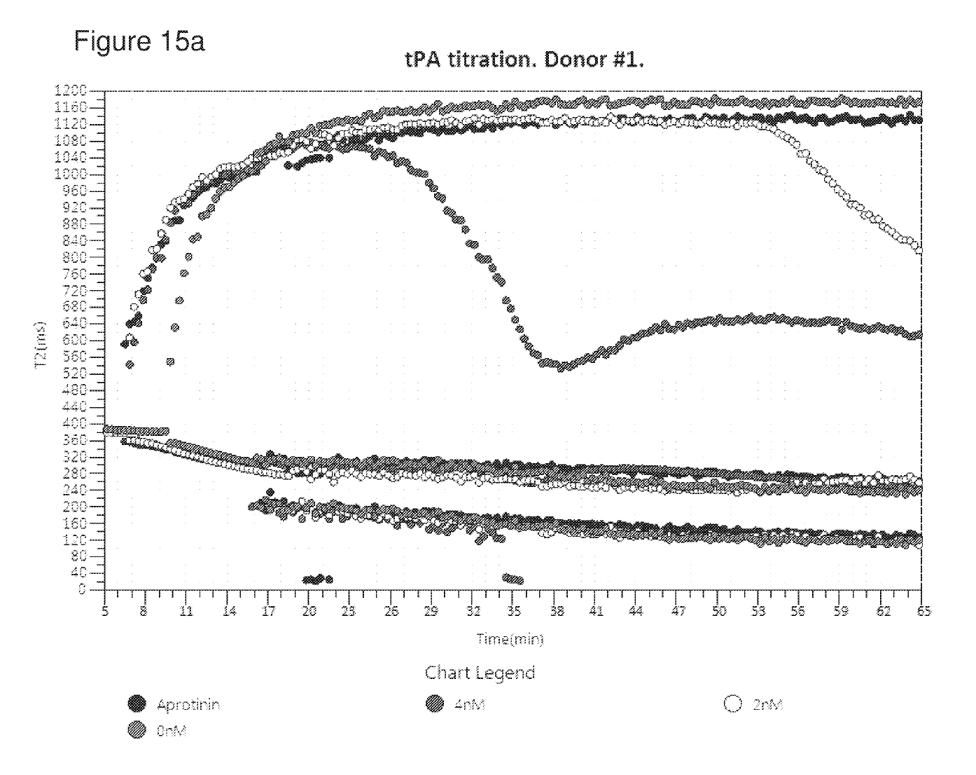

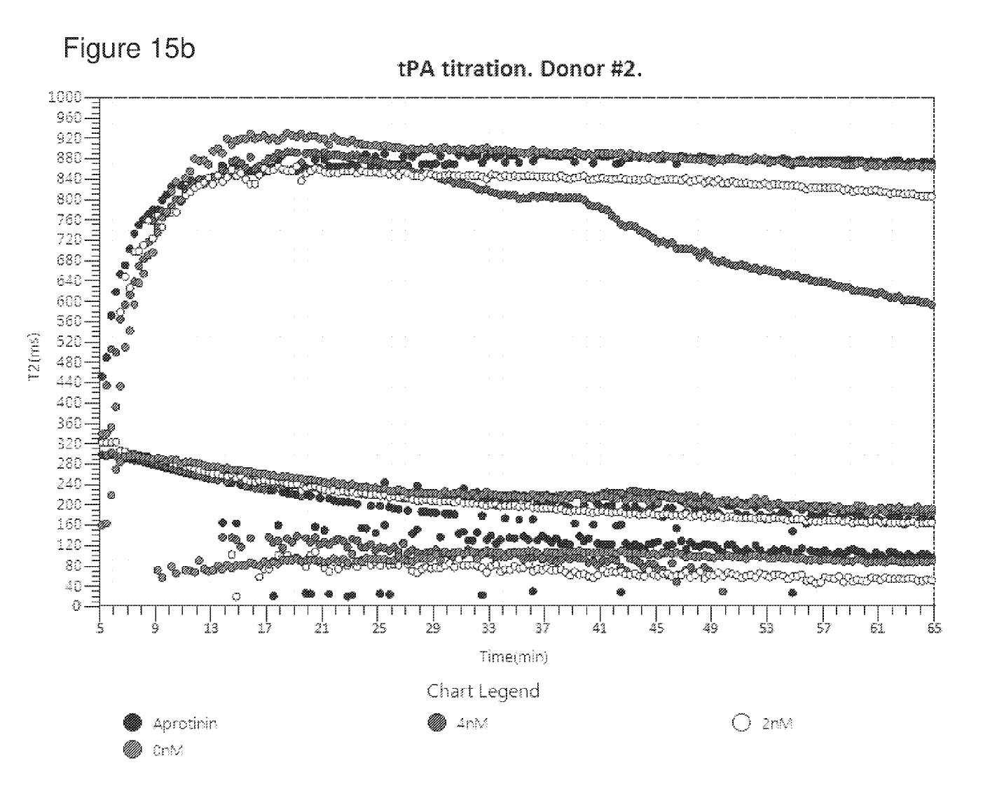

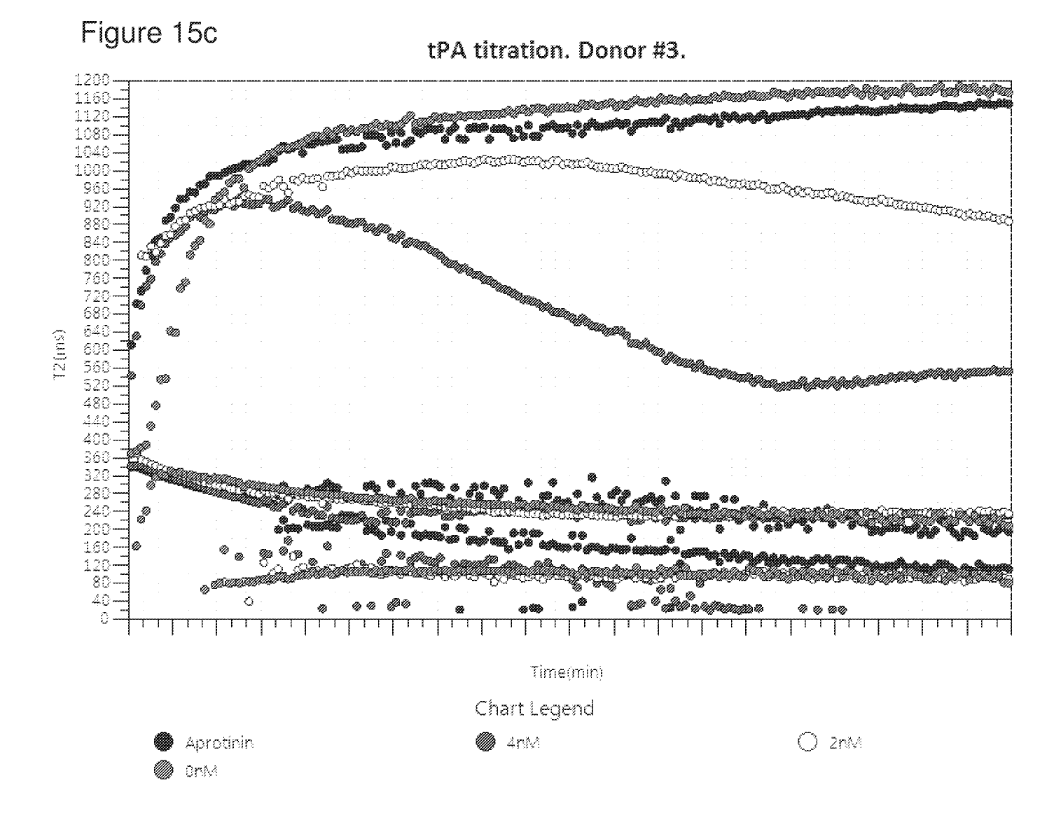

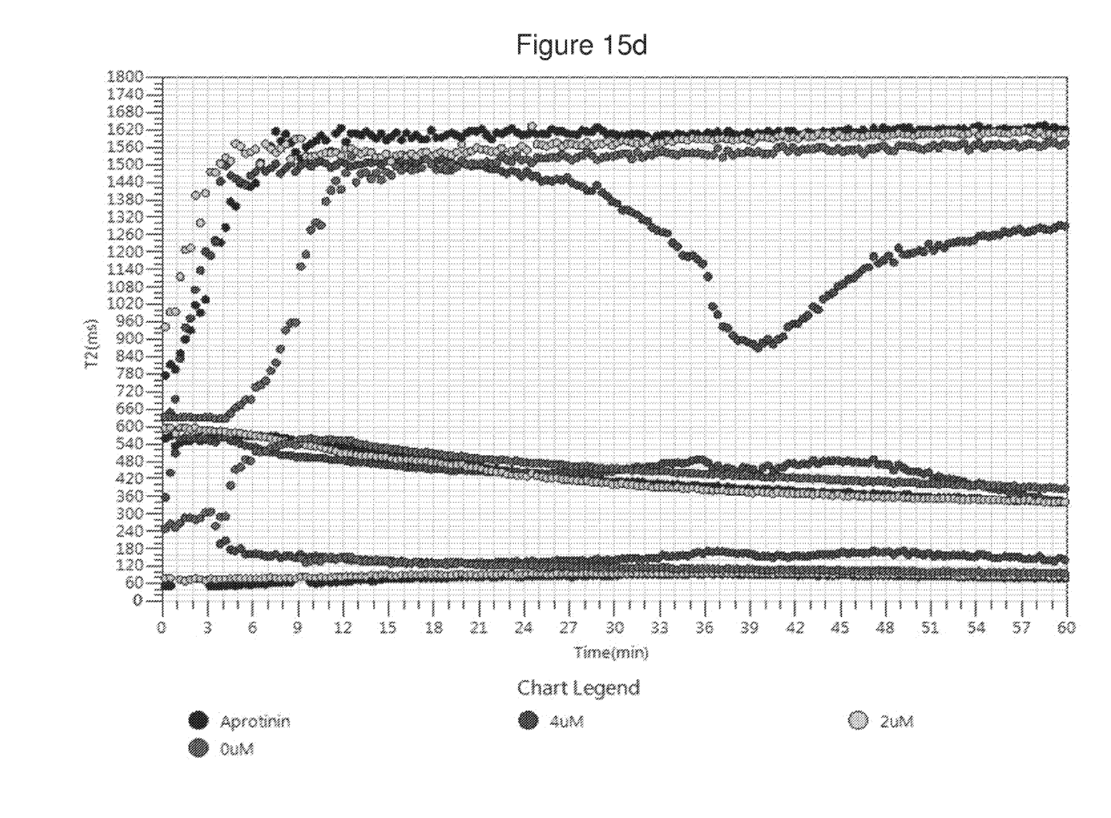

Platelet activity and fibrinolysis measurements for the 3 donor samples in the T2In assay are shown in FIG. 15a-15b. tPA levels of 0 nM, 2 nM or 4 nM were added to citrated whole blood. Blood samples (36.7 μL) were activated by addition of 5 μL ellagic acid solution with CaCl₂. T2MR data was collected for 65 min. The addition of tPA cleaves the fibrin mesh of the clot and results in clot dissolution over time. This is detected by T2MR by the change in T2 value of the serum peak (third phase). Increasing concentrations of tPA resulted in a larger change between the maximum T2 value and the minimum value of the serum peak. As a control aprotinin was added to the blood sample prior to activation with the ellagic acid solution. In these samples there was no detected change in serum T2 values. For this data, the similarity of aprotinin with the 0 nM tPA shows that no native fibrinolysis is present. If the sample had a curve like those from higher levels of tPA then that would indicate fibrinolysis and indicate that the patient needs appropriate therapy, such as administration of trans-examinic acid.

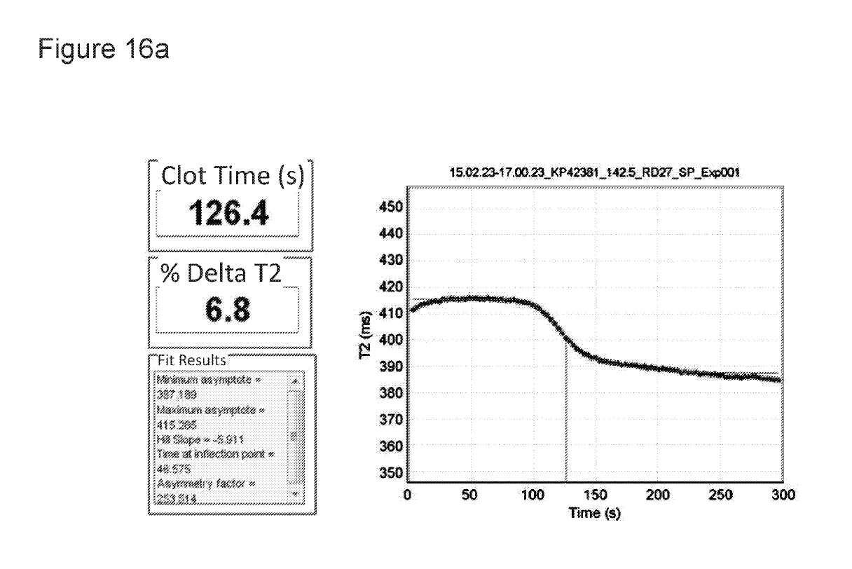

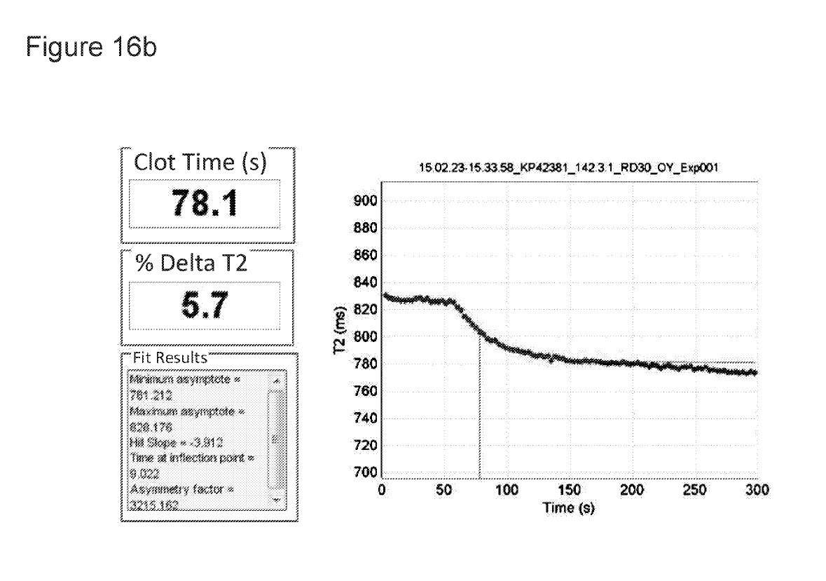

Clotting time data for two samples with different amounts of intrinsic activator added are shown in FIGS. 16a and 16b. It can be observed that the clotting time varies with activator concentration.

It was observed that T2MR detects a range of clinically valuable parameters associated with the hemostatic condition of a blood sample that correlate with gold-standard methods. Providing these measurements could impact patient management by providing rapid, comprehensive coagulation data.

The panel is a rapid point-of-care in vitro diagnostic product that measures prothrombin time and fibrinogen quantitatively and platelet function and fibrinolysis semi-quantitatively using a whole blood sample volume of less than 100 μL that can be sampled, for example, from a citrate vacutainer.

### Example 11. Fibrinogen Assay Calibration Curves: T2Ex and T2In

Fibrinogen calibration curves were created using red blood cells from at least three donors. The red blood cells were reconstituted with fibrinogen depleted plasma spiked with a known amount of fibrinogen. Activator of the extrinsic or intrinsic pathways was added. The fibrinogen range of 117-888 mg/dL is determined by Clauss method (normal range 150-500 mg/dL). Ten replicate T2MR measurements were taken for each fibrinogen level. About a 2% CV on % deltaT2 for 200 mg/dL when % deltaT2 is 5%.

Reported Assay Results

Average % CV on the T2MR signal was 3.4% for tissue factor and 2.9% for ellagic acid. The tissue factor results for a fibrinogen level of 117 mg/dL resulted in a calibration curve with 5.1% CV and with a fibrinogen level of 311 mg/dL the calibration curve has a 1.6% CV. For ellagic acid 3 independent donors were tested. The percent error from the Stago comparator was 3-36%. For the T2Ex assay, the fibrinogen values were within 8.7% of the STAGO reported value in plasma across the four donor samples that were tested. For the T2In assay, values were within 9.4% of the STAGO values across the four donor samples that were tested.

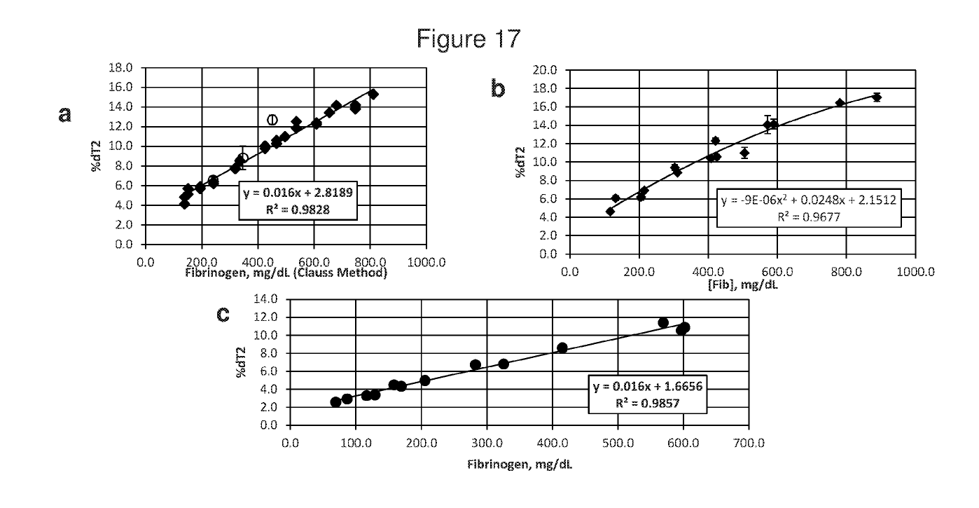

The calibration curves are shown in FIGS. 17a-17c.

### Example 12. Clotting Time Assay Evaluation with an Extrinsic Pathway Activator

The T2Ex Panel is specifically indicated as a quantitative and semi-quantitative in vitro diagnostic panel that rapidly monitors the coagulation process via the extrinsic pathway in citrated whole blood samples. Functional parameters measured with this assay include prothrombin time (PT), fibrinogen, platelet function, and fibrinolysis.

Samples from three donors were provided. Rivaroxaban spiked citrated whole blood samples were used to generate a range of clotting time measurements. An extrinsic pathway activator, such as tissue factor, was added. The T2MR clotting time (CT) range used was 32-259 seconds, and the STAGO plasma prothrombin time (PT) range was 11-47 seconds. Five replicate T2MR measurements were taken for each clotting time.

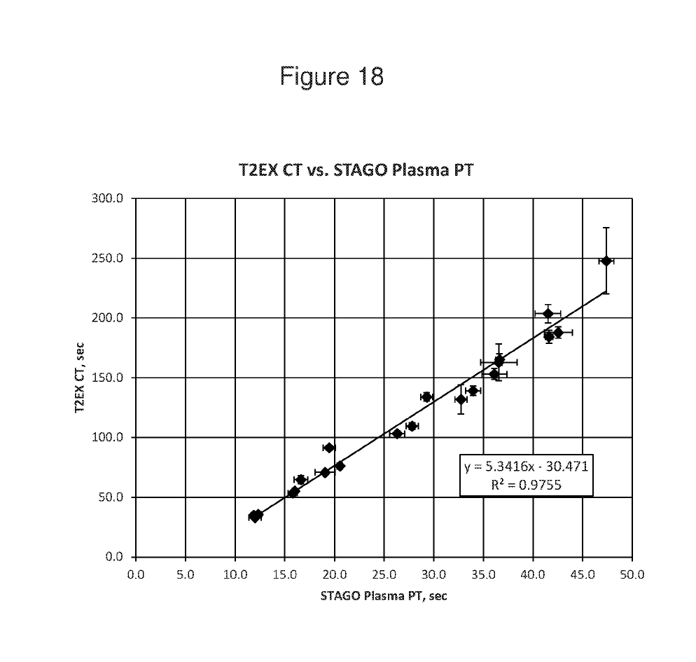

The average % CV on T2EX CT assay was 3.6%. The average % CV on the STAGO CT was 2.7%. The linear correlation with the STAGO assay resulted in an R² value of 0.98 (see FIG. 18).

### Example 13. Clotting Time Assay Evaluation with an Intrinsic Pathway Activator

Citrated whole blood samples from six donors were provided and spiked with rivaroxaban to generate a range of clotting times. An activator of the intrinsic coagulation pathway, (e.g. ellagic acid), was added. T2MR clotting time range was between 73-202 seconds, and the STAGO aPTT range was between 29-86 seconds. Five replicate T2MR measurements were taken for each clotting time. The specification desired was between 0.7-5% CV with 100-200 seconds of clotting time and a detection range of 60-450 seconds.

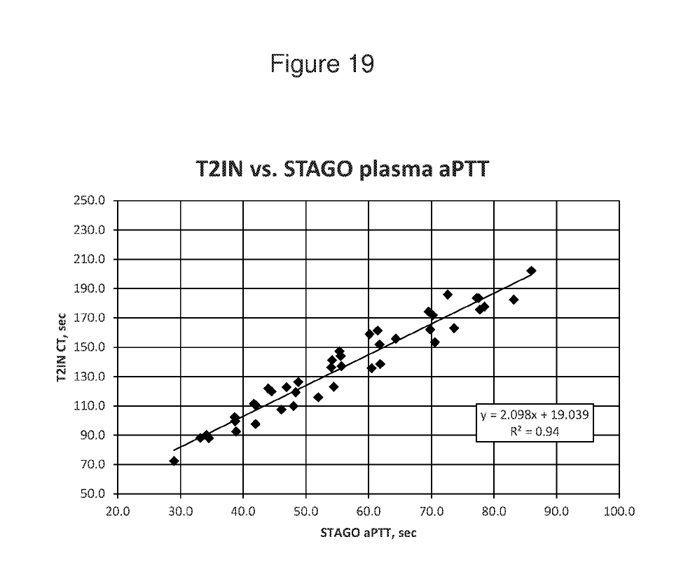

The linear correlation with the STAGO protocol yielded an R² value of 0.94 (see FIG. 19). The performance requirements of the assay can now be tested.

### Example 14. Platelet Activity T2MR Assay with an Extrinsic Pathway Activator

This example demonstrates the use of T2MR detection to monitor varying levels of platelet activation and correlate activity to whole blood aggregometry.

Citrated whole blood samples from three donors were obtained. Titrations were performed with increasing amounts of the actin polymerization inhibitor cytochalasin D (CCD) or the platelet inhibitor abciximab (ReoPro). The inhibitor was added to the samples prior to activation with tissue factor, or another extrinsic coagulation pathway activator.

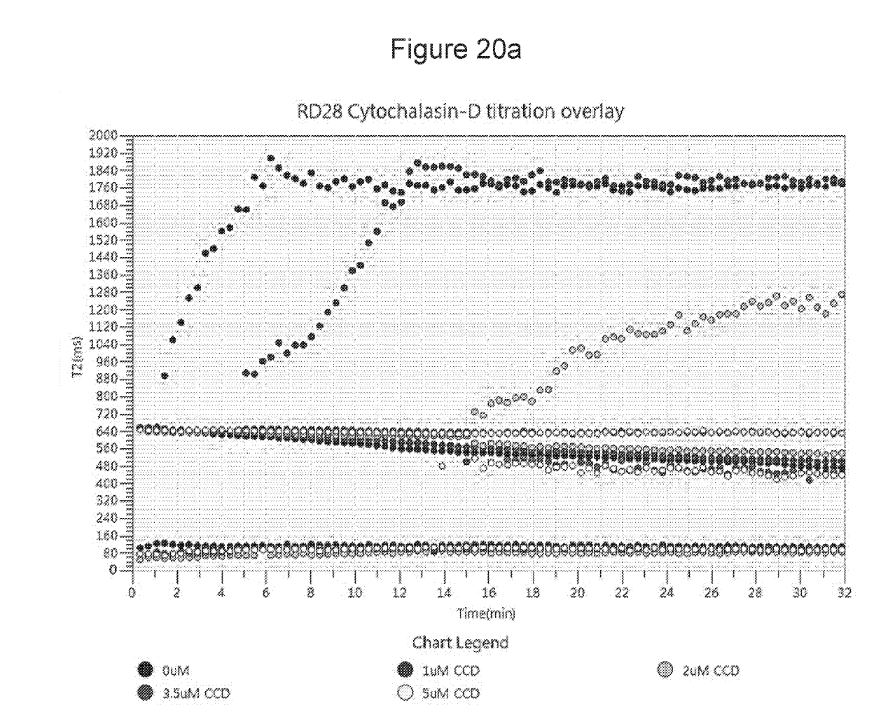

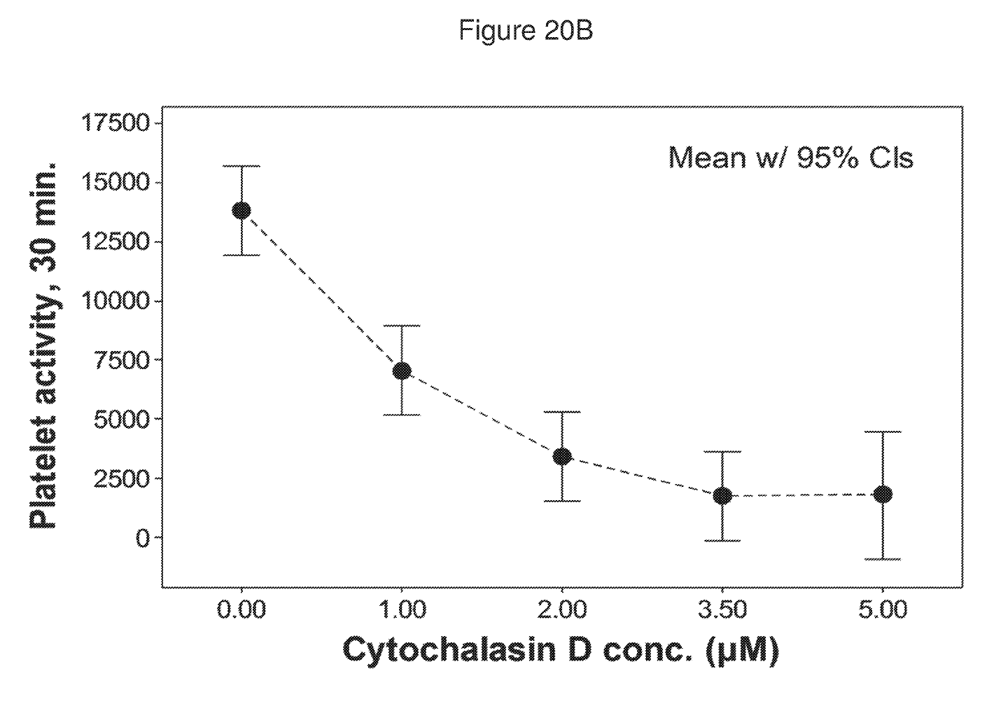

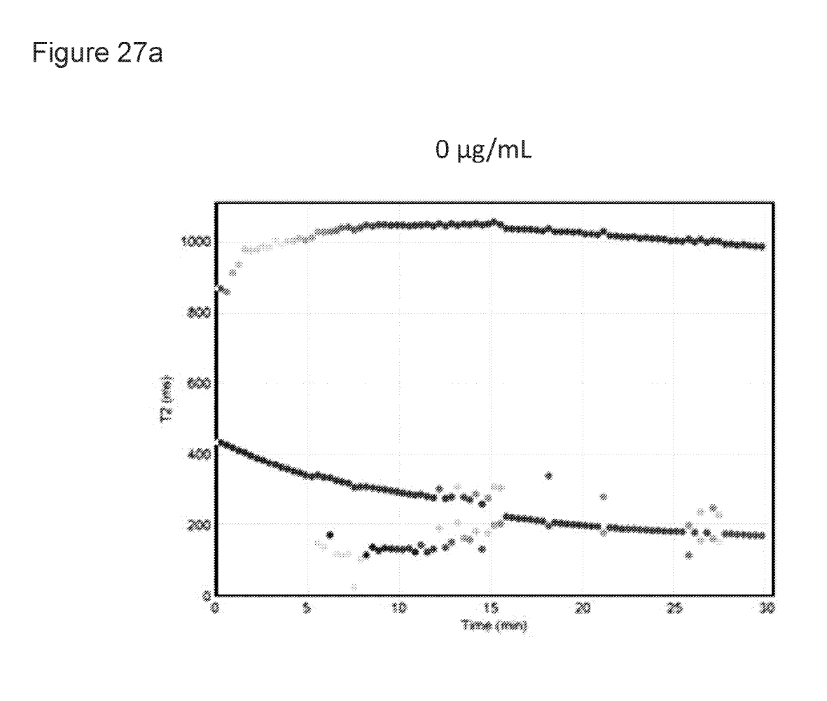

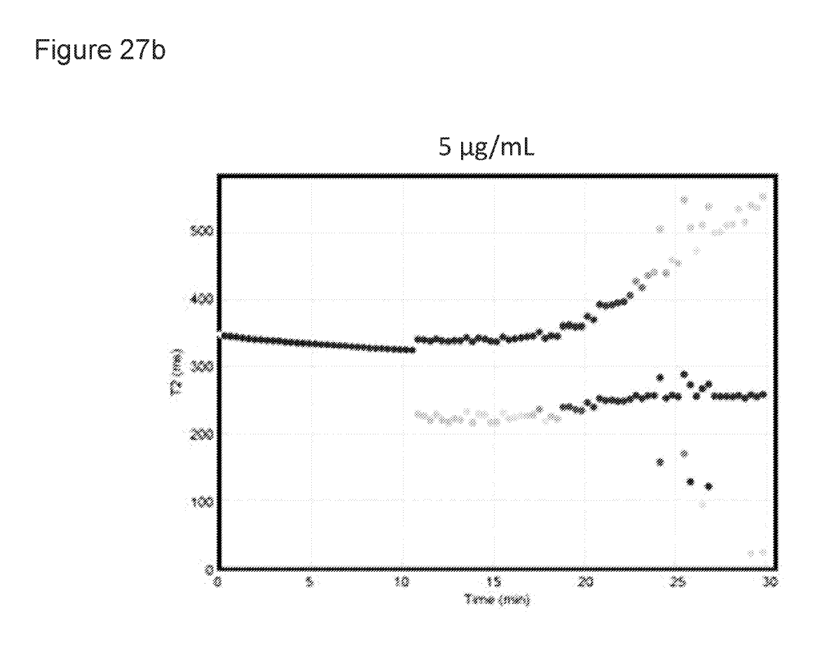

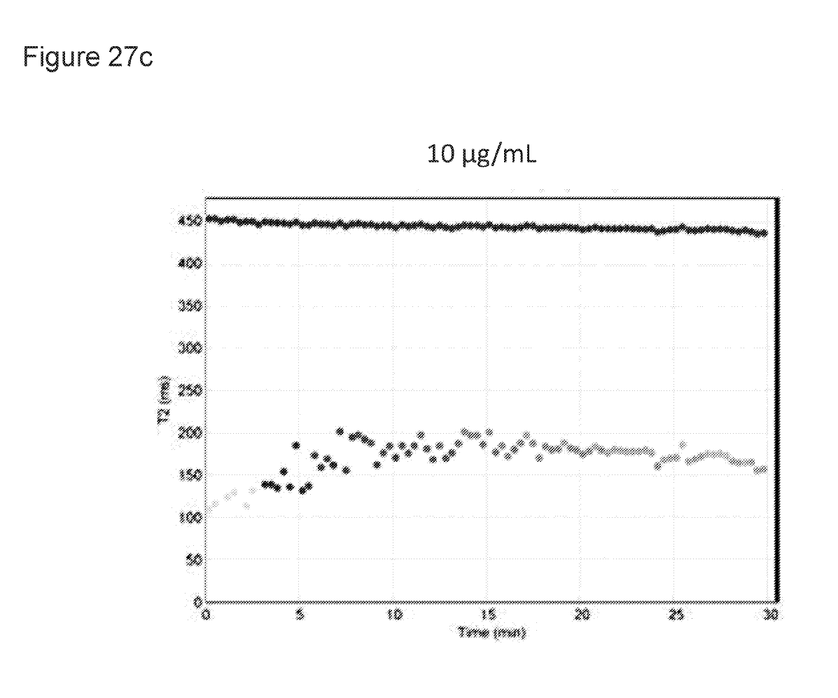

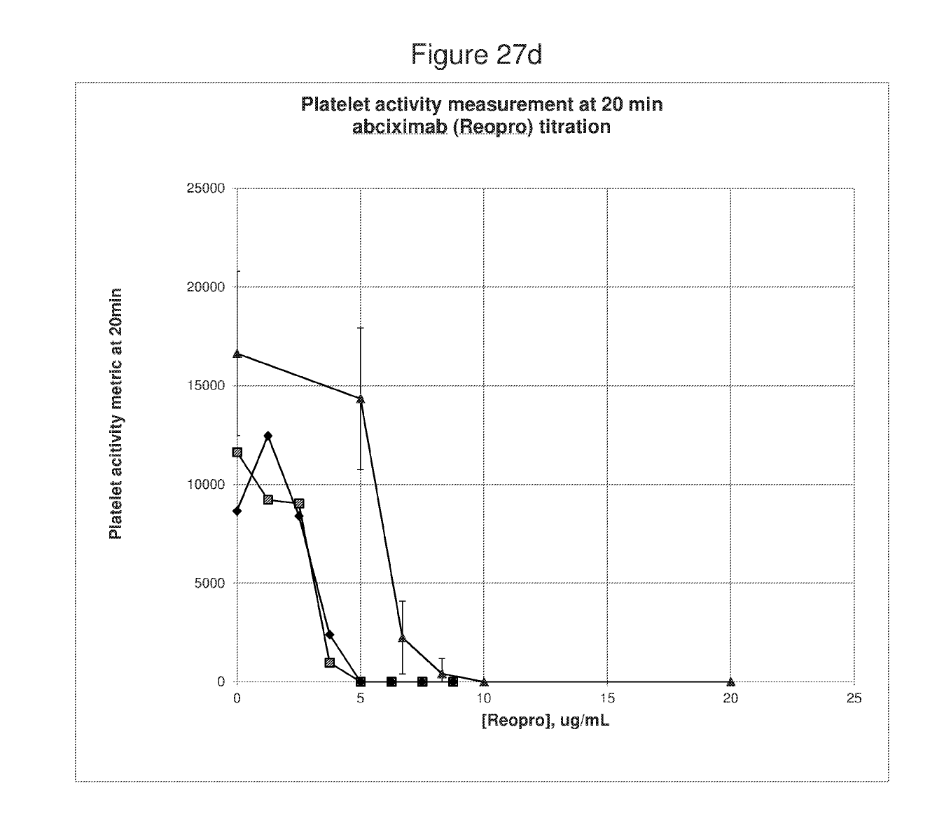

The CCD had no reproducible effect on platelet aggregation using whole blood aggregometry (WBA) or light transmission aggregometry (LTA) (see FIGS. 20a and 20b). These results led to the use of platelet inhibitor drugs such as abciximab (ReoPro) to develop other platelet activation assays (see Examples 8 and 26, and FIGS. 5 and 27).

### Example 15. Detection of Clot Time, Platelet Activity, and Fibrinolysis and Correlation with ROTEM®

This example demonstrates the use of T2MR to detect fibrinolysis and correlated results to ROTEM® rheological data.

Normal citrated whole blood samples from three donors were obtained. Different levels of tissue plasminogen activator (tPA) were spiked into the samples prior to activation with tissue factor or ellagic acid. ROTEM® was used as a method for comparison. For the intrinsic pathway measurement (T2In), intrinsic activator solution was prepared by adding 1 mL of ellagic acid stock to 24 μL of 1 M CaCl₂. For the intrinsic pathway measurement that includes aprotinin (T2In-A) 500 μL of the BMI aprotinin stock was added to 500 μL of 0.2 M CaCl₂. 35.2 μL citrated whole blood (warmed to approximately 37° C. for 3 minutes) was added and mixed with 4.8 μL of extrinsic activator solution (warmed to approximately 37° C. for one minute prior to addition). T2MR measurements were made using a T2reader.

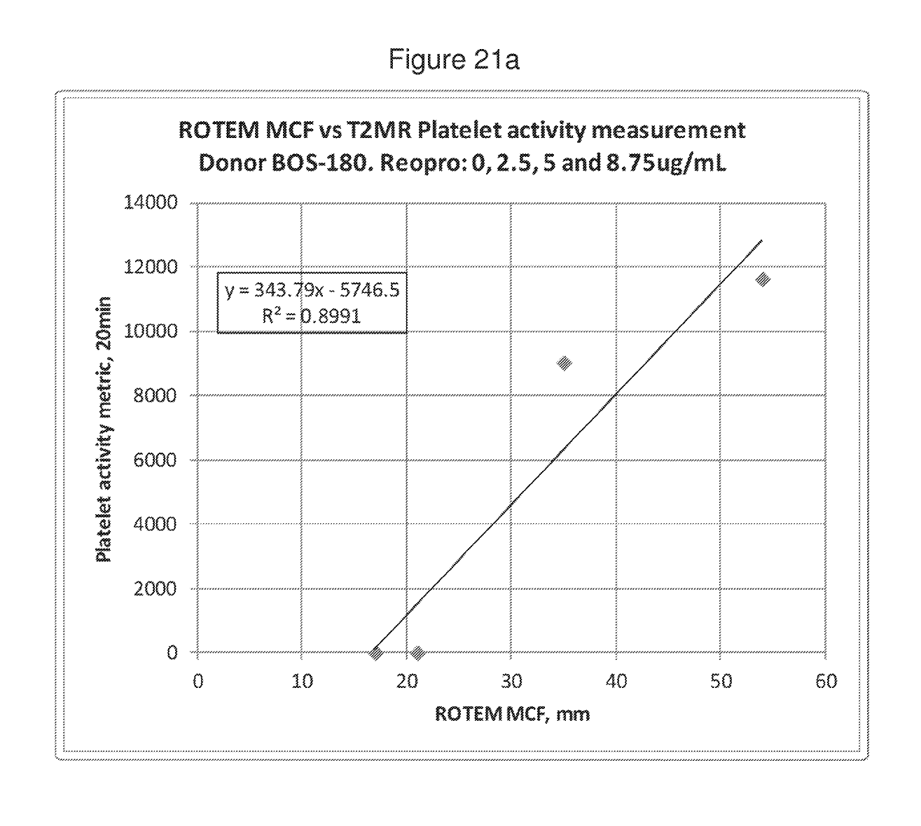

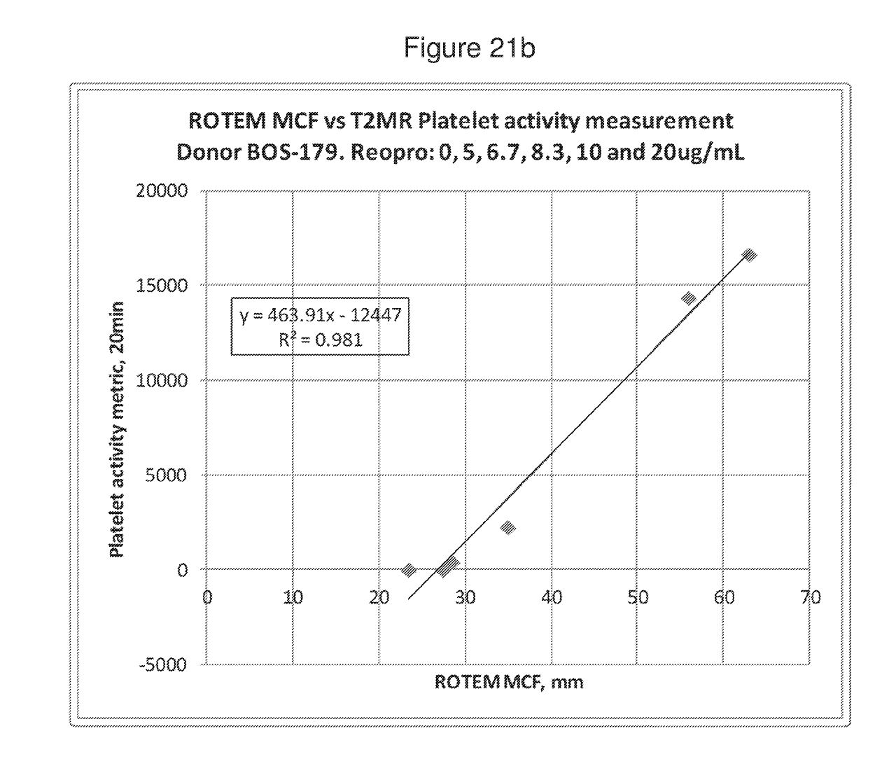

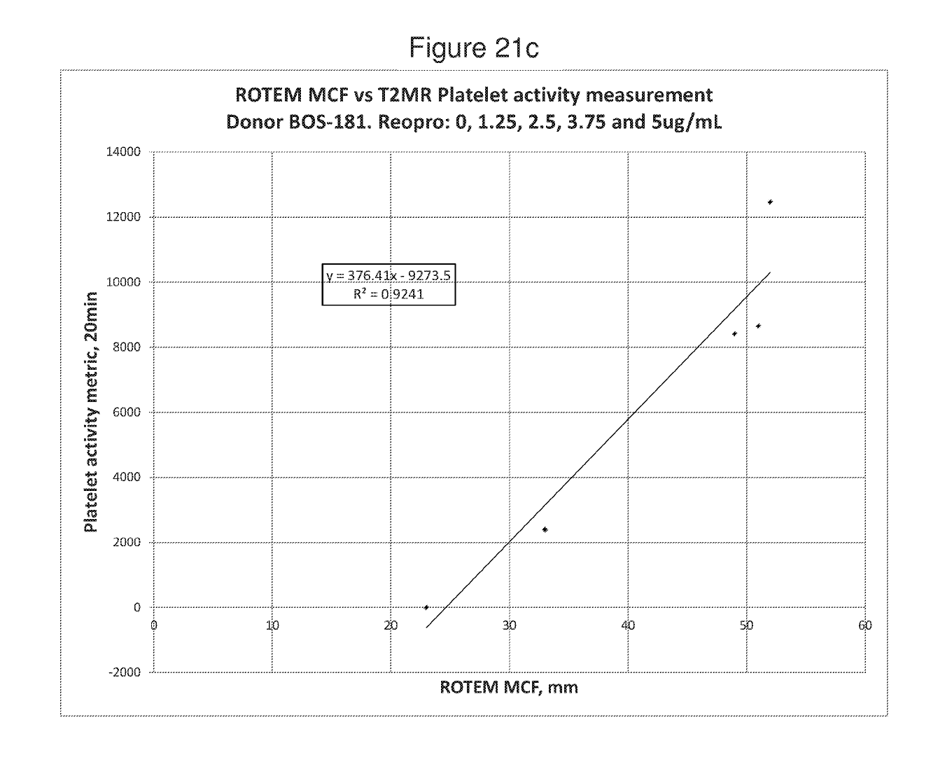

Platelet activity measures based upon T2MR were observed to be correlated to ROTEM® (see FIGS. 21a-21c). Fibrinolysis measures based upon T2MR showed that there is substantial donor variation in sensitivity to tPA.

### Example 16. 5 Parameter Logistic Fitting of the Clotting Time

This example demonstrates the use of T2MR to detect a clotting time, hematocrit, and fibrinogen level using a fitting procedure to identify T2MR features, such as the transition during the coagulation reaction from a sample comprised substantially of the first phase to a sample comprised substantially of the second phase. Once the CPMG curves are deconvoluted to produce raw T2 data (i.e., in the first component of the T2MR curve), the raw T2 data is fitted using a population function with a 5 parameter logistic regression (PL) fitting procedure.

The 5-PL or 5 Parameter Logistic is a nonlinear regression model used for prediction of the probability of occurrence of an event by fitting data to a logistic curve. It differs from the 4-PL or 4 Parameter Logistic model in that it is an asymmetric function which is a better fit for immunoassay or bioassay data. As the name suggests, there are 5 parameters in the 5-PL model equation, equation 20:

F(x)=D+(A−D)/((1+(x/C)̂B)̂E) (20)

where A is value for the minimum asymptote; B is the Hill slope (the Hill slope refers to the steepness of the curve and can be either be positive or negative); C is the time at the inflection point (i.e., the point on the curve where the curvature changes direction or sign); D is the value for the maximum asymptote (also referred to as “T2Max”); and E is the asymmetry factor (i.e., when E=1 we have a symmetrical curve around inflection point and so we have a four-parameters logistic equation).

Using the 5-PL fit, the inflection point is correlated to the clotting time, the maximum asymptote is correlated to the Hematocrit, and the % deltaT2, calculated as ((D−A)/D)*100, is proportional to the fibrinogen level in the whole blood sample.

### Example 17. Plasma Prothrombin Time (PT) Assay

This example demonstrates the use of T2MR detection to measure a prothrombin time.

Plasma was diluted 43% with saline or citrate. Subsequently 35.2 μL of diluted sample and 4.8 μL of PT reagent comprised of tissue factor and phospholipids were pre-heated separately at 37° C. prior to mixing. The sample was placed in a T2MR reader and the data was fitted as described in Example 16. The prothrombin time of the sample was determined.

### Example 18. Hematocrit Assays

This example demonstrates the use of T2MR detection to measure the hematocrit of a sample.

The measurement of hematocrit (HCT) can occur prior to the addition of activator to the blood sample. In such an instance, 20 μL of blood could be moved from the Vacutainer® to the sample tube. The sample tube could then be moved into the T2MR and read for 10 to 60 seconds to acquire the HCT measurement.

Alternatively, the HCT measurement is made as described in Example 16.

### Example 19. Fibrinogen Assay with Correction for Hematocrit

This example demonstrates the use of T2MR detection to measure a fibrinogen level. As mentioned above, hematocrit can interfere with the measurement of fibrinogen. To provide an accurate measure of fibrinogen in whole blood, it is necessary to have an independent characterization of hematocrit, to compensate for the changes in available blood volume with cell density. Using T2MR relaxation measurements, the absolute relaxation time (T2) of water in whole blood tends to be primarily a function of hematocrit, whereas the change in T2 during the fibrin clot tends to be primarily a function of fibrinogen. While fibrinogen concentration is a significant contributor to the hematocrit measurement, and vice versa, by solving a system of equations, it is possible to correct for hematocrit and arrive at an accurate measure of fibrinogen in whole blood.

The T2MR measurements acquired are a “T2Max” and “% deltaT2” measurement, which primarily depend on hematocrit and fibrinogen, respectively. To acquire these data, blood clotting is initiated by adding calcium and one or a combination of several activators to citrated whole blood. The activators may include any agent that initiates the extrinsic, intrinsic, or other pathways, including tissue factor, ellagic acid, or kaolin, or may be agents that bypass these pathways and directly convert fibrinogen to fibrin, including batroxobin or ecarin. During fibrin formation in whole blood, the T2 relaxivity time of water decreases in a rapid and non-linear fashion, due to differences in water diffusion interactions with the newly formed gel-like matrix (see FIG. 12). To quantify the change, a 5 parameter logistic regression function is fit to the raw T2 data as described in Example 16. The % deltaT2 and T2Max values are simply divided by each other to simultaneously account for changes in fibrinogen and hematocrit and their effects on T2 and relaxivity.

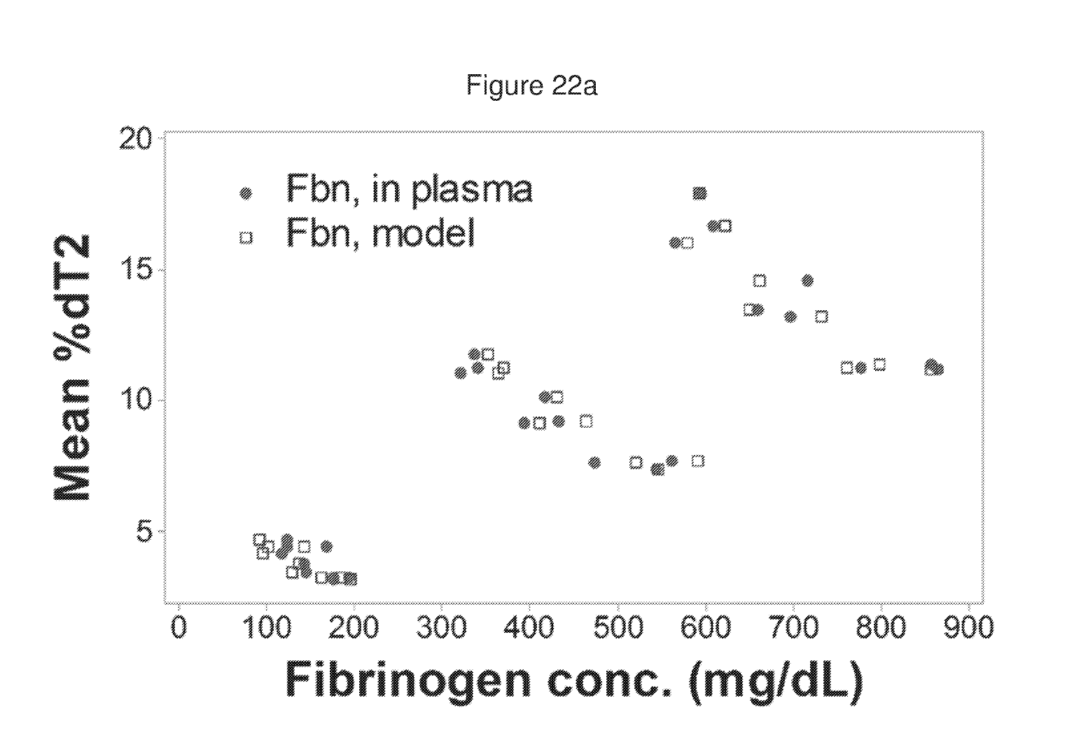

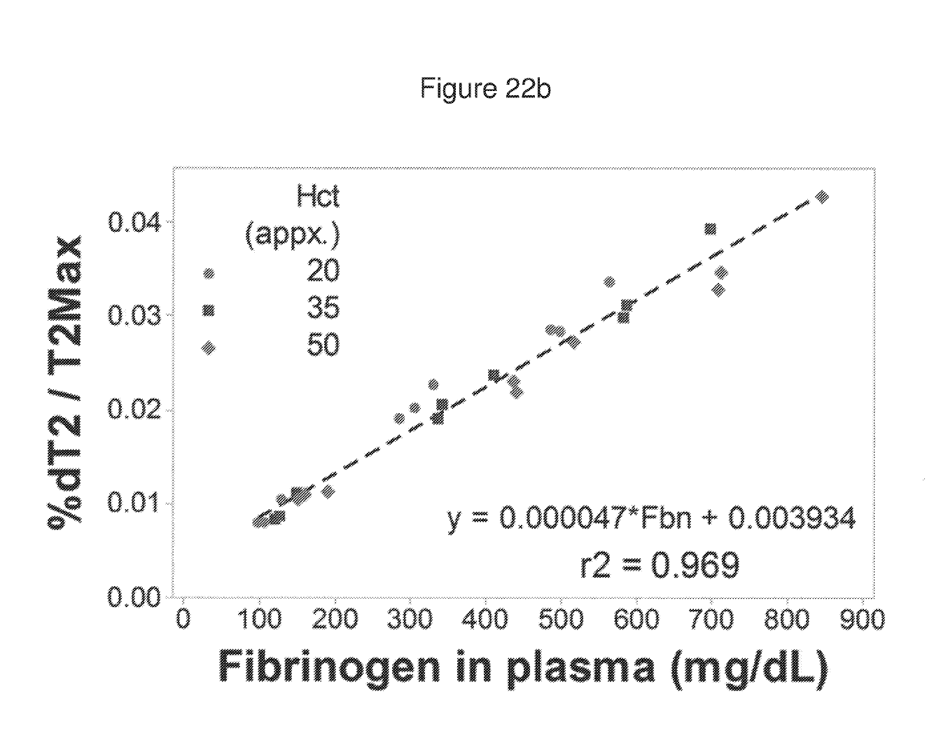

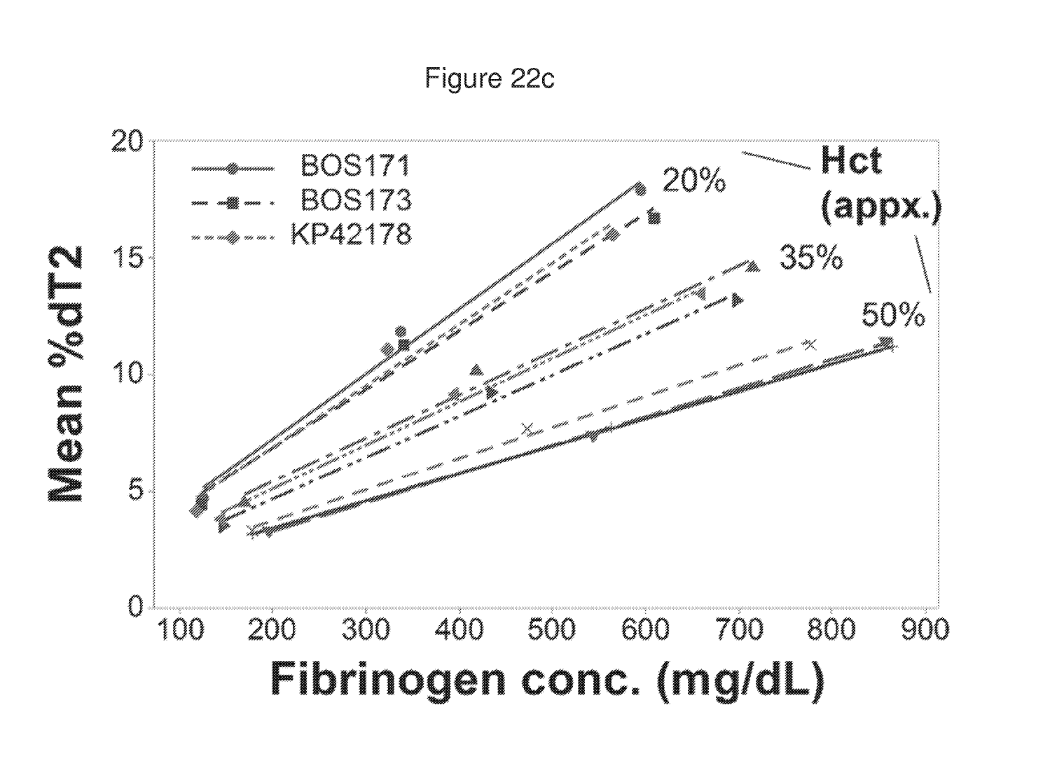

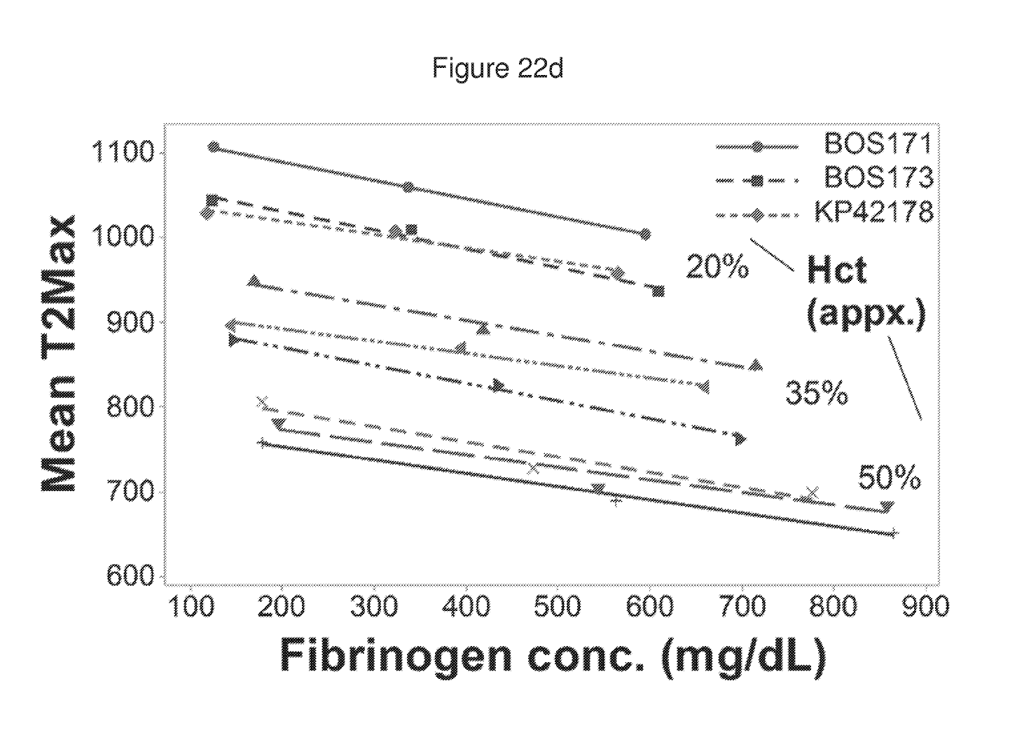

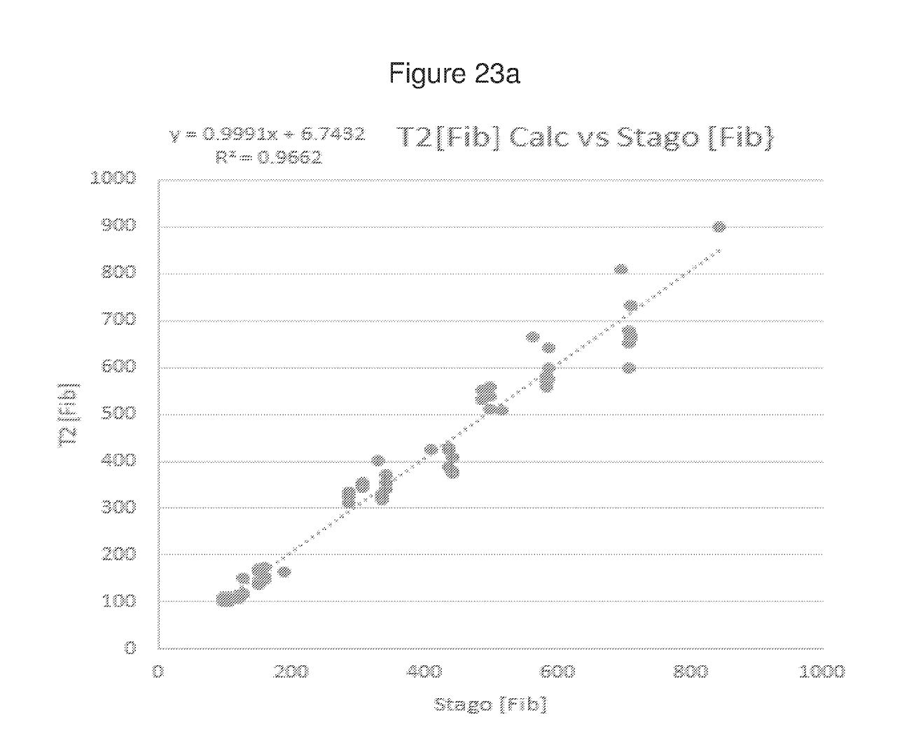

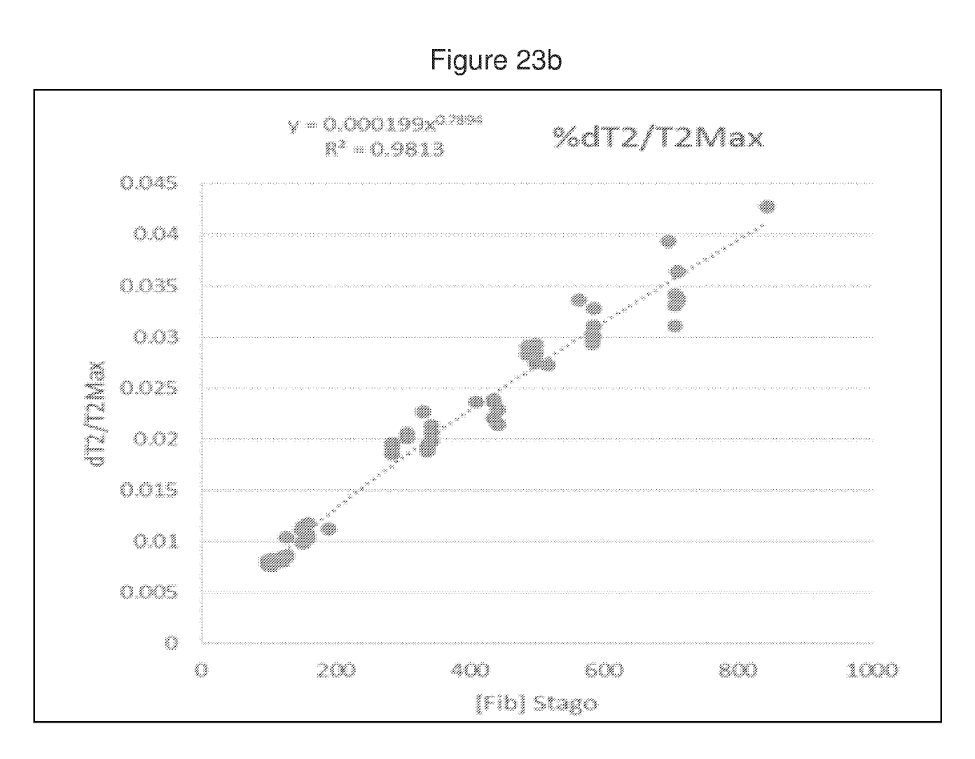

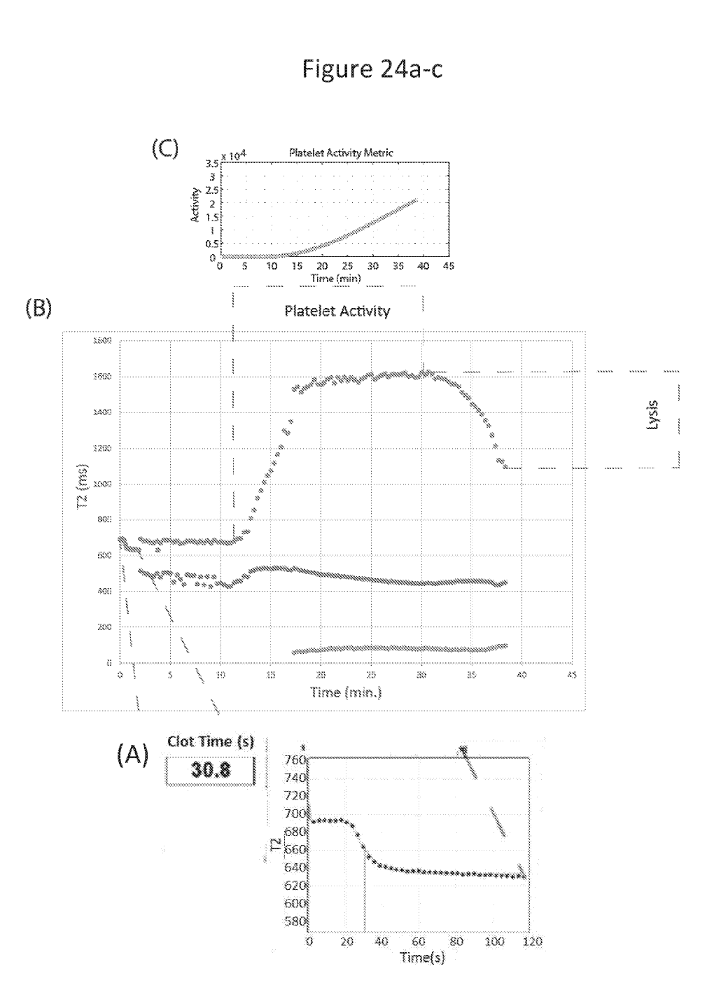

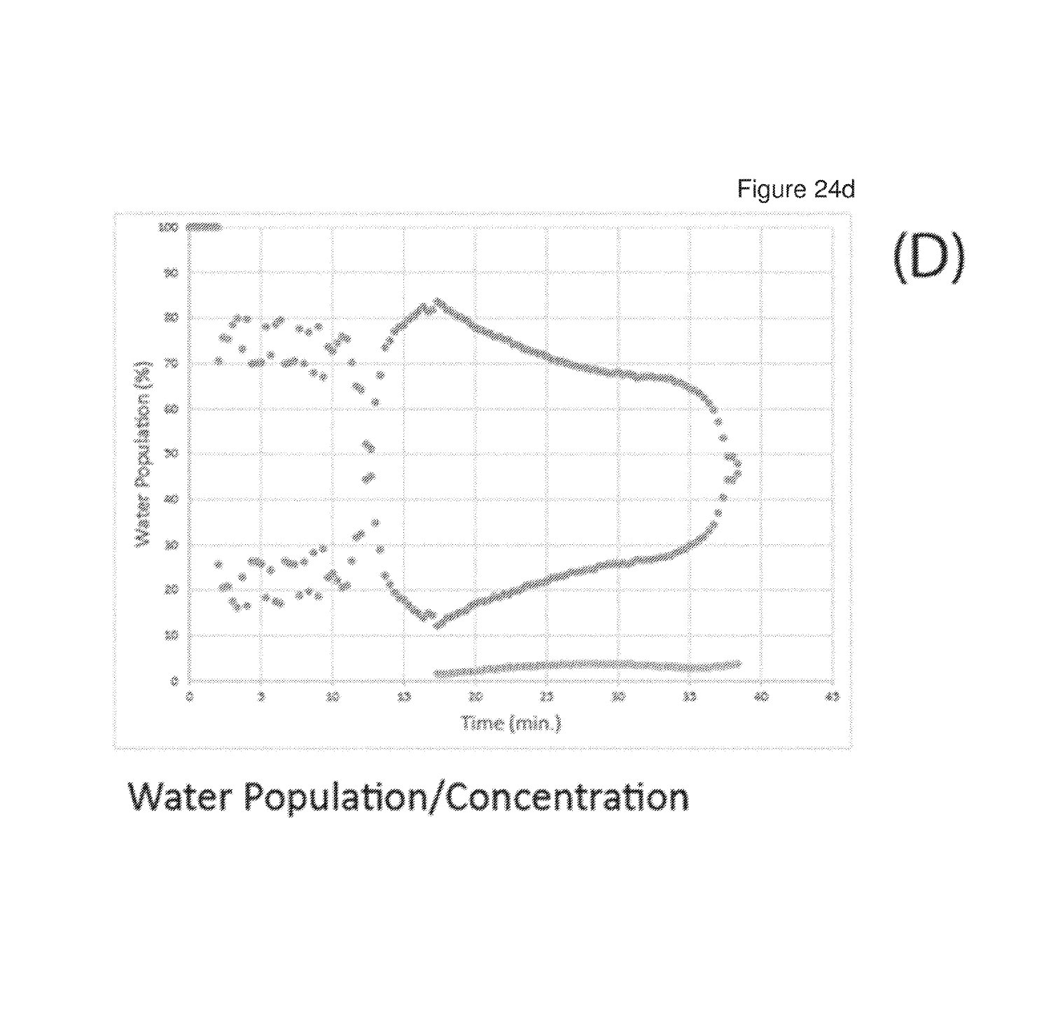

T2MR measurements can be used that were acquired on the same or different aliquot of the blood sample. For example, a second measurement can be taken with a blood sample that is at a different dilution than the sample used to activate fibrin formation. T2MR signals from this measurement can be used to normalize the % deltaT2 measurement to correct for the hematocrit interference. In short, hematocrit interference with a fibrinogen measurement can be corrected using T2MR measurements on a patient sample using one or more aliquots from the blood sample at a same or different sample dilution. Comparisons of fibrinogen concentration data with the HCT corrected versus plasma lacking hematocrit is found in FIG. 22a. FIG. 22b demonstrates that data that has been corrected for hematocrit concentration using % deltaT2/T2Max. The dependence of the % deltaT2 on hematocrit and fibrinogen is depicted in FIG. 22c and the dependence of T2Max on hematocrit and fibrinogen is depicted in FIG. 2d. Varying amounts of hematocrit in a blood sample from the same donor affects the % deltaT2, and this data reinforces the importance of the hematocrit correction. FIG. 23a and FIG. 23b are representative of the calibration of the hematocrit correction for fibrinogen.

### Example 20. Multiplexed Panel for Evaluating Samples from Neonatal Patients

A correlation between multiplex panel parameters and clinical outcomes, such as bleeding and thrombosis, in pediatric patients undergoing complex congenital cardiac surgical procedures can be determined. These patients can be prospectively followed during their hospital stay for up to 30 days (less if discharged) to determine incidence of specific clinical and/or imaging evidence for major thrombosis events and bleeding. The study can provide a tool for risk stratification by coagulation profiling in full-term neonates and infants undergoing cardiac surgery, and identify potential targets for intervention. The multiplexed panel parameters assessed in patients undergoing cardiac surgery can be compared to non-cardiac surgery patient controls (100 additional full-term neonates and infant patients that are <12 months of age undergoing a surgery that is unrelated to congenital cardiac defects).

Bleeding events that can be evaluated include chest tube output and blood product volume during the first 4, 12, and 24 hours post-surgery, as well as reoperation for bleeding in the first 24 hours post-surgery. Thrombosis events that can be evaluated include stroke, shunt thrombosis and ischemia, evidence of thrombosis by imaging studies (echocardiography or cardiac catheterization), and thrombosis complications during the hospital stay and up to 30 days post-surgery.

The fibrinogen function, clot formation and stability at each of the 4 time points previously mentioned can be correlated to clinical events. Correlation with other clinical laboratory parameters (including PT/PTT, fibrinogen, platelet count, and TEG values when available) can be performed.

The above-mentioned outcomes of the multiplexed panel parameters to clinical laboratory parameters and markers of the coagulation system can demonstrate correlation between T2MR signals and clinical outcomes such as bleeding and thrombosis events.

T2 data collected can include:
T2Ex: T2MR measurements can be performed after activation with tissue factor. Reported results include clot time, fibrinogen level, platelet activity, fibrinolysis, and the T2MR signature.
T2In: T2RM measurements can be performed after activation with contact factor. Reported results include clot time, fibrinogen level, platelet activity, fibrinolysis, and the T2MR signature.
T2Ex-A or T2In-A (+aprotinin): T2RM measurement can be performed using T2Ex or T2In with the addition of aprotinin. This is to be used as a control for fibrinolysis. Reported results include clot time, fibrinogen level, platelet activity, fibrinolysis, and the T2MR signature.

### Example 21. Sequential Blood Samples from Traumatically Injured Patients Study (TIPS)

The clinically significant mechanisms and pathways by which the inflammatory and coagulation pathways are activated immediately following major and low patient trauma, how they produce organ injury, and how they affect outcome in terms of organ failure and death. In addition, the data collected from the trauma patients can be compared to healthy control patients. The multiplexed hemostasis panel will be used to monitor coagulation parameters that can be correlated to other measured patient variables.

The multiplexed hemostasis panel can be evaluated and compared with other functional testing devices. Clotting times, fibrinogen levels, platelet function and fibrinolysis can be measured using the multiplexed hemostasis panel in tandem with current devices and biological assays currently being run by a collaborating research lab. Results from all devices can be compared and reviewed for correlation to current and approved devices. In addition, the T2 hemostat multiplexed panel and current devices in the research labs can be used to determine the incidence, prevalence and phenotypes thrombin potential and coagulopathy after severe injury. Lastly, the T2 hemostat multiplexed panel data can be compared with clinical observations including medications, transfusion therapy, and outcomes.

This prospective cohort study can review the results from sequential blood sampling using the T2 hemostat multiplexed panel and current devices in the research lab in three patient subsets: major trauma, low trauma and healthy controls.

Trauma patients can be enrolled into the study in the Emergency Department. Blood samples will be collected immediately and over the following 5 day period (if the subject remains eligible) to characterize the nature, extent and duration of the response of the inflammation and coagulation systems to trauma. These results can be correlated with patient injuries, their resulting physiological disturbances and their subsequent clinical course and outcome. In addition, clinical data on organ system failure, morbidity, ICU & hospital stay and mortality will also be collected where applicable.

Study participants who meet the criteria for major trauma and who are admitted or expected to be admitted to the Intensive Care Unit will be included. This subgroup of civilian trauma patients represent the best approximation for the types of injuries, magnitude of hemorrhage, and the degree of physiologic alteration that are encountered in severely injured civilian and battlefield casualties. Patients can be fluid resuscitated using standard advanced trauma life support (ATLS) resuscitation protocols and SFGH massive transfusion protocols.

Additional participant subsets will include blood samples drawn from both low trauma subjects and healthy control subjects for comparison purposes to the major trauma participant subset.

If the subject is admitted to the ICU, and remains in the ICU, samples can be obtained and tested at 2, 4, 6, 12, and 24-hours and daily thereafter (every 24 hours) for a total of 5 days and up to 10 total samples. If the subject is expected to be admitted to the ICU, samples can be obtained and tested until such time that it is determined that the patient is not admitted to the ICU. Healthy control patients can have a single blood sample drawn at the time of enrollment.

All variables can be tabulated using descriptive statistics. Continuous variables will be presented as means and standard deviations with 95% confidence intervals (CIs), as well as medians, minimums and maximums. For categorical variables, relative frequencies and 95% CIs can be provided.

### Example 22. T2MR Clotting Time Measurements

The T2MR based hemostasis assays described herein can be used to discriminate normal from abnormal patient samples for each of the assay readouts. For example, the clotting time readout can be used with either an extrinsic or intrinsic pathway activation to identify abnormalities in clotting time that may result in the presence of a therapeutic compound such as a vitamin K agonist like Coumadin (warfarin), a direct thrombin inhibitor like riveroxiban, the thrombin inhibitor heparin, or the deficiency of specific clotting factors such as factor VIII.

Coumadin Therapy

To demonstrate this capability, blood samples from normal donors not on therapy were compared with samples from donors on Coumadin therapy. The donors on Coumadin had prolonged clotting times for citrated plasma, citrated blood and fresh whole blood without an anticoagulant.

All samples were sourced from the coagulation laboratory at a local hospital. Lab protocol was to collect discarded citrated blood samples (venous draw, 1 mL minimum) from patients on Coumadin therapy with both normal and abnormal PT values. Citrated blood samples were stored at RT and within 4-5 hours of the draw time samples were spun down at 1500×g for 15 min to prepare plasma. Plasma samples were stored frozen at −70° C.

For citrated plasma, a tissue factor reagent was prepared by reconstituting the lyophilized Dade® Innovin® (tissue factor) reagent. Citrated plasma (13 μL) was mixed with 27 μL of tissue factor reagent. All reagents and disposables were adequately pre-heated. T2 values were collected with a 1 second dwell time and used to collect the T2 values as a function of time. The T2 vs. time data were collected for 120 successive measurements and fit with a five-parameter logistic fit to calculate the EC50, from which the clotting time was derived. A method comparison against the Stago ST4 system and a precision study was completed with 22 samples from normal donors not on anti-coagulant medications and 50 samples from donors on Coumadin. The average % CV within sample was 2.7%, 90% of samples had a % CV of <3.5% and 95% of samples had a % CV<4%. 96% of samples had an accuracy of <16%, 94% of samples had an accuracy of <12%. INR (international normalized ratio, a normalized PT value) values were calculated from a pool of normal healthy donors. A patient who was on Coumadin had a T2MR clotting time of 51.3 seconds and an INR of 5.3, and is considered in a hypocoagulable state. This corresponds to a clotting time value that is prolonged compared to standard therapeutic ranges. A patient who was undergoing Coumadin therapy but was hypercoagulable had a T2MR clotting time of 8.4 seconds and an INR of 0.8, which shorter than the typical normal range and closer to hypercoagulable.

Similar results were obtained for citrated whole blood. The same tissue factor raw material was used and the reference method remained plasma PT values obtained on the Stago ST4 system. In this case a different dilution of tissue factor and sample were used. The Tissue factor was diluted with calcium chloride (25 mM) saline at a ratio of 3 parts saline to 1 part tissue factor. A second reagent of 11 mM citrated and 2 mg/mL fibrinogen was used. The citrated blood sample was pre-diluted at a ratio of 1:1 with the citrated fibrinogen diluent to yield a final diluted blood sample with a fibrinogen concentration of 1 mg/mL. For this assay format, 40 μL of diluted citrated blood was combined with 20 μL of pre-heated Innovin® reagent and T2 vs time values were collected for 120 successive measurements and fit with a 5PL fit to determine clotting time values. An alternative assay format was demonstrated using a different second reagent of 60 mg/mL fibrinogen without addition of citrate. This reagent was added at a volume of 2.6 μL to 150 μL of citrated blood for a final fibrinogen addition of 1 mg/mL in undiluted citrated blood. A volume of 40 μL of this fibrinogen supplemented citrated blood was combined with 20 μL of Innovin.

The former formulation was characterized for precision by measuring 13 samples. An average precision of 4.7% was observed. The latter formulation yielded much greater accuracy. A total of 12 normal and 12 abnormal samples were measured to yield overall accuracy of 6.8%. 96% of samples had an accuracy of <16% and an R value of 0.97 was observed for the method comparison. An ISI (International sensitivity index) was derived to be 1.29 for this method from a scatter plot with a Deming fit. Similar clinical observations were made as with citrated plasma.

Lastly, a similar protocol and results were obtained with fresh whole blood without the presence of an anticoagulant. In this study assay workflows described above for citrated blood were used with and without addition of extra fibrinogen were evaluated. A total of 11 normal donor samples and 10 anti-coagulated donor samples were measured. Reference measurements were obtained on the Roche CoaguChek XS. Clotting time as determined from a 5PL curve fitting algorithm applied to 120 successive T2 measurements (1 second dwell) obtained on the sample after mixing. HCT determined from the initial T2 values of the PT measurements. Correlation (R) values between T2MR and CoaguChek for mean INR were 0.97-0.99 for both assay workflows. Correlation (R) values between T2MR and SysMex for HCT were 0.95-1.0 for the calibration curve. Average accuracy for HCT was 6%-8% with as high as 94% of samples within 16% accuracy for HCT. Correlation values for HCT range from 0.52-0.82 due to narrow HCT range of samples. This demonstrates the capability of T2MR to provide a multiplexed fresh whole blood assay.

All reagents and disposables were adequately pre-heated to 37° C. to avoid artifacts from non-equilibrated temperatures.

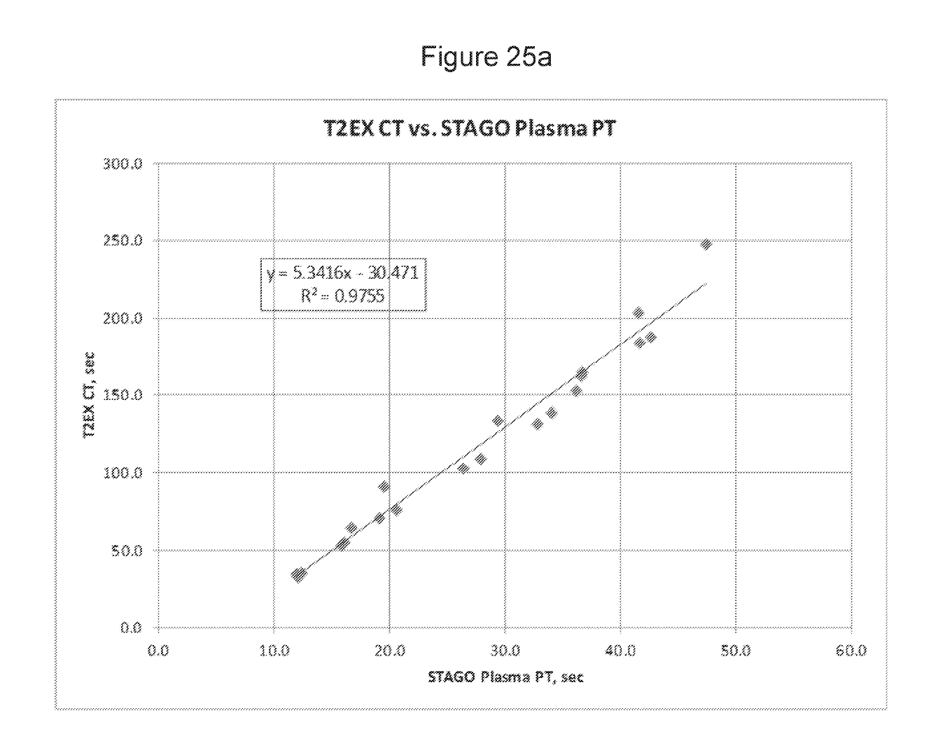

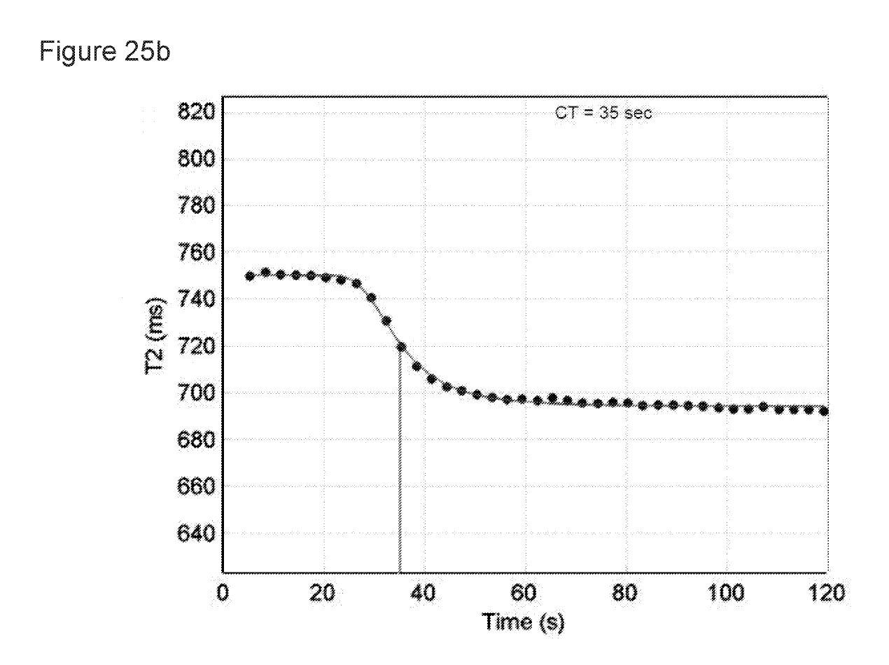

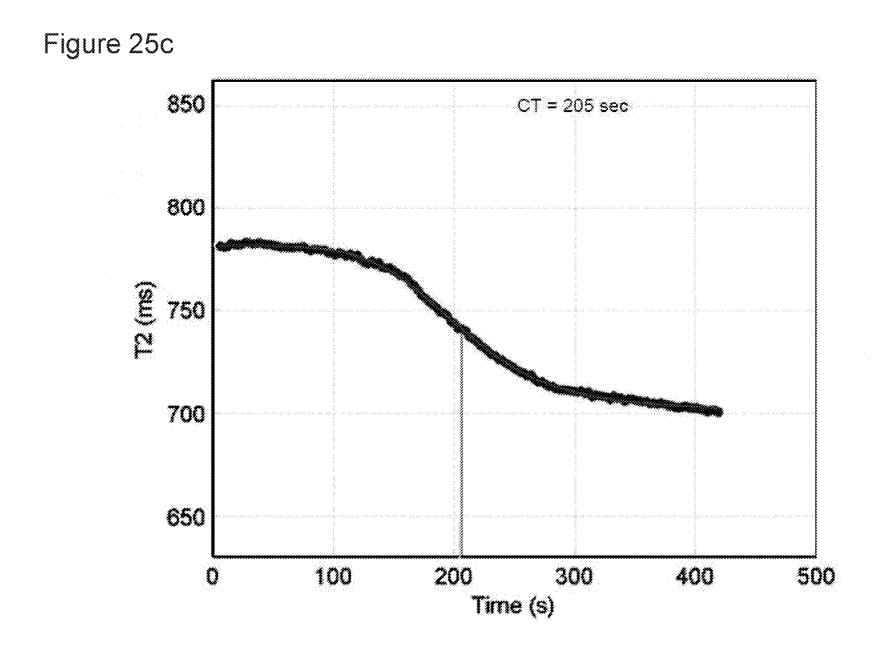

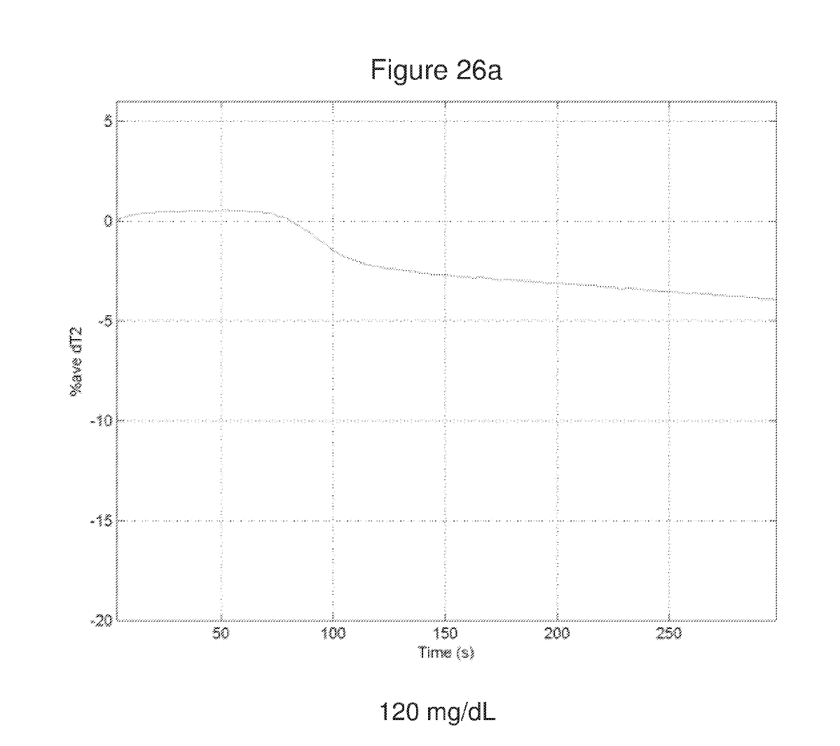

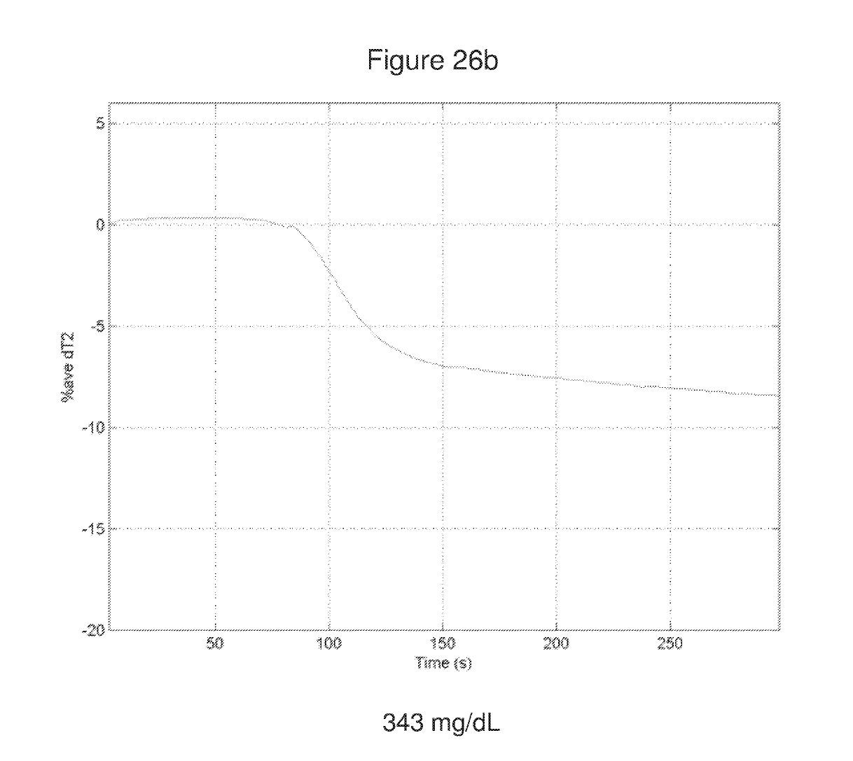

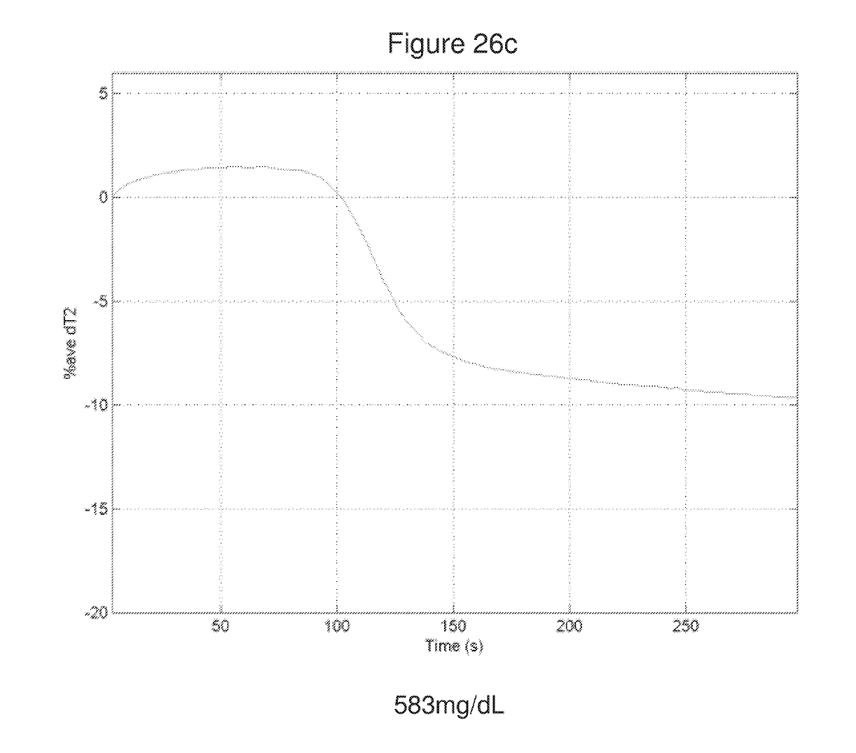

Direct Thrombin Inhibitor Another demonstration of this capability was a measurement of the effect of Rivaroxaban on the clotting time of tissue factor activator solution. Rivaroxaban was spiked into citrated blood from normal donors at increasing levels. Aliquots of spiked blood were activated with tissue factor activator solution and clot times determined by T2MR. Addition of 800 ng/mL of Rivaroxaban was able to extend clot times by approximately 80%. T2MR was able to detect clot times with tissue factor activation that ranged from 35 to 205 seconds (FIGS. 25b and 25c). These data correlated to plasma prothrombin times with a R²=0.97 (FIG. 25a).

Factor Deficiency

T2MR may be used to resolve differences in hemostasis resulting from clotting factor deficiencies, such as blood factor type 8 (FVIII) deficiency as found in hemophilia type A. Factor VIII deficiencies may be probed with T2MR clotting time or platelet activity using an intrinsic pathway activator, such as ellagic acid, or with an extrinsic pathway activator, such as a dilute formulation of tissue factor. In either case, the diagnostic principal is that a decrease in either concentration or activity of FVIII may result in a decrease in thrombin production, either rate of thrombin production or total thrombin molecules, which may be detected as an elongation of clot time, or as a reduction in platelet activity and contraction, due to a reduction in platelet activation. In one embodiment, a FVIII deficiency may be generated using a cocktail of monoclonal antibodies against FVIII, which may involve one or more antibody types targeted at different functional sites of FVIII, and may be raised in one or more animals. In another embodiment, FVIII may be eliminated from the genome using genetic knockout techniques, such as in murine or chick models. In other embodiments, FVIII deficiency may arise naturally, such as in human patients diagnosed with hemophilia type A or other coagulation diseases.

To probe factor deficiencies, such as FVIII, blood is collected in a manner to minimize endogenous tissue factor contamination, such as using venipuncture with vacutainers coated with sodium citrate, and by discarding the first vacutainer. In one embodiment, the blood is either naturally FVIII deficient, or in another embodiment, is rendered functionally deficient, using antibodies. To render functionally deficient, antibodies are added directly to whole blood, or to plasma, and incubated for several minutes prior to assessment, such as 30 minutes. Afterward, the naturally or functionally deficient FVIII blood is compared to either the same sample without antibody treatment, a healthy normal donor blood sample, or against a healthy normal quantitative range.

Using intrinsic pathway activation is a direct method to assess the effect of FVIII deficiency. If the sample is truly functionally deficient in FVIII, one result will be less thrombin production, either rate or total molecules, and will be resolved in T2MR as different from normal samples in several ways, including a longer clot time, lower platelet activity, and possibly other effects, such as a change in fibrinolysis. This pathway may be probed using intrinsic pathway activators, such as ellagic acid or kaolin, in the presence of calcium ions.

Using extrinsic pathway activation is another means to probe FVIII deficiency. However, to ensure that coagulation is not dominated by activated factor 7 (FVIIa), the sample is activated with a diluted form of tissue factor, which in one embodiment, is at a concentration that is several thousand times more diluted than normal assessments to probe the extrinsic pathway. An application of this may be in drug development, to evaluate the efficacy of FVIIa drugs to restore hemostasis in patients with hemophilia type A, or in other embodiments, patients with hemophilia type B or C, or other coagulation diseases.

The interrogation of factor deficiency using dilute tissue factor or extrinsic pathway activation may produce a change in the tight clot (phase 6), where with reduced FVIII or other factors in the intrinsic pathway (e.g., FIX or FXI), the tight clot signal may appear later after the start of the reaction, may have a higher T2 value, or may have a lower T2 intensity, or a combination of these. This change in the tight clot response to FVIII or other deficiencies of the intrinsic pathway may represent a reduced capacity of platelets to contract the clot into a tight clot with polyhedral-shaped erythrocytes.

To demonstrate this method, whole blood from a normal, healthy human donor was treated with a cocktail of four monoclonal antibodies against FVIII to render it functionally FVIII deficient, and incubated for 30 minutes for antibody treatment. Using a diluted tissue factor activation with calcium ions, the native and treated blood were run on the same instrument to evaluate clot time and platelet activity. The native blood clot time was 273 seconds, compared to 350 seconds with FVIII treatment, indicating a 28% increase in clotting time after functional FVIII deficiency. Similarly, the platelet activity metric (PAMj; Table 1) decreased from 8940 PAM units in native blood to 7552 PAM units after FVIII antibody treatment, a reduction in platelet activity of 16% after FVIII treatment. In addition, after anti-FVIII antibody treatment, the appearance of the lowest T2 signal (representing the tight clot or phase 6, “T2_clotB”) was extended out from 20 to 25 min. in native blood to over 45 minutes after FVIII treatment. These findings suggest dependence on FVIII level on clot time and the kinetics of platelet-induced clot contraction as represented by the platelet activity metric and tight clot metric using T2MR.

In another demonstration, FVIII was titrated into whole blood from a Hemophilia type A (HemA) donor. By additions of 0.1, 1, 10 and 100% FVIII and using a diluted tissue factor assay, the appearance of the lowest T2 tight clot signature (“T2_clotB”) accelerated from 30 min. to 18 min., and the serum T2 value at 40 min. increased from 600 to 1000 ms. A metric was developed to quantify this effect, called the “Tight clot metric” (TC) which was the summation of the difference in 1/T2 value from the serum to tight clot T2 divided by the serum T2, according to equation 21:

$$TC = {\sum }_{0 min .}^{x mi n .} \frac{1 / T {2}_{tightclot} - 1 / T {2}_{serum}}{1 / T 2 \_ serum} \qquad ( 21 )$$

where this TC metric is zero at any time in the summation if the tight clot is not detected, and may be summed to any amount of time, but typically is between 20 to 40 minutes. In addition, a filter may be imposed where if the intensity of the tight clot is not above a value of y, then the equation is zero at that time; i.e., the metric would only sum when the tight clot intensity value is above y. Results are then dependent on both the T2 and intensities, and typically the intensity filter for the tight clot may vary between 0.05 and 0.10 in relative units where 1.0 represents 100% intensity. By employing T2MR with this assay and data analysis strategy, it is possible to quantify the effect of added FVIII on platelet induced clot contraction in Hemophilia A blood.

The magnitude of the change due to factor deficiency will depend on the pathway of activation (intrinsic or extrinsic) and the concentration of activator, particularly for the extrinsic pathway activation. However, by performing a controlled study, T2MR may be used to probe factor deficiencies in clotting factors, and may be used in many applications in hemostasis, including clinical diagnoses of blood factor deficiencies, drug development to restore coagulation activity for patients with blood factor deficiencies, pharmacokinetics of pro-coagulant drug dosing in humans or animal models, and characterization of coagulation and platelet biology (e.g., using selective knockout of single or combinations of blood factors).

### Example 23. Extrinsic Activator Assay for Fibrinogen Measurements (T2Ex)

The T2MR based hemostasis assays described herein can be used to discriminate normal from abnormal patient samples for fibrinogen measurements using either an extrinsic or intrinsic pathway activation. To demonstrate this capability blood from normal donors was depleted of fibrinogen and known amounts of fibrinogen were added back at levels below (<200 mg/dL), within (200-500 mg/dL) and above (>500 mg/dL) the normal range of fibrinogen. Citrated whole blood from normal donors was centrifuged and serum removed. Packed red blood cells were washed with fibrinogen depleted plasma. Fibrinogen depleted plasma was spiked with known amounts of fibrinogen; these spiked plasmas were used to reconstitute washed red blood cells to produce blood samples with varying fibrinogen levels. Blood samples were activated with ellagic acid activator solution and clot time and % deltaT2 were measured. Fibrinogen levels in each sample were determined by the Clauss method. FIGS. 25a-25c show that with increasing fibrinogen levels there was an observed increase in % deltaT2. Low fibrinogen (120 mg/dL) gave an average % deltaT2 of 4, normal level fibrinogen (343 mg/dL) gave an average % deltaT2 of 8.3 and high fibrinogen (583 mg/dL) gave an average % deltaT2 of 10.7.

### Example 24. Intrinsic Assay for Platelet Activity Measurements (T2In)

The T2MR based hemostasis assays described herein can be used to discriminate normal from abnormal patient samples for platelet activity measurements using either an extrinsic or intrinsic pathway activation. To demonstrate this capability blood from normal donors was titrated with a direct platelet inhibitor.

Citrated whole blood from normal donors was spiked with varying concentrations of the platelet inhibitor ReoPro (Abcixirnab). 5 μL of ellagic acid solution with CaCl₂ was used to activate 36.7 μL of blood with or without ReoPro inhibition. T2MR data was collected for 35 minutes. Addition of platelet inhibitor had no effect on T2MR measured clot time or % deltaT2. FIG. 27b shows data from three different donors. There was some donor to donor differences in responsiveness to ReoPro. Increasing amounts of inhibitor resulted in a titration of platelet activity. Addition of the platelet inhibitor resulted in not only a decrease in the T2 values for the serum peak but also a delay in serum peak initiation. Inhibitor levels between 5 and 10 μg/mL resulted in complete inhibition of platelet activity (FIG. 27a). In samples where there was no measured platelet activity there was no noted change in clot time or % dT2, indicating that these measurements are independent of platelet function.

### Example 25. Fibrinolysis Measurements

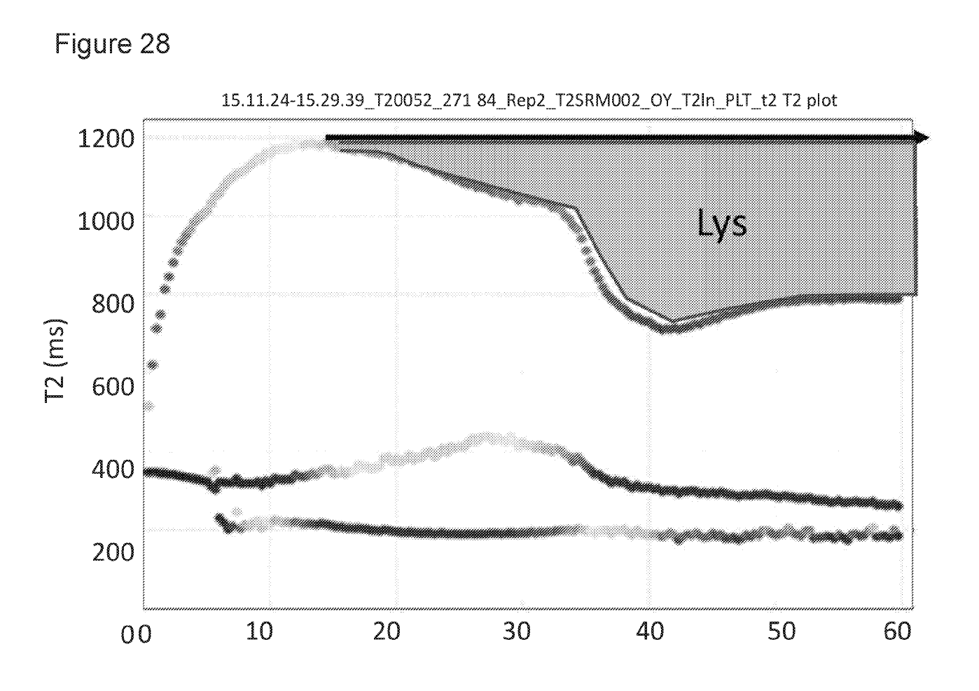

The T2MR based assays described herein can be used to quantify the breakdown of a clot (e.g., fibrin) in the process of fibrinolysis. To determine a lysis matrix, the area of the upper T2 population (T2up) drop was calculated as a reaction rate according to equation 22, and the lysis matrix (R2 area) is indicated in FIG. 28.

$$Lys 5 \text{:} Lysis ( t ) = {\int }_{maxT 2 time}^{t} ( \frac{1}{{T}_{2 up ( {t}^{′} )}} - \frac{1}{{maxT}_{2}} ) {dt}^{′} \qquad ( 22 )$$

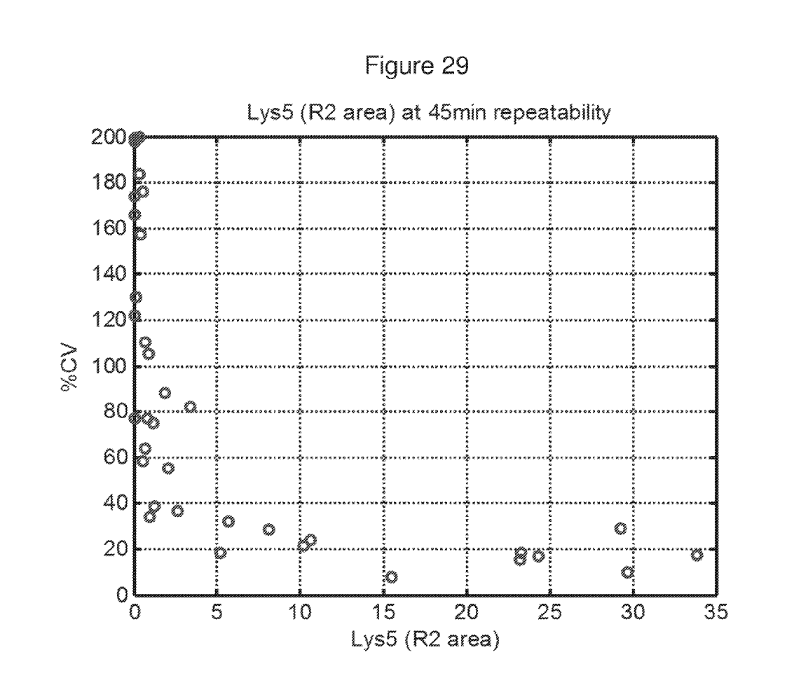

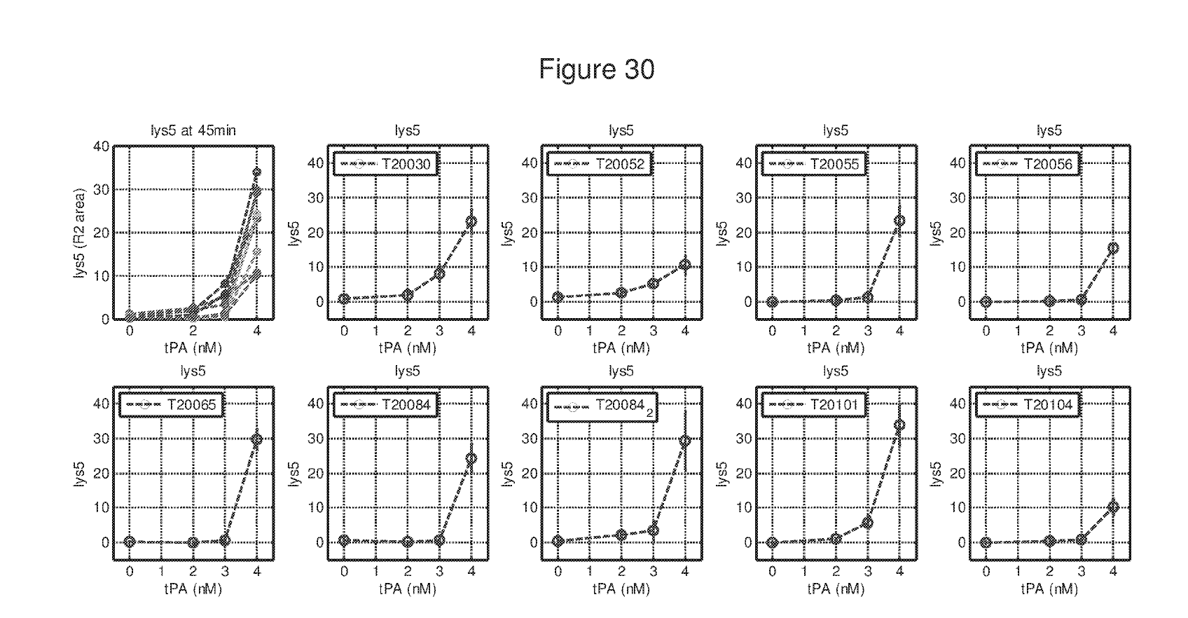

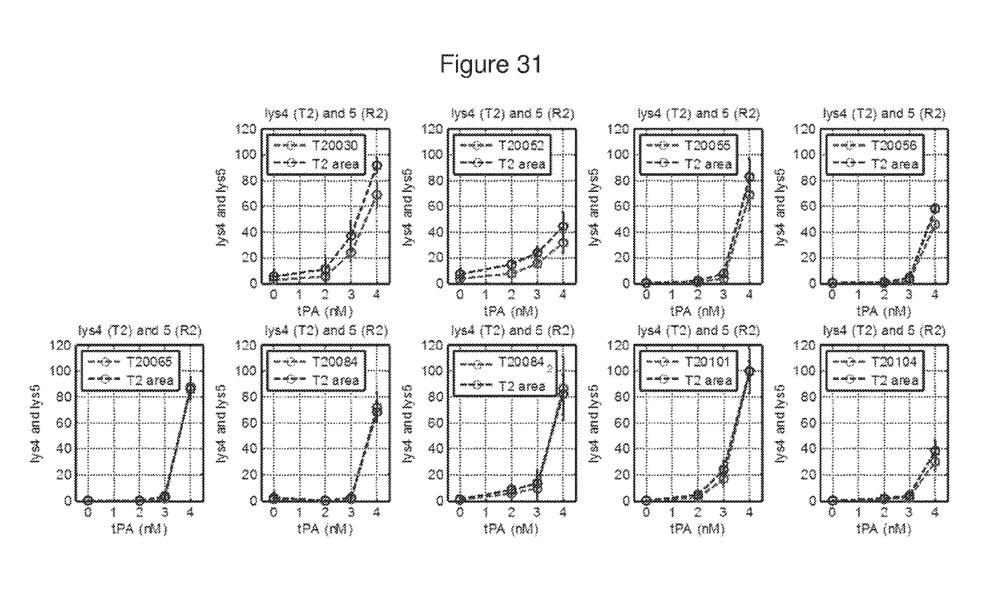

To demonstrate this quantification, blood from normal donors was titrated with an inducer of fibrinolysis, tissue plasminogen activator (tPA), and the R2 area was measured at 45 minutes. Results from replicate experiments, shown in FIG. 29, indicate that the R2 area increases with higher doses of tPA. Repeatability of lysis quantification by R2 area is shown in FIG. 30. FIG. 31 shows a comparison between T2 area and R2 area, where T2 area is used to define Lys4,

Lys4: Lysis(t)=∫_{maxT2 time}^{t}(maxT₂−T_{2up(t′)})dt′ (23)

### Example 26. Fibrinogen Measurements

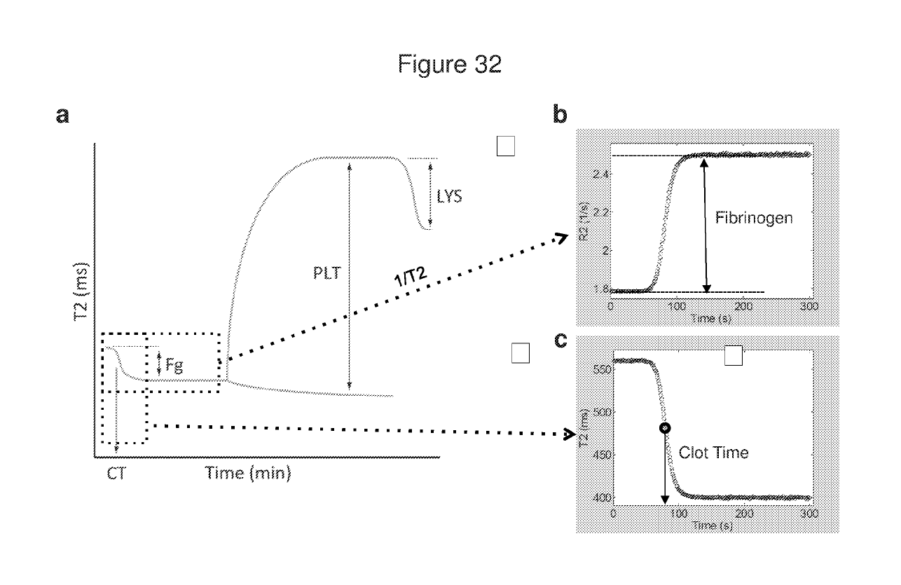

Fibrinogen content of a blood sample can be quantified using the T2MR based assays described herein. T2MR measures clotting time and fibrinogen through the change in R2 values due to fibrin formation (e.g., clot formation), which results in an increase in R2 values over time and is proportional to ΔR2, which is calculated by R2ₘᵢₙ−R2ₘₐₓ. FIGS. 32a-32c show the relationship between R2 and T2 at the early region (e.g., fibrin formation) of the coagulation process.

Analysis of T2MR data using the methods described herein enables quantification of fibrinogen concentration in a blood sample without the need to determine a hematocrit value, as conventional methods require. The instrument response metric, ΔR2, used in the T2In assay to quantify fibrinogen, is designed to not be substantially affected by either random or systematic variations in the concentration of other plasma proteins or other endogenous or exogenous small molecules.

The estimated fibrinogen concentration in a T2In assay is calculated by equation 24.

$$EFF = \frac{800 * ( \frac{1000}{{T}_{2 min}} - \frac{1000}{{T}_{2 max}} )}{1 - HCT / 100} = \frac{800 * ( \frac{1000}{{T}_{2 min}} - \frac{1000}{{T}_{2 max}} )}{1.2605 - \frac{267.5}{{T}_{2 max}}} \qquad ( 24 )$$

Equation 25 describes T2max when T2max is taken as the T2 value before clotting.

$$\frac{1}{{T}_{2 max}} = {R}_{rc} * {c}_{rc} + {R}_{Fg} * {c}_{Fg} + {\sum }_{i} {R}_{i} * {c}_{i} \qquad ( 25 )$$

The summation ΣᵢRᵢ*cᵢ accounts for all other blood components beyond fibrinogen and red cells. T2min is the T2 value after clotting where fibrinogen is converted to fibrin, which has been shown to have a larger T2 relaxivity. Equation 26 describes T2min after clotting.

$$\frac{1}{{T}_{2 min}} = {R}_{rc} * {c}_{rc} + {R}_{Fibrin} * {c}_{Fibrin} + {\sum }_{i} {R}_{i} * {c}_{i} \qquad ( 26 )$$

In the first approximation it can be assumed that fibrinogen is fully converted to fibrin during clotting, leading to c_{Fibrin}=c_{Fg}. It follows that subtracting Equation 25 from Equation 26 leads to Equations 27 and 28.

$$\begin{matrix}\frac{1}{{T}_{2 min}} - \frac{1}{{T}_{2 max}} = Δ {R}_{2} = ( {R}_{Fibrin} - {R}_{Fg} ) * {c}_{Fg} & ( 27 ) \\ {c}_{Fg} = a * Δ {R}_{2} & ( 28 )\end{matrix}$$

This demonstrates that the numerator in Equation 24 is independent of serum protein and other molecules. To compare with a reference method, dilution and hematocrit correction are necessary. Such a correction could be determined using Equation 29.

$${c}_{Fg\_T 2} = \frac{a * Δ {R}_{2}}{k * ( 1 - HCT )} \qquad ( 29 )$$

where HCT=hematocrit, k=whole blood volume fraction in the final assay and a=the coefficient in Equation 28 relating fibrinogen concentration and ΔR₂.

The NMR response parameter T₂ₘₐₓ used to correct T2In Effective Functional Fibrinogen (EFF) for variation in sample hematocrit can be affected by variation in plasma protein content. This effect of serum protein and other molecules on the denominator was estimated by testing 39 plasma samples. T₂ₚₗₐₛₘₐ mean was 1214 ms with a standard deviation of 141 ms, while the standard deviation of

$$\frac{1}{{T}_{2 plasma}}$$

was 0.092 s⁻¹. The uncertainty propagated from

$$\frac{1}{{T}_{2 plasma}}$$

to

$$\frac{1}{{T}_{2 max}}$$

can be calculated by Equation 30.

$$δ ( \frac{1}{{T}_{2 max}} ) = ( 1 - {f}_{rc} ) * δ ( \frac{1}{{T}_{2 plasma}} ) \qquad ( 30 )$$

The solution of Equation 30 is shown by Equation 31, indicating that the relative uncertainty of fibrinogen due to serum protein in this case was 2.5%.

$$\frac{δ ( EFF )}{EFF} = \frac{0.2675 * δ ( \frac{1}{{T}_{2 max}} )}{1 - HCT / 100} = 0.2675 * δ ( \frac{1}{{T}_{2 plasma}} ) = 2.5 \% \qquad ( 31 )$$

Thus, plasma proteins not involved in the coagulation process do not represent a significant source of bias in the determination of T2In EFF.

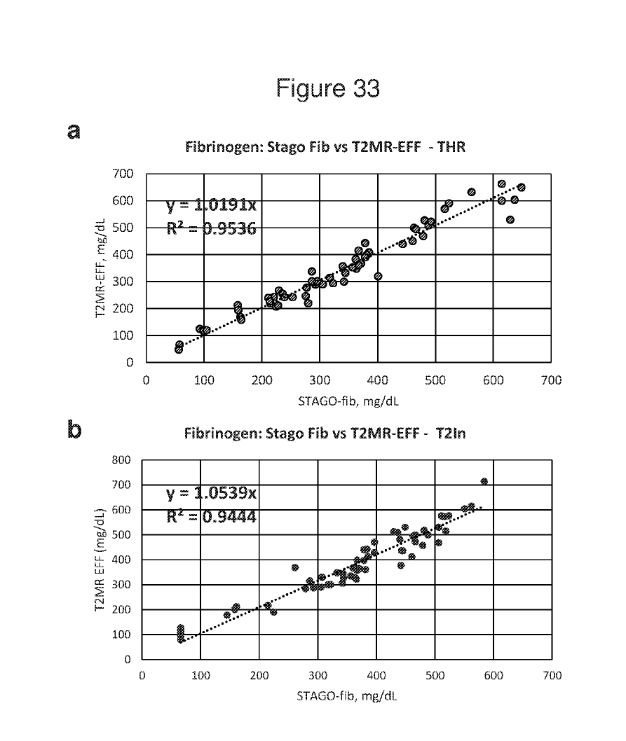

Fibrinogen standard curves were produced by the method described herein using whole blood samples prepared from a high fibrinogen donor. T2MR measurements of fibrinogen concentration (EEF) in intrinsic experiments (T2In, FIG. 33a) and thrombin-induced experiments (THR, FIG. 33b) were compared to a reference measurement. In this case, the reference measurement was a Clauss Fibrinogen measurement obtained using standard methods. The hematocrit collected had a range of 20% to 40% and a variation of 10%.

Factory calibration of T2In EFF is achieved due to the inherent stability of the magnetic measuring system and its ability to produce nearly identical instrument responses across time and different instruments. This allows for the generation of a master calibration function that can be used without the need for normalization for instrument-to-instrument and reagent lot-to-lot variation.

### Example 27. Smart-Switch Between Fully-Formed Fibrin Formation and Clot Retraction

The above methods describe how suitable acquisition parameters (e.g., CPMG dwell times) can be applied to different regions over the coagulation process. This example describes a method of automatically switching to a different CPMG sequence upon detecting a transition between the completion of fibrin formation (e.g., from fibrinogen) and platelet-driven clot retraction.

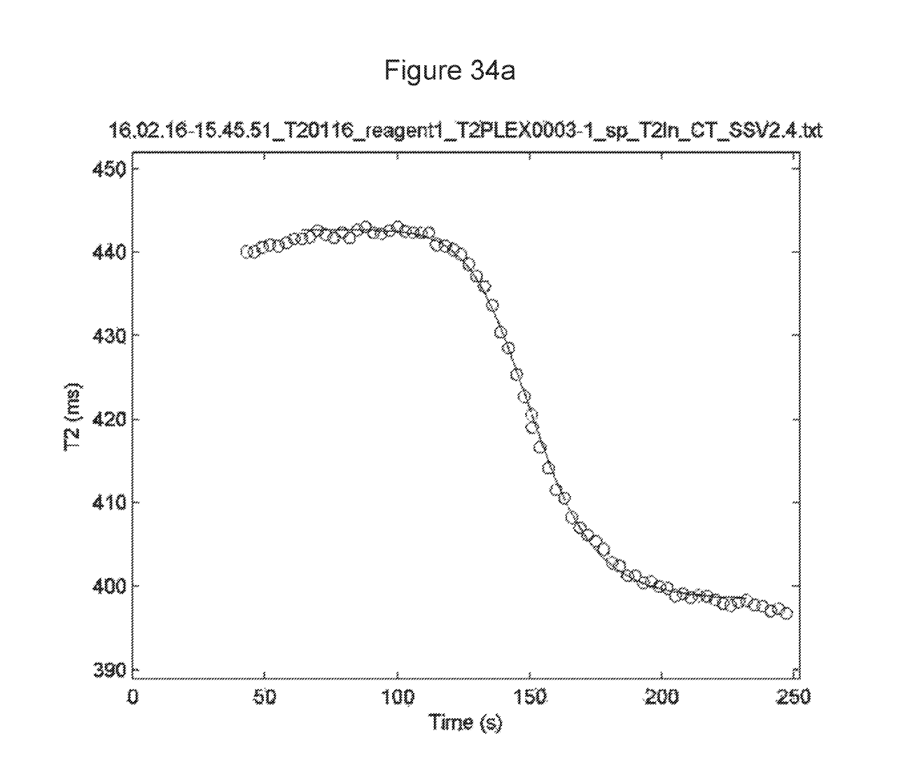

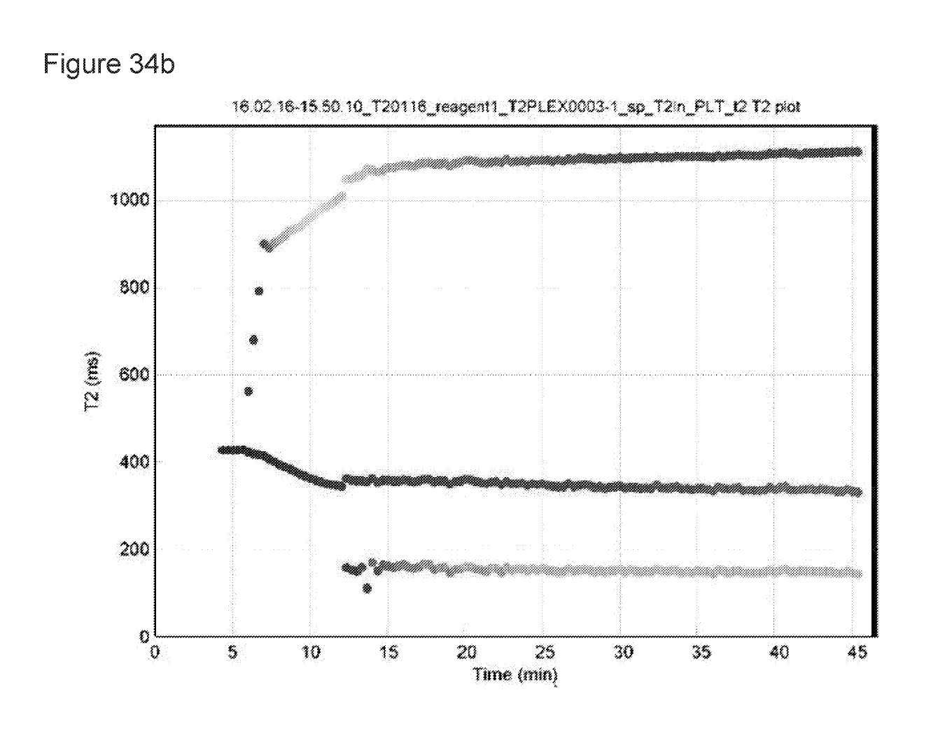

As described above, the shape of the raw T2MR has characteristics that can be fit with a 4 parameter logistic (4PL) function. The derivative of the 4PL function is a Gaussian distribution. FIG. 34 shows that platelet driven clot retraction began after full fibrin formation.

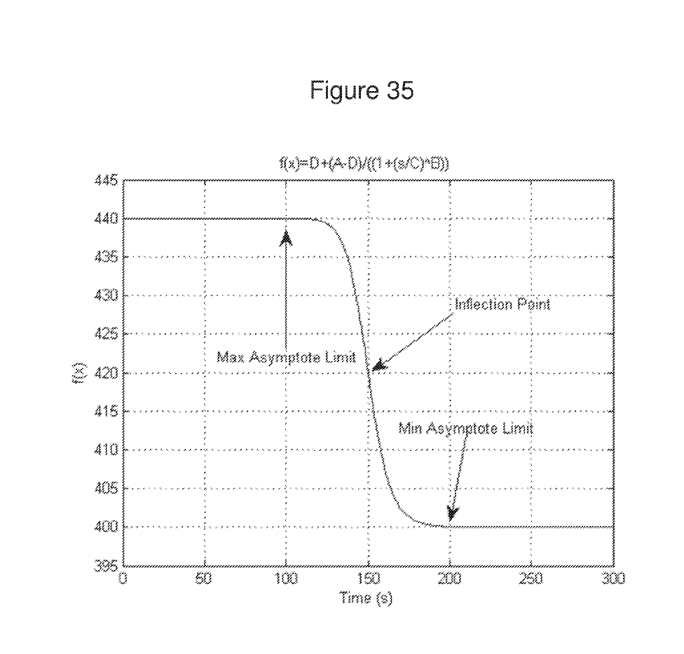

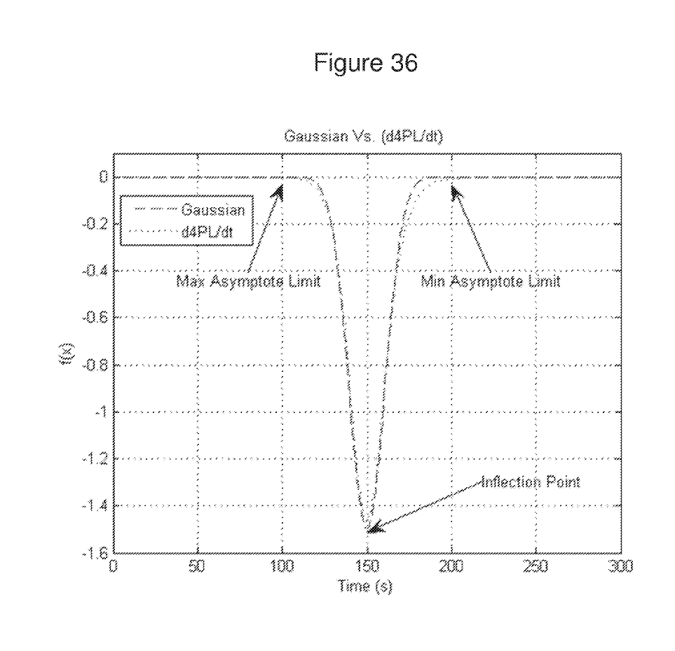

As indicated in FIG. 35, the 4PL function has a minimum asymptote, an inflection point, a Hill slope (slope of the inflection), and a maximum asymptote. The derivative of the initial point of the max asymptote and the min asymptote are zero, and the derivative of the function is at its minimum value at the inflection point. Fitting the derivative of the 4PL function over time with a Gaussian distribution reveals the initial and final limits of the 4PL, as indicated in FIG. 36. The clotting time begins when the max asymptote ends (i.e., where the first minimus of d(4PL)/dt is equal to zero), and the clotting time ends when the min asymptote begins (i.e., where the second minimum of d(4PL)/dt is equal to zero). The smart switch detects the end of fibrin formation, before the platelets being to retract, i.e., when the second minimum of d(4PL)/dt is equal to zero.

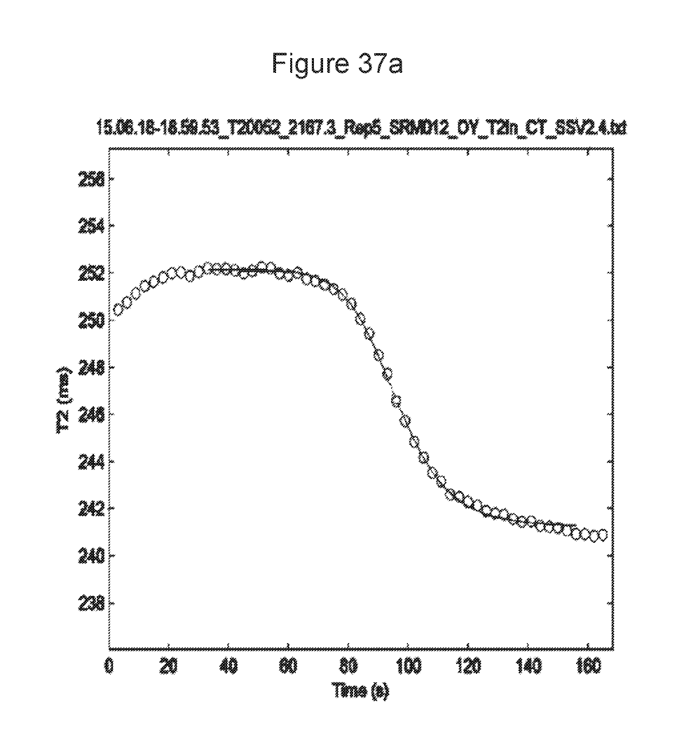

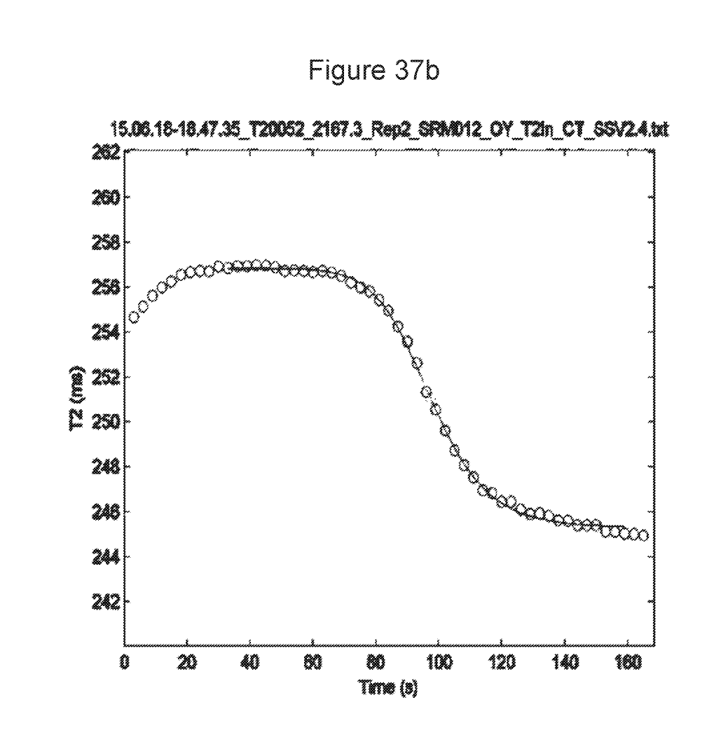

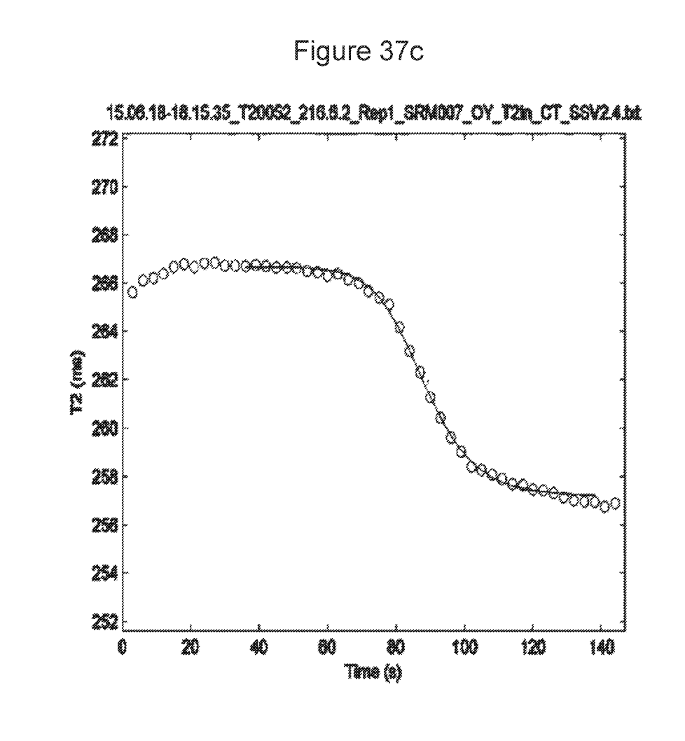

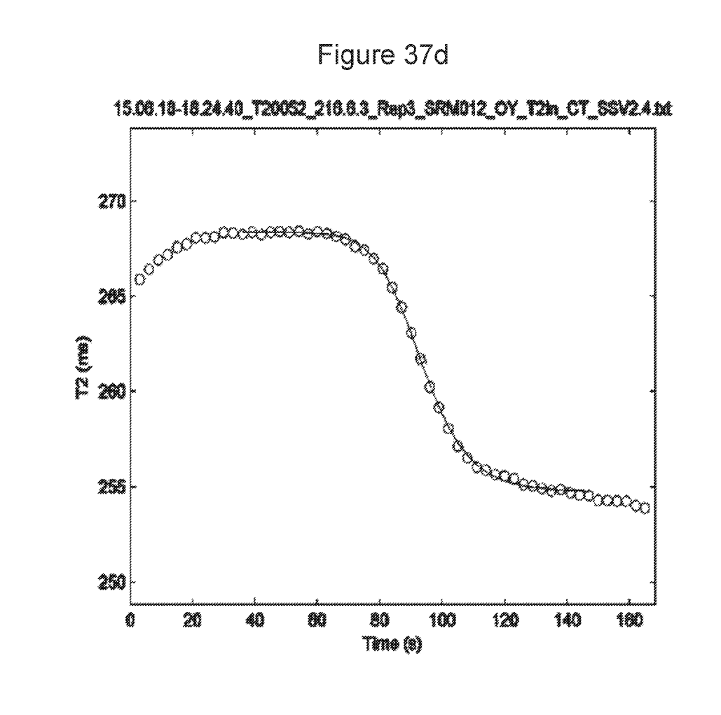

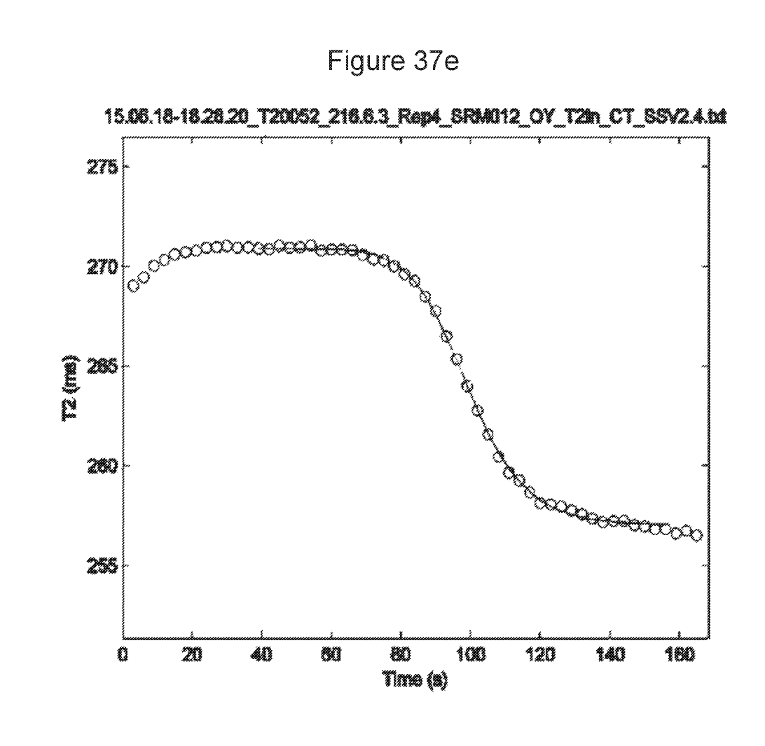

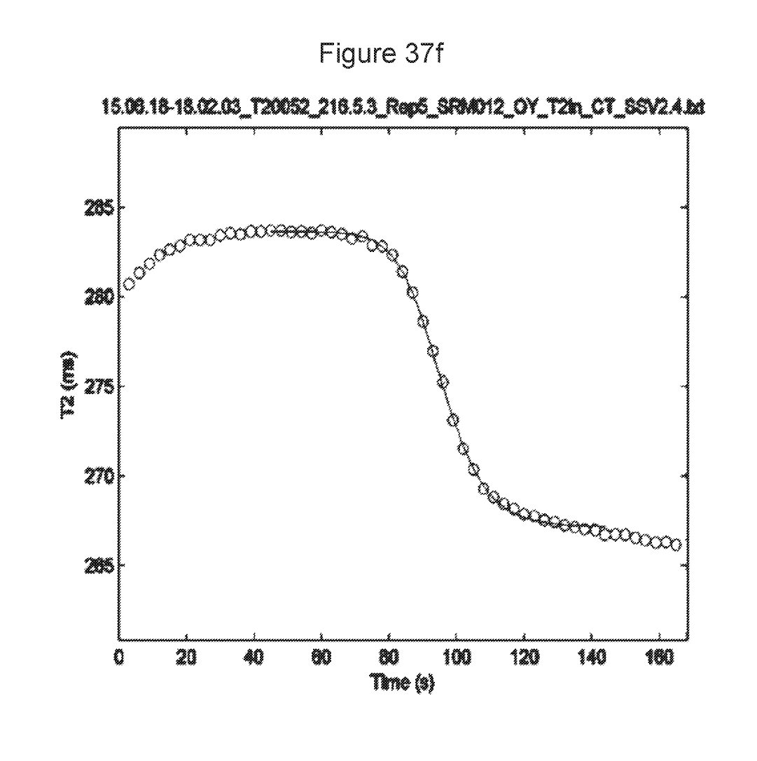

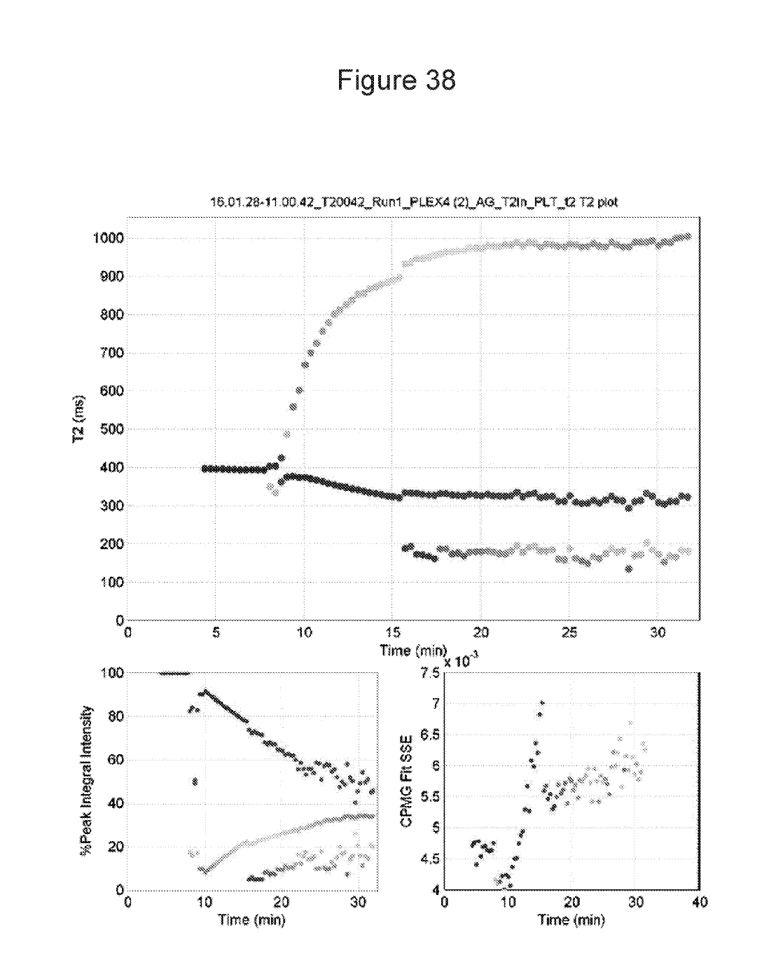

The Gaussian fitting was performed every 21 seconds, starting at 3 minutes from the beginning of the experiment. Once the second minimum was determined, the algorithm chose the points required for the 4PL fitting between the first and the second minimum obtained by the Gaussian fitting. This step enabled compensation for drift (e.g., raising or lowering) of the upper asymptote, as shown in FIG. 37. Upon detection of the end of fibrin formation, and prior to the activation of platelets, the CPMG sequence was switched (FIG. 38).

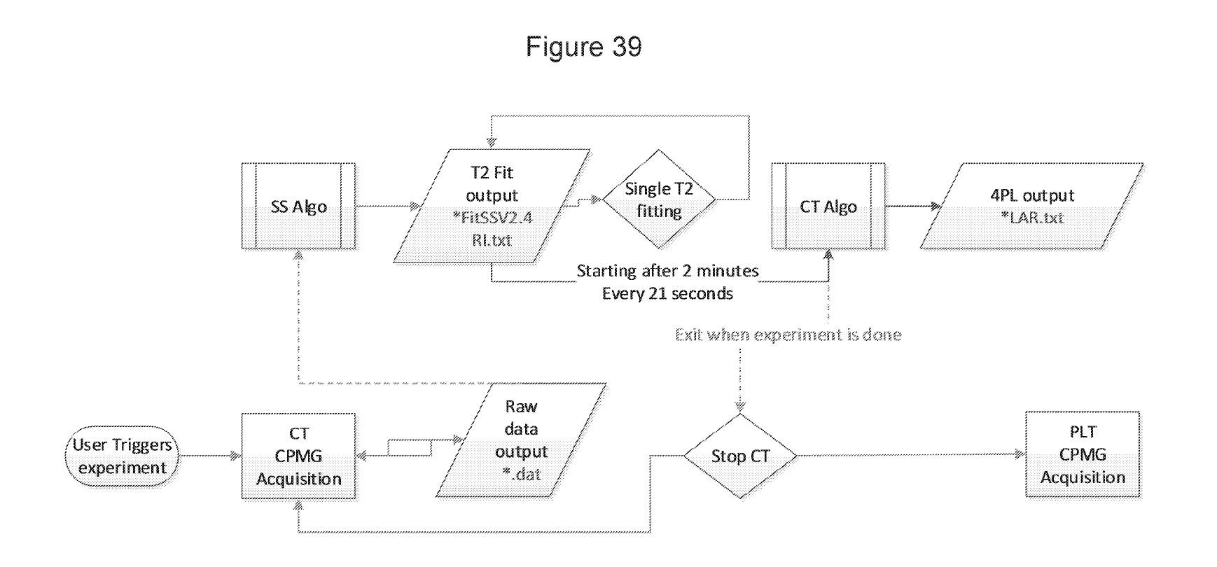

A working diagram of the smart switch is provided by FIG. 39.

### OTHER EMBODIMENTS

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

1. A method of monitoring water in a coagulating blood sample comprising the steps of:
(i) providing a blood sample from a test subject and mixing a clotting activation reagent with the blood sample to initiate a coagulation process,
(ii) making a series of T2 relaxation rate measurements of the water in the blood sample, wherein the measurements provide a plurality of decay curves, each decay curve measured using a Carr-Purcell-Meiboom-Gill (CPMG) sequence having a predetermined dwell time and each decay curve characteristic of a time point in the coagulation process,
(iii) applying a mathematical transform to the plurality of decay curves to identify one or more water populations in the blood sample at one or more time points in the coagulation process to produce one or more T2 values and/or T2 intensities,
wherein decay curves for time points at the beginning of the process are measured using a CPMG sequence having a predetermined dwell time that is faster than the predetermined dwell time of the CPMG sequence used to measure decay curves at time points at the end of the process.

2. The method of claim 1, wherein the predetermined dwell time of the CPMG sequence used to measure decay curves at the beginning of the process is a fast dwell time of from 1 to 6 seconds.

3. The method of claim 2, wherein the predetermined dwell time of the CPMG sequence used to measure decay curves at the beginning of the process is a fast dwell time of from 2 to 4 seconds.

4. The method of claim 2 or 3, wherein the fast dwell time is used to measure decay curves during the coagulation process for at least 1 minute following the completion of step (i).

5. The method of claim 4, wherein the fast dwell time is used to measure decay curves during the coagulation process for at least 2 minutes following the completion of step (i), wherein the clotting activation reagent is an extrinsic pathway activator.

6. The method of claim 4, wherein the fast dwell time is used to measure decay curves during the coagulation process for at least 5 minutes following the completion of step (i), wherein the clotting activation reagent is an intrinsic pathway activator.

7. The method of any one of claims 1-6, wherein the predetermined dwell time of the CPMG sequence used to measure decay curves at the end of the process is a long dwell time of from 8 to 20 seconds.

8. The method of claim 7, wherein the long dwell time is used to measure decay curves during the coagulation process after more than 2 minutes following the completion of step (i), wherein the clotting activation reagent is an extrinsic pathway activator.

9. The method of claim 7, wherein the long dwell time is used to measure decay curves during the coagulation process after more than 5 minutes following the completion of step (i), wherein the clotting activation reagent is an intrinsic pathway activator.

10. The method of any one of claims 1-9, wherein, at each time point, each T2 intensity for a given water population is proportional to the amount of the water population in its micro environment within blood sample at the time point.

11. The method of any one of claims 7-10, further comprising detecting a clotting event at a time point in the coagulation process, wherein the long dwell time is used to measure decay curves within 30 seconds of the clotting event.

12. The method of claim 11, wherein detecting a clotting event at a time point in the coagulation process comprising detecting a T2 value or T2 intensity for a water population characteristic of a clot microenvironment.

13. The method of any one of claims 1-12, further comprising:
(a) identifying from the series of T2 relaxation rate measurements a first phase, wherein the first phase comprises one or more T2 values measured during the clotting process prior to any observable clot formation;
(b) identifying from the series of T2 relaxation rate measurements a second phase, wherein the second phase comprises one or more T2 values measured during the clotting process at time points during which the sample comprises a loosely bound clot; and
(c) during the first phase, using a CPMG sequence having a predetermined dwell time that is faster than the predetermined dwell time of the CPMG sequence used to measure decay curves during the second phase.

14. The method of any one of claims 1-12, further comprising:
(a) collecting the series of T2 relaxation rate measurements during a first phase and prior to a second phase, wherein the first phase comprises one or more T2 values measured during the clotting process prior to any observable clot formation, and the prior to a second phase comprises one or more T2 values measured during the clotting process prior to time points during which the sample comprises a loosely bound clot;
(b) fitting the one or more T2 values to a curve having a maximum asymptote from the one or more T2 values of the first phase and slope of inflection in the T2 values defining the transition from the first phase to the second phase;
(c) calculating the second derivative of the curve to identify the inflection point of the transition from the first phase to the second phase;
(d) following the inflection point, identifying the time at which the slope of the second derivative reaches zero; and
(e) prior to a second phase, using a CPMG sequence having a predetermined dwell time that is faster than the predetermined dwell time of the CPMG sequence used to measure decay curves after the time identified in step (d).

15. The method of any one of claims 1-14, further comprising, for one or more decay curves characteristic of a time point in the coagulation process, (iv) fitting the decay curve to a mono-exponential, bi-exponential, and tri-exponential fit; and (v) using a non-linear least squares algorithm to identify the best fit.

16. The method of claim 15, wherein step (v) comprises selecting the bi-exponential fit over the mono-exponential fit if the error sum of squares (SSE) of the two calculated T2 values for the bi-exponential fit is less than 75% of the SSE calculated for the mono-exponential fit.

17. The method of claim 15, wherein step (v) comprises selecting the tri-exponential fit over the bi-exponential fit if the SSE of the tri-exponential fit is less than 90% of the SSE of the bi-exponential fit.

18. The method of any one of claims 15-17, wherein step (v) comprises constraining the non-linear least squares analysis to consider only T2 values not less than 50 ms and not greater than 2000 ms.

19. The method of any one of claims 15-18, wherein, following step (i), if step (v) identifies a bi-exponential or tri-exponential fit as the best fit for one or more decay curves measured using a CPMG sequence having a fast dwell time of from 1 to 6 seconds for one or more consecutive time points in the coagulation process, then the CPMG sequence used to measure one or more additional decay curves during the clotting process is characterized by a long dwell time of from 8 to 20 seconds.

20. The method of any one of claims 15-18, wherein, following step (i), if (a) step (v) identifies a bi-exponential or tri-exponential fit as the best fit for one or more decay curves measured using a CPMG sequence having a fast dwell time of from 1 to 6 seconds for one or more consecutive time points in the coagulation process, and (b) a T2 intensity for a water population characteristic of a clot micro environment reaches a predetermined threshold, then the CPMG sequence used to measure one or more additional decay curves during the clotting process is characterized by a long dwell time of from 8 to 20 seconds.

21. The method of any one of claims 1-14, further comprising, one the basis of the T2 values determining whether a clot has formed and, if so, then the CPMG sequence used to measure one or more additional decay curves during the clotting process is characterized by a long dwell time of from 8 to 20 seconds.

22. The method of any one of claims 1-21, further comprising:
(ia) identifying from the series of T2 relaxation rate measurements a first phase, wherein the first phase comprises one or more T2 values measured during the clotting process prior to any observable clot formation;
(ib) identifying from the series of T2 relaxation rate measurements a second phase, wherein the second phase comprises one or more T2 values measured during the clotting process at time points during which the sample comprises a loosely bound clot;
(ic) calculating the difference between the one or more T2 values of the first phase and the one or more T2 values of the second phase; and
(id) on the basis of step (ic), determining the fibrinogen level of the blood sample.

23. The method of claim 22, comprising:
(xi) fitting the one or more T2 values of the first phase and the one or more T2 values of the second phase to a curve;
(xii) identifying a maximum asymptote from the one or more T2 values of the first phase;
(xiii) identifying a minimum asymptote from the one or more T2 values of the second phase; and
(xiv) calculating the difference between the maximum asymptote and the minimum asymptote; and
(xv) on the basis of step (xiv), determining the fibrinogen level of the blood sample.

24. The method of any one of claims 1-23, further comprising:
(yi) identifying from the series of T2 relaxation rate measurements a first phase, wherein the first phase comprises one or more T2 values measured during the clotting process prior to any observable clot formation;
(yii) identifying from the series of T2 relaxation rate measurements a second phase, wherein the second phase comprises one or more T2 values measured during the clotting process at time points during which the sample comprises a loosely bound clot;
(yiii) calculating the minimum value of the first derivative or the inflection point from T2 values spanning the transition from first phase to the second phase of the clotting process; and
(yiv) on the basis of step (yiii), determining the clotting time of the blood sample.

25. The method of claim 24, comprising:
(zi) fitting the one or more T2 values of the first phase and the one or more T2 values of the second phase to a curve;
(zii) identifying a maximum asymptote from the one or more T2 values of the first phase;
(ziii) identifying a minimum asymptote from the one or more T2 values of the second phase; and
(ziv) calculating a minimum value of the first derivative or the inflection point from the curve spanning the transition from first phase to the second phase of the clotting process; and
(zv) on the basis of step (ziv), determining the clotting time of the blood sample.

26. The method of any one of claims 1-25, further comprising:
(ai) identifying from the series of T2 relaxation rate measurements a third phase, wherein the third phase comprises one or more T2 values having one or more T2 intensities greater than a predetermined threshold and characteristic of a serum-like environment, and where each of the one or more T2 values is measured during the clotting process at time points during which the sample comprises a mixture of serum and clot microenvironments; and
(aii) on the basis of at least one of the one or more T2 values and the one or more T2 intensities, determining the platelet activity of the blood sample.

27. The method of claim 26 comprising:
(bi) identifying from the series of T2 relaxation rate measurements a third phase, wherein the third phase comprises one or more T2 values characteristic of a serum-like environment, each of the one or more T2 values having a T2 intensity greater than a predetermined threshold, and where each of the one or more T2 values is measured during the clotting process at time points during which the sample comprises a mixture of serum and clot microenvironments;
(bii) calculating the difference between the one or more T2 values of the third phase and a predetermined lower bound at a predetermined time, or calculating a T2 curve characteristic of the third phase and calculating an area between that curve and a predetermined lower bound for a predetermined time period during the clotting process; and
(biii) on the basis of step (bii), determining the platelet activity of the blood sample.

28. The method of claim 27 wherein the platelet activity is determined by calculating the difference between the one or more T2 values of the third phase and a predetermined lower bound at a predetermined time and the lower bound and the lower bound comprises one of the following:
(ci) one or more T2 values of a first phase, wherein the first phase comprises one or more T2 values measured during the clotting process prior to any observable clot formation;
(cii) one or more T2 values of a second phase, wherein the second phase comprises one or more T2 values measured during the clotting process at time points during which the sample comprises a loosely bound clot;
(ciii) one or more T2 values of a fourth phase, wherein the fourth phase comprises one or more T2 values characteristic of a clot microenvironment, wherein each of the one or more T2 values is measured during the clotting process at time points during which the sample comprises a mixture of serum and clot microenvironments;
(civ) a maximum asymptote of the first phase, wherein the maximum asymptote is determined by fitting the one or more T2 values of the first phase and the one or more T2 values of the second phase to a curve, and identifying a maximum asymptote from the one or more T2 values of the first phase, or identifying a minimum asymptote from the one or more T2 values of the second phase; and
(cv) a minimum asymptote of the second phase, wherein the maximum asymptote is determined by fitting the one or more T2 values of the first phase and the one or more T2 values of the second phase to a curve, and identifying a maximum asymptote from the one or more T2 values of the first phase, or identifying a minimum asymptote from the one or more T2 values of the second phase.

29. The method of claim 27 wherein the platelet activity is determined by calculating an area under a T2MR curve for the third phase and a predetermined lower bound for a predetermined time period during the clotting process and the lower bound comprises one of the following:
(di) one or more T2 values of a first phase, wherein the first phase comprises one or more T2 values measured during the clotting process prior to any observable clot formation;
(dii) one or more T2 values of a second phase, wherein the second phase comprises one or more T2 values measured during the clotting process at time points during which the sample comprises a loosely bound clot;
(diii) one or more T2 values of a fourth phase, wherein the fourth phase comprises one or more T2 values characteristic of a clot microenvironment, and where each of the one or more T2 values is measured during the clotting process at time points during which the sample comprises a mixture of serum and clot microenvironments;
(div) a maximum asymptote of the first phase, wherein the maximum asymptote is determined by fitting the one or more T2 values of the first phase and the one or more T2 values of the second phase to a curve, and identifying a maximum asymptote from the one or more T2 values of the first phase, or identifying a minimum asymptote from the one or more T2 values of the second phase; and
(dv) a minimum asymptote of the second phase, wherein the maximum asymptote is determined by fitting the one or more T2 values of the first phase and the one or more T2 values of the second phase to a curve, and identifying a maximum asymptote from the one or more T2 values of the first phase, or identifying a minimum asymptote from the one or more T2 values of the second phase.

30. The method of claim 28, comprising
(ei) identifying the difference between the T2 values of the third phase and a predetermined lower bound;
(eii) identifying one or more T2 intensities of the third phase or the lower bound; and
(eiii) on the basis of step (ei) and step (eii) determining the platelet activity.

31. The method of claim 29, further comprising
(fi) identifying an area defined by a T2MR curve of the third phase and a predetermined lower bound;
(fii) identifying one or more T2 intensities of the third phase or the lower bound; and
(fiii) on the basis of step (fi) and step (fii), determining the platelet activity.

32. The method of claim 31, comprising
(gi) identifying the maximum T2 intensity of the third phase;
(gii) identifying an area defined by a T2MR curve of the third phase and a predetermined lower bound; and
(giii) on the basis of step (gi) and step (gii), determining the platelet activity.

33. The method of claim 31, comprising
(hi) identifying the maximum T2 intensity of the third phase;
(hii) identifying the time associated with the maximum T2 intensity of the third phase;
(hiii) identifying an area defined by a T2MR curve of the third phase and a predetermined lower bound that terminates at the time associated with the maximum T2 intensity of the third phase; and
(hiv) on the basis of step (hiii), determining the platelet activity.

34. The method of any one of claims 26-32, wherein the one or more T2 values of the third phase comprise the maximum T2 values observed during the clotting process.

35. The method of any one of claims 26-32, further comprising multiplying the difference by the T2 intensity of the one or more T2 values of the third phase to produce a value, and on the basis of the value determining the platelet activity of the blood sample.

36. The method of any one of claims 1-35, further comprising:
(i-i) identifying from the series of T2 relaxation rate measurements a third phase, wherein the third phase comprises one or more T2 values characteristic of a serum-like environment, each of the one or more T2 values having a T2 intensity greater than a predetermined threshold, and where each of the one or more T2 values is measured during the clotting process at time points during which the sample comprises a mixture of serum and clot microenvironments;
(i-ii) identifying from the series of T2 relaxation rate measurements a fifth phase, wherein the fifth phase comprises one or more T2 values characteristic of a serum-like environment, and where each of the one or more T2 values is measured during the clotting process at time points during which the sample comprises a clot micro environment undergoing lysis;
(i-iii) calculating the difference between the one or more T2 values of the third phase and the one or more T2 values of the fifth phase; and
(i-iv) on the basis of step (i-iii), determining the level of the fibrinolysis of the blood sample.

37. The method of any one of claims 1-35, further comprising:
(ji) identifying from the series of T2 relaxation rate measurements a third phase, wherein the third phase comprises one or more T2 values characteristic of a serum-like environment, each of the one or more T2 values having a T2 intensity greater than a predetermined threshold, and where each of the one or more T2 values is measured during the clotting process at time points during which the sample comprises a mixture of serum and clot microenvironments;
(jii) identifying from the series of T2 relaxation rate measurements a fifth phase, wherein the fifth phase comprises one or more T2 values characteristic of a serum-like environment, and where each of the one or more T2 values is measured during the clotting process at time points during which the sample comprises a clot micro environment undergoing lysis;
(jiii) calculating an area above a T2MR curve for the fifth phase and a predetermined upper bound for a predetermined time period during the clotting process, wherein the upper bound is defined by the one or more T2 values of the third phase projected over the curve for the fifth phase; and
(jiv) on the basis of step (jii), determining the level of the fibrinolysis of the blood sample.

38. The method of claim 37, wherein the upper bound is defined by the maximum T2 value of the third phase projected over the curve for the fifth phase.

39. The method of any one of claims 1-38, further comprising:
(ki) measuring the T2 of the blood sample prior to step (i) to produce a first T2 value; and
(kii) on the basis of the measured first T2 value, determining the hematocrit level of the blood sample.

40. The method of any one of claims 1-38, further comprising
(ki) identifying from the series of T2 relaxation rate measurements a first phase, wherein the first phase comprises one or more T2 values measured during the clotting process prior to any observable clot formation; and
(kii) on the basis of the T2 values of the first phase determining a value for a hematocrit parameter, wherein the value of the hematocrit parameter is characteristic of the hematocrit concentration.

41. The method of claim 39 or 40, further comprising:
(kiii) identifying from the series of T2 relaxation rate measurements a first phase, wherein the first phase comprises one or more T2 values measured during the clotting process prior to any observable clot formation;
(kiv) identifying from the series of T2 relaxation rate measurements a second phase, wherein the second phase comprises one or more T2 values measured during the clotting process at time points during which the sample comprises a loosely bound clot;
(kv) calculating the difference between the one or more T2 values of the first phase and the one or more T2 values of the second phase; and
(kvi) calculating a corrected fibrinogen value based on the difference between the first phase and the second phase and the hematocrit parameter.

42. The method of claim 41, further comprising:
(kvii) fitting the one or more T2 values of the first phase and the one or more T2 values of the second phase to a curve; identifying a maximum asymptote from the one or more T2 values of the first phase; and identifying a minimum asymptote from the one or more T2 values of the second phase;
(kviii) calculating the difference between the maximum asymptote and the minimum asymptote;
(kix) on the basis of the hematocrit and step (kviii), determining the fibrinogen concentration.

43. The method of any one of claims 1-42, wherein the blood sample is diluted from 0.1-60% prior to step (i).

44. The method of claim 43, wherein the blood sample is diluted about 50% prior to step (i).

45. The method of any one of claims 1-44, wherein the blood sample is a whole blood sample.

46. The method of claim 45, wherein the blood sample comprises anticoagulant.

47. The method of claim 46, wherein the whole blood sample comprises at least one of citrate, heparin, and corn trypsin inhibitor.

48. The method of claim 47, wherein the whole blood sample comprises citrate.

49. The method of any one of claims 1-25 or 36-48, wherein the blood sample is a plasma sample.

50. The method of claim 49, wherein the plasma sample comprises citrate.

51. The method of any of claims 1-50, wherein the clotting activation reagent is a dried clotting activation reagent.

52. A method of monitoring water in a coagulating blood sample comprising the steps of:
(i) providing a blood sample from a test subject and mixing a clotting activation reagent with the blood sample to initiate a coagulation process,
(ii) making a series of T2 relaxation rate measurements of the water in the blood sample, wherein the measurements provide a plurality of decay curves, each decay curve measured using a Carr-Purcell-Meiboom-Gill (CPMG) sequence having a predetermined dwell time and each decay curve characteristic of a time point in the coagulation process,
(iii) applying a mathematical transform to the plurality of decay curves to identify one or more water populations in the blood sample at one or more time points in the coagulation process to produce one or more T2 values and/or T2 intensities,
(iv) identifying from the series of T2 relaxation rate measurements a first phase, wherein the first phase comprises one or more T2 values measured during the clotting process prior to any observable clot formation;
(v) identifying from the series of T2 relaxation rate measurements a second phase, wherein the second phase comprises one or more T2 values measured during the clotting process at time points during which the sample comprises a loosely bound clot;
(vi) fitting the one or more T2 values of the first phase and the one or more T2 values of the second phase to a curve, and identifying a maximum asymptote from the one or more T2 values of the first phase, or identifying a minimum asymptote from the one or more T2 values of the second phase;
(vii) calculating the difference between the maximum asymptote and the minimum asymptote; and
(viii) on the basis of step (vii), determining the fibrinogen value of the blood sample.

53. The method of claim 52, further comprising:
(ix) calculating the maximum value of the second derivative or the inflection point from T2 values spanning the transition from first phase to the second phase of the clotting process; and
(x) mathematically combining the maximum value determined in step (ix) with the value determined in step (viii)
(xi) on the basis of step (x) determining a fibrinogen value of the blood sample.

54. The method of claim 52, further comprising:
(ix) mathematically combining the value of the maximum asymptote with the value from step (vii)
(x) on the basis of the value of step (ix), determining a fibrinogen value.

55. The method of claim 52, comprising
(a) identifying from the series of T2 relaxation rate measurements a first phase, wherein the first phase comprises one or more T2 values measured during the clotting process prior to any observable clot formation;
(b) identifying from the series of T2 relaxation rate measurements a second phase, wherein the second phase comprises one or more T2 values measured during the clotting process at time points during which the sample comprises a loosely bound clot;
(c) fitting the one or more T2 values of the first phase and the one or more T2 values of the second phase to a curve, and identifying a maximum asymptote from the one or more T2 values of the first phase, or identifying a minimum asymptote from the one or more T2 values of the second phase;
(d) calculating the difference between the maximum asymptote and the minimum asymptote; and
(e) mathematically combining the value of the maximum asymptote, the maximum T2 value and the value from step (vii);
(f) on the basis of the value of step (e), determining a fibrinogen value.

56. The method of any of claims 52-55, further comprising
(1) on the basis of step (iv) determining a value for a hematocrit parameter, wherein the value of the hematocrit parameter is characteristic of the hematocrit concentration;
(2) on the basis of the fibrinogen value and the hematocrit parameter, determining a corrected fibrinogen concentration.

57. A method of monitoring water in a coagulating blood sample comprising the steps of:
(i) providing a blood sample from a test subject and mixing a clotting activation reagent with the blood sample to initiate a coagulation process,
(ii) making a series of T2 relaxation rate measurements of the water in the blood sample, wherein the measurements provide a plurality of decay curves, each decay curve measured using a Carr-Purcell-Meiboom-Gill (CPMG) sequence having a predetermined dwell time and each decay curve characteristic of a time point in the coagulation process,
(iii) applying a mathematical transform to the plurality of decay curves to identify one or more water populations in the blood sample at one or more time points in the coagulation process to produce one or more T2 values and/or T2 intensities,
(iv) identifying from the series of T2 relaxation rate measurements a first phase, wherein the first phase comprises one or more T2 values measured during the clotting process prior to any observable clot formation;
(v) identifying from the series of T2 relaxation rate measurements a second phase, wherein the second phase comprises one or more T2 values measured during the clotting process at time points during which the sample comprises a loosely bound clot;
(vi) fitting the one or more T2 values of the first phase and the one or more T2 values of the second phase to a curve, and identifying a maximum asymptote from the one or more T2 values of the first phase, or identifying a minimum asymptote from the one or more T2 values of the second phase;
(vii) calculating the value of the second derivative or the inflection point of the curve between the maximum asymptote and the minimum asymptote; and
(viii) on the basis of step (vii), determining the clotting time of the blood sample.

58. The method of claim 52, further comprising:
(ix) calculating the maximum value of the second derivative or the inflection point from T2 values spanning the transition from first phase to the second phase of the clotting process; and
(x) on the basis of step (ix), determining the clotting time of the blood sample.

59. The method of claim 52 or 57, further comprising:
(xi) identifying from the series of T2 relaxation rate measurements a third phase, wherein the third phase comprises one or more T2 values characteristic of a serum-like environment, each of the one or more T2 values having a T2 intensity greater than a predetermined threshold, and where each of the one or more T2 values is measured during the clotting process at time points during which the sample comprises a mixture of serum and clot microenvironments;
(xii) calculating the difference between the maximum asymptote and the one or more T2 values of the third phase, or calculating the difference between the minimum asymptote and the one or more T2 values of the third phase;
(xiii) on the basis of step (xii), determining the platelet activity of the blood sample.

60. The method of claim 52, further comprising:
(xiii) identifying from the series of T2 relaxation rate measurements a third phase, wherein the third phase comprises one or more T2 values having one or more T2 intensities greater than a predetermined threshold and characteristic of a serum-like environment, and where each of the one or more T2 values is measured during the clotting process at time points during which the sample comprises a mixture of serum and clot microenvironments; and
(xiv) on the basis of the one or more T2 values or the one or more T2 intensities, determining the platelet activity of the blood sample.

61. The method of claim 59 or 60, further comprising:
(xv) identifying from the series of T2 relaxation rate measurements a fifth phase, wherein the fifth phase comprises one or more T2 values characteristic of a serum-like environment, and where each of the one or more T2 values is measured during the clotting process at time points during which the sample comprises a clot micro environment undergoing lysis;
(xvi) calculating the difference between the one or more T2 values of the third phase and the one or more T2 values of the fifth phase; and
(xvii) on the basis of step (xvi), determining the level of the fibrinolysis of the blood sample.

62. The method of claim 59 or 60, further comprising determining the level of the fibrinolysis of the blood sample by:
(xv) identifying from the series of T2 relaxation rate measurements a third phase, wherein the third phase comprises one or more T2 values characteristic of a serum-like environment, each of the one or more T2 values having a T2 intensity greater than a predetermined threshold, and where each of the one or more T2 values is measured during the clotting process at time points during which the sample comprises a mixture of serum and clot microenvironments;
(xvi) identifying from the series of T2 relaxation rate measurements a fifth phase, wherein the fifth phase comprises one or more T2 values characteristic of a serum-like environment, and where each of the one or more T2 values is measured during the clotting process at time points during which the sample comprises a clot micro environment undergoing lysis;
(xvii) calculating an area above a T2MR curve for the fifth phase and a predetermined upper bound for a predetermined time period during the clotting process, wherein the upper bound is defined by the one or more T2 values of the third phase projected over the curve for the fifth phase; and
(xviii) on the basis of step (xvii), determining the level of the fibrinolysis of the blood sample.

63. A method of monitoring water in a coagulating blood sample comprising the steps of:
(i) providing a blood sample from a test subject and mixing a clotting activation reagent with the blood sample to initiate a coagulation process,
(ii) making a series of T2 relaxation rate measurements of the water in the blood sample, wherein the measurements provide a plurality of decay curves, each decay curve measured using a Carr-Purcell-Meiboom-Gill (CPMG) sequence having a predetermined dwell time and each decay curve characteristic of a time point in the coagulation process,
(iii) applying a mathematical transform to the plurality of decay curves to identify one or more water populations in the blood sample at one or more time points in the coagulation process to produce one or more T2 values and/or T2 intensities,
(iv) identifying from the series of T2 relaxation rate measurements a third phase, wherein the third phase comprises one or more T2 values characteristic of a serum-like environment, each of the one or more T2 values having a T2 intensity greater than a predetermined threshold, and where each of the one or more T2 values is measured during the clotting process at time points during which the sample comprises a mixture of serum and clot microenvironments;
(v) identifying from the series of T2 relaxation rate measurements a fifth phase, wherein the fifth phase comprises one or more T2 values characteristic of a serum-like environment, and where each of the one or more T2 values is measured during the clotting process at time points during which the sample comprises a clot micro environment undergoing lysis;
(vi) calculating an area above a T2MR curve for the fifth phase and a predetermined upper bound for a predetermined time period during the clotting process, wherein the upper bound is defined by the one or more T2 values of the third phase projected over the curve for the fifth phase; and
(vii) on the basis of step (xvi), determining the level of the fibrinolysis of the blood sample.

64. The method of claim 62 or 63, wherein the upper bound is defined by the maximum T2 value of the third phase projected over the curve for the fifth phase.

65. The method of any of claims 1-64, wherein CaCl₂ is added during step (i).

66. The method of any of claims 1-65, wherein the clotting activation reagent comprises an extrinsic activator.

67. The method of 66, wherein the extrinsic activator comprises at least one of tissue factor, thromboplastin, Innovin®, Readiplastin®, and EXTEM®.

68. The method of any of claims 1-65, wherein the clotting activation reagent comprises an intrinsic activator.

69. The method of claim 68, wherein the intrinsic activator comprises at least one of ellagic acid, celite, kaolin, and INTEM.

70. The method of any of claims 1-65, wherein the clotting activation reagent comprises a global activator.

71. The method of claim 70, wherein the activator is thrombin.

72. The method of any of claims 1-71, wherein the blood sample is derived from a pediatric or neonatal subject.

73. The method of claim 72, wherein the pediatric subject exhibits symptoms associated with a hemostatic disorder.

74. The method of claim 73, wherein the hemostatic disorder is at least one of the group of hemophilia, von Willebrand disease, hypercoagulable state, thromotic thrombocytopenic purpura, thrombocytopenia, primary thrombocythemia, induced thrombocytopenia, disseminated intravascular coagulation, procoagulant afibrinogenemia/dysfibrinogenemia, protein C deficiency, protein S deficiency, antithrombin III deficiency, factor V Leiden deficiency, activated protein C resistance (aPCR), Anticoagulant afibrinogenemia/dysfibrinogenemia, Factor V deficiency, Factor VII deficiency, Factor X deficiency, Factor XI deficiency, Factor XII deficiency, Factor XIII deficiency, hypoprothrombinemias, cryoglobulinemias, multiple myeloma, Waldenstrom macroglobulinemia, Henoch-Schonlein purpura, hyperglobulinemic purpura, cavernous hemangioma, hereditary hemorrhagic telangiectasia, pseudoxanthoma elasticum, Vitamin K deficiency, Shwartzman phenomenon, Wiskott-Aldrich syndrome, sepsis, and hemolytic disease of the newborn.

75. The method of any of claims 1-74, wherein the method is completed in 65 minutes or less.

76. The method of claim 75, wherein the method is completed in 45 minutes or less.

77. The method of claim 75, wherein the method comprises calculating a clotting time within 5 minutes of initiating clotting.

78. The method of claim 4, wherein the clotting activation reagent is a combination of RPF and a platelet activator.

79. The method of any of claims 1-74, wherein the T2MR values are reported in real time.

80. The method of any of claims 1-75, wherein at least one of the following is reported in real time: percent hematocrit, clotting time, fibrinogen concentration, platelet activity, and fibrinolysis.

81. The method of claim 75, wherein the method comprises calculating the platelet activity within 20 minutes, within 25 minutes, within 30 minutes, within 35 minutes, or within 40 minutes.

82. A method of monitoring water in a coagulating blood sample comprising the steps of:
(i) providing a blood sample from a test subject and mixing a clotting activation reagent with the blood sample to initiate a coagulation process;
(ii) making a series of T2 relaxation rate measurements of the water in the blood sample, wherein the measurements provide a plurality of decay curves, each decay curve measured using a Carr-Purcell-Meiboom-Gill (CPMG) sequence having a predetermined dwell time and each decay curve characteristic of a time point in the coagulation process;
(iii) applying a mathematical transform to the plurality of decay curves to identify one or more water populations in the blood sample at one or more time points in the coagulation process to produce one or more T2 values and/or T2 intensities;
(iv) identifying from the series of T2 relaxation rate measurements a third phase, wherein the third phase comprises one or more T2 values having one or more T2 intensities greater than a predetermined threshold and characteristic of a serum-like environment, and where each of the one or more T2 values is measured during the clotting process at time points during which the sample comprises a mixture of serum and clot microenvironments; and
(v) on the basis of the one or more T2 values or the one or more T2 intensities, determining the platelet activity of the blood sample.

83. The method of claim 82, wherein the platelet activity is determined by calculating the difference between the one or more T2 values of the third phase and a predetermined lower bound at a predetermined time and the lower bound and the lower bound comprises one of the following:
(vi) one or more T2 values of a first phase, wherein the first phase comprises one or more T2 values measured during the clotting process prior to any observable clot formation;
(vii) one or more T2 values of a second phase, wherein the second phase comprises one or more T2 values measured during the clotting process at time points during which the sample comprises a loosely bound clot;
(viii) one or more T2 values of a fourth phase, wherein the fourth phase comprises one or more T2 values characteristic of a clot microenvironment, wherein each of the one or more T2 values is measured during the clotting process at time points during which the sample comprises a mixture of serum and clot microenvironments;
(ix) a maximum asymptote of the first phase, wherein the maximum asymptote is determined by fitting the one or more T2 values of the first phase and the one or more T2 values of the second phase to a curve, and identifying a maximum asymptote from the one or more T2 values of the first phase, or identifying a minimum asymptote from the one or more T2 values of the second phase; and
(x) a minimum asymptote of the second phase, wherein the maximum asymptote is determined by fitting the one or more T2 values of the first phase and the one or more T2 values of the second phase to a curve, and identifying a maximum asymptote from the one or more T2 values of the first phase, or identifying a minimum asymptote from the one or more T2 values of the second phase.

84. The method of claim 82, wherein the platelet activity is determined by calculating an area under a T2MR curve for the third phase and a predetermined lower bound for a predetermined time period during the clotting process and the lower bound comprises one of the following:
(vi) one or more T2 values of a first phase, wherein the first phase comprises one or more T2 values measured during the clotting process prior to any observable clot formation;
(vii) one or more T2 values of a second phase, wherein the second phase comprises one or more T2 values measured during the clotting process at time points during which the sample comprises a loosely bound clot;
(viii) one or more T2 values of a fourth phase, wherein the fourth phase comprises one or more T2 values characteristic of a clot microenvironment, and where each of the one or more T2 values is measured during the clotting process at time points during which the sample comprises a mixture of serum and clot microenvironments;
(ix) a maximum asymptote of the first phase, wherein the maximum asymptote is determined by fitting the one or more T2 values of the first phase and the one or more T2 values of the second phase to a curve, and identifying a maximum asymptote from the one or more T2 values of the first phase, or identifying a minimum asymptote from the one or more T2 values of the second phase; and
(x) a minimum asymptote of the second phase, wherein the maximum asymptote is determined by fitting the one or more T2 values of the first phase and the one or more T2 values of the second phase to a curve, and identifying a maximum asymptote from the one or more T2 values of the first phase, or identifying a minimum asymptote from the one or more T2 values of the second phase.

85. The method of claim 83, comprising
(a) identifying the difference between the T2 values of the third phase and a predetermined lower bound;
(b) identifying one or more T2 intensities of the third phase or the lower bound; and
(c) on the basis of step (a) and step (b) determining the platelet activity.

86. The method of claim 84, further comprising
(a) identifying an area defined by a T2MR curve of the third phase and a predetermined lower bound;
(b) identifying one or more T2 intensities of the third phase or the lower bound; and
(c) on the basis of step (a) and step (b), determining the platelet activity.

87. The method of claim 86, comprising
(d) identifying the maximum T2 intensity of the third phase;
(e) identifying an area defined by a T2MR curve of the third phase and a predetermined lower bound; and
(f) on the basis of step (d) and step (e), determining the platelet activity.

88. The method of claim 87, comprising
(g) identifying the maximum T2 intensity of the third phase;
(h) identifying the time associated with the maximum T2 intensity of the third phase;
(i) identifying an area defined by a T2MR curve of the third phase and a predetermined lower bound that terminates at the time associated with the maximum T2 intensity of the third phase; and
(j) on the basis of step (i), determining the platelet activity.

89. The method of any one of claims 1-88, wherein the blood sample has a volume of from 5 μL to 50 μL and the clotting activation reagent has a volume of from 5 μL to 25 μL.
